(19) United States
(12) Patent Application Publication
(10) Pub. No.: US 2015/0315188 A1
(43) Pub. Date: Nov. 5, 2015

(54) COMPOUNDS AND METHODS FOR TREATMENT OF HYPERTENSION

(71) Applicant: MEDIVATION TECHNOLOGIES, INC., San Francisco, CA (US)

(72) Inventors: Andrew Asher PROTTER, Palo Alto, CA (US); Sarvajit CHAKRAVARTY, Mountain View, CA (US); Michael John GREEN, Half Moon Bay, CA (US)

(21) Appl. No.: 14/423,027

(22) PCT Filed: Mar. 15, 2013
(86) PCT No.: PCT/US2013/032084

§ 371 Date: Feb. 20, 2015

Related U.S. Application Data
(60) Provisional application No. 61/692,161, filed on Aug. 22, 2012.
(60) Provisional application No. 61/692,178, filed on Aug. 22, 2012.

(51) Int. Cl. C07D 471/22; C07D 471/18; C07D 519/00; C07D 471/04; C07D 471/14

(52) U.S. Cl. CPC C07D 471/22; C07D 471/04; C07D 471/14; C07D 519/00; C07D 471/18

(57) ABSTRACT

Hydrogenated pyrido[4,3-b]indoles and pyrido[3,4-b]indoles are described. The compounds may bind to and are adrenergic receptor α_{2B} antagonists. The compounds may also bind to and antagonize adrenergic receptor α_{1B}. in The compounds may find use in therapy, e.g., to (i) reduce blood pressure and/or (ii) promote renal blood flow and/or (iii) decrease or inhibit sodium reabsorption. The compounds may also be used to treat diseases or conditions that are, or are expected to be, responsive to a decrease in blood pressure. Use of the compounds to treat cardiovascular and renal disorders is particularly described.

### CROSS-REFERENCE TO RELATED APPLICATIONS

This application claims priority to U.S. Provisional Patent Application No. 61/692,178 filed Aug. 22, 2012, and U.S. Provisional Patent Application No. 61/692,161 filed Aug. 22, 2012, the disclosures of each of which are incorporated herein by reference in their entireties.

### BACKGROUND OF THE INVENTION

Hypertension is a serious condition that can damage vital organs, such as the heart and kidneys, and other parts of the body, such as the central nervous system. Individuals who have hypertension may have, or be at risk of developing, dangerous diseases such as coronary heart disease and kidney failure. Hypertension, which is the leading modifiable risk factor for cardiovascular disease mortality, causes more than 7 million deaths every year worldwide.

Hypertension is the most common chronic medical condition in developed countries as well as the most common indication for physician visits and prescription medication use. Hypertension affects more than 50 million individuals in the United States and over one billion individuals worldwide, and overall prevalence may continue to increase with the advancing age of the population.

Unfortunately, despite the importance of blood pressure control and the availability of multiple classes of antihypertensive agents, the treatment of hypertension remains suboptimal. Data from the most recent National Health and Nutrition Examination Survey demonstrate that only 34% of patients with hypertension have blood pressures at their therapeutic goal. Additionally, it was shown that the majority of patients with hypertension will require two or more antihypertensive agents to achieve their goal blood pressure. Even with optimal compliance with multiple antihypertensive agents of different classes, a significant fraction of patients will not be able to achieve their goal blood pressure. The overall prevalence of resistant hypertension, defined as elevated blood pressure in spite of the use of three or more antihypertensive agents, is unknown, but small studies suggest that it ranges from 5%-16% in primary care settings to greater than 50% in nephrology clinics. Given data suggesting that increasing age and obesity are important risk factors for the development of resistant hypertension, it is expected that the overall prevalence of this condition is likely to increase due to demographic changes in the population.

Systolic blood pressure tends to increase with age and systolic hypertension is an important health issue, prominent in the elderly (Duprez, Am. J. Med. 121:179-184 (2008)). It has been suggested that this occurs as large vessels such as the aorta lose their elasticity with age and is less able to buffer the pulsative nature of cardiac output. There exists a need for a treatment for patients in such clinical setting, for example, patients with systolic hypertension accompanied with low diastolic pressure (Franklin et al. J. Hypertension 29:1101-1108 (2011).

Metabolic syndrome is a cluster of disorders including obesity, hypertension, hypertrigleridemia, hypercholesterolemia and elevated blood sugar. Individuals with this spectrum of disorders are at increased risk of diabetes, heart disease and stroke. Agents capable of treating more than one of these disorders are desirable.

Hypertensive emergencies are defined as severe elevations in blood pressure associated with resultant organ damage (i.e. pulmonary edema, renal impairment, visual impairment, intracranial hemorrhage, or encephalopathy). The treatment of hypertensive emergencies involves aggressive and controlled blood pressure lowering in a highly monitored intensive care setting using intravenous blood pressure lowering agents. Therapeutic agents and methods of treatment are needed to gradually lower blood pressure and minimize damage of end organs such as the brain, kidney, heart, and eye.

The frequency of chronic kidney disease also continues to increase worldwide as does the prevalence of end-stage renal disease. Although chronic kidney disease is often caused by hypertension, other factors such as a decrease in renal blood flow and increase in sodium retention or reabsorption can lead to renal diseases. Increased age and diabetes can also contribute to renal disease. Especially the elderly, which are a growing segment of the world population, are at increased risk for renal disease. The presence of chronic kidney disease is also associated with a large increase in cardiovascular morbidity and mortality. Consequently, the identification and reduction of chronic kidney disease has become a vital public health priority.

Thus, there remains a need for new and useful agents that are capable of (i) reducing an individual's blood pressure and/or (ii) promoting renal blood flow and/or (iii) inhibiting or decreasing sodium reabsorption.

### BRIEF SUMMARY OF THE INVENTION

Hydrogenated pyrido[4,3-b]indoles and pyrido[3,4-b]indoles are described. Compositions and kits comprising the compounds are also provided, as are methods of using and making the compounds. Compounds provided herein may find use in treating a disease or condition that is, or is believed to be, responsive to any one or more of: (i) a decrease in blood pressure; (ii) an increase in renal blood flow and (iii) a decrease or inhibition of sodium reabsorption. In one aspect, compounds provided herein are selective adrenergic receptor α_{2B} antagonists that may find use in treating a disease or condition that is, or is believed to be, responsive to any one or more of: (i) a decrease in blood pressure; (ii) an increase in renal blood flow and (iii) a decrease or inhibition of sodium reabsorption Compounds provided may also find use in treating diseases and/or conditions such as hypertension, congestive heart failure or a renal disease or condition.

In another aspect, compounds that promote mitochondrial health and cellular viability are also described. The compounds provided herein are selective adrenergic receptor α_{2B} antagonists that may find use in treating a disease or condition that is associated with dysfunction of mitochondria in a renal or cardiac cell. Compounds provided may also find use in treating diseases and/or conditions selected from the group consisting of acute renal failure, chronic renal failure, coronary ischemia, acute congestive heart failure, chronic congestive heart failure, coronary artery disease, sleep apnea, respiratory distress, hypertension, and peripheral vascular disease.

In one aspect, compounds of the invention are compounds described in Table 1, such as Compound Nos. 1-178, or a salt, solvate or N-oxide thereof. It is understood that compounds as detailed herein include all stereoisomeric forms. For example, reference to Compound Nos. 1-178 includes and intends all “a,” “b,” etc. forms of Compound Nos. 1-178 per se. Provided herein is a compound selected from the group consisting of Compound Nos. 1-178, or a salt, solvate or N-oxide thereof. In a particular variation, provided herein is a compound selected from the group consisting of Compound Nos. 1-178 or a pharmaceutically acceptable salt thereof. In some embodiments, the compound or salt (e.g., pharmaceutically acceptable salt), solvate or N-oxide thereof, is a compound selected from the group consisting of Compound Nos. 1-64. In some embodiments, the compound, or a salt (e.g., a pharmaceutically acceptable salt), solvate or N-oxide thereof, is a compound selected from a group consisting of Compound Nos. 1-133. In some embodiments, the compound, or a salt (e.g., a pharmaceutically acceptable salt), solvate or N-oxide thereof, is a compound selected from a group consisting of Compound Nos. 1-177.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention. For example, in some embodiments, provided is a compound selected from a group consisting of any one or more of Compound Nos. 1-178, such as a group consisting of any one or any two or any three or more of Compound Nos. 1-178, or a salt (e.g., a pharmaceutically acceptable salt), solvate or N-oxide thereof. A selection of any combination of Compound Nos. 1-178, or salt, solvate or N-oxide thereof, is intended the same as if each and every combination were specifically and individually listed.

In one aspect, provided is a method of lowering blood pressure in an individual in need thereof comprising administering to the individual an effective amount of a compound of the invention, such as a compound described in Table 1 (e.g., a compound selected from the group consisting of Compound Nos. 1-178), or a salt, solvate or N-oxide thereof. In a particular variation, provided herein is a method of lowering blood pressure in an individual in need thereof, comprising administering to the individual a compound selected from the group consisting of Compound Nos. 1-178 or a pharmaceutically acceptable salt thereof.

In some embodiments, the individual has high blood pressure. In other embodiments, the method reduces one or more of any of the following: systolic blood pressure, diastolic blood pressure, mean arterial blood pressure, and pulse pressure of the individual, following administration of the compound. In other embodiments, the method does not substantially increase heart rate of the individual. In yet other embodiments, the individual has one or more risk factors for developing high blood pressure.

Provided is also a method of (i) increasing renal blood flow, and/or (ii) decreasing sodium reabsorption, in an individual in need thereof comprising administering to the individual an effective amount of a compound of the invention, such as a compound described in Table 1 (e.g., a compound selected from the group consisting of Compound Nos. 1-178) or a salt, solvate or N-oxide thereof. In a particular variation, provided herein is a method of (i) increasing renal blood flow, and/or (ii) decreasing sodium reabsorption in an individual in need thereof, comprising administering to the individual a compound selected from the group consisting of Compound Nos. 1-178 or a pharmaceutically acceptable salt thereof. In some embodiments, the method results in one or more of any of the following: increase in renal blood flow, decrease in sodium reabsorption, increase in urine sodium content and/or increase in urine volume, reduction in edema, reduction in elevated blood urea nitrogen to creatinine (BUN/Cr) ratio, and decrease in creatinine levels.

In some embodiments, the individual has or is at risk of developing acute or chronic congestive heart failure, acute decompensated congestive heart failure, acute or chronic renal failure, or acute or chronic renal failure due to renal insufficiency.

Provided is also a method of treating a disease or condition that is responsive to any one or more of: (i) a decrease in blood pressure; (ii) an increase in renal blood flow; and (iii) a decrease of sodium reabsorption, comprising administering to an individual in need thereof an effective amount of a compound of the invention, such as a compound described in Table 1 (e.g., a compound selected from the group consisting of Compound Nos. 1-178), or a salt, solvate or N-oxide thereof. In a particular variation, provided herein is a method of treating a disease or condition that is responsive to any one or more of: (i) a decrease in blood pressure; (ii) an increase in renal blood flow; and (iii) a decrease of sodium reabsorption, in an individual in need thereof, comprising administering to the individual a compound selected from the group consisting of Compound Nos. 1-178 or a pharmaceutically acceptable salt thereof. In some embodiments, the disease or condition is hypertension. In certain embodiments, the disease or condition is treatment-resistant hypertension, or hypertensive emergency. In yet other embodiments, the disease or condition is a cardiac or renal disease or condition.

In some embodiments, the compound is an adrenergic receptor α_{2B} antagonist. In other embodiments, the compound is also an adrenergic receptor α_{1D} antagonist. In yet other embodiments, the compound is also an adrenergic receptor α_{1D} antagonist.

Further provided is a kit comprising (i) a compound detailed herein, such as a compound described in Table 1 (e.g., a compound selected from the group consisting of Compound Nos. 1-178), or a salt (e.g., a pharmaceutically acceptable salt), solvate or N-oxide thereof, and (ii) instructions for use according to a method described above.

Also provided is use of a compound detailed herein, such as a compound described in Table 1 (e.g., a compound selected from the group consisting of Compound Nos. 1-178), or a salt (e.g., a pharmaceutically acceptable salt), solvate or N-oxide thereof, in lowering blood pressure, increasing renal blood flow, and/or decreasing or inhibiting sodium reabsorption. Further provided are uses of a compound detailed herein, such as a compound described in Table 1 (e.g., a compound selected from the group consisting of Compound Nos. 1-176), or a salt (e.g., a pharmaceutically acceptable salt), solvate or N-oxide thereof, for the manufacturing of a medicament for the treatment of a disease or condition that is responsive to any one or more of: (i) a decrease in blood pressure; (ii) an increase in renal blood flow; and (iii) a decrease of sodium reabsorption.

### DETAILED DESCRIPTION OF THE INVENTION

### Definitions

Unless clearly indicated otherwise, the terms “a”, “an”, and the like, refer to one or more.

It is also understood and clearly conveyed by this disclosure that reference to “the compound” or “a compound” includes and refers to any compounds (e.g., selective adrenergic receptor α_{2B} antagonists) or pharmaceutically acceptable salt or other form thereof as described herein.

Reference to “about” a value or parameter herein includes (and describes) embodiments that are directed to that value or parameter per se. For example, description referring to “about X” includes description of “X”.

Unless clearly indicated otherwise, “an individual” as used herein intends a mammal, including but not limited to a human. The invention may find use in both human medicine and in the veterinary context.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

As used herein, “treatment” or “treating” is an approach for obtaining a beneficial or desired result, including clinical results.

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

As used herein, “unit dosage form” refers to physically discrete units, suitable as unit dosages, each unit containing a predetermined quantity of active ingredient, or compound which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects or interacting in a deleterious manner with any of the other components of the composition in which it is contained. Pharmaceutically acceptable carriers or excipients have preferably thus in some embodiments met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth metal ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al., Pharmaceutical Salts, J. Pharm. Sci. 1977 January; 66(1):1-19. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “excipient” as used herein includes an inert or inactive substance that may be used in the production of a drug or pharmaceutical, such as a tablet containing a compound detailed herein, or a pharmaceutically acceptable salt thereof, as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

An inverse agonist is a compound that binds to a receptor and inhibits the activity of the receptor in the absence of an agonist. An inverse agonist requires that the receptor have some constitutive basal activity in the absence of an agonist. While an agonist increases activity of the receptor over basal level an inverse agonist reduces receptor activity below basal level.

### Receptor Binding Profile

In some embodiments, compounds that bind to and are antagonists of the adrenergic receptor α_{2B}, but which are not antagonists of the adrenergic receptor α_{2A}, and pharmaceutically acceptable salts thereof, are provided. The compounds may find use in therapy for decreasing blood pressure in an individual and in treating diseases or conditions which are responsive to (i) a decrease in blood pressure and/or (ii) an increase in renal blood flow and/or (iii) a decrease or inhibition of sodium reabsorption or sodium retention. Thus, an individual who has a disease or condition that is responsive to (i) a decrease in blood pressure and/or (ii) an increase in renal blood flow and/or (iii) a decrease or inhibition of sodium reabsorption or sodium retention will experience, or is expected to experience, one or more beneficial or desirable results upon administration of a compound provided herein, or pharmaceutically acceptable salt thereof. In one aspect, the beneficial or desirable result is a reduction in the individual's mean arterial blood pressure for a period of time following administration of the compound or pharmaceutically acceptable salt thereof. In another aspect, the beneficial or desirable result is a reduction in the individual's systolic blood pressure for a period of time following administration of the compound or pharmaceutically acceptable salt thereof. In a further aspect, the beneficial or desirable result is an increase in renal blood flow (e.g., by altering the vascular tone of renal efferent and afferent arterioles) for a period of time following administration of the compound or pharmaceutically acceptable salt thereof. In another aspect, the beneficial or desirable result is a decrease or inhibition in sodium reabsorption (e.g., thereby exerting a natriuretic and diuretic effect) for a period of time following administration of the compound or pharmaceutically acceptable salt thereof. In another aspect, the beneficial or desirable result is an increase in urine sodium and/or urine volume for a period of time following administration of the compound or pharmaceutically acceptable salt thereof. In one variation, the compounds may find use in therapy in treating diseases or conditions which are responsive to (i) a decrease in blood pressure and (ii) an increase in renal blood flow. In one variation, the compounds my find use in therapy in treating diseases or conditions which are responsive to (i) a decrease in blood pressure and (ii) a decrease or inhibition of sodium reabsorption. In one variation, the compounds may find use in treating diseases or conditions which are responsive to (i) an increase in renal blood flow and (ii) a decrease or inhibition of sodium reabsorption. In one variation, the compounds may find use in therapy in treating diseases or conditions which are responsive to (i) a decrease in blood pressure and (ii) an increase in renal blood flow and (iii) a decrease or inhibition of sodium reabsorption.

Compounds that bind to and are antagonists of the adrenergic receptor α_{2B} should reduce an individual's blood pressure. However, compounds that antagonize the adrenergic receptor α_{2A} in some instances may actually increase an individual's blood pressure. Thus, compounds that antagonize the adrenergic receptor α_{2B} but do not antagonize the adrenergic receptor α_{2A} (compounds referred to herein as “selective adrenergic receptor α_{2B} antagonists”) are desirable agents in therapy. Selective adrenergic receptor α_{2B} antagonists find further use in therapy of cardiovascular and renal indications. The selective adrenergic receptor α_{2B} antagonists provided herein (i) bind to and are antagonists of the adrenergic receptor α_{2B}, and (ii) are not antagonists of the adrenergic receptor α_{2A}.

The selective adrenergic receptor α_{2B} antagonists may in some variations also bind to and be agonists of the adrenergic receptor α_{2A}. The selective adrenergic receptor α_{2B} antagonists may also be used in conjunction with other agents that are agonists of the adrenergic receptor α_{2A}.

The selective adrenergic receptor α_{2B} antagonists may in some variations also bind to and be antagonists of the adrenergic receptor α_{1A}. The selective adrenergic receptor α_{2B} antagonists may also be used in conjunction with other agents that are antagonists of the adrenergic receptor α_{2A}.

The selective adrenergic receptor α_{2B} antagonists may in some variations also bind to and be antagonists of the adrenergic receptor α_{2A}. The selective adrenergic receptor α_{2B} antagonists may also be used in conjunction with other agents that are antagonists of the adrenergic receptor α_{1B}.

The selective adrenergic receptor α_{2B} antagonists may in some variations both (i) bind to and be agonists of the adrenergic receptor α_{2A} and (ii) bind to and be antagonists of the adrenergic receptor α_{1B} and/or α_{1D}.

In one variation, a selective adrenergic receptor α_{2B} antagonist exhibits (i) equal to or greater than about 60% inhibition of α_{2B} ligand binding at 0.03 μM and antagonist activity to adrenergic receptor α_{2B} and (ii) equal to or less than about 30% inhibition of α_{2A} ligand binding at 0.1 μM and absence of antagonist activity to adrenergic receptor α_{2A}. In one variation, a selective adrenergic receptor α_{2B} antagonist exhibits (i) equal to or greater than about any one of 60%, 65%, 70%, 75%, 80%, 85%, 90%, or 95%, or between about 60% and about 90%, between about 70% and about 90%, or between about 80% and about 100% inhibition of α_{2B} ligand binding at 0.03 μM and antagonist activity to adrenergic receptor α_{2B}, and (ii) equal to or less than about any one of 30%, 25%, 20%, 15%, 10%, or 5%, or between about 0% and about 30%, between about 10% and about 30%, or between about 20% and about 30% inhibition of α_{2A} ligand binding at 0.1 μM and absence of antagonist activity to adrenergic receptor α_{2A}. In one variation, a selective adrenergic receptor α_{2B} antagonist exhibits (i) equal to or greater than about 60% inhibition of α_{2B} ligand binding at 0.1 μM and antagonist activity to adrenergic receptor α_{2B} and (ii) equal to or less than about 30% inhibition of α_{2A} ligand binding at 0.1 μM and absence of antagonist activity to adrenergic receptor α_{2A}. In one variation, a selective adrenergic receptor α_{2B} antagonist exhibits (i) equal to or greater than about any one of 60%, 65%, 70%, 75%, 80%, 85%, 90%, or 95%, or between about 60% and about 90%, between about 70% and about 90%, or between about 80% and about 100% inhibition of α_{2B} ligand binding at 0.1 μM and antagonist activity to adrenergic receptor α_{2B}, and (ii) equal to or less than about any one of 30%, 25%, 20%, 15%, 10%, or 5%, or between about 0% and about 30%, between about 10% and about 30%, or between about 20% and about 30% inhibition of α_{2A} ligand binding at 0.1 μM and absence of antagonist activity to adrenergic receptor α_{2A}. It is understood and clearly conveyed herein that a selective adrenergic receptor α_{2B} antagonist can exhibit any of the adrenergic receptor α_{2B} binding profiles described herein in combination with any of the adrenergic receptor α_{2A} binding profiles described herein, as if each and every combination were listed separately. For example, a selective adrenergic receptor α_{2B} antagonist may exhibit (i) equal to or greater than about 65% inhibition of α_{2B} ligand binding at 0.03 μM and antagonist activity to adrenergic receptor α_{2B}, and (ii) equal to or less than about 25% inhibition of α_{2A} ligand binding at 0.1 μM and absence of antagonist activity to adrenergic receptor α_{2A}.

In one variation, a selective adrenergic receptor α_{2B} antagonist exhibits (i) equal to or greater than about 60% inhibition of α_{2B} ligand binding at 0.03 μM and antagonist activity to adrenergic receptor α_{2B} and (ii) equal to or less than about 30% inhibition of α_{2A} ligand binding at 0.03 μM and absence of antagonist activity to adrenergic receptor α_{2A}. In one variation, a selective adrenergic receptor α_{2B} antagonist exhibits (i) equal to or greater than about any one of 60%, 65%, 70%, 75%, 80%, 85%, 90%, or 95%, or between about 60% and about 90%, between about 70% and about 90%, or between about 80% and about 100% inhibition of α_{2B} ligand binding at 0.03 μM and antagonist activity to adrenergic receptor α_{2B}, and (ii) equal to or less than about any one of 30%, 25%, 20%, 15%, 10%, or 5%, or between about 0% and about 30%, between about 10% and about 30%, or between about 20% and about 30% inhibition of α_{2A} ligand binding at 0.03 μM and absence of antagonist activity to adrenergic receptor α_{2A}. In one variation, a selective adrenergic receptor α_{2B} antagonist exhibits (i) equal to or greater than about 60% inhibition of α_{2B} ligand binding at 0.1 μM and antagonist activity to adrenergic receptor α_{2B} and (ii) equal to or less than about 30% inhibition of α_{2A} ligand binding at 0.03 μM and absence of antagonist activity to adrenergic receptor α_{2A}. In one variation, a selective adrenergic receptor α_{2B} antagonist exhibits (i) equal to or greater than about any one of 60%, 65%, 70%, 75%, 80%, 85%, 90%, or 95%, or between about 60% and about 90%, between about 70% and about 90%, or between about 80% and about 100% inhibition of α_{2B} ligand binding at 0.1 μM and antagonist activity to adrenergic receptor α_{2B}, and (ii) equal to or less than about any one of 30%, 25%, 20%, 15%, 10%, or 5%, or between about 0% and about 30%, between about 10% and about 30%, or between about 20% and about 30% inhibition of α_{2A} ligand binding at 0.03 μM and absence of antagonist activity to adrenergic receptor α_{2A}. It is understood and clearly conveyed herein that a selective adrenergic receptor α_{2B} antagonist can exhibit any of the adrenergic receptor α_{2B} binding profiles described herein in combination with any of the adrenergic receptor α_{2A} binding profiles described herein, as if each and every combination were listed separately. For example, a selective adrenergic receptor α_{2B} antagonist may exhibit (i) equal to or greater than about 65% inhibition of α_{2B} ligand binding at 0.03 μM and antagonist activity to adrenergic receptor α_{2B}, and (ii) equal to or less than about 25% inhibition of α_{2A} ligand binding at 0.03 μM and absence of antagonist activity to adrenergic receptor α_{2A}.

In another variation, a selective adrenergic receptor α_{2B} antagonist has a Ki ratio of α_{2A} to α_{2B} that is greater than about any one of 5 or 15 or 50. Ki is the binding affinity from the Cheng-Prusoff equation: Ki=IC₅₀/(1+[S]/Kd), wherein [S] is the concentration of the radioligand and Kd is dissociation constant (affinity) of the radioligand for the protein (Cheng, Y., Prusoff, W. H., Biochem. Pharmacol. 22:3099-3108, 1973). It is understood that the Ki ratio of α_{2A} to α_{2B} may be combined with any binding and/or other activity profile details described herein for selective adrenergic receptor α_{2B} antagonists the same as if each were specifically and individually listed. For example, in one variation, a selective adrenergic receptor α_{2B} antagonist may exhibit (i) equal to or greater than about 65% inhibition of α_{2B} ligand binding at 0.03 μM and antagonist activity to adrenergic receptor α_{2B}, and (ii) equal to or less than about 25% inhibition of α_{2A} ligand binding at 0.1 μM and absence of antagonist activity to adrenergic receptor α_{2A}; and a Ki ratio of α_{2A} to α_{2B} that is greater than about any one of 5 or 15 or 50.

The selective adrenergic receptor α_{2B} antagonists may in some variations also bind to and be antagonists of the adrenergic receptor α_{1B}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about 60% inhibition of α_{1B} ligand binding at 0.03 μM and antagonist activity to adrenergic receptor α_{1B}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about any one of 60%, 65%, 70%, 75%, 80%, 85%, 90%, or 95%, or between about 60% and 90%, between about 70% and 90%, or between about 80% and about 100% inhibition of α_{1B} ligand binding at 0.03 μM and antagonist activity to adrenergic receptor α_{1B}. In one variation, the selective adrenergic receptor α_{2B} antagonists may exhibit (i) equal to or greater than about 60% inhibition of α_{2B} ligand binding at 0.1 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about 60% inhibition of α_{1B} ligand binding at 0.1 μM and antagonist activity to adrenergic receptor α_{1B}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about 60% inhibition of α_{1B} ligand binding at 0.1 μM and antagonist activity to adrenergic receptor α_{1B}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about any one of 60%, 65%, 70%, 75%, 80%, 85%, 90%, or 95%, or between about 60% and 90%, between about 70% and 90%, or between about 80% and about 100% inhibition of α_{1B} ligand binding at 0.1 μM and antagonist activity to adrenergic receptor α_{1B}. It is understood and clearly conveyed herein that a selective adrenergic receptor α_{2B} antagonist can exhibit any of the adrenergic receptor α_{2B} binding profiles described herein in combination with any of the adrenergic receptor α_{2A} binding profiles described herein and any of the adrenergic receptor α_{1B} binding profiles, as if each and every combination were listed separately. For example, a selective adrenergic receptor α_{2B} antagonist may exhibit (i) equal to or greater than about 65% inhibition of α_{2B} ligand binding at 0.03 μM and antagonist activity to adrenergic receptor α_{2B}, (ii) equal to or less than about 25% inhibition of α_{2A} ligand binding at 0.1 μM and absence of antagonist activity to adrenergic receptor α_{2A}, and (iii) equal to or greater than about 65% inhibition of α_{1B} ligand binding at 0.03 μM and antagonist activity to adrenergic receptor α_{1B}. The selective adrenergic receptor α_{2B} antagonists may also be used in conjunction with other agents that antagonize the adrenergic receptor α_{1B}. Administration in conjunction with another compound includes administration in the same or different composition, either sequentially, simultaneously, or continuously.

The selective adrenergic receptor α_{2B} antagonists may in some variations also bind to and be antagonists of the adrenergic receptor α_{1D}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about 60% inhibition of α_{1D} ligand binding at 0.03 μM and antagonist activity to adrenergic receptor α_{1D}. In another variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, (iii) equal to or greater than about 60% inhibition of α_{1B} ligand binding at 0.03 μM and antagonist activity to adrenergic receptor α_{1B} and (iv) equal to or greater than about 60% inhibition of α_{1D} ligand binding at 0.03 μM and antagonist activity to adrenergic receptor α_{1D}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about any one of 60%, 65%, 70%, 75%, 80%, 85%, 90%, or 95%, or between about 60% and 90%, between about 70% and 90%, or between about 80% and about 100% inhibition of α_{2A} and/or α_{1B} ligand binding at 0.03 μM and antagonist activity to adrenergic receptor α_{1D} and/or α_{1B}. In one variation, the selective adrenergic receptor α_{2B} antagonists may exhibit (i) equal to or greater than about 60% inhibition of α_{2B} ligand binding at 0.1 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about 60% inhibition of α_{1B} and/or α_{1D} ligand binding at 0.1 μM and antagonist activity to adrenergic receptor α_{1B} and/or α_{1D}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about 60% inhibition of α_{1B} and/or α_{1D} ligand binding at 0.1 μM and antagonist activity to adrenergic receptor α_{1B} and/or α_{1D}. In one variation, the selective adrenergic receptor α_{2B} antagonists may exhibit (i) equal to or greater than about 60% inhibition of α_{2B} ligand binding at 0.03 μM and antagonist activity to adrenergic receptor α_{2B}, (ii) equal to or less than about 30% inhibition of α_{2A} ligand binding at 0.1 μM and absence of antagonist activity to adrenergic receptor α_{2A}, and (iii) equal to or greater than about any one of 60%, 65%, 70%, 75%, 80%, 85%, 90%, or 95%, or between about 60% and 90%, between about 70% and 90%, or between about 80% and about 100% inhibition of α_{1B} and/or α_{1D} ligand binding at 0.1 μM and antagonist activity to adrenergic receptor α_{1B} and/or α_{1D}. It is understood and clearly conveyed herein that a selective adrenergic receptor α_{2B} antagonist can exhibit any of the adrenergic receptor α_{2B} binding profiles described herein in combination with any of the adrenergic receptor α_{2A} binding profiles described herein and any of the adrenergic receptor α_{1B} and/or α_{1D} binding profiles, as if each and every combination were listed separately. For example, a selective adrenergic receptor α_{2B} antagonist may exhibit (i) equal to or greater than about 65% inhibition of α_{2B} ligand binding at 0.03 μM and antagonist activity to adrenergic receptor α_{2B}, (ii) equal to or less than about 25% inhibition of α_{2A} ligand binding at 0.1 μM and absence of antagonist activity to adrenergic receptor α_{2A}, and (iii) equal to or greater than about 65% inhibition of α_{2B} ligand binding at 0.03 μM and antagonist activity to adrenergic receptor α_{1D}. The selective adrenergic receptor α_{2B} antagonists may also be used in conjunction with other agents that antagonize the adrenergic receptor α_{1D}. Administration in conjunction with another compound includes administration in the same or different composition, either sequentially, simultaneously, or continuously.

In some instances, compounds provided herein bind to and are antagonists of adrenergic receptor α_{2B} and may also be antagonists for the adrenergic receptor α_{2A}. In such instances, it is preferable that the compound is more potent at inhibiting the adrenergic receptor α_{2B} compared to the adrenergic receptor α_{2A} In one variation, the compound inhibit both the adrenergic receptor α_{2B} and the adrenergic receptor α_{2A}, and wherein the compound has limited or no brain bioavailability and so cannot easily activate adrenergic α_{2A} receptors in the brain. In one variation, the compound inhibits both the adrenergic receptor α_{2B} and the adrenergic receptor α_{2A}, and wherein the compound has brain bioavailability. In some other instances, compounds provided herein bind to and are antagonists of adrenergic receptor α_{2B} and may be inverse agonists for the adrenergic receptor α_{2A}. In some embodiments, the compound (1) binds to and is an antagonist of adrenergic receptor α_{2B}, and (2) binds to and is an antagonist and/or inverse agonist of the adrenergic receptor α_{2A}. In some embodiments, the compound (1) binds to and is an antagonist of adrenergic receptor α_{2B}, (2) binds to and is an antagonist and/or inverse agonist of the adrenergic receptor α_{2A}, and (3) binds to and is antagonist of the adrenergic receptor α_{1B} and/or the adrenergic receptor α_{1D}. It is understood and clearly conveyed herein that an adrenergic receptor α_{2B} antagonist can exhibit any of the adrenergic receptor α_{2B} binding profiles (in terms of % inhibition at a given concentration and/or in terms of Kᵢ) described herein in combination with any of the adrenergic receptor α_{1B} and/or α_{1D} binding profiles, as if each and every combination were listed separately.

The binding properties to adrenergic receptors of compounds disclosed herein may be assessed by methods known in the art, such as competitive binding assays. In one variation, compounds are assessed by the binding assays detailed herein. In one variation, inhibition of binding of a ligand to a receptor is measured by the assays described herein. In another variation, inhibition of binding of a ligand is measured in an assay known in the art.

### Functional Assay Profile

Antagonist activity to the adrenergic receptor α_{2B} receptor may be assessed by methods known in the art, such as standard α_{2B} receptor cell membrane-based or intact cell-based activity assays. For example, the GTPγS binding or Aequorin-based assays may be used. In one variation, the selective adrenergic receptor α_{2B} antagonists exhibit an IC₅₀ value equal to or less than about any one of 100 nM, 30 nM or 10 nM at a given concentration of agonist (e.g., concentration corresponding to EC₈₀ of oxymetazoline (for Aequorin assay) or guanfacine (for GTPγS assay)) in an α_{2B} antagonist assay. In one variation, a selective adrenergic receptor α_{2B} antagonist exhibits an IC₅₀ value in an α_{2B} antagonist assay equal to or less than about 10 nM at a given concentration of agonist (e.g., concentration corresponding to EC₈₀ of oxymetazoline (for Aequorin assay) or guanfacine (for GTPγS assay)) in an α_{2B} antagonist assay. In one variation, a selective adrenergic receptor α_{2B} antagonist exhibits an IC₅₀ value in an α_{2B} antagonist assay equal to or less than about any one of 100 nM, 30 nM or 10 nM at a concentration of oxymetazoline corresponding to its EC₈₀ concentration as obtained by assay protocols described herein. In one variation, a selective adrenergic receptor α_{2B} antagonist exhibits an IC₅₀ value in an α_{2B} antagonist assay equal to or less than about any one of 100 nM, 30 nM or 10 nM at a concentration of oxymetazoline between about 50 nM and about 5000 nM. In one variation, a selective adrenergic receptor α_{2B} antagonist exhibits an IC₅₀ value in an α_{2B} antagonist assay equal to or less than about any one of 100 nM, 30 nM or 10 nM at a concentration of about 480 nM oxymetazoline. In one variation, a selective adrenergic receptor α_{2B} antagonist exhibits an IC₅₀ value in an α_{2B} antagonist assay equal to or less than about any one of 100 nM, 30 nM or 10 nM at a concentration of guanfacine between about 50 nM and about 5000 nM. In one variation, a selective adrenergic receptor α_{2B} antagonist exhibits an IC₅₀ value in an α_{2B} antagonist assay equal to or less than about any one of 100 nM, 30 nM or 10 nM at a concentration of about 500 nM guanfacine, which in a particular variation is 504 nM guanfacine.

The absence of antagonist activity to the adrenergic receptor α_{2A} may be assessed by methods known in the art, such as standard α_{2A} receptor intact cell-based activity assays. For example, the Aequorin-based assay may be used. It is understood and clearly conveyed that absence of antagonist activity to the adrenergic receptor α_{2A} intends activity that is sufficiently reduced, but not necessarily eliminated or undetectable, at the adrenergic receptor α_{2A}. In one variation, a compound will exhibit an undetectable amount of antagonist activity to the adrenergic receptor α_{2A} In another variation, a compound will lack antagonist activity to the adrenergic receptor α_{2A} if it exhibits an IC₅₀ value in an α_{2A} antagonist assay that is greater than about any one of 50 nM, 100 nM or 200 nM at a given concentration of agonist (e.g., concentration corresponding to EC₈₀ of UK14304). In one variation, the adrenergic receptor α_{2A} exhibits an IC₅₀ value in an α_{2A} antagonist assay that is greater than about 200 nM at a given concentration of agonist (e.g., concentration corresponding to EC₈₀ of UK14304). In one variation, a selective adrenergic receptor α_{2B} antagonist exhibits an IC₅₀ value in an α_{2A} antagonist assay greater than about any one of 50 nM, 100 nM or 200 nM at a concentration of UK14304 corresponding to its EC₈₀ concentration as obtained by assay protocols described herein. In one variation, a selective adrenergic receptor α_{2B} antagonist exhibits an IC₅₀ value in an α_{2A} antagonist assay greater than about any one of 50 nM, 100 nM or 200 nM at a concentration of UK14304 between about 0.4 nM and about 40 nM. In one variation, a selective adrenergic receptor α_{2B} antagonists exhibits an IC₅₀ value in an α_{2A} antagonist assay greater than about any one of 50 nM, 100 nM or 200 nM at a concentration of about 5 nM UK14304, which in a particular variation is 4.57 nM UK14304. Alternatively, a compound that does not bind the α_{2A} receptor will be neither an agonist nor antagonist of the α_{2A} receptor.

In some variations, regardless of IC₅₀ values obtained from α_{2B} and α_{2A} assays, a compound may nonetheless be a selective adrenergic receptor α_{2B} antagonist if it exhibits a Ki ratio of α_{2A} to α_{2B} that is higher than about any one of 5, 10, or 15. For example, where a compound exhibits an IC₅₀ value between about 50-100 nM in an α_{2B} antagonist assay at a given concentration of agonist (e.g., concentration corresponding to EC₈₀ of oxymetazoline) and an IC₅₀ value between about 50 and 100 nM in an α_{2A} antagonist assay at a given concentration of agonist (e.g., concentration corresponding to EC₈₀ of UK14304), the compound is considered, in one variation, a selective adrenergic receptor α_{2B} antagonist if it exhibits a Ki ratio of α_{2A} to α_{2B} higher than about any one of 5, 10, or 15.

Antagonist activity to adrenergic receptor α_{1B} may be assessed by methods known in the art, such as standard α_{1B} receptor intact cell-based activity assays, including the Aequorin-based assay. In one variation, a selective adrenergic receptor α_{2B} antagonist will also antagonize the adrenergic receptor α_{1B} and exhibit an IC₅₀ value equal to or less than about any one of 100 nM or 30 nM or 10 nM at a given concentration of agonist (e.g., concentration corresponding to EC₈₀ of cirazoline) in an adrenergic receptor α_{1B} antagonist assay. In one variation, a selective adrenergic receptor α_{2B} antagonist will also antagonize the adrenergic receptor α_{1B} and exhibit an IC₅₀ value equal or less than about 10 nM at a given concentration of agonist (e.g., concentration corresponding to EC₈₀ of cirazoline) in an adrenergic receptor α_{1B} antagonist assay. In one variation, the selective adrenergic receptor α_{2B} antagonists exhibit an IC₅₀ value in an α_{1B} antagonist assay equal to or less than about any one of 100 nM, 30 nM or 10 nM at a concentration of cirazoline corresponding to its EC₈₀ concentration as obtained by assay protocols described herein. In one variation, the selective adrenergic receptor α_{2B} antagonists exhibit an IC₅₀ value in an α_{1B} antagonist assay equal to or less than about any one of 100 nM, 30 nM or 10 nM at a concentration of cirazoline between about 2.3 nM and about 230 nM. In one variation, the selective adrenergic receptor α_{2B} antagonists exhibit an IC₅₀ value in an α_{1B} antagonist assay equal to or less than about any one of 100 nM, 30 nM or 10 nM at a concentration of about 25 nM cirazoline, which in a particular variation is 23.56 nM cirazoline.

Antagonist activity to adrenergic receptor α_{1D} may be assessed by methods known in the art, such as standard α_{1D} receptor intact cell-based activity assays, including the Aequorin-based assay. In one variation, a selective adrenergic receptor α_{2B} antagonist will also antagonize the adrenergic receptor α_{1D} and exhibit an IC₅₀ value equal to or less than about any one of 100 nM or 30 nM or 10 nM at a given concentration of agonist (e.g., concentration corresponding to EC₈₀ of cirazoline) in an adrenergic receptor α_{1D} antagonist assay. In one variation, a selective adrenergic receptor α_{2B} antagonist will also antagonize the adrenergic receptor α_{1D} and exhibit an IC₅₀ value equal or less than about 10 nM at a given concentration of agonist (e.g., concentration corresponding to EC₈₀ of cirazoline) in an adrenergic receptor α_{1D} antagonist assay. In one variation, the selective adrenergic receptor α_{2B} antagonists exhibit an IC₅₀ value in an α_{1D} antagonist assay equal to or less than about any one of 100 nM, 30 nM or 10 nM at a concentration of cirazoline corresponding to its EC₈₀ concentration as obtained by assay protocols described herein. In one variation, the selective adrenergic receptor α_{2B} antagonists exhibit an IC₅₀ value in an α_{1D} antagonist assay equal to or less than about any one of 100 nM, 30 nM or 10 nM at a concentration of cirazoline between about 2.3 nM and about 230 nM. In one variation, the selective adrenergic receptor α_{2B} antagonists exhibit an IC₅₀ value in an α_{1D} antagonist assay equal to or less than about any one of 100 nM, 30 nM or 10 nM at a concentration of about 25 nM cirazoline, which in a particular variation is 23.56 nM cirazoline.

In one variation, the selective adrenergic receptor α_{2B} antagonists exhibit (i) equal to or greater than about 60% inhibition of α_{2B} ligand binding at 0.03 μM and an IC₅₀ value in an α_{2B} antagonist assay equal to or less than about any one of 100 nM, 30 nM or 10 nM at a given concentration of agonist (e.g., concentration corresponding to EC₈₀ of oxymetazoline (for Aequorin assay) or guanfacine (for GTPγS assay)), and (ii) equal to or less than about 30% inhibition of α_{2A} ligand binding at 0.1 μM and an IC₅₀ value in an α_{2A} antagonist assay that is greater than about any one of 50 nM, 100 nM or 200 nM at a given concentration of agonist (e.g., concentration corresponding to EC₈₀ of UK14304). In some variations, the selective adrenergic receptor α_{2B} antagonists exhibit (i) equal to or greater than about 60% inhibition of α_{2B} ligand binding at 0.03 μM and an IC₅₀ value in an α_{2B} antagonist assay equal to or less than about any one of 100 nM, 30 nM or 10 nM at a given concentration of agonist (e.g., concentration corresponding to EC₈₀ of oxymetazoline (for Aequorin assay) or guanfacine (for GTPγS assay)), and (ii) equal to or less than about 30% inhibition of α_{2A} ligand binding at 0.1 μM and an IC₅₀ value in an α_{2A} antagonist assay that is greater than about any one of 50 nM, 100 nM or 200 nM at a given concentration of agonist (e.g., concentration corresponding to EC₈₀ of UK14304), and (iii) equal to or greater than about 60% inhibition of α_{1B} ligand binding at 0.03 μM and an IC₅₀ value in an α_{1B} antagonist assay equal or less than about any one of 100 nM or 30 nM or 10 nM at a given concentration of agonist (e.g., concentration corresponding to EC₈₀ of cirazoline). In some variations, the selective adrenergic receptor α_{2B} antagonists exhibit (i) equal to or greater than about 60% inhibition of α_{2B} ligand binding at 0.03 μM and an IC₅₀ value in an α_{2B} antagonist assay equal to or less than about any one of 100 nM, 30 nM or 10 nM at a given concentration of agonist (e.g., concentration corresponding to EC₈₀ of oxymetazoline (for Aequorin assay) or guanfacine (for GTPγS assay)), and (ii) equal to or less than about 30% inhibition of α_{2A} ligand binding at 0.1 μM and an IC₅₀ value in an α_{2A} antagonist assay that is greater than about any one of 50 nM, 100 nM or 200 nM at a given concentration of agonist (e.g., concentration corresponding to EC₈₀ of UK14304), and (iii) equal to or greater than about 60% inhibition of α_{1D} ligand binding at 0.03 μM and an IC₅₀ value in an α_{1D} antagonist assay equal or less than about any one of 100 nM or 30 nM or 10 nM at a given concentration of agonist (e.g., concentration corresponding to EC₈₀ of cirazoline). In some variations, the selective adrenergic receptor α_{2B} antagonists exhibit (i) equal to or greater than about 60% inhibition of α_{2B} ligand binding at 0.03 μM and an IC₅₀ value in an α_{2B} antagonist assay equal to or less than about any one of 100 nM, 30 nM or 10 nM at a given concentration of agonist (e.g., concentration corresponding to EC₈₀ of oxymetazoline (for Aequorin assay) or guanfacine (for GTPγS assay)), and (ii) equal to or less than about 30% inhibition of α_{2A} ligand binding at 0.1 μM and an IC₅₀ value in an α_{2A} antagonist assay that is greater than about any one of 50 nM, 100 nM or 200 nM at a given concentration of agonist (e.g., concentration corresponding to EC₈₀ of UK14304), (iii) equal to or greater than about 60% inhibition of α_{1B} ligand binding at 0.03 μM and an IC₅₀ value in an α_{1B} antagonist assay equal or less than about any one of 100 nM or 30 nM or 10 nM at a given concentration of agonist (e.g., concentration corresponding to EC₈₀ of cirazoline); and (iv) equal to or greater than about 60% inhibition of α_{1D} ligand binding at 0.03 μM and an IC₅₀ value in an α_{1D} antagonist assay equal or less than about any one of 100 nM or 30 nM or 10 nM at a given concentration of agonist (e.g., concentration corresponding to EC₈₀ of cirazoline).

In another variation, the selective adrenergic receptor α_{2B} antagonists exhibit (i) equal to or greater than about 60% inhibition of α_{2B} ligand binding at 0.03 μM and an IC₅₀ value in an α_{2B} antagonist assay equal to or less than any about one of 100 nM, 30 nM or 10 nM at a given concentration of agonist (e.g., concentration corresponding to EC₈₀ of oxymetazoline (for Aequorin assay) or guanfacine (for GTPγS assay)), and (ii) binding to and agonist activity to adrenergic receptor α_{2A}.

In another variation, the adrenergic receptor α_{2B} antagonists exhibit (i) equal to or greater than about 60% inhibition of α_{2B} ligand binding at 0.03 μM and an IC₅₀ value in an α_{2B} antagonist assay equal to or less than any about one of 100 nM, 30 nM or 10 nM at a given concentration of agonist (e.g., concentration corresponding to EC₈₀ of oxymetazoline (for Aequorin assay) or guanfacine (for GTPγS assay)), and (ii) greater than or equal to about 30% inhibition of α_{2A} ligand binding at 0.1 μM and IC₅₀ value in an adrenergic receptor α_{2A} antagonist assay equal to or less than about any one of 100 nM, 30 nM or 10 nM at a concentration of UK14304 (for Aequorin assay) corresponding to its EC₈₀ concentration obtained by assay protocols described herein.

It is understood and clearly conveyed herein that compounds provided herein, including selective adrenergic receptor α_{2B} antagonists provided herein can exhibit any of the binding profiles and any of the antagonist or agonist activity profiles detailed herein, the same as if each and every combination were individually listed. For example, in one variation, the selective adrenergic receptor α_{2B} antagonists exhibit (i) greater than about 65% inhibition of α_{2B} ligand binding at 0.03 μM and an IC₅₀ value in an α_{2B} antagonist assay equal to or less than about 10 nM at a concentration of oxymetazoline corresponding to its EC₈₀ concentration as obtained by assay protocols described herein, and (ii) less than about 25% inhibition of α_{2A} ligand binding at 0.1 μM and an IC₅₀ value in an α_{2A} antagonist assay that is greater than 200 nM at a concentration of UK14304 corresponding to its EC₈₀ concentration as obtained by assay protocols described herein, and (iii) equal to or greater than about 60% inhibition of α_{2A} ligand binding at 0.03 μM and an IC₅₀ value in an α_{2A} antagonist assay equal or less than 10 nM at a concentration of cirazoline corresponding to its EC₈₀ concentration as obtained by assay protocols described herein. In one aspect, such a compound will also exhibit a Ki ratio of α_{2A} to α_{2B} that is greater than about any one of 5 or 15 or 50.

### Medical Use

Without being bound by theory, it is believed that the compounds provided herein are capable of (i) reducing blood pressure and/or (ii) promoting renal blood flow and/or (iii) decreasing or inhibiting sodium reabsorption. In some embodiments, the compounds are adrenergic receptor α_{2B} antagonists (e.g., selective adrenergic receptor α_{2B} antagonists). In some embodiments, it is believed that the selective adrenergic receptor α_{2B} antagonists provided herein are capable of (i) reducing blood pressure and/or (ii) promoting renal blood flow and/or (iii) decreasing or inhibiting sodium reabsorption without concomitantly antagonizing the α_{2A} receptor, which would reduce or potentially eliminate the beneficial blood pressure lowering and renal effects modulated by antagonizing α_{2B} Furthermore, the selective adrenergic receptor α_{2B} antagonists provided herein may be capable of decreasing blood pressure sensitivity to salt, decreasing sodium retention, decreasing vasoconstriction in small arteries and veins, increasing insulin secretion, increasing basal metabolic rate, decreasing platelet aggregation and/or enhancing mitochondrial function. However, in certain cases where the compound has strong antagonist activities against adrenergic receptor α_{2B} and/or adrenergic receptor α_{1B}, some antagonist activity against adrenergic receptor α_{2A} may be tolerated and even beneficial.

Compounds provided herein may be capable of mediating control of the renal function. Adrenergic α_{2B} receptors are located within the kidney. Regard et al. (Cell 2008; 135:561) have demonstrated that the gene for the adrenergic α_{2B} receptor is most abundantly expressed in the kidney. Meister et al. (J. Pharmacol. Exp. Therapeutics 1994; 268:1605) have shown by in situ hybridization that expression predominates in the medulla outer stripe with extensions into the cortical S3 segment of the proximal tubules. Adrenergic α_{2B} receptor antagonists provided herein may be capable of disrupting sodium reabsorption resulting in natriuresis and diuresis. Methods to determine effects of adrenergic α_{2B} antagonists on renal function in a rabbit model of hypertension have been described by Burke et al. (J Hypertens. 29:945-952).

In addition to reducing blood pressure, compounds disclosed herein, including adrenergic α_{2B} antagonists, are capable of a reduction in blood volume that might result from diuresis and/or the movement of fluid from the vascular space to the extravascular space. Reduction of blood volume results in increase in hematocrit levels which can be measured by methods known in the art, for example, by estimation of erythrocyte volume fraction. Characterization of the effect of α_{2B} antagonists on renal function are determined by measuring urine volume, urine sodium and urine potassium using methods described by Burke et al. (Effects of chronic sympatho-inhibition on renal excretory function in renovascular hypertension Sandra L. Burke, Roger G. Evans and Geoffrey A. Head. Journal of Hypertens. 29:945-952 (2011).

The compounds detailed herein are expected to find use in therapy, particularly in cardiac and renal diseases and conditions, in addition to hypertension and other conditions in which a (i) reduction in blood pressure and/or (ii) increase in renal blood flow and/or (iii) decrease in sodium reabsorption would be beneficial. In the methods provided herein, an effective amount of a compound detailed herein is administered to an individual. Methods of using compounds as described herein to (i) reduce blood pressure and/or (ii) promote renal blood flow and/or (iii) decrease or inhibit sodium reabsorption in an individual in need thereof are provided. The compounds may also find use in treating a disease or condition that is, or is expected to be, responsive to (i) a reduction in an individual's blood pressure and/or (ii) an increase in renal blood flow and/or (iii) a decrease or inhibition of sodium reabsorption. The individual may be a human who has been diagnosed with or is suspected of having high blood pressure or a disease or condition that is, or is expected to be, responsive to (i) a reduction in an individual's blood pressure and/or (ii) an increase in renal blood flow and/or (iii) a decrease or inhibition of sodium reabsorption. The individual may be a human who exhibits one or more symptoms associated with high blood pressure or a disease or condition that is, or is expected to be, responsive to (i) a reduction in an individual's blood pressure and/or (ii) an increase in renal blood flow and/or (iii) a decrease or inhibition of sodium reabsorption. The individual may be a human who is genetically or otherwise predisposed to developing high blood pressure or a disease or condition that is, or is expected to be, responsive to (i) a reduction in an individual's blood pressure and/or (ii) an increase in renal blood flow and/or (iii) a decrease or inhibition of sodium reabsorption. In one variation, the compounds may find use in treating metabolic syndrome. In some embodiments, the compounds are adrenergic receptor α_{2B} antagonists. In one variation, the adrenergic receptor α_{2B} antagonists are selective adrenergic receptor α_{2B} antagonists. In one variation, a compound that is an adrenergic receptor α_{2B} antagonist also showing adrenergic receptor α_{2A} antagonist and/or inverse agonist activity may find use reducing blood pressure in an individual with hypertension who is also suffering from obesity, type-2 diabetes and/or metabolic syndrome. Thus, provided is a method for lowering blood pressure in hypertensive patients with a disease or condition that is responsive to treatment using an antagonist or inverse agonist of adrenergic receptor α_{2A}, such as obesity and/or type-2 diabetes and/or metabolic syndrome.

Compounds detailed herein may be used in a method of treating a disease or condition that is responsive to (i) a reduction in an individual's blood pressure and/or (ii) an increase in renal blood flow and/or (iii) a decrease or inhibition of sodium reabsorption. For example, the compounds may find use in treating hypertension, including treatment-resistant hypertension. In some embodiments, the compounds may be used in a method of treating hypertension in an individual not suffering from obesity or type-2 diabetes. In some embodiments, the compounds are adrenergic receptor α_{2B} antagonists. In some embodiments, the compounds are selective adrenergic receptor α_{2B} antagonists.

In one aspect, the disease or indication is a cardiac or renal disease or indication for which (i) a reduction in an individual's blood pressure and/or (ii) an increase in renal blood flow and/or (iii) a decrease or inhibition of sodium reabsorption would be, or would be expected to be, beneficial. Such cardiac indications include, but are not limited to, heart failure, such as compensated heart failure, decompensated heart failure, acute decompensated congestive heart failure and chronic congestive heart failure, coronary heart disease, cardiac arrhythmias, myocardial ischemia, and hypertrophy. Such renal indications include, but are not limited to, renal failure such as chronic renal failure, acute renal failure and endstage renal failure, renal ischemia and chronic kidney disease. Other indications for which (i) a reduction in an individual's blood pressure and/or (ii) an increase in renal blood flow and/or (iii) a decrease or inhibition of sodium reabsorption would be, or would be expected to be, beneficial include but are not limited to sleep apnea and ischemic attacks.

Compounds detailed herein may also ameliorate symptoms of a disease or condition that have a cardiac or renal component in which (i) a reduction in an individual's blood pressure and/or (ii) an increase in renal blood flow and/or (iii) a decrease or inhibition of sodium reabsorption would be, or would be expected to be, beneficial. For example, the compounds may reduce elevated blood pressure, improve shortness of breath, reduce tachycardia, reduce edema, reduce elevated blood urea nitrogen to creatinine (BUN/Cr) ratio, improve creatinine levels, improve the ability to lie flat, reduce the incidence or severity of high blood pressure, reduce the risk and/or number of acute cardiac events (e.g., acute decompensation or myocardial infarction) an individual experiences over a period of time (e.g., one year, 2 years, 5 years, etc.), reduce the incidence of acute heart failure an individual experiences over a period of time (e.g., one year, 2 years, 5 years, etc.), reduce the severity and/or incidence of pulmonary congestion and/or reduce the risk of stroke, reduce shortness of breath and/or tachycardia in individuals after myocardial infarction, improve left ventricular ejection fraction (LVEF) post infarct and/or lower weight and blood pressure in obese individuals (e.g., men and women) with pre-hypertension. In some embodiments, the compounds are adrenergic receptor α_{2B} antagonists. In some embodiments, the compounds are selective adrenergic receptor α_{2B} antagonists.

Compounds detailed herein (such as the adrenergic receptor α_{2B} antagonists detailed herein) may find use in the treatment of hypertensive emergencies. Provided is a method of treating hypertensive emergencies, comprising administering intravenously an effective amount of an adrenergic receptor α_{2B} antagonist to an individual in need thereof. In some embodiments, the method comprises administering intravenously an effective amount of an adrenergic receptor α_{2B} antagonist to an individual in need thereof in a highly monitored intensive care setting, wherein the administration results in aggressive and controlled blood pressure lowering in the individual. In some embodiments, intravenous administration of an adrenergic receptor α_{2B} antagonist in an individual results in gradually lowering of blood pressure in the individual and minimizing damage of end organs such as the brain, kidney, heart, and eye. Particularly useful in the treatment of hypertensive emergencies or crisis are parenteral formulations of an adrenergic receptor α_{2B} antagonist detailed herein. In one variation, the compound is an adrenergic receptor α_{2B} antagonist. In some variations, the compound is a selective adrenergic receptor α_{2B} antagonist. In one variation, the adrenergic receptor α_{2B} antagonist also exhibits adrenergic receptor α_{2A} antagonist and/or inverse agonist activity.

In one variation, a method of decreasing the severity and/or incidence of shortness of breath, tachycardia, edema, and/or the inability to lie flat is provided, comprising administering an effective amount of a compound detailed herein to an individual who has or is suspected of having heart failure (e.g., compensated heart failure and decompensated heart failure). In another variation, a method of decreasing the severity and/or incidence of elevated BUN/Cr, and/or edema is provided comprising administering an effective amount of a compound detailed herein to an individual who has or is suspected of having renal failure (e.g., acute or chronic renal failure). In another variation, a method of reducing blood pressure in an individual is provided comprising administering an effective amount of a compound detailed herein to an individual who has or is suspected of having hypertension (e.g., treatment-resistant hypertension). In another variation, a method of decreasing the severity and/or incidence of shortness of breath, tachycardia, and/or improving LVEF post infarct in an individual is provided comprising administering an effective amount of a compound detailed herein to an individual who has experienced myocardial infarction (e.g., an individual who has recently experienced myocardial infarction such as within 30 minutes, 1 hour, 3 hours, 6 hours, 12 hours, or 24 hours of treatment). In some of the variations, the adrenergic receptor α_{2B} antagonist is a selective adrenergic receptor α_{2B} antagonist. In some of the variations, the adrenergic receptor α_{2B} antagonist also exhibits antagonist activity for the adrenergic receptor α_{2A}. In some embodiments, the compounds are adrenergic receptor α_{2B} antagonists. In some embodiments, the compounds are selective adrenergic receptor α_{2B} antagonists.

In one variation, provided is method for lowering the blood pressure in an individual in need thereof comprising administering to the individual a compound described herein, or a pharmaceutically acceptable salt thereof. Administration of an adrenergic receptor α_{2B} antagonist detailed herein lowers the blood pressure in the individual from a level considered above the desired level for such individual. The blood pressure lowering therapy such as administration of compounds detailed herein is intended to help hypertensive individuals reach their blood pressure goals defined by their individual cardiovascular risk factors. For example, for otherwise healthy individuals without diabetes or known cardiovascular disease, goal blood pressure is less than about 140/90 mmHg; for patients with known cardiovascular disease (e.g., prior myocardial infarction, peripheral vascular disease) goal blood pressure is less than about 130-135/85 mmHg; for patients with diabetes, goal blood pressure is less than about 130/80 mmHg.

In one variation, compounds provided herein may have any one or more of the following beneficial effects on an individual: (1) reduce arterial blood pressure (e.g., in an individual with hypertension, certain forms of heart failure and/or renal failure); (2) reduce pulse pressure (e.g., in an individual with hypertension, certain forms of heart failure and/or renal failure); (3) tachycardia-preserved baroreceptor activity (e.g., in an individual whose systolic blood pressure is expected to or does fall in response to an α_{2B} antagonist), which may suggest a lack of orthostatic hypotension; and (4) bradycardia-reduced cardiac work load and added reduction on blood pressure reduction by further reducing cardiac output (e.g., in an individual who has been administered a therapy that is an α_{2B} and α_{1B} mixed antagonist).

In another variation, compounds provided herein may exert their therapeutic effect with no or reduced side-effects, such as when compared to other therapies used in the treatment of the same or similar indication. In one aspect, compounds provided herein exhibit no or reduced side effects upon administration to an individual, wherein the side effects may be any one or more of: (i) reduced libido, (ii) orthostatic hypotension, (iii) muscle weakness, (iv) fatigue, (v) erectile dysfunction, (vi) constipation, (vii) depression, (viii) dizziness, (ix) dry mouth, (x) impaired thinking, (xi) weight gain, (xii) persistent cough, (xiii) chest pain, (xiv) headache, (xv) fluid retention, (xvi) racing pulse, and (xvii) emesis.

In one aspect, compounds are provided that do not bind appreciably any one or more of the histamine, dopamine and serotonin receptors. In any of the methods detailed herein, in one variation the individual does not have a cognitive disorder, psychotic disorder, neurotransmitter-mediated disorder and/or neuronal disorder. As used herein, the term “cognitive disorders” refers to and intends diseases and conditions that are believed to involve or be associated with or do involve or are associated with progressive loss of structure and/or function of neurons, including death of neurons, and where a central feature of the disorder may be the impairment of cognition (e.g., memory, attention, perception and/or thinking). These disorders include pathogen-induced cognitive dysfunction, e.g., HIV associated cognitive dysfunction and Lyme disease associated cognitive dysfunction. Examples of cognitive disorders include Alzheimer's Disease, Huntington's Disease, Parkinson's Disease, schizophrenia, amyotrophic lateral sclerosis (ALS), autism, mild cognitive impairment (MCI), stroke, traumatic brain injury (TBI) and age-associated memory impairment (AAMI). As used herein, the term “psychotic disorders” refers to and intends mental diseases or conditions that are believed to cause or do cause abnormal thinking and perceptions. Psychotic disorders are characterized by a loss of reality which may be accompanied by delusions, hallucinations (perceptions in a conscious and awake state in the absence of external stimuli which have qualities of real perception, in that they are vivid, substantial, and located in external objective space), personality changes and/or disorganized thinking. Other common symptoms include unusual or bizarre behavior, as well as difficulty with social interaction and impairment in carrying out the activities of daily living. Exemplary psychotic disorders are schizophrenia, bipolar disorders, psychosis, anxiety and depression. As used herein, the term “neurotransmitter-mediated disorders” refers to and intends diseases or conditions that are believed to involve or be associated with or do involve or are associated with abnormal levels of neurotransmitters such as histamine, serotonin, dopamine, norepinephrine or impaired function of aminergic G protein-coupled receptors. Exemplary neurotransmitter-mediated disorders include spinal cord injury, diabetic neuropathy, allergic diseases and diseases involving geroprotective activity such as age-associated hair loss (alopecia), age-associated weight loss and age-associated vision disturbances (cataracts). Abnormal neurotransmitter levels are associated with a wide variety of diseases and conditions including, but not limited, to Alzheimer's disease, Parkinson's Disease, autism, Guillain-Barré syndrome, mild cognitive impairment, schizophrenia, anxiety, multiple sclerosis, stroke, traumatic brain injury, spinal cord injury, diabetic neuropathy, fibromyalgia, bipolar disorders, psychosis, depression and a variety of allergic diseases. As used herein, the term “neuronal disorders” refers to and intends diseases or conditions that are believed to involve, or be associated with, or do involve or are associated with neuronal cell death and/or impaired neuronal function or decreased neuronal function. Exemplary neuronal indications include neurodegenerative diseases and disorders such as Alzheimer's disease, Huntington's disease, amyotrophic lateral sclerosis (ALS), Parkinson's disease, canine cognitive dysfunction syndrome (CCDS), Lewy body disease, Menkes disease, Wilson disease, Creutzfeldt-Jakob disease, Fahr disease, an acute or chronic disorder involving cerebral circulation, such as ischemic or hemorrhagic stroke or other cerebral hemorrhagic insult, age-associated memory impairment (AAMI), mild cognitive impairment (MCI), injury-related mild cognitive impairment (MCI), post-concussion syndrome, post-traumatic stress disorder, adjuvant chemotherapy, traumatic brain injury (TBI), neuronal death mediated ocular disorder, macular degeneration, age-related macular degeneration, autism, including autism spectrum disorder, Asperger syndrome, and Rett syndrome, an avulsion injury, a spinal cord injury, myasthenia gravis, Guillain-Barré syndrome, multiple sclerosis, diabetic neuropathy, fibromyalgia, neuropathy associated with spinal cord injury, schizophrenia, bipolar disorder, psychosis, anxiety or depression.

Individuals who have high blood pressure, or a disease or condition that is, or is expected to be, responsive to (i) a reduction in an individual's blood pressure and/or (ii) an increase in renal blood flow and/or (iii) a decrease or inhibition of sodium reabsorption may benefit from the compounds detailed herein, including the adrenergic receptor α_{2B} antagonists (e.g., the selective adrenergic receptor α_{2B} antagonist) detailed herein.

An individual who does not have high blood pressure or a disease or condition that is, or is expected to be, responsive to (i) a reduction in an individual's blood pressure and/or (ii) an increase in renal blood flow and/or (iii) a decrease or inhibition of sodium reabsorption may nevertheless benefit from the compounds detailed herein if the individual has one or more risk factors for high blood pressure, or a disease or condition that is, or is expected to be, responsive to (i) a reduction in an individual's blood pressure and/or (ii) an increase in renal blood flow and/or (iii) a decrease or inhibition of sodium reabsorption. Risk factors for developing high blood pressure may include gender, race, ethnicity, age, family history, weight and/or lifestyle. For example, African-Americans, men (particularly if over age 45), woman over age 55, anyone over age 60, pre-hypertension individuals (individuals with a blood pressure of 120-130/80-89 mmHg), individuals who are overweight or obese, individuals with sleep apnea (such as obstructive sleep apnea), individuals who smoke, individuals who have a high salt diet, individuals who have a low potassium diet, individuals with chronic heavy alcohol use, individuals with a sedentary lifestyle, individuals with moderate to high stress, individuals with compromised renal function or renal failure and individuals with close relatives who have high blood pressure are each at an increased risk of developing high blood pressure themselves, or diseases or conditions associated with high blood pressure. Individuals with more than one such risk factor are particularly susceptible to developing high blood pressure. Risk factors for developing kidney disease may include diabetes, high blood pressure (hypertension), cardiovascular diseases, smoking, obesity, high cholesterol, a family history of kidney disease, and/or age 65 or older. Members of certain ethnic groups are also at higher risk for kidney disease including people of Aboriginal, Asian, south Asian, Pacific Island, African/Caribbean, American Indian and Hispanic origin.

### Cell Viability and Mitochondrial Health

Methods of promoting cellular viability by promoting mitochondrial health are provided, the methods comprising contacting the cell with a compound detailed herein. The methods are applicable to various cells, such as neuronal and non-neuronal cells. In one variation, the cell is a non-neuronal cell, such as a renal or cardiac cell (e.g., myocardial muscle cell). In one aspect, methods of promoting cellular viability are provided wherein the cell is one whose viability would be, or would be expected to be, promoted by nutrient influx and/or oxygenation. Methods of promoting cellular viability in a cell experiencing, or exhibiting symptoms of, mitochondrial stress are also provided.

Methods of treating a disease or condition that is, or is expected to be, responsive to promoting mitochondrial health and cell viability are also described, the methods comprising administering to an individual in need thereof an effective amount of a compound provided herein. In one variation, the disease or condition is one which is associated with dysfunction of mitochondria in a non-neuronal cell. In a particular variation, the disease or condition is one which is associated with dysfunction of mitochondria in a renal or cardiac cell (e.g., myocardial muscle cell). In another variation, the disease or condition is one which would benefit from cellular (e.g., renal or cardiac) nutrient influx and/or oxygenation.

Thus, individuals who have a disease or condition that is associated with, or believed to be associated with, mitochondrial dysfunction may benefit from the compounds detailed herein, or pharmaceutically acceptable salts thereof. An individual who has a disease or condition that is associated with mitochondrial dysfunction should experience one or more beneficial or desirable results upon administration of an effective amount of a compound provided herein, or pharmaceutically acceptable salt thereof. In one aspect, the beneficial or desirable result is an increase in nutrient influx and/or oxygenation of a cell. In another aspect, the beneficial or desirable result is a reduction in the number and/or severity of symptoms associated with a disease or condition that is associated with mitochondrial dysfunction.

In one variation, a method of treating a renal or cardiac condition is provided, comprising administering to an individual in need thereof a compound as detailed herein. Such conditions include, but are not limited to, renal failure, such as acute renal failure and chronic renal failure, coronary (e.g., myocardial) ischemia, heart failure, such as acute and chronic congestive heart failure (including the muscle fatigue associated with these conditions), and coronary artery disease. Methods of treating other diseases and conditions are also described, such as methods of treating sleep apnea, acute respiratory distress syndrome (adult and infant) and peripheral vascular disease. The compounds as provided herein may also be used in a method of delaying the onset and/or development of a disease or condition associated with mitochondrial dysfunction, comprising administering a compound as provided herein, or a pharmaceutical salt thereof, to an individual who is at risk of developing a disease or condition associated with mitochondrial dysfunction.

Compounds that do not bind appreciably to neurotransmitter receptors but nevertheless enhance mitochondrial function, e.g., when administered to cells in the setting of mitochondrial stress (e.g., excess intracellular calcium), may be used in the methods herein to promote cell survival. In one aspect, the compounds exhibit the ability to enhance mitochondrial function by protecting against cell death mediated by mitochondrial dysfunction in an assay detailed herein. Thus, it is understood and clearly conveyed that enhancing mitochondrial function includes protecting a cell against cell death mediated by mitochondrial dysfunction. The compounds may also be assessed in assays known in the art.

It is understood and clearly conveyed that the binding and activity profiles detailed herein (e.g., in the disclosure above) in one variation apply to the compounds provided herein (e.g., a compound for use in the methods). In one aspect, selective adrenergic receptor α_{2B} antagonists are of the compounds described in Table 1 (e.g., a compound selected from the group consisting of Compound Nos. 1-178), or a salt (e.g., a pharmaceutically acceptable salt), solvate or N-oxide thereof.

### Compounds of the Invention

Compounds according to the invention are detailed herein, including in the Brief Summary of the Invention and elsewhere. The invention includes the use of all of the compounds described herein, including any and all stereoisomers, including geometric isomers (cis/trans or E/Z isomers), tautomers, salts, N-oxides, and solvates of the compounds described herein, as well as methods of making such compounds.

In some embodiments, the invention relates to Compounds described in Table 1 (e.g., a compound selected from the group consisting of Compound Nos. 1-178), or a salt (e.g., a pharmaceutically acceptable salt), solvate or N-oxide thereof, and uses thereof.

Representative examples of compounds detailed herein, including intermediates and final compounds according to the invention are depicted in the tables below. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the invention embraces all salts and solvates of the compounds depicted here, as well as the non-salt and non-solvate form of the compound, as is well understood by the skilled artisan. In some embodiments, the salts of the compounds of the invention are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. For example, when a 2-hydroxypyridyl moiety is depicted, the corresponding 2-pyridone tautomer is also intended. The tautomeric forms specifically depicted may or may not be the predominant forms in solution or when used according to the methods described herein.

The invention also includes any or all of the stereochemical forms, including any enantiomeric or diasteriomeric forms of the compounds described. The structure or name is intended to embrace all possible stereoisomers of a compound depicted, and each unique stereoisomer has a compound number bearing a suffix “a”, “b”, etc. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. Taking compound 1 as an example, a composition of substantially pure compound 1 intends a composition that contains no more than 35% impurity, wherein the impurity denotes a compound other than compound 1 or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the invention embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Kits comprising a compound of the invention, or a salt or solvate thereof, and suitable packaging are provided. In one embodiment, a kit further comprises instructions for use. In one aspect, a kit comprises a compound of the invention, or a salt or solvate thereof, and instructions for use of the compounds in the treatment of a disease or condition for which a reduction in blood pressure and/or promoting renal blood flow and/or inhibiting or decreasing sodium reabsorption is expected to be or is beneficial.

Articles of manufacture comprising a compound of the invention, or a salt or solvate thereof, in a suitable container are provided. The container may be a vial, jar, ampoule, preloaded syringe, i.v. bag, and the like.

In one aspect, a compounds detailed herein as provided herein exhibits the ability to cross the blood-brain barrier. In another aspect, a compounds detailed herein as provided herein is not able to cross the blood-brain barrier. In one aspect, a compounds detailed herein as provided herein exerts its therapeutic effect in the brain only. In one aspect, a compounds detailed herein as provided herein exerts its therapeutic effect in the periphery only. In one aspect, a compounds detailed herein as provided herein exerts its therapeutic effect both in the brain and peripherally. In some embodiments, the adrenergic receptor α_{2B} antagonist is a selective adrenergic receptor α_{2B} antagonist. In some embodiments, the adrenergic receptor α_{2B} antagonist also exhibits adrenergic receptor α_{2A} antagonist and/or inverse agonist activity.

Blood brain barrier permeability can be measured in rodents or dog by administering the compound orally or intravenously, recovering a blood and brain tissue sample at different time points and comparing how much compound is in each sample. Blood fraction is typically processed to plasma for determination of compound content. Brain exposure can be described from the ratio of brain to plasma levels of drug. In one variation, a compound that poorly crosses the blood brain barrier has a brain to plasma ratio of compound of about 0.1 or less. In another variation, the compound has a brain to plasma ratio of about 0.2 or less, about 0.3 or less, about 0.4 or less, about 0.5 or less, about 0.8 or less, or about 1.0 or less.

Preferably, the compounds detailed herein are orally bioavailable. However, the compounds may also be formulated for parenteral (e.g., intravenous) administration. In some settings, parenteral administration of an adrenergic receptor α_{2B} antagonists (e.g., selective adrenergic receptor α_{2B} antagonist) may be desired. For example, intra-renal delivery may offer treatment options for acute and chronic renal failure, end stage renal failure and acute decompensated congestive heart failure. Parenteral formulation may be preferred in the treatment of hypertensive urgency and emergency. In some embodiments, the adrenergic receptor α_{2B} antagonist is a selective adrenergic receptor α_{2B} antagonist. In some embodiments, the adrenergic receptor α_{2B} antagonist also exhibits adrenergic receptor α_{2A} antagonist and/or inverse agonist activity.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., reducing the blood pressure of an individual, promoting renal blood flow and/or decreasing or inhibiting sodium reabsorption.

Methods as provided herein may comprise administering to an individual a pharmacological composition that contains an effective amount of a compound and a pharmaceutically acceptable carrier. The effective amount of the compound may in one aspect be a dose of between about 0.01 and about 100 mg, between about 0.1 and about 100 mg, between about 1 and about 100 mg, between about 10 and about 100 mg, between about 0.01 and about 10 mg, between about 0.01 and about 1 mg, between about 0.01 and about 0.1 mg, between about 0.1 and about 10 mg, between about 0.1 and about 1 mg, or between about 1 and about 10 mg.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants.

Any of the compounds described herein can be formulated in a tablet in any dosage form described, for example, a compound as described herein or a pharmaceutically acceptable salt thereof can be formulated as a 10 mg, a 5 mg, a 1 mg, or a 20 mg tablet.

The compound may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

Compositions comprising a compound provided herein are also described. In one variation, the composition comprises a compound and a pharmaceutically acceptable carrier or excipient. In another variation, a composition of substantially pure compound is provided.

The invention further provides kits for carrying out the methods of the invention, which comprises one or more compounds described herein or a pharmacological composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for any one or more of the following uses: treating, preventing, and/or delaying the onset and/or development of hypertension and/or a disease or condition which is responsive, or expected to be responsive, to (i) a reduction in an individual's blood pressure and/or (ii) an increase in renal blood flow and/or (iii) a decrease or inhibition of sodium reabsorption.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein (e.g., hypertension) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention also provides compositions (including pharmacological compositions) as described herein for the use in treating, preventing, and/or delaying the onset and/or development of hypertension and/or a disease or condition which is responsive, or expected to be responsive, to (i) a reduction in an individual's blood pressure and/or (ii) an increase in renal blood flow and/or (iii) a decrease or inhibition of sodium reabsorption and other methods described herein. In certain embodiments, the composition comprises a pharmaceutical formulation which is present in a unit dosage form. As used herein, the term “unit dosage form” refers to a formulation that contains a predetermined dose of a compound as disclosed herein and optionally a second pharmaceutically active compound useful for treatment of a disease or condition detailed herein (e.g., hypertension).

For compounds bearing one or more chiral centers, each unique stereoisomer has a compound number bearing a suffix “a”, “b”, etc. As examples, racemic compound 2, bearing one chiral center, can be resolved into its individual enantiomers 2a and 2b.

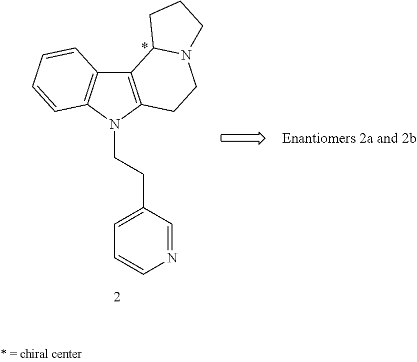 [SMILES: c1cncc(CCn2c3c(c4ccccc42)C2CCCN2CC3)c1]

Similarly, racemic compound 14, bearing two chiral centers, can be resolved into its individual diastereomers 14a, 14b, 14c and 14d.

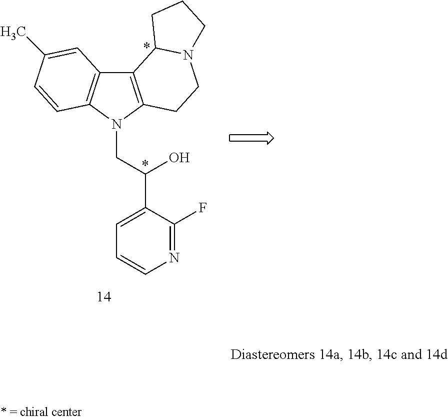 [SMILES: Cc1ccc2c(c1)c1c(n2CC(O)c2cccnc2F)CCN2CCCC12]

It is known that pure enantiomers and pure diastereomers can sometimes be susceptible to epimerization, depending upon a number of factors such as the nature of the chemical structure, and the environmental conditions under which it is stored. In the case of diastereomers, one chiral center may be more susceptible than another to epimerization. Once a stereoisomer is isolated in its pure chiral form, it is preferable to minimize any later epimerization either by modifying storage conditions or use of certain salt forms or other formulation techniques known to those in the art. Alternatively, the chemical structure itself is modified by addition or removal of substituents, such that epimerization is disfavored, typically through a combination of steric and electronic effects.

Certain compounds of the invention, such as for example Compound No. 178d, suggest a tendency to epimerize at the chiral center indicated:

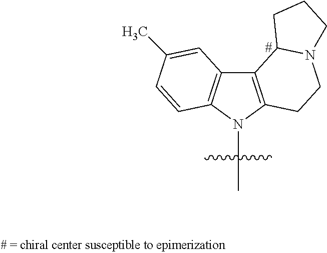 [SMILES: *n1c2c(c3cc(C)ccc31)C1CCCN1CC2]

It is presented herein that particular substitution on the aromatic ring comprising X⁷-X¹⁰ tends to minimize or eliminate epimerization. Addition of electron withdrawing groups, such as in one example the modification of molecule A below, to analogs B, C, or D, results in compounds with more epimerization-resistant characteristics, such as Compound No. 79a:

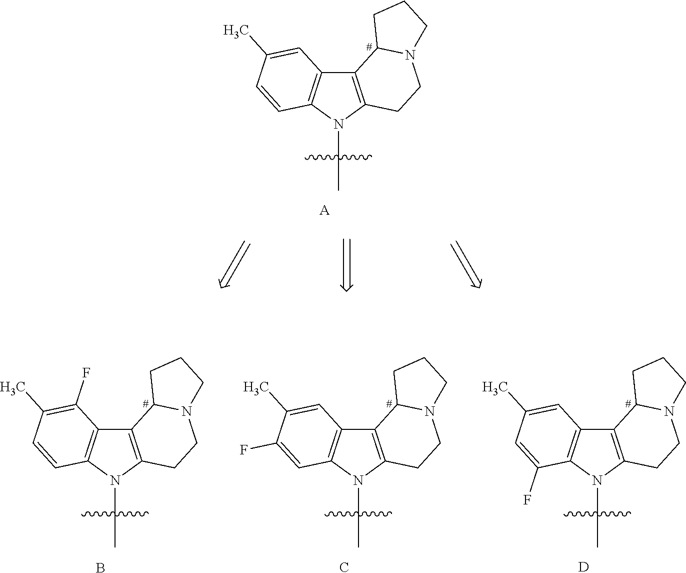

Representative compounds of the invention are shown in Table 1.

TABLE 1

| | |
|---|---|
| 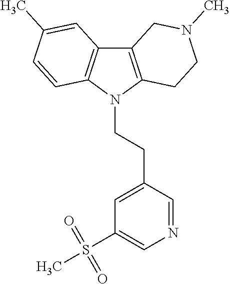 | 1 |
| 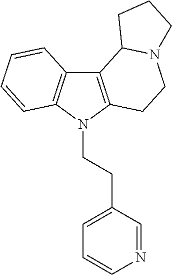 [SMILES: c1cncc(CCn2c3c(c4ccccc42)C2CCCN2CC3)c1] | 2 2a, 2b |
| 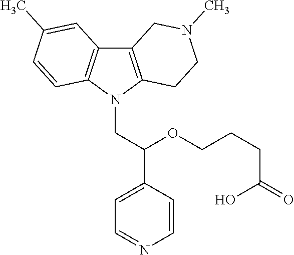 [SMILES: Cc1ccc2c(c1)c1c(n2CC(OCCCC(=O)O)c2ccncc2)CCN(C)C1] | 3 3a, 3b |
| 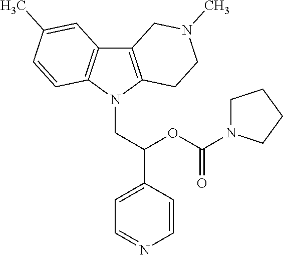 [SMILES: Cc1ccc2c(c1)c1c(n2CC(OC(=O)N2CCCC2)c2ccncc2)CCN(C)C1] | 4 4a, 4b |
| 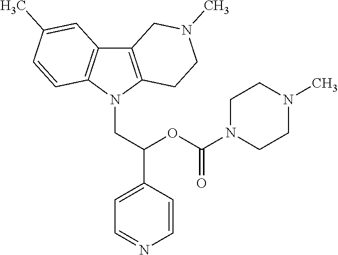 | 5 5a, 5b |
| 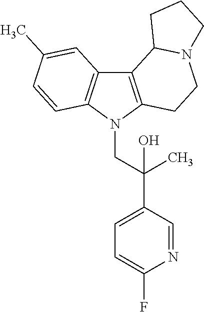 [SMILES: Cc1ccc2c(c1)c1c(n2CC(C)(O)c2ccc(F)nc2)CCN2CCCC12] | 6 6a, 6b, 6c, 6d |
| 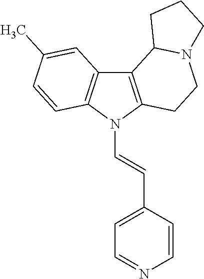 [SMILES: Cc1ccc2c(c1)c1c(n2/C=C/c2ccncc2)CCN2CCCC12] | 7 7a, 7b |
| 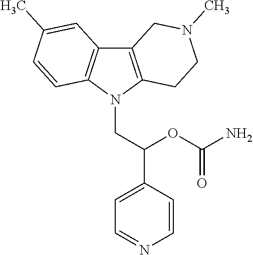 [SMILES: Cc1ccc2c(c1)c1c(n2CC(OC(N)=O)c2ccncc2)CCN(C)C1] | 8 8a, 8b |
| 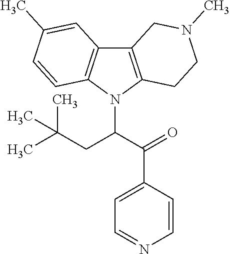 | 9 9a, 9b |
| 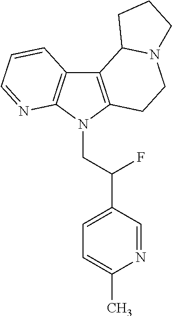 [SMILES: Cc1ccc(C(F)Cn2c3c(c4cccnc42)C2CCCN2CC3)cn1] | 10 10a, 10b, 10c, 10d |
| 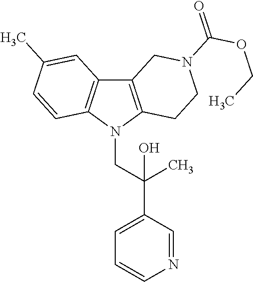 [SMILES: CCOC(=O)N1CCc2c(c3cc(C)ccc3n2CC(C)(O)c2cccnc2)C1] | 11 11a, 11b |
| 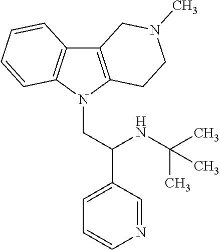 [SMILES: CN1CCc2c(c3ccccc3n2CC(NC(C)(C)C)c2cccnc2)C1] | 12 12a, 12b |
| 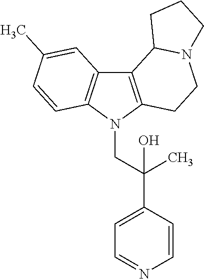 [SMILES: Cc1ccc2c(c1)c1c(n2CC(C)(O)c2ccncc2)CCN2CCCC12] | 13 13a, 13b, 13c, 13d |
| 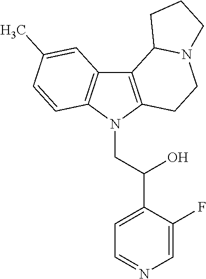 [SMILES: Cc1ccc2c(c1)c1c(n2CC(O)c2ccncc2F)CCN2CCCC12] | 14 14a, 14b, 14c, 14d |
| 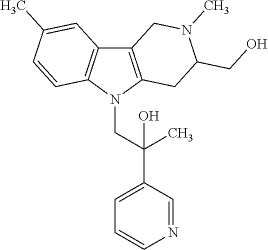 [SMILES: Cc1ccc2c(c1)c1c(n2CC(C)(O)c2cccnc2)CC(CO)N(C)C1] | 15 15a, 15b, 15c, 15d |
| 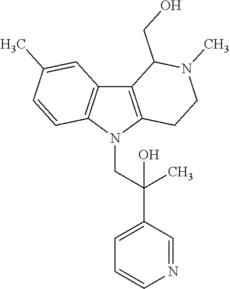 | 16 16a, 16b, 16c, 16d |
| 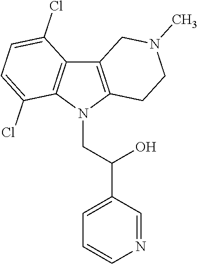 [SMILES: CN1CCc2c(c3c(Cl)ccc(Cl)c3n2CC(O)c2cccnc2)C1] | 17 17a, 17b |
| 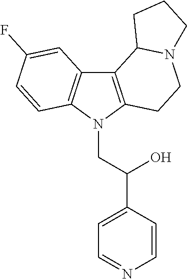 [SMILES: OC(Cn1c2c(c3cc(F)ccc31)C1CCCN1CC2)c1ccncc1] | 18 18a, 18b, 18c, 18d |
| 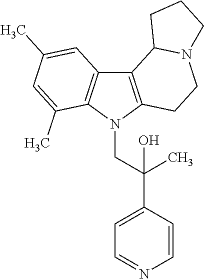 [SMILES: Cc1cc(C)c2c(c1)c1c(n2CC(C)(O)c2ccncc2)CCN2CCCC12] | 19 19a, 19b, 19c, 19d |
| 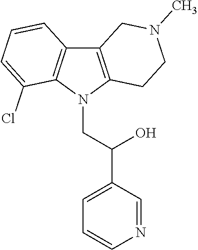 [SMILES: CN1CCc2c(c3cccc(Cl)c3n2CC(O)c2cccnc2)C1] | 20 20a, 20b |
| 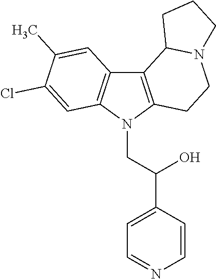 [SMILES: Cc1cc2c3c(n(CC(O)c4ccncc4)c2cc1Cl)CCN1CCCC31] | 21 21a, 21b, 21c, 21d |
| 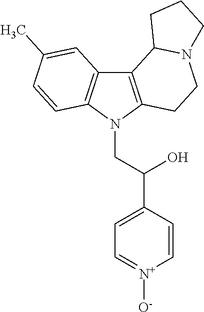 [SMILES: Cc1ccc2c(c1)c1c(n2CC(O)c2cc[n+]([O-])cc2)CCN2CCCC12] | 22 22a, 22b, 22c, 22d |
| 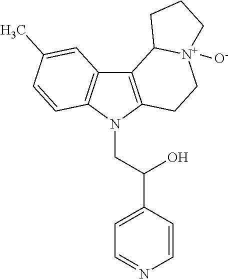 [SMILES: Cc1ccc2c(c1)c1c(n2CC(O)c2ccncc2)CC[N+]2([O-])CCCC12] | 23 23a, 23b, 23c, 23d |
| 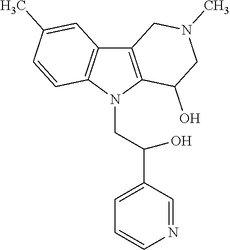 | 24 24a, 24b, 24c, 24d |
| 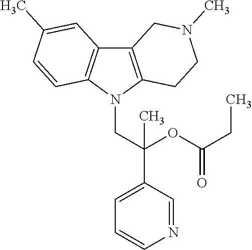 [SMILES: CCC(=O)OC(C)(Cn1c2c(c3cc(C)ccc31)CN(C)CC2)c1cccnc1] | 25 25a, 25b |
| 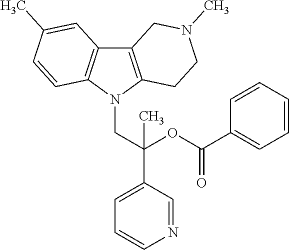 [SMILES: Cc1ccc2c(c1)c1c(n2CC(C)(OC(=O)c2ccccc2)c2cccnc2)CCN(C)C1] | 26 26a, 26b |
| 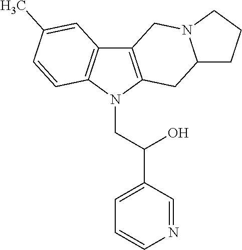 [SMILES: Cc1ccc2c(c1)c1c(n2CC(O)c2cccnc2)CC2CCCN2C1] | 27 27a, 27b, 27c, 27d |
| 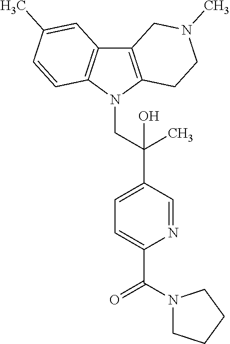 [SMILES: Cc1ccc2c(c1)c1c(n2CC(C)(O)c2ccc(C(=O)N3CCCC3)nc2)CCN(C)C1] | 28 28a, 28b |
| 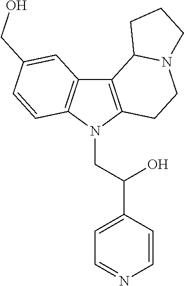 [SMILES: OCc1ccc2c(c1)c1c(n2CC(O)c2ccncc2)CCN2CCCC12] | 29 29a, 29b, 29c, 29d |
| 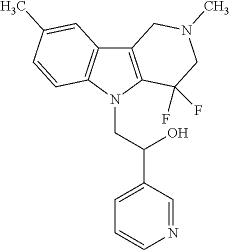 | 30 30a, 30b |
| 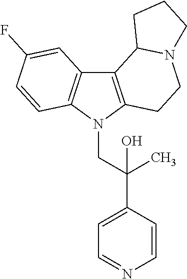 [SMILES: CC(O)(Cn1c2c(c3cc(F)ccc31)C1CCCN1CC2)c1ccncc1] | 31 31a, 31b, 31c, 31d |
| 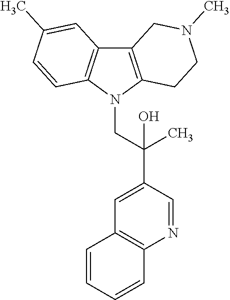 | 32 32a, 32b |
| 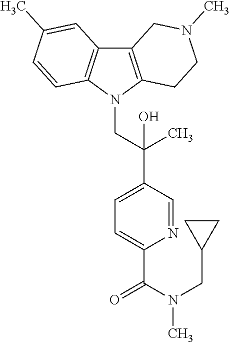 [SMILES: Cc1ccc2c(c1)c1c(n2CC(C)(O)c2ccc(C(=O)N(C)CC3CC3)nc2)CCN(C)C1] | 33 33a, 33b |
| 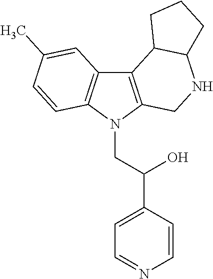 [SMILES: Cc1ccc2c(c1)c1c(n2CC(O)c2ccncc2)CNC2CCCC12] | 34 34a, 34b, 34c, 34d |
| 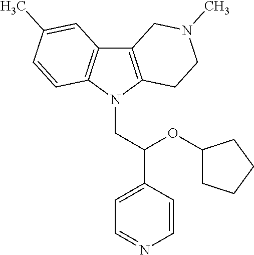 [SMILES: Cc1ccc2c(c1)c1c(n2CC(OC2CCCC2)c2ccncc2)CCN(C)C1] | 35 35a, 35b |
| 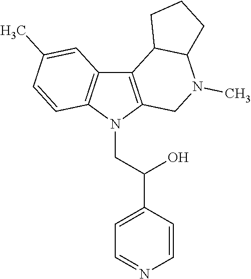 | 36 36a, 36b, 36c, 36d |
| 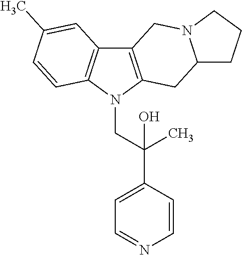 [SMILES: Cc1ccc2c(c1)c1c(n2CC(C)(O)c2ccncc2)CC2CCCN2C1] | 37 37a, 37b, 37c, 37d |
| 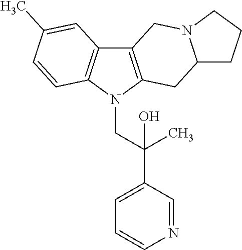 [SMILES: Cc1ccc2c(c1)c1c(n2CC(C)(O)c2cccnc2)CC2CCCN2C1] | 38 38a, 38b, 38c, 38d |
| 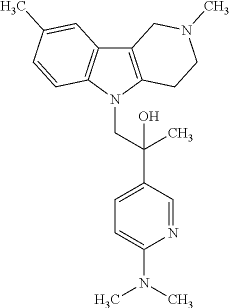 | 39 39a, 39b |
| 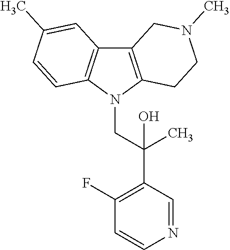 | 40 40a, 40b |
| 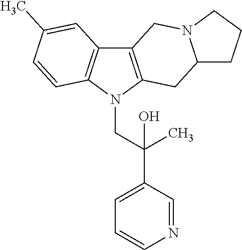 [SMILES: Cc1ccc2c(c1)c1c(n2CC(C)(O)c2cccnc2)CC2CCCN2C1] | 41 41a, 41b, 41c, 41d |
| 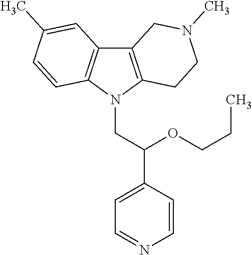 [SMILES: CCCOC(Cn1c2c(c3cc(C)ccc31)CN(C)CC2)c1ccncc1] | 42 42a, 42b |
| 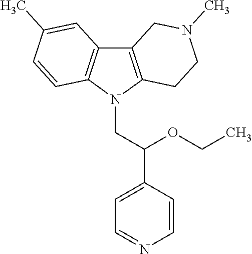 [SMILES: CCOC(Cn1c2c(c3cc(C)ccc31)CN(C)CC2)c1ccncc1] | 43 43a, 43b |
| 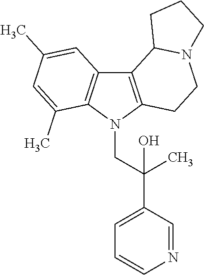 [SMILES: Cc1cc(C)c2c(c1)c1c(n2CC(C)(O)c2cccnc2)CCN2CCCC12] | 44 44a, 44b, 44c, 44d |
| 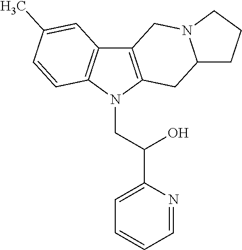 [SMILES: Cc1ccc2c(c1)c1c(n2CC(O)c2ccccn2)CC2CCCN2C1] | 45 45a, 45b, 45c, 45d |
| 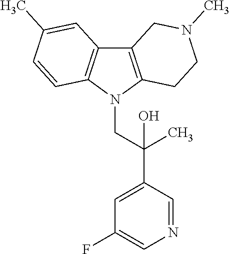 | 46 46a, 46b |
| 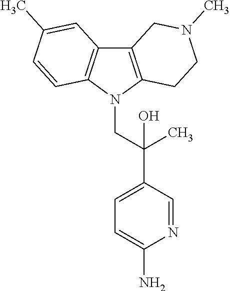 | 47 47a, 47b |
| 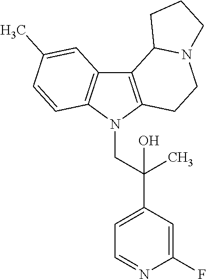 [SMILES: Cc1ccc2c(c1)c1c(n2CC(C)(O)c2ccnc(F)c2)CCN2CCCC12] | 48 48a, 48b, 48c, 48d |
| 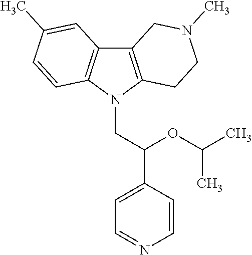 [SMILES: Cc1ccc2c(c1)c1c(n2CC(OC(C)C)c2ccncc2)CCN(C)C1] | 49 49a, 49b |
| 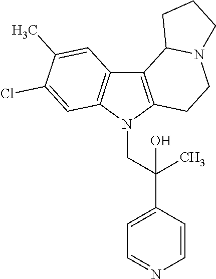 [SMILES: Cc1cc2c3c(n(CC(C)(O)c4ccncc4)c2cc1Cl)CCN1CCCC31] | 50 50a, 50b, 50c, 50d |
| 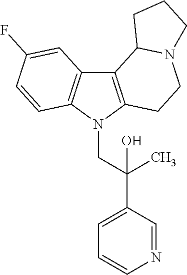 [SMILES: CC(O)(Cn1c2c(c3cc(F)ccc31)C1CCCN1CC2)c1cccnc1] | 51 51a, 51b, 51c, 51d |
| 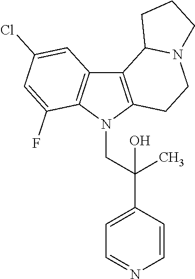 [SMILES: CC(O)(Cn1c2c(c3cc(Cl)cc(F)c31)C1CCCN1CC2)c1ccncc1] | 52 52a, 52b, 52c, 52d |
| 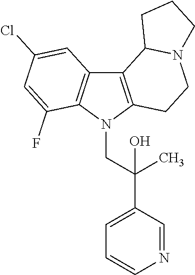 [SMILES: CC(O)(Cn1c2c(c3cc(Cl)cc(F)c31)C1CCCN1CC2)c1cccnc1] | 53 53a, 53b, 53c, 53d |
| 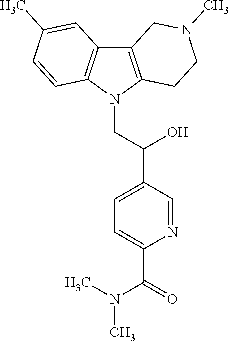 | 54 54a, 54b |
| 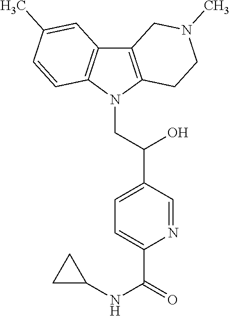 | 55 55a, 55b |
| 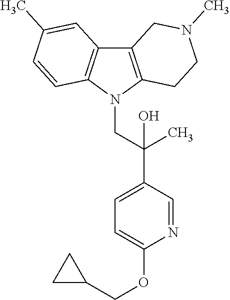 | 56 56a, 56b |
| 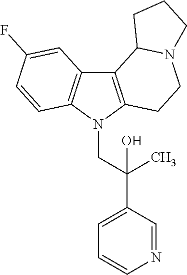 [SMILES: CC(O)(Cn1c2c(c3cc(F)ccc31)C1CCCN1CC2)c1cccnc1] | 57 57a, 57b, 57c, 57d |
| 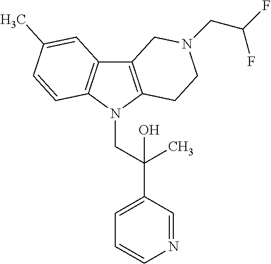 [SMILES: Cc1ccc2c(c1)c1c(n2CC(C)(O)c2cccnc2)CCN(CC(F)F)C1] | 58 58a, 58b |
| 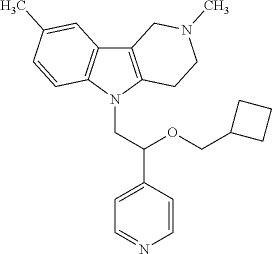 [SMILES: Cc1ccc2c(c1)c1c(n2CC(OCC2CCC2)c2ccncc2)CCN(C)C1] | 59 59a, 59b |
| 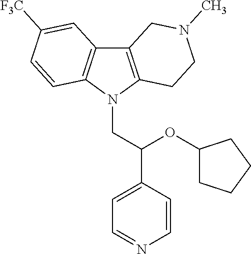 [SMILES: CN1CCc2c(c3cc(C(F)(F)F)ccc3n2CC(OC2CCCC2)c2ccncc2)C1] | 60 60a, 60b |
| 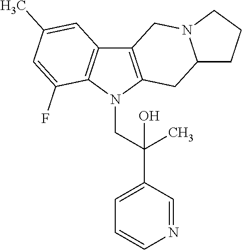 [SMILES: Cc1cc(F)c2c(c1)c1c(n2CC(C)(O)c2cccnc2)CC2CCCN2C1] | 61 61a, 61b, 61c, 61d |
| 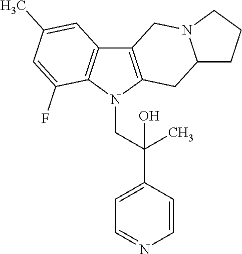 [SMILES: Cc1cc(F)c2c(c1)c1c(n2CC(C)(O)c2ccncc2)CC2CCCN2C1] | 62 62a, 62b, 62c, 62d |
| 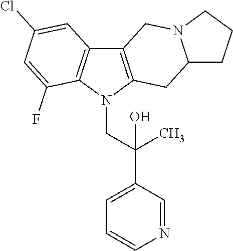 [SMILES: CC(O)(Cn1c2c(c3cc(Cl)cc(F)c31)CN1CCCC1C2)c1cccnc1] | 63 63a, 63b, 63c, 63d |
| 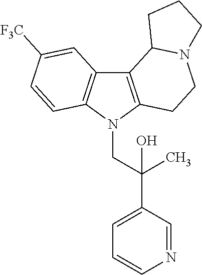 [SMILES: CC(O)(Cn1c2c(c3cc(C(F)(F)F)ccc31)C1CCCN1CC2)c1cccnc1] | 64 64a, 64b, 64c, 64d |
| 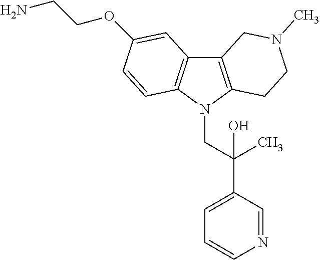 [SMILES: CN1CCc2c(c3cc(OCCN)ccc3n2CC(C)(O)c2cccnc2)C1] | 65 65a, 65b |
| 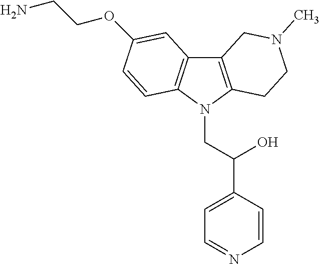 [SMILES: CN1CCc2c(c3cc(OCCN)ccc3n2CC(O)c2ccncc2)C1] | 66 66a, 66b |
| 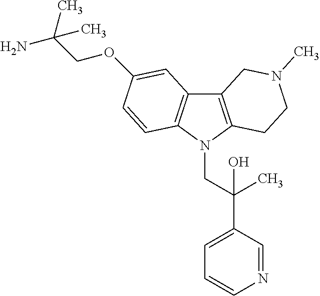 [SMILES: CN1CCc2c(c3cc(OCC(C)(C)N)ccc3n2CC(C)(O)c2cccnc2)C1] | 67 67a, 67b |
| 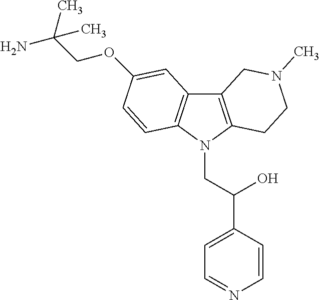 [SMILES: CN1CCc2c(c3cc(OCC(C)(C)N)ccc3n2CC(O)c2ccncc2)C1] | 68 68a, 68b |
| 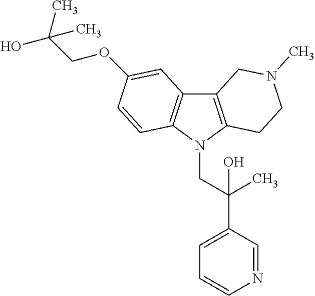 [SMILES: CN1CCc2c(c3cc(OCC(C)(C)O)ccc3n2CC(C)(O)c2cccnc2)C1] | 69 69a, 69b |
| 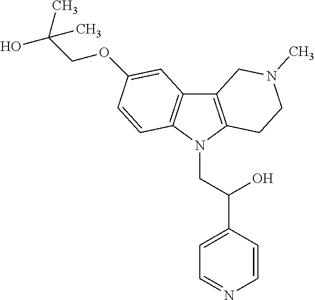 [SMILES: CN1CCc2c(c3cc(OCC(C)(C)O)ccc3n2CC(O)c2ccncc2)C1] | 70 70a, 70b |
| 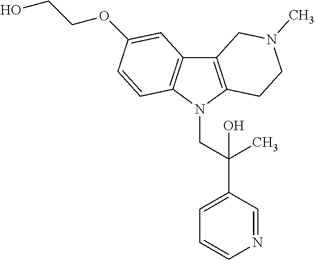 [SMILES: CN1CCc2c(c3cc(OCCO)ccc3n2CC(C)(O)c2cccnc2)C1] | 71 71a, 71b |
| 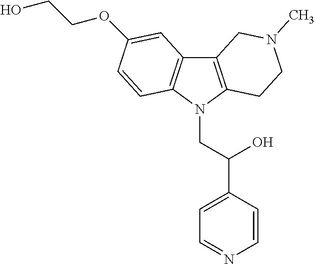 [SMILES: CN1CCc2c(c3cc(OCCO)ccc3n2CC(O)c2ccncc2)C1] | 72 72a, 72b |
| 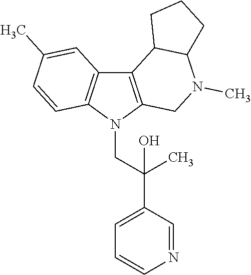 | 73 73a, 73b, 73c, 73d, 73e, 73f, 73g, 73h |
| 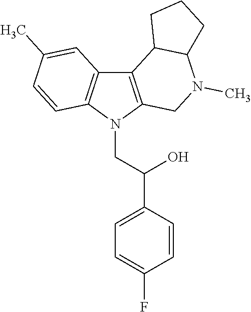 | 74 74a, 74b, 74c, 74d, 74e, 74f, 74g, 74h |
| 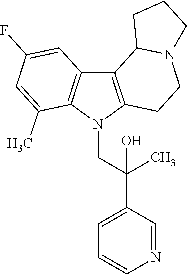 [SMILES: Cc1cc(F)cc2c3c(n(CC(C)(O)c4cccnc4)c12)CCN1CCCC31] | 75 75a, 75b, 75c, 75d |
| 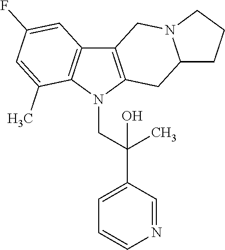 [SMILES: Cc1cc(F)cc2c3c(n(CC(C)(O)c4cccnc4)c12)CC1CCCN1C3] | 76 76a, 76b, 76c, 76d |
| 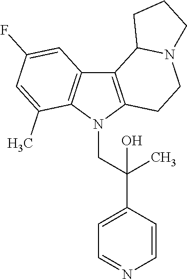 [SMILES: Cc1cc(F)cc2c3c(n(CC(C)(O)c4ccncc4)c12)CCN1CCCC31] | 77 77a, 77b, 77c, 77d |
| 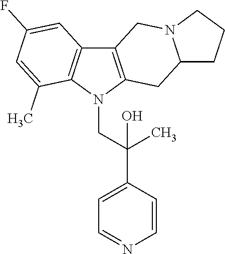 [SMILES: Cc1cc(F)cc2c3c(n(CC(C)(O)c4ccncc4)c12)CC1CCCN1C3] | 78 78a, 78b, 78c, 78d |
| 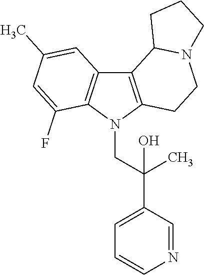 [SMILES: Cc1cc(F)c2c(c1)c1c(n2CC(C)(O)c2cccnc2)CCN2CCCC12] | 79 79a, 79b, 79c, 79d |
| 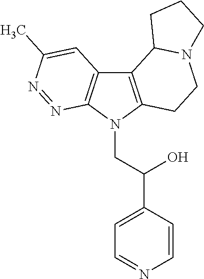 [SMILES: Cc1cc2c3c(n(CC(O)c4ccncc4)c2nn1)CCN1CCCC31] | 80 80a, 80b, 80c, 80d |
| 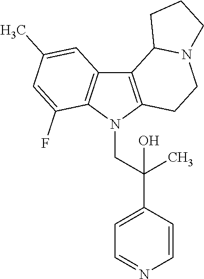 [SMILES: Cc1cc(F)c2c(c1)c1c(n2CC(C)(O)c2ccncc2)CCN2CCCC12] | 81 81a, 81b, 81c, 81d |
| 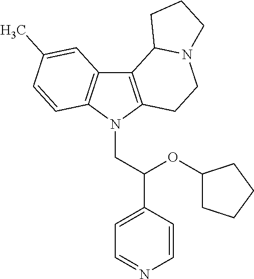 [SMILES: Cc1ccc2c(c1)c1c(n2CC(OC2CCCC2)c2ccncc2)CCN2CCCC12] | 82 82a, 82b, 82c, 82d |
| 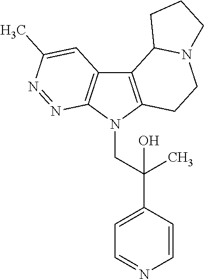 [SMILES: Cc1cc2c3c(n(CC(C)(O)c4ccncc4)c2nn1)CCN1CCCC31] | 83 83a, 83b, 83c, 83d |
| 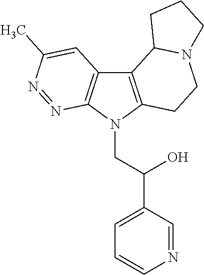 [SMILES: Cc1cc2c3c(n(CC(O)c4cccnc4)c2nn1)CCN1CCCC31] | 84 84a, 84b, 84c, 84d |
| 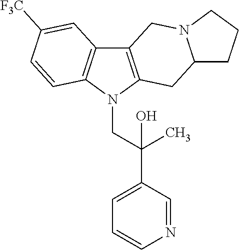 [SMILES: CC(O)(Cn1c2c(c3cc(C(F)(F)F)ccc31)CN1CCCC1C2)c1cccnc1] | 85 85a, 85b, 85c, 85d |
| 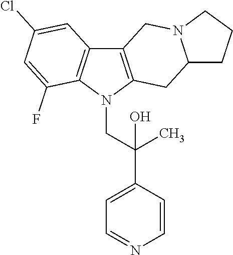 [SMILES: CC(O)(Cn1c2c(c3cc(Cl)cc(F)c31)CN1CCCC1C2)c1ccncc1] | 86 86a, 86b, 86c, 86d |
| 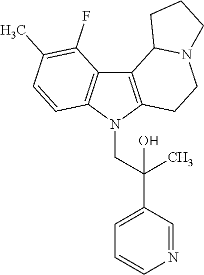 [SMILES: Cc1ccc2c(c1F)c1c(n2CC(C)(O)c2cccnc2)CCN2CCCC12] | 87 87a, 87b, 87c, 87d |
| 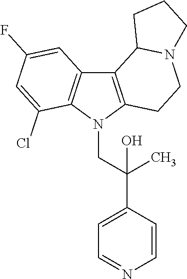 [SMILES: CC(O)(Cn1c2c(c3cc(F)cc(Cl)c31)C1CCCN1CC2)c1ccncc1] | 88 88a, 88b, 88c, 88d |
| 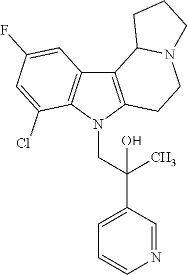 [SMILES: CC(O)(Cn1c2c(c3cc(F)cc(Cl)c31)C1CCCN1CC2)c1cccnc1] | 89 89a, 89b, 89c, 89d |
| 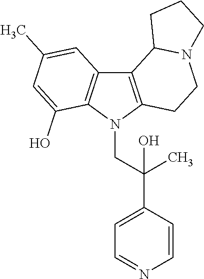 [SMILES: Cc1cc(O)c2c(c1)c1c(n2CC(C)(O)c2ccncc2)CCN2CCCC12] | 90 90a, 90b, 90c, 90d |
| 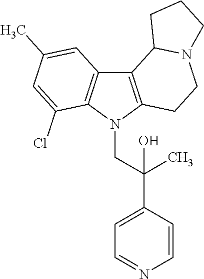 [SMILES: Cc1cc(Cl)c2c(c1)c1c(n2CC(C)(O)c2ccncc2)CCN2CCCC12] | 91 91a, 91b, 91c, 91d |
| 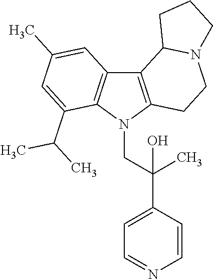 [SMILES: Cc1cc(C(C)C)c2c(c1)c1c(n2CC(C)(O)c2ccncc2)CCN2CCCC12] | 92 92a, 92b, 92c, 92d |
| 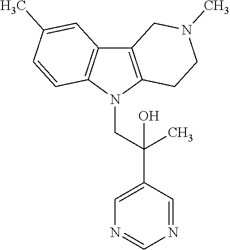 | 93 93a, 93b |
| 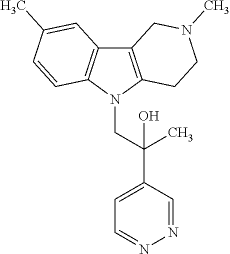 | 94 94a, 94b |
| 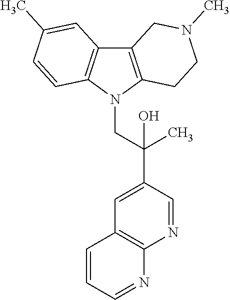 | 95 95a, 95b |
| 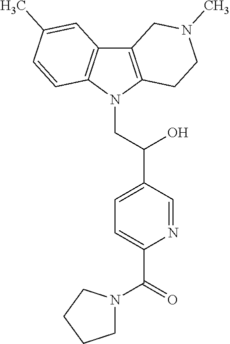 | 96 96a, 96b |
| 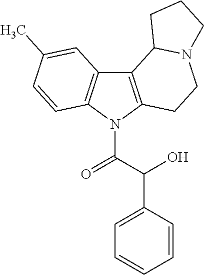 [SMILES: Cc1ccc2c(c1)c1c(n2C(=O)C(O)c2ccccc2)CCN2CCCC12] | 97 97a, 97b, 97c, 97d |
| 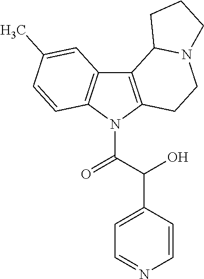 [SMILES: Cc1ccc2c(c1)c1c(n2C(=O)C(O)c2ccncc2)CCN2CCCC12] | 98 98a, 98b, 98c, 98d |
| 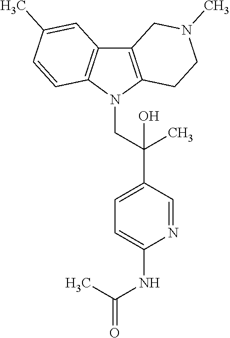 | 99 99a, 99b |
| 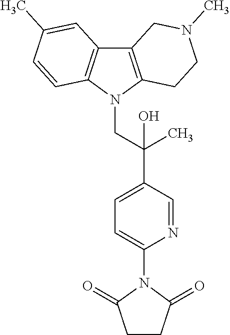 | 100 100a, 100b |
| 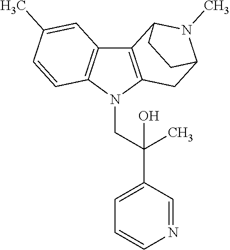 | 101 101a, 101b, 101c, 101d |
| 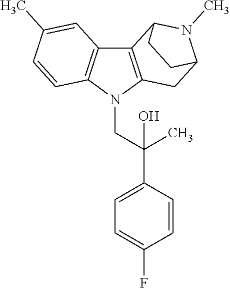 | 102 102a, 102b, 102c, 102d |
| 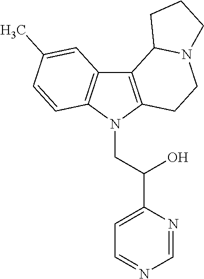 [SMILES: Cc1ccc2c(c1)c1c(n2CC(O)c2ccncn2)CCN2CCCC12] | 103 103a, 103b, 103c, 103d |
| 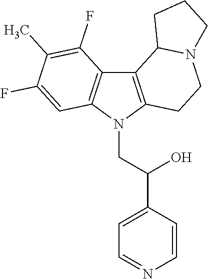 [SMILES: Cc1c(F)cc2c(c1F)c1c(n2CC(O)c2ccncc2)CCN2CCCC12] | 104 104a, 104b, 104c, 104d |
| 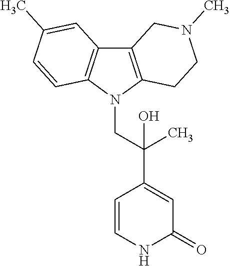 | 105 105a, 105b |
| 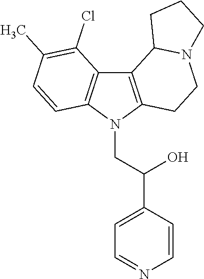 [SMILES: Cc1ccc2c(c1Cl)c1c(n2CC(O)c2ccncc2)CCN2CCCC12] | 106 106a, 106b, 106c, 106d |
| 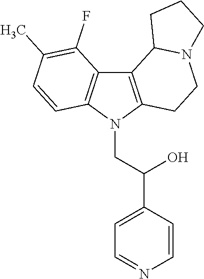 [SMILES: Cc1ccc2c(c1F)c1c(n2CC(O)c2ccncc2)CCN2CCCC12] | 107 107a, 107b, 107c, 107d |
| 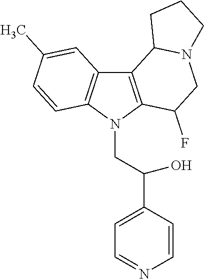 [SMILES: Cc1ccc2c(c1)c1c(n2CC(O)c2ccncc2)C(F)CN2CCCC12] | 108 108a, 108b, 108c, 108d |
| 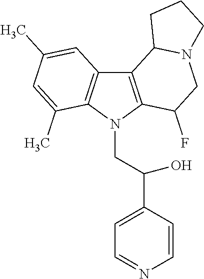 [SMILES: Cc1cc(C)c2c(c1)c1c(n2CC(O)c2ccncc2)C(F)CN2CCCC12] | 109 109a, 109b, 109c, 109d |
| 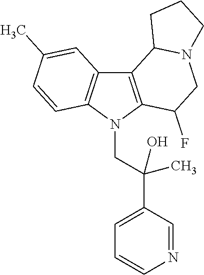 [SMILES: Cc1ccc2c(c1)c1c(n2CC(C)(O)c2cccnc2)C(F)CN2CCCC12] | 110 110a, 110b, 110c, 110d |
| 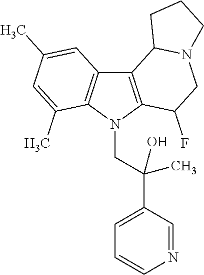 [SMILES: Cc1cc(C)c2c(c1)c1c(n2CC(C)(O)c2cccnc2)C(F)CN2CCCC12] | 111 111a, 111b, 111c, 111d |
| 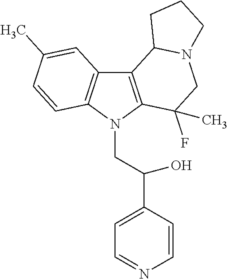 | 112 112a, 112b, 112c, 112d |
| 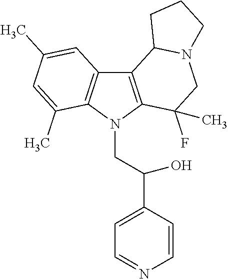 | 113 113a, 113b, 113c, 113d |
| 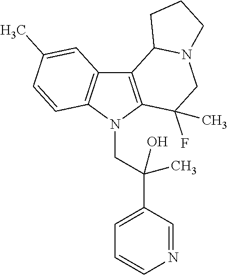 | 114 114a, 114b, 114c, 114d |
| 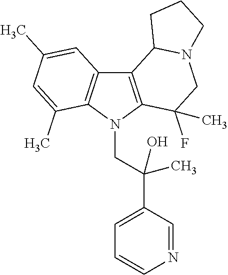 | 115 115a, 115b, 115c, 115d |
| 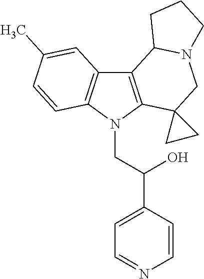 [SMILES: Cc1ccc2c(c1)c1c(n2CC(O)c2ccncc2)C2(CC2)CN2CCCC12] | 116 116a, 116b, 116c, 116d |
| 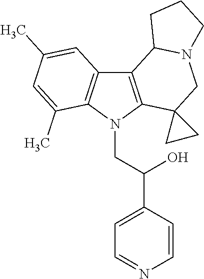 [SMILES: Cc1cc(C)c2c(c1)c1c(n2CC(O)c2ccncc2)C2(CC2)CN2CCCC12] | 117 117a, 117b, 117c, 117d |
| 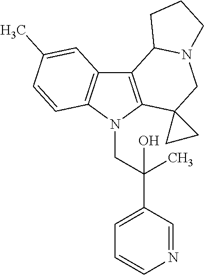 [SMILES: Cc1ccc2c(c1)c1c(n2CC(C)(O)c2cccnc2)C2(CC2)CN2CCCC12] | 118 118a, 118b, 118c, 118d |
| 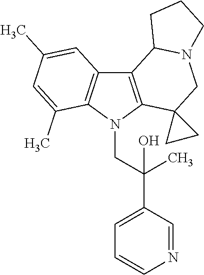 [SMILES: Cc1cc(C)c2c(c1)c1c(n2CC(C)(O)c2cccnc2)C2(CC2)CN2CCCC12] | 119 119a, 119b, 119c, 119d |
| 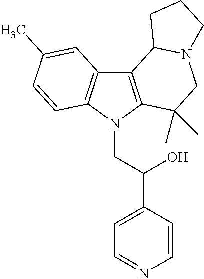 [SMILES: Cc1ccc2c(c1)c1c(n2CC(O)c2ccncc2)C(C)(C)CN2CCCC12] | 120 120a, 120b, 120c, 120d |
| 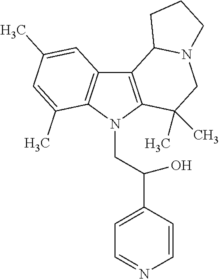 [SMILES: Cc1cc(C)c2c(c1)c1c(n2CC(O)c2ccncc2)C(C)(C)CN2CCCC12] | 121 121a, 121b, 121c, 121d |
| 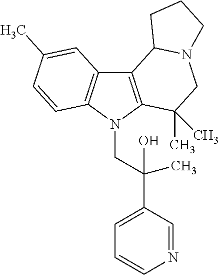 [SMILES: Cc1ccc2c(c1)c1c(n2CC(C)(O)c2cccnc2)C(C)(C)CN2CCCC12] | 122 122a, 122b, 122c, 122d |
| 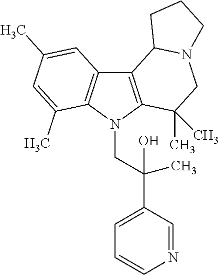 [SMILES: Cc1cc(C)c2c(c1)c1c(n2CC(C)(O)c2cccnc2)C(C)(C)CN2CCCC12] | 123 123a, 123b, 123c, 123d |
| 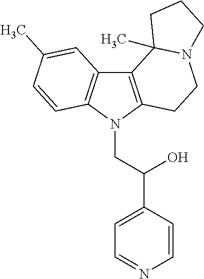 [SMILES: Cc1ccc2c(c1)c1c(n2CC(O)c2ccncc2)CCN2CCCC12C] | 124 124a, 124b, 124c, 124d |
| 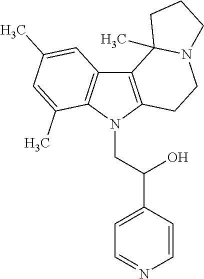 [SMILES: Cc1cc(C)c2c(c1)c1c(n2CC(O)c2ccncc2)CCN2CCCC12C] | 125 125a, 125b, 125c, 125d |
| 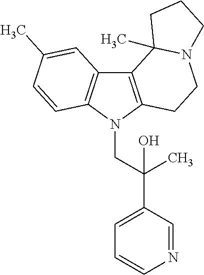 [SMILES: Cc1ccc2c(c1)c1c(n2CC(C)(O)c2cccnc2)CCN2CCCC12C] | 126 126a, 126b, 126c, 126d |
| 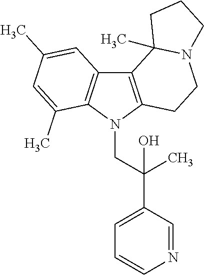 [SMILES: Cc1cc(C)c2c(c1)c1c(n2CC(C)(O)c2cccnc2)CCN2CCCC12C] | 127 127a, 127b, 127c, 127d |
| 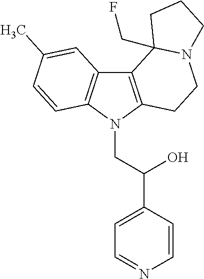 [SMILES: Cc1ccc2c(c1)c1c(n2CC(O)c2ccncc2)CCN2CCCC12CF] | 128 128a, 128b, 128c, 128d |
| 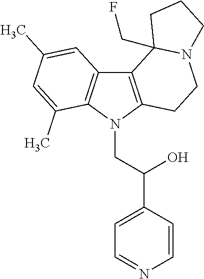 [SMILES: Cc1cc(C)c2c(c1)c1c(n2CC(O)c2ccncc2)CCN2CCCC12CF] | 129 129a, 129b, 129c, 129d |
| 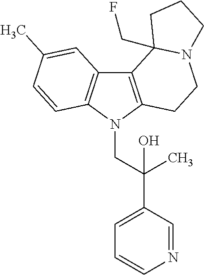 [SMILES: Cc1ccc2c(c1)c1c(n2CC(C)(O)c2cccnc2)CCN2CCCC12CF] | 130 130a, 130b, 130c, 130d |
| 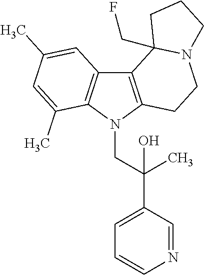 [SMILES: Cc1cc(C)c2c(c1)c1c(n2CC(C)(O)c2cccnc2)CCN2CCCC12CF] | 131 131a, 131b, 131c, 131d |
| 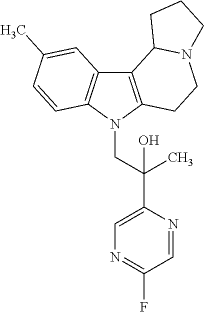 [SMILES: Cc1ccc2c(c1)c1c(n2CC(C)(O)c2cnc(F)cn2)CCN2CCCC12] | 132 132a, 132b, 132c, 132d |
| 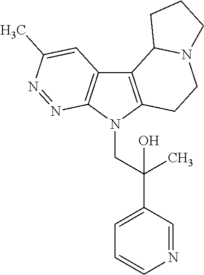 [SMILES: Cc1cc2c3c(n(CC(C)(O)c4cccnc4)c2nn1)CCN1CCCC31] | 133 133a, 133b, 133c, 133d |
| 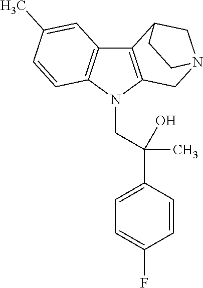 [SMILES: Cc1ccc2c(c1)c1c(n2CC(C)(O)c2ccc(F)cc2)CN2CCC1C2] | 134 134a, 134b, 134c, 134d |
| 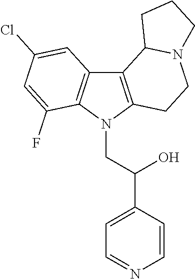 [SMILES: OC(Cn1c2c(c3cc(Cl)cc(F)c31)C1CCCN1CC2)c1ccncc1] | 135 135a, 135b, 135c, 135d |
| 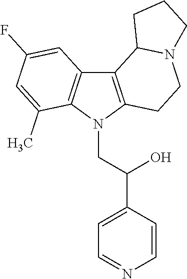 [SMILES: Cc1cc(F)cc2c3c(n(CC(O)c4ccncc4)c12)CCN1CCCC31] | 136 136a, 136b, 136c, 136d |
| 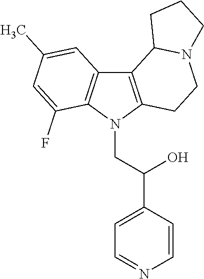 [SMILES: Cc1cc(F)c2c(c1)c1c(n2CC(O)c2ccncc2)CCN2CCCC12] | 137 137a, 137b, 137c, 137d |
| 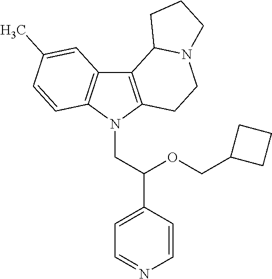 [SMILES: Cc1ccc2c(c1)c1c(n2CC(OCC2CCC2)c2ccncc2)CCN2CCCC12] | 138 138a, 138b, 138c, 138d |
| 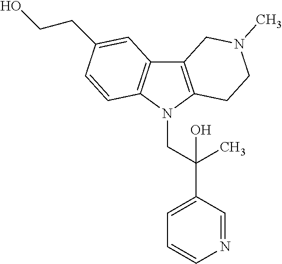 [SMILES: CN1CCc2c(c3cc(CCO)ccc3n2CC(C)(O)c2cccnc2)C1] | 139 139a, 139b |
| 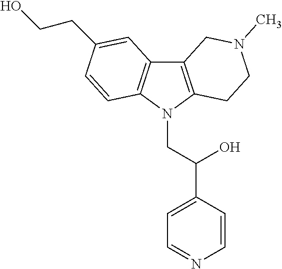 [SMILES: CN1CCc2c(c3cc(CCO)ccc3n2CC(O)c2ccncc2)C1] | 140 140a, 140b |
| 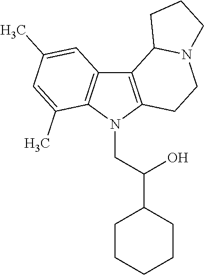 [SMILES: Cc1cc(C)c2c(c1)c1c(n2CC(O)C2CCCCC2)CCN2CCCC12] | 141 141a, 141b, 141c, 141d |
| 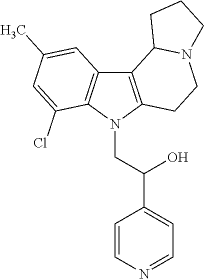 [SMILES: Cc1cc(Cl)c2c(c1)c1c(n2CC(O)c2ccncc2)CCN2CCCC12] | 142 142a, 142b, 142c, 142d |
| 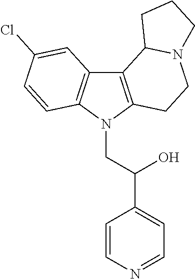 [SMILES: OC(Cn1c2c(c3cc(Cl)ccc31)C1CCCN1CC2)c1ccncc1] | 143 143a, 143b, 143c, 143d |
| 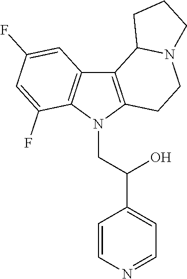 [SMILES: OC(Cn1c2c(c3cc(F)cc(F)c31)C1CCCN1CC2)c1ccncc1] | 144 144a, 144b, 144c, 144d |
| 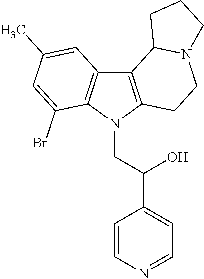 [SMILES: Cc1cc(Br)c2c(c1)c1c(n2CC(O)c2ccncc2)CCN2CCCC12] | 145 145a, 145b, 145c, 145d |
| 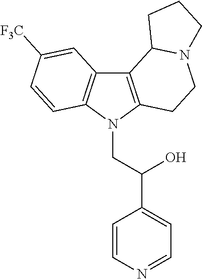 [SMILES: OC(Cn1c2c(c3cc(C(F)(F)F)ccc31)C1CCCN1CC2)c1ccncc1] | 146 146a, 146b, 146c, 146d |
| 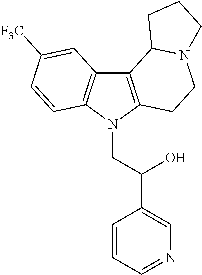 [SMILES: OC(Cn1c2c(c3cc(C(F)(F)F)ccc31)C1CCCN1CC2)c1cccnc1] | 147 147a, 147b, 147c, 147d |
| 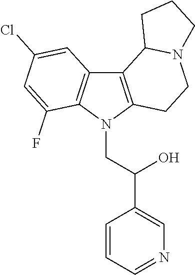 [SMILES: OC(Cn1c2c(c3cc(Cl)cc(F)c31)C1CCCN1CC2)c1cccnc1] | 148 148a, 148b, 148c, 148d |
| 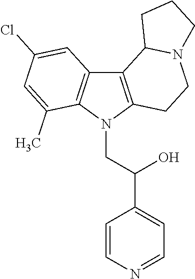 [SMILES: Cc1cc(Cl)cc2c3c(n(CC(O)c4ccncc4)c12)CCN1CCCC31] | 149 149a, 149b, 149c, 149d |
| 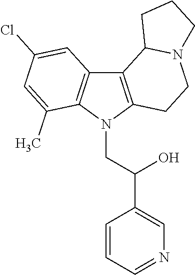 [SMILES: Cc1cc(Cl)cc2c3c(n(CC(O)c4cccnc4)c12)CCN1CCCC31] | 150 150a, 150b, 150c, 150d |
| 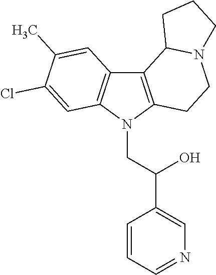 [SMILES: Cc1cc2c3c(n(CC(O)c4cccnc4)c2cc1Cl)CCN1CCCC31] | 151 151a, 151b, 151c, 151d |
| 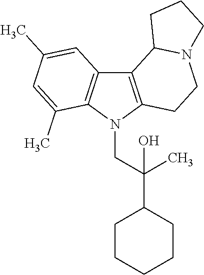 [SMILES: Cc1cc(C)c2c(c1)c1c(n2CC(C)(O)C2CCCCC2)CCN2CCCC12] | 152 152a, 152b, 152c, 152d |
| 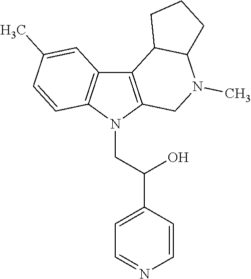 | 153 153a, 153b, 153c, 153d |
| 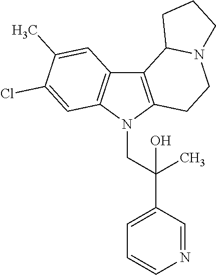 [SMILES: Cc1cc2c3c(n(CC(C)(O)c4cccnc4)c2cc1Cl)CCN1CCCC31] | 154 154a, 154b, 154c, 154d |
| 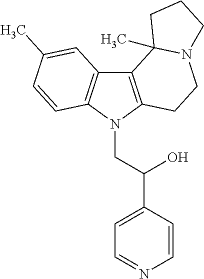 [SMILES: Cc1ccc2c(c1)c1c(n2CC(O)c2ccncc2)CCN2CCCC12C] | 155 155a, 155b, 155c, 155d |
| 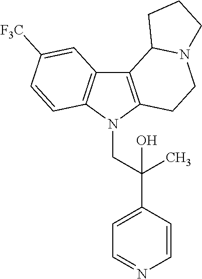 [SMILES: CC(O)(Cn1c2c(c3cc(C(F)(F)F)ccc31)C1CCCN1CC2)c1ccncc1] | 156 156a, 156b, 156c, 156d |
| 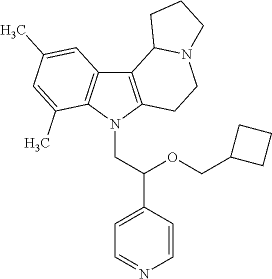 [SMILES: Cc1cc(C)c2c(c1)c1c(n2CC(OCC2CCC2)c2ccncc2)CCN2CCCC12] | 157 157a, 157b, 157c, 157d |
| 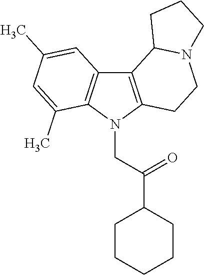 [SMILES: Cc1cc(C)c2c(c1)c1c(n2CC(=O)C2CCCCC2)CCN2CCCC12] | 158 158a, 158b, 158c, 158d |
| 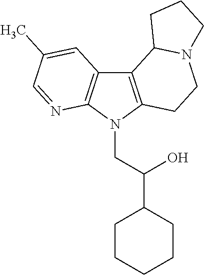 [SMILES: Cc1cnc2c(c1)c1c(n2CC(O)C2CCCCC2)CCN2CCCC12] | 159 159a, 159b, 159c, 159d |
| 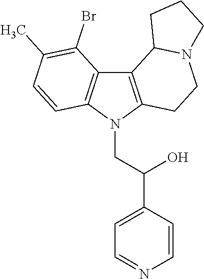 [SMILES: Cc1ccc2c(c1Br)c1c(n2CC(O)c2ccncc2)CCN2CCCC12] | 160 160a, 160b, 160c, 160d |
| 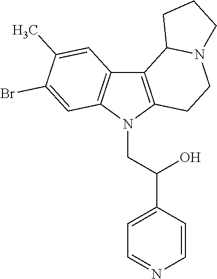 [SMILES: Cc1cc2c3c(n(CC(O)c4ccncc4)c2cc1Br)CCN1CCCC31] | 161 161a, 161b, 161c, 161d |
| 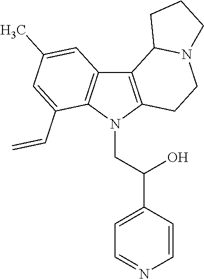 [SMILES: C=Cc1cc(C)cc2c3c(n(CC(O)c4ccncc4)c12)CCN1CCCC31] | 162 162a, 162b, 162c, 162d |
| 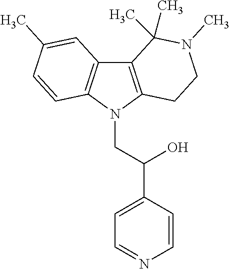 | 163 163a, 163b |
| 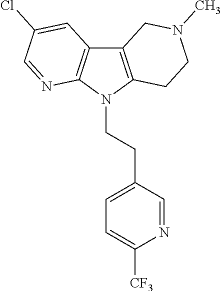 [SMILES: CN1CCc2c(c3cc(Cl)cnc3n2CCc2ccc(C(F)(F)F)nc2)C1] | 164 |
| 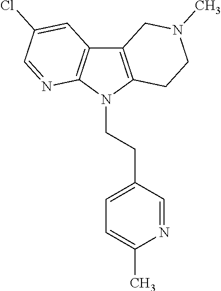 [SMILES: Cc1ccc(CCn2c3c(c4cc(Cl)cnc42)CN(C)CC3)cn1] | 165 |
| 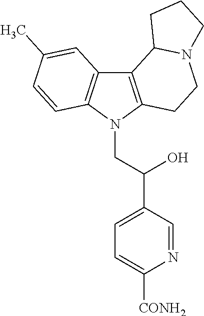 [SMILES: Cc1ccc2c(c1)c1c(n2CC(O)c2ccc(C(N)=O)nc2)CCN2CCCC12] | 166 166a, 166b, 166c, 166d |
| 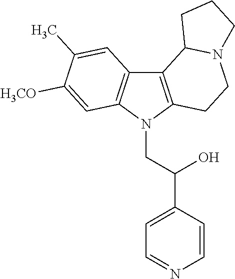 [SMILES: COc1cc2c(cc1C)c1c(n2CC(O)c2ccncc2)CCN2CCCC12] | 167 167a, 167b, 167c, 167d |
| 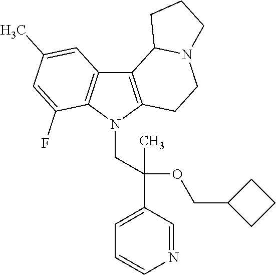 [SMILES: Cc1cc(F)c2c(c1)c1c(n2CC(C)(OCC2CCC2)c2cccnc2)CCN2CCCC12] | 168 168a, 168b, 168c, 168d |
| 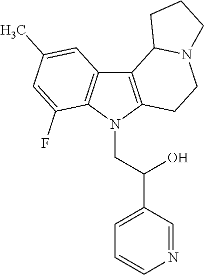 [SMILES: Cc1cc(F)c2c(c1)c1c(n2CC(O)c2cccnc2)CCN2CCCC12] | 169 169a, 169b, 169c, 169d |
| 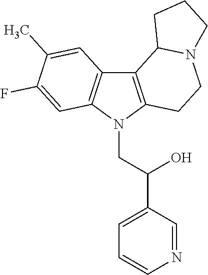 [SMILES: Cc1cc2c3c(n(CC(O)c4cccnc4)c2cc1F)CCN1CCCC31] | 170 170a, 170b, 170c, 170d |
| 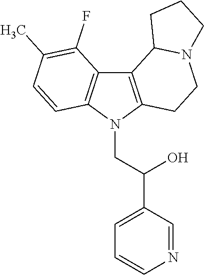 [SMILES: Cc1ccc2c(c1F)c1c(n2CC(O)c2cccnc2)CCN2CCCC12] | 171 171a, 171b, 171c, 171d |
| 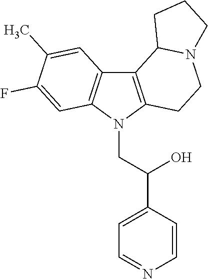 [SMILES: Cc1cc2c3c(n(CC(O)c4ccncc4)c2cc1F)CCN1CCCC31] | 172 172a, 172b, 172c, 172d |
| 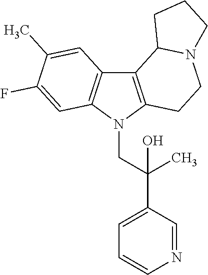 [SMILES: Cc1cc2c3c(n(CC(C)(O)c4cccnc4)c2cc1F)CCN1CCCC31] | 173 173a, 173b, 173c, 173d |
| 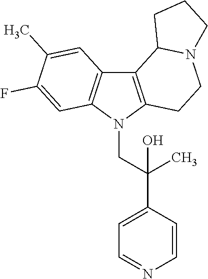 [SMILES: Cc1cc2c3c(n(CC(C)(O)c4ccncc4)c2cc1F)CCN1CCCC31] | 174 174a, 174b, 174c, 174d |
| 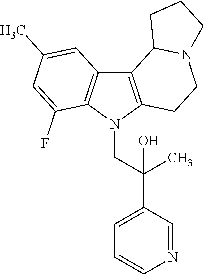 [SMILES: Cc1cc(F)c2c(c1)c1c(n2CC(C)(O)c2cccnc2)CCN2CCCC12] | 175 175a, 175b, 175c, 175d |
| 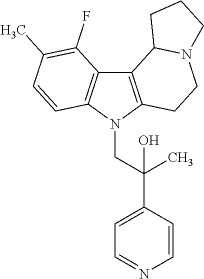 [SMILES: Cc1ccc2c(c1F)c1c(n2CC(C)(O)c2ccncc2)CCN2CCCC12] | 176 176a, 176b, 176c, 176d |
| 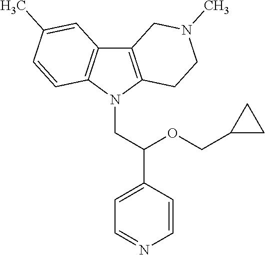 [SMILES: Cc1ccc2c(c1)c1c(n2CC(OCC2CC2)c2ccncc2)CCN(C)C1] | 177 177a, 177b, 177c, 177d |
| 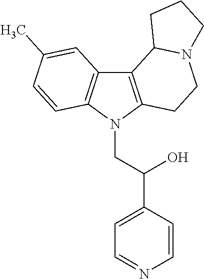 [SMILES: Cc1ccc2c(c1)c1c(n2CC(O)c2ccncc2)CCN2CCCC12] | 178 178a, 178b, 178c, 178d |

### General Synthetic Methods

The compounds of the invention may be prepared by a number of processes as generally described below and more specifically in the Examples hereinafter. In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

### General Protocol for Chiral Preparative HPLC Separation of Racemic Compounds

For chiral separations, samples were dissolved in MeOH and EtOH according to the solubility of sample and filtered through 0.22μ. PTFE filters. The columns used were CHIRALPAK-AD; 20*250 mm, 10μ and CHIRALCEL-ODH; 20*250 mm, 5μ. A flow rate of 12 mL/min-17 mL/min was used according to the resolution. Alkanes such as n-Pentane, Hexane and Heptane (40%-95%) and alcohols such as EtOH, Isopropyl alcohol and t-Butanol (5%-60%) were used as mobile phase. In some cases alcohol combinations i.e. (EtOH+MeOH), (EtOH+IPA), (IPA+MeOH), (t-Butanol+MeOH), (t-Butanol+EtOH) were used instead of a single alcohol. Diethyl amine (up to 0.3%) was used as modifier in the mobile phase.

### Example H1

### General Method for the Chiral HPLC Separation and Characterization of Compounds that were Synthesized Initially as a Mixture of Enantiomers

Crude or in some cases partially purified (normal or reverse phase HPLC) mixtures of enantiomers are analyzed by analytical chiral HPLC methods. Once adequate separation is achieved, larger quantities of the mixtures are separated using preparative scale columns. Separation is followed by removal of solvents on a rotary evaporator to accomplish the isolation of the individual single enantiomers. In some cases where appropriate, after removal of solvent, the samples are lyophilized. After isolation, each individual enantiomer is further analyzed by analytical (reverse phase and chiral) HPLC, LCMS and NMR. When final products are converted to salts, final characterization of the compounds is carried out after conversion to the salt for each enantiomer.

Analytical Chiral HPLC of Compounds of the Invention.

### Column: Chiralcel OD-H; Column ID: 4.6*250 mm, 5μ. Mobile Phase:

Hexane:(EtOH:MeOH 80:20)-93:7. Flow rate: 1 mL/min.

Chiral Preparative Data of Compounds of the Invention.

### Column: Chiralcel OD-H. Column ID: 20*250 mm, 5μ. Mobile Phase: Hexane:

(EtOH:MeOH 80:20)-95:5. Flow rate: 15 mL/min.

### Example H2

### General Method for the Chiral HPLC Separation and Characterization of Compounds that are Synthesized Initially as a Mixture of Diastereomers

Crude or in some cases partially purified (normal or reverse phase HPLC) mixtures of diastereomers are analyzed by analytical chiral HPLC methods. Once adequate separation is achieved, larger quantities of the mixtures are separated using preparative scale columns. Separation is followed by removal of solvents on a rotary evaporator to accomplish the isolation of the individual single diastereomers. In some cases where appropriate, after removal of solvent, the samples are lyophilized. Once each individual diastereomer is isolated they are further analyzed by analytical (reverse phase and chiral) HPLC, LCMS and NMR. When final products are converted to salts, final characterization of the compounds is carried out after conversion to the salt for each diastereomer.

Analytical Chiral HPLC Data of Compounds of the Invention.

Column: Chiral Pak AD-H. Column ID: 4.6*250 mm, 5μ. Mobile Phase: Hexane (0.2% diethylamine):Isopropanol-93:7. Flow rate: 1 mL/min.

Chiral Preparative Data of Compounds of the Invention.

Column: Chiral PAK-AD-H. Column ID: 20*250 mm, 5μ. Mobile Phase: Hexane (0.2% diethylamine):Isopropanol-93:7. Flow rate: 15 mL/min.

### Example H3

### Epimerization Method for Studying Chiral Compounds in Simulated Gastric Fluid (SGF) and Stimulated Intestinal Fluid (SIF)

### Incubation:

A measured quantity of sample was dissolved in SGF or SIF at the concentration of 1 mg/mL in a volumetric flask and appropriate number of aliquots of this solution were transferred to incubation vials as per the given time points. For the sample of zero hour, the appropriate volume of saturated Bicarbonate solution was added immediately to the sample, and was stirred for 5-10 mins. The compound was extracted in a suitable solvent (e.g. Ethyl acetate), decanting the organic layer. The organic solvent was evaporated, and the residue was dissolved in an appropriate solvent (Methanol/Ethanol), filtered through a 0.22 μm membrane filter and analyzed by chiral HPLC. The remaining aliquots were incubated at different temperatures i.e. 25° C. and 37° C. in a water bath as per the given time points. The respective samples were retrieved from the incubator/Water bath at different time intervals as per the given time points: 90 mins, 4 h, 6 h, 12 h and 24 h, and the same procedure for sample preparation was followed for the zero hour sample.

### Data Compilation:

All the chromatograms were obtained at the specified wavelength, compiled and the curves between Time Vs % Area were plotted.

### Results:

Compound No. 178d showed epimerization of up to 41.29% in SGF after 24 h @ 25° C., whereas Compound No. 79a showed only 2% epimerzation under the same conditions. Running the same study at the higher temperature of 37° C. resulted in 50.13% epimerization of Compound No. 178d, and 21.39% epimerization of Compound No. 79a.

The following abbreviations are used herein: thin layer chromatography (TLC); hour (h); minute (min); second (sec); ethanol (EtOH); dimethylsulfoxide (DMSO); N,N-dimethylformamide (DMF); trifluoroacetic acid (TFA salt); tetrahydrofuran (THF); Normal (N); aqueous (aq.); methanol (MeOH); dichloromethane (DCM); ethyl acetate (EtOAc); Retention factor (Rf); room temperature (RT).

Compounds detailed herein may be prepared by those of skill in the art by referral to General Methods and Examples described in published PCT applications WO2009/055828 (see e.g., General Methods 1-24 and Examples 1-325), WO2010/127177 (General Methods 1-3 and Examples 1-58), WO2009/120720 (General Methods 1-15C and Examples 1-134), WO2009/120717 (General Methods 1-17 and Examples 1-134), WO2010/051501 (General Methods 1-10 and Examples 1-450) and WO2010/051503 (General Methods 1-15 and Examples 1-111), WO2011/019417 (General Methods 1-9 and Examples 1-10), WO2011/038164 (General Methods 1-19), WO2011/038162 (General Methods 1-21 and Examples 1-6), WO2011/038163 (General Methods 1-19 and Examples 1-49), WO2011/038161 (General Methods 1-15B and Examples 1-22), WO2012/112966 (General Synthetic Methods and Examples 1-243), and WO2012/154261 (General Synthetic Methods and Examples 1-243). The PCT publications described above are incorporated herein by reference in their entireties. Particular examples of each of the General Methods and Examples are provided in the Examples below.

### General Method 1

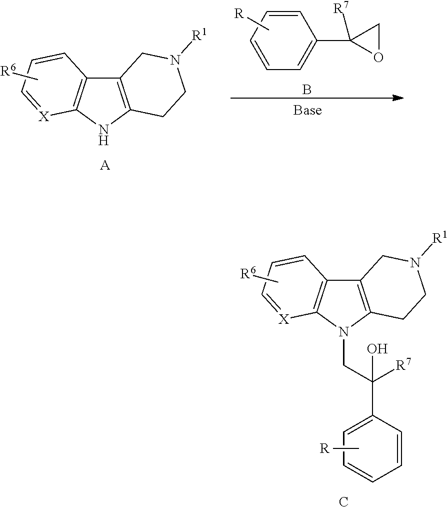

In certain examples of the compounds provided herein, and as similarly described in the publications presented above, alcohols of the type C can be prepared by treating appropriately functionalized carboline A with functionalized epoxide B, in the presence of a base. A selection of bases effective for this reaction will be apparent to those skilled in the art, such as for example, sodium hydride, sodium tert-butoxide, potassium tert-butoxide, lithium tert-butoxide, lithium diisopropylamide, lithium hexamethyldisilazide, sodium ethoxide, sodium methoxide, and the like. In some instances, one or more of the bases may be used interchangeably; for example, other bases such as sodium tert-butoxide, potassium tert-butoxide, lithium tert-butoxide, lithium diisopropylamide, lithium hexamethyldisilazide, sodium ethoxide or sodium methoxide may be substituted where sodium hydride is specifically described. It is understood that modifications to the specific materials shown are intended, e.g., where Compound B can be a heteroaryl group such as pyridyl, and Compound A can comprise structures such as pyrido[3,4-b]indoles, and the like.

The following Examples are provided to illustrate but not to limit the invention.

The Examples below, where appropriate, describe the preparation of compounds bearing stereocenters. In those Examples, the procedure describes the preparation of the racemate, wherefrom individual stereoisomers can be isolated, as described above. Analytical data of certain stereoisomers is presented.

### EXAMPLES

### Example 1

### Preparation of Compound No. 1

To a solution of 2-(2,8-dimethyl-3,4-dihydro-1H-pyrido[4,3-b]indol-5 (2H)-yl)-1-(pyridin-3-yl)ethyl methanesulfonate (0.5 g, 1.25 mmol) in DMSO (5 mL) was added sodium methanesulfinate (1.02 g, 10 mmol) and the reaction mixture was allowed to stir at 80° C. for 24 h. The progress of the reaction was monitored by LCMS. The reaction mixture was cooled to RT, diluted with water (50 mL) and extracted with EtOAc (3×25 mL). The combined organic layer was washed with water (6×50 mL) and dried over anhydrous sodium sulfate. Removal of EtOAc under reduced pressure gave a crude product (400 mg) that was purified by reverse phase HPLC to afford 40 mg of pure 2,8-dimethyl-5-(2-(5-(methylsulfonyl)pyridin-3-yl)ethyl)-2,3,4,5-tetrahydro-1H-pyrido[4,3-b]indole as a solid. ¹H NMR (CDCl₃, freebase) δ (ppm): 8.78 (s, 1H), 8.30 (s, 1H), 7.82 (d, 1H), 7.21 (m, 2H), 7.01 (d, 1H), 4.40 (t, 2H), 3.80 (m, 1H), 3.40 (t, 2H), 3.02 (t, 3H), 2.90-3.05 (m, 2H), 2.65-2.71 (m, 2H), 2.62 (s, 3H), 2.45 (s, 4H).

### Example 2

### Preparation of Compound Nos. 2, 2a and 2b

To a solution of 2,3,5,6,7,11c-hexahydro-1H-indolizino[7,8-b]indole (400 mg, 1.88 mmol) in 60% aq. NaOH (5 mL) was added 3-vinyl pyridine (396 mg, 3.77 mmol) and tetrabutyl ammonium bromide (607 mg, 1.88 mmol). The reaction mixture was allowed to stir at 100° C. for 18 h. The progress of reaction was monitored by TLC and LCMS. The reaction mixture was diluted with water (50 mL) and extracted with EtOAc (2×100 mL). The combined organic layer was washed with water (4×50 mL) and dried over sodium sulfate. Removal of EtOAc under reduced pressure gave a crude product that was purified by reverse phase HPLC to afford 60 mg of 2,3,5,6,7,11c-hexahydro-7-(2-(pyridin-3-yl)ethyl)-1H-indolizino[7,8-b]indole. The stereoisomers were separated by chiral HPLC. ¹H NMR (CDCl₃, freebase) δ (ppm): 8.46 (s, 1H), 8.30 (s, 1H), 7.50 (d, 1H), 7.15-7.25 (m, 3H), 7.06-7.18 (m, 2H), 4.21-4.36 (m, 1H), 3.18-3.30 (m, 1H), 2.98-3.10 (m, 2H), 2.71-2.95 (m, 2H), 2.38-2.58 (m, 2H), 2.15-2.28 (m, 1H), 1.80-1.95 (m, 2H), 1.20-1.27 (m, 3H).

### Example 3

### Preparation of Compound Nos. 3, 3a and 3b

To a solution of (E)-4-(2-(2,8-dimethyl-3,4-dihydro-1H-pyrido[4,3-b]indol-5 (2H)-yl)-1-(pyridin-4-yl)ethoxy)but-2-enoic acid (0.5 g, 1.7 mmol) in water (20 mL) was added Pd/C (0.2 g). Hydrogen gas was purged to the reaction mixture for 30 min. The progress of reaction was monitored by LCMS. The reaction mixture was filtered through a Celite bed and washed with water (10 mL). The filtrate was concentrated under reduced pressure to yield a crude product that was purified by reverse phase HPLC to afford 18 mg of 4-(2-(2,8-dimethyl-3,4-dihydro-1H-pyrido[4,3-b]indol-5 (2H)-yl)-1-(pyridin-4-yl)ethoxy)butanoic acid. ¹H NMR (CDCl₃, freebase) δ (ppm): 8.60 (d, 2H), 7.35 (d, 2H), 7.20 (s, 1H), 7.12 (d, 1H), 7.00 (d, 1H), 4.56 (dd, 1H), 4.36 (dd, 1H), 4.21 (dd, 1H), 3.95-4.15 (m, 2H), 3.56-3.64 (m, 1H), 3.40-3.54 (m, 1H), 3.25-3.38 (m, 1H), 3.10-3.24 (m, 1H), 2.92-3.02 (m, 2H), 2.84 (s, 3H), 2.42 (s, 3H), 2.02-2.21 (m, 2H), 1.59-1.81 (m, 2H).

### Example 4

### Preparation of Compound Nos. 4, 4a and 4b

To a solution of 2-(2,8-dimethyl-3,4-dihydro-1H-pyrido[4,3-b]indol-5 (2H)-yl)-1-(pyridin-4-yl)ethanol (200 mg, 0.62 mmol) in DMF (2 mL) was added NaH (60% dispersion in mineral oil, 74 mg, 1.8 mmol) at RT and the mixture was allowed to stir for 10 min. To this mixture was then added pyrrolidine-1-carbonyl chloride (165 mg, 1.2 mmol) and the reaction mixture was allowed to stir for 1 h. The progress of the reaction was monitored by LCMS. The reaction was quenched with ice cooled water (200 mL). The aqueous layer was extracted with EtOAc (2×200 mL). The combined organic layer was washed with water (3×100 mL), dried over sodium sulfate and concentrated to get the crude product that was purified by column chromatography on silica gel (100-200 mesh) using 0-10% MeOH-DCM system as eluent to afford 43 mg of 2-(2,8-dimethyl-3,4-dihydro-1H-pyrido[4,3-b]indol-5(2H)-yl)-1-(pyridin-4-yl)ethyl pyrrolidine-1-carboxylate as a free base. ¹H NMR (CDCl₃, freebase) δ (ppm): 8.58 (d, 2H), 7.25 (d, 1H), 7.18 (s, 1H), 7.08 (d, 2H), 6.96 (d, 1H), 5.95-6.00 (m, 1H), 4.45 (dd, 1H), 4.18 (dd, 1H), 3.55-3.59 (m, 2H), 3.25-3.43 (m, 4H), 2.47-2.81 (m, 3H), 2.56 (s, 3H), 2.39 (s, 3H), 2.21-2.39 (m, 1H), 1.78-1.98 (m, 4H).

### Example 5

### Preparation of Compound Nos. 5, 5a and 5b

To a solution of 2-(2,8-dimethyl-3,4-dihydro-1H-pyrido[4,3-b]indol-5 (2H)-yl)-1-(pyridin-4-yl)ethanol (200 mg, 0.62 mmol) in DMF (5 mL) was added NaH (60% dispersion in mineral oil) (74.4 mg, 1.86 mmol) at RT. The reaction mixture was stirred at the same temperature for 10 min. then 4-methylpiperazine-1-carbonyl chloride (162 mg, 1.2 mmol) was added and the reaction mixture further stirred for 1 h. The progress of the reaction was monitored by TLC and LCMS. The reaction was quenched with cold water (200 mL). The aqueous layer was extracted with EtOAc (3×200 mL). The combined organic layer was washed with water (3×100 mL), dried over sodium sulfate and concentrated to get the crude product which was purified by reverse phase HPLC to afford 44 mg of 2-(2,8-dimethyl-3,4-dihydro-1H-pyrido[4,3-b]indol-5 (2H)-yl)-1-(pyridin-4-yl)ethyl 4-methylpiperazine-1-carboxylate as a free base. ¹H NMR (DMSO-d₆, freebase) δ (ppm): 8.62 (brs, 2H), 7.22-7.39 (m, 3H), 7.15 (s, 1H), 6.91 (d, 1H), 5.78-5.92 (m, 1H), 4.35-4.45 (m, 2H), 4.00 (brs, 2H), 2.98-3.55 (m, 4H), 2.59-2.81 (m, 8H), 2.40 (s, 3H), 2.22 (s, 3H), 2.18-2.39 (m, 3H).

### Example 6

### Preparation of Compound Nos. 6, 6a, 6b, 6c and 6d

To a stirred solution of 10-methyl-2,3,5,6,7,11c-hexahydro-1H-indolizino[7,8-b]indole (250 mg, 1.106 mmol) in dry DMF (7 mL) at 0° C. was added NaH (110 mg, 2.765 mmol) portionwise. After 5 min. of stirring, a solution of 2-fluoro-5-(2-methyloxiran-2-yl) pyridine (304 mg, 1.99 mmol) in DMF (3 mL) was added dropwise. The reaction mixture was stirred at RT for 3 h. The reaction mixture was diluted with ice water and extracted with EtOAc (2×150 mL). The combined organic layer was washed with water (5×40 mL) and dried over anhydrous sodium sulfate. Removal of EtOAc under reduced pressure gave a crude product that was purified by reverse phase HPLC to obtain 200 mg of 2-(6-fluoropyridin-3-yl)-1-(10-methyl-2,3,5,6-tetrahydro-1H-indolizino[7,8-b]indol-7(11cH)-yl)propan-2-ol. ¹H NMR (CDCl₃, freebase) δ (ppm): 8.34 (s, 1H), 7.59-7.68 (m, 1H), 7.168 (s, 1H), 7.034 (d, 1H), 6.91 (d, 1H), 6.58-6.68 (m, 1H), 4.22 (dd, 1H), 4.12 (dd, 1H), 3.81-3.91 (m, 1H), 3.15-3.25 (m, 1H), 2.78-2.89 (m, 2H), 2.74-2.79 (m, 1H), 2.58-2.64 (m, 2H), 2.41 (s, 3H), 2.36-2.42 (m, 1H), 1.82-1.95 (m, 3H), 1.67 (s, 3H).

### Example 7

### Preparation of Compound Nos. 7, 7a and 7b

To a solution of 2-(10-methyl-2,3,5,6-tetrahydro-1H-indolizino[7,8-b]indol-7(11cH)-yl)-1-(pyridin-4-yl)ethyl methanesulfonate (1.0 g, 2.88 mmol) in NMP (20 mL) was added KOH powder (806 mg, 14.3 mmol) at 0° C. The reaction mixture was stirred for 15 min. at 0° C. then at RT for 90 min. The progress of reaction was monitored with LCMS. The reaction mixture was diluted with ice-cold water (100 mL) and extracted with EtOAc (3×50 mL). The combined organic layer was washed with water (3×100 mL), dried over sodium sulfate and concentrated under reduced pressure to obtain the crude product, which was purified by reverse phase HPLC to obtain 150 mg of (E)-10-methyl-7-(2-(pyridin-4-yl)vinyl)-2,3,5,6,7,11c-hexahydro-1H-indolizino[7,8-b]indole as a solid. ¹H NMR (CDCl₃, freebase) δ (ppm): 8.56 (d, 2H), 7.66 (d, 1H), 7.54 (d, 1H), 7.31 (d, 2H), 7.24 (d, 1H), 7.18 (d, 1H) 6.65 (d, 1H), 4.62-4.72 (m, 1H), 3.34-3.45 (m, 3H), 3.09-3.18 (m, 2H), 2.91-3.12 (m, 1H), 2.61-2.69 (m, 1H), 2.46 (s, 3H), 2.15-2.22 (m, 2H).

### Example 8

### Preparation of Compound Nos. 8, 8a and 8b

To a solution of 2-(2,8-dimethyl-1,2,3,4-tetrahydro-pyrido[4,3-b]indol-5-yl)-1-pyridin-4-yl-ethanol (321 mg, 1.00 mmol) in THF (3 mL) was added dropwise trichloroacetyl isocyanate (375 mg, 2 mmol) at RT. The reaction mixture was stirred at RT for 2 h. The progress of reaction was monitored by LCMS. The reaction mixture was basified with 10% potassium carbonate solution and extracted with EtOAc (3×50 mL) and dried over sodium sulfate. Removal of solvent under reduced pressure gave a crude product that was purified by reverse phase HPLC to afford 70 mg of 2-(2,8-dimethyl-3,4-dihydro-1H-pyrido[4,3-b]indol-5 (2H)-yl)-1-(pyridin-4-yl)ethyl carbamate. ¹H NMR (CDCl₃, freebase) δ (ppm): 8.58 (d, 2H), 7.19 (s, 1H), 7.25 (d, 1H), 7.18 (d, 2H), 6.61 (brs, 2H), 5.95-6.00 (m, 1H), 4.98 (s, 1H), 4.38 (dd, 1H), 4.18 (dd, 1H), 3.82 (dd, 1H), 3.75 (dd, 1H), 2.80-2.89 (m, 2H), 2.65-2.71 (m, 2H), 2.60 (s, 3H), 2.39 (s, 3H).

### Example 9

### Preparation of Compound Nos. 9, 9a and 9b

To a solution of 2-(2,8-dimethyl-1,2,3,4-tetrahydro-pyrido[4,3-b]indol-5-yl)-1-pyridin-4-yl-ethanol (2.0 g, 6.23 mmol) in DMSO (20 mL) was added methanesulfonic acid 2,2-dimethyl-propyl ester (2.01 g, 12.40 mmol) and potassium tertiary butoxide (2.09 g, 18.66 mmol) at RT. The reaction mixture was heated at 120° C. for 24 h. The progress of reaction was monitored by LCMS. The reaction mixture was poured in to ice-cold water and extracted with EtOAc (3×100 mL). The combined organic layer was dried over sodium sulfate. Removal of solvent under reduced pressure gave a crude product that was purified by reverse phase HPLC to obtain 2-(2,8-dimethyl-3,4-dihydro-1H-pyrido[4,3-b]indol-5 (2H)-yl)-4,4-dimethyl-1-(pyridin-4-yl)pentan-1-one as TFA salt. ¹H NMR (CDCl₃, TFA salt) δ (ppm): 8.82 (d, 2H), 8.22 (d, 2H), 8.15 (d, 1H), 7.65 (s, 1H), 7.38 (s, 1H), 7.18 (s, 1H), 4.8 (dd, 1H), 3.15 (s, 3H), 2.35 (s, 3H), 1.5 (d, 2H), 0.99 (s, 9H).

### Example 10

### Preparation of Compound Nos. 10, 10a, 10b, 10c and 10d

To a solution of 8-aza-2,3,5,6,7,11c-hexahydro-1H-indolizino[7,8-b]indole (180 mg, 0.52 mmol) in DCM (5 mL) was added a solution of Diethylaminosulfur trifluoride (166 mg, 1.03 mmol) in DCM (1 mL) dropwise at −78° C. The reaction mixture was stirred at the same temperature for 1 h. The reaction was quenched with saturated sodium bicarbonate solution and extracted with DCM (2×100 mL). The combined organic layer was dried over sodium sulfate. Removal of solvent gave a crude product, which was purified by reverse phase HPLC to afford 6 mg of 7-(2-fluoro-2-(6-methylpyridin-3-yl)ethyl)-8-aza-2,3,5,6,7,11c-hexahydro-1H-indolizino[7,8-b]indole. ¹H NMR (CDCl₃, TFA salt) δ (ppm): 8.62 (s, 1H), 8.21 (s, 1H), 7.98 (d, 1H), 7.74 (d, 1H), 7.18-7.21 (m, 1H), 6.05 (dd, 1H), 5.22 (brs, 1H), 4.70-4.95 (m, 4H), 3.75-3.82 (m, 2H), 3.35-3.45 (m, 2H), 2.74-2.81 (m, 1H), 2.15 (s, 3H), 2.22-2.39 (m, 4H).

### Example 11

### Preparation of Compound Nos. 11, 11a and 11b

To a suspension of 1-(8-methyl-1,2,3,4-tetrahydro-pyrido[4,3-b]indol-5-yl)-2-pyridin-3-yl-propan-2-ol (750 mg, 2.33 mmol) and potassium carbonate (967 mg, 7.0 mmol) in acetonitrile (10 mL) was added ethyl chloroformate (3.5 mg, 0.79 mmol) at RT and the reaction mixture was stirred for 1 h. The progress of reaction was monitored by TLC and LCMS. The reaction mixture was filtered, the filtrate was concentrated, and the residue obtained was diluted with water and extracted with EtOAc (3×50 mL). The combined organic layer was dried over sodium sulfate. Removal of solvent gave a crude product that was purified by column chromatography on silica gel (100-200 mesh) using 4% MeOH-DCM as eluent to obtain 600 mg of 5-(2-hydroxy-2-pyridin-3-yl-propyl)-8-methyl-1,3,4,5-tetrahydro-pyrido[4,3-b]indole-2-carboxylic acid ethyl ester. ¹H NMR (CDCl₃, freebase) δ (ppm): 8.75 (s, 1H), 8.54 (d, 1H), 7.61-7.79 (m, 1H), 7.24-7.35 (m, 2H), 7.21 (d, 1H), 6.99 (d, 1H), 4.61-4.71 (m, 2H), 4.21 (q, 2H), 4.23-4.25 (m, 2H), 3.65-3.79 (m, 2H), 2.46-2.62 (m, 1H), 2.44 (s, 3H), 2.12-2.18 (m, 1H), 1.66 (s, 3H), 1.28 (t, 3H).

### Example 12

### Preparation of Compound Nos. 12, 12a and 12b

A solution of methanesulfonic acid 2-(2-methyl-1,2,3,4-tetrahydro-pyrido[4,3-b]indol-5-yl)-1-pyridin-3-yl-ethylester (0.2 g, 0.519 mmol) in tertiary butyl amine (2 mL) was stirred at 100° C. for 18 h. The reaction was monitored by LCMS. After completion of reaction mixture it was concentrated to give crude product which was purified by reverse phase HPLC to obtain 5 mg of 2-methyl-N-(2-(2-methyl-3,4-dihydro-1H-pyrido[4,3-b]indol-5 (2H)-yl)-1-(pyridin-3-yl)ethyl)propan-2-amine. ¹H NMR (CDCl₃, freebase) δ (ppm): 8.5 (s, 1H), 8.47 (d, 1H), 7.56 (d, 1H), 7.4 (d, 1H), 7.27-7.31 (m, 1H), 7.19 (t, 2H), 7.08 (t, 1H), 4.3 (t, 1H), 4.15 (dd, 1H), 4.1 (dd, 1H), 3.76 (dd, 2H), 2.79-2.85 (m, 3H), 2.50 (s, 3H), 2.35-2.31 (m, 1H), 0.847 (s, 9H).

### Example 13

### Preparation of Compound Nos. 13, 13a, 13b, 13c and 13d

To a solution of 10-methyl-2,3,5,6,7,11c-hexahydro-1H-indolizino[7,8-b]indole (500 mg, 2.21 mmol) in DMF (5 mL) was added sodium hydride (159 mg, 6.63 mmol) at 0° C. After 5 min. of stirring, a solution of 4-(2-methyl-oxiranyl) pyridine (590 mg, 4.37 mmol) in DMF (2 mL) was added dropwise. The reaction mixture was allowed to stir at RT for 18 h. The progress of reaction was monitored by TLC and LCMS. After completion of the reaction ice-cold water was added to the reaction mixture and it was extracted with EtOAc (3×50 mL). The combined organic layer was washed with water (4×100 mL) and dried over sodium sulfate. Removal of the solvent under reduced pressure afforded a crude product which was recrystallized in ether-hexane to obtain 360 mg of 1-(10-methyl-2,3,5,6-tetrahydro-1H-indolizino[7,8-b]indol-7 (11cH)-yl)-2-(pyridin-4-yl)propan-2-ol. The optical isomers were separated by chiral HPLC. ¹H NMR (CDCl₃, freebase) δ (ppm): 8.56 (d, 2H), 7.34 (d, 2H), 7.23 (s, 1H), 7.19 (d, 1H), 6.96 (d, 1H), 4.19 (dd, 2H), 3.98-4.12 (m, 1H), 3.22-3.26 (m, 1H), 2.83-2.95 (m, 3H), 2.61-2.71 (m, 1H), 2.45-2.51 (m, 2H), 2.43 (s, 3H), 1.91-2.01 (m, 3H), 1.6 (s, 3H).

### Example 14

### Preparation of Compound Nos. 14, 14a, 14b, 14c and 14d

To a solution of 10-methyl-2,3,5,6,7,11c-hexahydro-1H-indolizino[7,8-b]indole (400 mg, 1.76 mmol) in DMF (5 ml) was added sodium hydride (212 mg, 5.3 mmol) portionwise at RT. After 10 min. of stirring, 3-fluoro-4-(oxiran-2-yl)pyridine (320 mg, 2.30 mmol) was added dropwise. The reaction mixture was stirred at RT for 12 h. The progress of reaction was monitored by TLC and LCMS. The reaction was quenched with Ice-cold water and the product was extracted with EtOAc (3×200 mL). The combined organic layer was washed with water (4×150 mL) and dried over sodium sulfate. Removal of solvent under reduced pressure gave a crude product that was purified by reverse phase HPLC to afford 400 mg of 1-(3-fluoropyridin-4-yl)-2-(10-methyl-2,3,5,6-tetrahydro-1H-indolizino[7,8-b]indol-7 (11cH)-yl)ethanol as a freebase. ¹H NMR (CD₃OD, freebase) δ (ppm): 8.39 (s, 1H), 8.31-8.38 (m, 1H), 7.61-6.71 (m, 1H), 6.98-7.23 (m, 2H), 6.99 (t, 1H), 5.37-5.44 (m, 1H), 4.36 (dd, 1H), 4.26 (dd, 1H), 3.57-3.65 (m, 3H), 3.25-3.32 (m, 1H), 3.15-3.24 (m, 2H), 2.61-2.68 (m, 1H), 2.4 (s, 3H), 2.17-2.22 (m, 4H).

### Example 15

### Preparation of Compound Nos. 15, 15a, 15b, 15c and 15d

To a solution of a mixture of (2,8-dimethyl-2,3,4,5-tetrahydro-1H-pyrido[4,3-b]indol-1-yl)methanol and (2,8-dimethyl-2,3,4,5-tetrahydro-1H-pyrido[4,3-b]indol-3-yl)methanol (500 mg, 2.17 mmol) in DMF (6 mL) was added sodium hydride (260 mg, 6.51 mmol) portionwise at 0° C. After stirring for 15 min. at 0° C., a solution of 3-(2-methyloxiran-2-yl) pyridine (439 mg, 3.25 mmol) in DMF (2 mL) was added dropwise. The reaction mixture was allowed to stir at RT for 16 h. The reaction mixture was poured on crushed ice-cold water (50 mL). The product was extracted with EtOAc (2×70 mL) and dried over anhydrous sodium sulfate. Removal of EtOAc under reduced pressure gave a crude mixture of regioisomers that were separated by reverse phase HPLC. These regioisomers were subjected to chiral HPLC to afford 20 mg of 1-(3-(hydroxymethyl)-2,8-dimethyl-3,4-dihydro-1H-pyrido[4,3-b]indol-5 (2H)-yl)-2-(pyridin-3-yl)propan-2-ol. ¹H NMR (CDCl₃, freebase) δ (ppm): 8.7 (s, 1H), 8.5 (d, 1H), 7.72 (d, 1H), 7.26 (t, 1H), 7.16-7.21 (m, 2H), 6.96 (d, 1H), 4.16 (dd, 2H), 3.93 (d, 1H), 3.8 (d, 1H), 3.62-3.75 (m, 2H), 2.95-3.11 (m, 1H), 2.50-2.68 (m, 2H), 2.41 (s, 6H), 1.36 (s, 3H).

### Example 16

### Preparation of Compound Nos. 16, 16a, 16b, 16c and 16d

To a solution of a mixture of (2,8-dimethyl-2,3,4,5-tetrahydro-1H-pyrido[4,3-b]indol-1-yl)methanol and (2,8-dimethyl-2,3,4,5-tetrahydro-1H-pyrido[4,3-b]indol-3-yl)methanol (500 mg, 2.17 mmol) in DMF (6 mL) was added sodium hydride (260 mg, 6.51 mmol) portionwise at 0° C. After stirring for 15 min. at 0° C., a solution of 3-(2-methyloxiran-2-yl) pyridine (439 mg, 3.25 mmol) in DMF (2 mL) was added dropwise. The reaction mixture was allowed to stir at RT for 16 h. The reaction mixture was poured on crushed ice-cold water (50 mL). The product was extracted with EtOAc (2×70 mL) and dried over anhydrous sodium sulfate. Removal of EtOAc under reduced pressure gave a crude mixture of regioisomers that were separated by reverse phase HPLC. These regioisomers were subjected to chiral HPLC to afford 30 mg of 1-(1-(hydroxymethyl)-2,8-dimethyl-3,4-dihydro-1H-pyrido[4,3-b]indol-5 (2H)-yl)-2-(pyridin-3-yl)propan-2-ol. ¹H NMR (CDCl₃, freebase) δ (ppm): 8.63 (s, 1H), 8.44 (s, 1H), 7.62 (dd, 1H), 7.21-7.29 (m, 3H), 6.97 (d, 1H), 4.17-4.25 (m, 2H), 3.81 (dd, 2H), 3.55-3.61 (m, 1H), 2.98-3.15 (m, 1H), 2.74-2.79 (m, 2H), 2.44 (s, 6H), 2.42-2.49 (m, 2H), 1.63 (d, 3H).

### Example 17

### Preparation of Compound Nos. 17, 17a and 17b

To a suspension of NaH (283 mg, 11.8 mmol, 60% dispersion in mineral oil) in DMF (15 mL) was added 6,9-dichloro-2-methyl-2,3,4,5-tetrahydro-1H-pyrido[4,3-b]indole (1 g, 3.9 mmol) slowly at 0° C. and the mixture was allowed stir for 30 min. Then, a solution of 3-(oxiran-2-yl)pyridine (952 mg, 7.8 mmol) in DMF (1 mL) was added at 0° C. The ice bath was removed and the reaction mixture was allowed to stir at RT for 20 h. The reaction mixture was poured into ice-cold water (150 mL) and the product was extracted with DCM (2×40 mL). The combined organic layer was washed with water (8×30 mL) and dried over anhydrous sodium sulfate. Removal of DCM under reduced pressure gave a crude product that was purified by reverse phase HPLC to afford 140 mg of 2-(6,9-dichloro-2-methyl-3,4-dihydro-1H-pyrido[4,3-b]indol-5 (2H)-yl)-1-(pyridin-3-yl)ethanol. ¹H NMR (CD₃OD, TFA salt) δ (ppm): 8.91 (brs, 1H), 8.81 (brs, 1H), 8.59-8.62 (m, 1H), 8.01 (brs, 1H), 7.16 (d, 1H), 7.06 (d, 1H), 5.35 (brs, 1H), 5.15-5.19 (m, 1H), 4.60 (t, 1H), 4.38-4.45 (m, 1H), 3.82-3.89 (m, 1H), 3.42-3.59 (m, 2H), 3.20-3.44 (m, 2H), 3.14 (s, 3H).

### Example 18

### Preparation of Compound Nos. 18, 18a, 18b, 18c and 18d

To a solution of 10-fluoro-2,3,5,6,7,11c-hexahydro-1H-indolizino[7,8-b]indole (250 mg, 1.08 mmol) in DMF (5 mL) was added sodium hydride (129 mg, 3.2 mmol) at 0° C. After 5 min. of stirring, 4-oxiranyl pyridine (210 mg, 1.7 mmol) was added and the reaction mixture was allowed to stir at RT for 3 h. The reaction mixture was poured in to ice-cold water and extracted with EtOAc (3×100 mL). The combined organic layer was washed with water (3×25 ml) and dried over sodium sulfate. Removal of solvent under reduced pressure gave a crude product that was purified by reverse phase HPLC to obtain 47 mg of 2-(10-fluoro-2,3,5,6-tetrahydro-1H-indolizino[7,8-b]indol-7 (11cH)-yl)-1-(pyridin-4-yl)ethanol. ¹H NMR (CD₃OD, TFA salt) δ (ppm): 8.68 (brs, 2H), 7.91 (d, 2H), 7.35-7.44 (m, 1H), 7.22 (d, 1H), 6.94-6.99 (m, 2H), 5.12-5.24 (m, 1H), 5.05-5.11 (m, 1H), 4.42-4.51 (m, 1H), 4.25-4.37 (m, 1H), 3.69-3.82 (m, 3H), 3.18-3.42 (m, 1H), 3.15-3.20 (m, 2H), 2.61-2.72 (m, 1H), 2.18-2.20 (m, 3H).

### Example 19

### Preparation of Compound Nos. 19, 19a, 19b, 19c and 19d

To a suspension of NaH (500 mg, 20.8 mmol, 60% dispersion in mineral oil) in DMF (20 mL) was added 8,10-dimethyl-2,3,5,6,7,11c-hexahydro-1H-indolizino[7,8-b]indole (1 g, 4.0 mmol) slowly at 0° C. and the mixture was allowed stir for 30 min. Then, a solution of 4-(2-methyloxiran-2-yl)pyridine (1.1 g, 90 mmol) in DMF (1 mL) was added at 0° C. The ice bath was removed and the reaction mixture was allowed to stir at RT overnight. The reaction mixture was poured into ice-cold water (150 mL) and the product was extracted with EtOAc (2×40). The combined organic layer was washed with water (8×30 mL) and dried over anhydrous sodium sulfate. Removal of solvent under reduced pressure gave a crude product that was washed with hexane (2×30 mL) followed by ether. The solid was filtered to afford 200 mg of 1-(8,10-dimethyl-2,3,5,6-tetrahydro-1H-indolizino[7,8-b]indol-7(11cH)-yl)-2-(pyridin-4-yl)propan-2-ol as off white solid. ¹H NMR (CDCl₃, freebase) δ (ppm): 8.57 (d, 2H), 7.26 (d, 2H), 7.18 (s, 1H), 6.74 (s, 1H), 4.42-4.58 (m, 1H), 4.21-4.38 (m, 1H), 4.07-4.15 (m, 1H), 3.13-3.19 (m, 1H), 2.75-2.78 (m, 2H), 2.74 (s, 3H), 2.39 (s, 6H), 2.15 (s, 1H), 1.87 (brs, 3H), 1.51 (s, 3H).

### Example 20

### Preparation of Compound Nos. 20, 20a and 20b

To a suspension of NaH (327 mg, 13.6 mmol, 60% dispersion in mineral oil) in DMF (12 mL) was added 6-chloro-2-methyl-2,3,4,5-tetrahydro-1H-pyrido[4,3-b]indole (1 g, 4.5 mmol) slowly at 0° C. and the mixture was allowed stir for 30 min. Then, a solution of 3-(oxiran-2-yl)pyridine (1.1 g, 90 mmol) in DMF (1 mL) was added at 0° C. The ice bath was removed and the reaction mixture was allowed to stir at RT overnight. The reaction mixture was poured into ice-cold water (150 mL) and the product was extracted with EtOAc (2×40 mL). The combined organic layer was washed with water (8×30 mL) and dried over anhydrous sodium sulfate. Removal of solvent under reduced pressure gave a crude product that was purified by reverse phase HPLC to afford 550 mg of 2-(6-chloro-2-methyl-3,4-dihydro-1H-pyrido[4,3-b]indol-5-(2H)-yl)-1-(pyridin-3-yl)ethanol. ¹H NMR (CD₃OD, freebase) δ (ppm): 8.43 (d, 1H), 8.31 (s, 1H), 7.76 (d, 1H), 7.39 (t, 1H), 7.33 (d, 1H), 7.10 (d, 1H), 6.98 (t, 1H), 5.18 (t, 1H), 4.63 (dd, 1H), 4.57 (dd, 1H), 3.72 (d, 1H), 3.57 (d, 1H), 3.29-3.35 (m, 1H), 3.03-3.15 (m, 1H), 2.78-2.88 (m, 1H), 2.59-2.65 (m, 1H), 2.5 (s, 3H).

### Example 21

### Preparation of Compound Nos. 21, 21a, 21b, 21c and 21d

To a solution of 9-chloro-10-methyl-2,3,5,6,7,11c-hexahydro-1H-indolizino[7,8-b]indole (200 mg, 0.76 mmol) in DMF (2 mL) was added sodium hydride (92 mg, 2.3 mmol) at 0° C. After 5 min. of stirring, 4-oxiranyl-pyridine (186 mg, 1.53 mmol) was added and the reaction mixture was allowed to stir at RT for 5 h. The reaction mixture was poured in to ice-cold water and the product was extracted with EtOAc (4×50 mL). The combined organic layer was washed with water (4×50 mL) and dried over sodium sulfate. Removal of solvent under reduced pressure gave a crude product that was purified by reverse phase HPLC to obtain 60 mg of 2-(8-chloro-9-methyl-1,2,3,4,5,10c-hexahydro-3a, 6-diaza-cyclopenta[c]fluoren-6-yl)-1-pyridin-4-yl-ethanol. ¹H NMR (CD₃OD, TFA salt) δ (ppm): 8.67 (brs, 2H), 7.89 (d, 2H), 7.41 (s, 1H), 7.25 (d, 1H), 5.21-5.25-5.24 (m, 1H), 5.04-5.12 (m, 1H), 4.41-4.46 (m, 1H), 4.21-4.36 (m, 1H), 3.66-3.78 (m, 3H), 3.25-3.41 (m, 1H), 3.15-3.25 (m, 1H), 2.63-2.72 (m, 1H), 2.41 (s, 3H), 2.12-2.22 (m, 4H).

### Example 22

### Preparation of Compound Nos. 22, 22a, 22b, 22c and 22d

To a solution of 10-methyl-2,3,5,6,7,11c-hexahydro-1H-indolizino[7,8-b]indole (100 mg, 0.442 mmol) in DMF (1 mL) was added NaH (53 mg, 1.325 mmol) portionwise at 0° C. After 5 min. of stirring, a solution of 4-4-(oxiran-2-yl)pyridine-1-oxide (121 mg, 0.883 mmol) in DMF (1 mL) was added dropwise at the same temperature. The reaction mixture was brought to RT and allowed to stir for 18 h. The reaction mixture was diluted with ice-cold water and concentrated and purified by reverse phase HPLC obtained 40 mg of 4-(1-hydroxy-2-(10-methyl-2,3,5,6-tetrahydro-1H-indolizino[7,8-b]indol-7 (11cH)-yl)ethyl)pyridine 1-oxide. ¹H NMR (CD₃OD, TFA salt) δ (ppm): 8.23 (t, 2H), 7.43 (d, 2H), 7.22 (d, 1H), 7.21 (d, 1H), 7.00 (t, 1H), 5.11 (t, 1H), 5.03 (m, 1H), 4.33 (m, 2H), 3.70 (m, 1H), 3.63 (m, 2H), 3.4 (m, 1H), 3.19 (m, 2H), 3.05 (m, 1H), 2.7 (m, 1H), 2.4 (s, 3H), 2.19 (m, 3H).

### Example 23

### Preparation of Compound Nos. 23, 23a, 23b, 23c and 23d

To a solution of 2-(10-methyl-2,3,5,6-tetrahydro-1H-indolizino[7,8-b]indol-7 (11cH)-yl)-1-(pyridin-4-yl)ethanol (150 mg, 0.43 mmol) in dichloromethane (10 mL) was added meta-chloroperbenzoic acid (75 mg, 0.43 mmol) and the resulting mixture was allowed to stir 30 min. The progress of reaction was monitored by LCMS. To the reaction mixture was added a saturated sodium bicarbonate solution (10 mL) was added and the product was extracted with DCM (2×25 mL). The combined organic layer was dried over sodium sulfate. Removal of solvent under reduced pressure gave a crude product that was purified by reverse phase HPLC to afford 100 mg of 7-(2-hydroxy-2-(pyridin-4-yl)ethyl)-10-methyl-1,2,3,4,5,6,7,11c-octahydroindolizino[7,8-b]indole 4-oxide compound. ¹H NMR (CD₃OD, TFA salt) δ (ppm): 8.671 (d, 2H), 7.92 (d, 2H), 7.25 (s, 1H), 7.2 (d, 1H), 7.01 (d, 1H), 5.22-5.78 (m, 2H), 4.38-4.45 (m, 2H), 4.12-4.21 (m, 4H), 3.42-3.45 (m, 1H), 3.20-3.27 (m, 1H), 2.98-3.17 (m, 1H), 2.48-2.56 (m, 1H), 2.42 (s, 3H), 2.25-2.35 (m, 1H).

### Example 24

### Preparation of Compound Nos. 24, 24a, 24b, 24c and 24d

A solution of 2-(3-{[(2,2-dimethoxy-ethyl)-methyl-amino]-methyl}-5-methyl-indol-1-yl)-1-pyridin-3-yl-ethanol (1.0 g, 2.61 mmol) in 6N HCl (26 ml) at 0° C. was stirred for 2 h. Then reaction mixture was brought to RT and the reaction mixture was allowed to stir for another 18 h. The progress of reaction was monitored by TLC. The reaction mixture was poured in to ice-cold water, basified with aq. ammonia and extracted with EtOAc (4×50 mL). The combined organic layer was dried over sodium sulfate. Removal of solvent under reduced pressure gave a crude product that was purified by reverse phase HPLC to afford 5-(2-Hydroxy-2-pyridin-3-yl-ethyl)-2,8-dimethyl-2,3,4,5-tetrahydro-1H-pyrido[4,3-b]indol-4-ol. ¹H NMR (CD₃OD, TFA salt) δ (ppm): 8.40-8.61 (m, 2H), 8.20-8.27 (m, 1H), 7.61-7.71 (m, 1H), 7.25 (s, 1H), 7.12-7.21 (s, 1H), 7.06 (m, 1H), 5.20-5.50 (m, 2H), 4.68-4.78 (m, 1H), 4.50-4.59 (m, 2H), 34.21-4.35 (m, 1H), 3.61-3.71 (m, 2H), 3.16 (s, 3H), 2.39 (s, 3H).

### Example 25

### Preparation of Compound Nos. 25, 25a and 25b

To a 500 mL three-neck round bottle flask was added 1-(2,8-dimethyl-3,4-dihydro-1H-pyrido[4,3-b]indol-5(2H)-yl)-2-(pyridin-3-yl)propan-2-ol (10.0 g, 29.8 mmol) in DCM (120 mL) at RT. Triethylamine (6.34 mL, 45.4 mmol) was charged to the mixture, followed by addition of 4-dimethylaminopyridine (0.87 g, 7.1 mmol). After 10 min, propionic anhydride (7.11 mL, 55.5 mmol) was added in one portion. The mixture was stirred at RT for 36 h. The reaction was diluted with sat. sodium bicarbonate (150 mL) to adjust the pH to 9-10. The mixture was extracted with DCM (3×50 mL). The combined organic layers were washed with brine (2×100 mL). The organic layers were dried over anhydrous sodium sulfate. After evaporation, the mixture was purified on the silica gel column (DCM-MeOH-triethylamine, 95:5:0.2, v/v/v). The compound was dried under vacuum for 16 h to afford 4.75 g (41% yield) of a light yellow solid.

### Example 26

### Preparation of Compound Nos. 26, 26a and 26b

To a 500 mL three-neck round bottle flask was added 1-(2,8-dimethyl-3,4-dihydro-1H-pyrido[4,3-b]indol-5(2H)-yl)-2-(pyridin-3-yl)propan-2-ol (10.0 g, 29.8 mmol) in DCM (120 mL) at RT. Triethylamine (6.34 mL, 45.4 mmol) was charged to the mixture, followed by addition of 4-dimethylaminopyridine (0.87 g, 7.1 mmol). After 10 min, benzoic anhydride (12.54 g, 55.5 mmol) was added in one portion. The mixture was stirred at RT for 36 h. The reaction was diluted with sat. sodium bicarbonate (150 mL) to adjust the pH to 9-10. The mixture was extracted with DCM (3×50 mL). The combined organic layers were washed with brine (2×100 mL). The organic layers were dried over anhydrous sodium sulfate. After evaporation, the mixture was purified on the silica gel column (DCM-MeOH-triehtylamine, 95:5:0.2, v/v/v). The compound was dried under vacuum for 16 h to afford 3.20 g of a light yellow solid.

### Example 27

### Preparation of Compound Nos. 27, 27a, 27b, 27c and 27d

To a solution of (R)-7-methyl-2,3,5,10,11,11a-hexahydro-1H-indolizino[7,6-b]indole (150 mg, 0.663 mmol) in DMF (10 mL) at 0° C. was added sodium hydride (79 mg, 1.989 mmol). After 5 min. of stirring, 3-(oxiran-2-yl)pyridine (96 mg, 0.796 mmol) was added and the reaction was allowed to stir at RT for 12 h. The reaction was quenched with ice-cold water and extracted with EtOAc (2×50 mL). The organic layer was washed with water (4×20 mL) and dried over anhydrous sodium sulfate. Removal of solvent under reduced pressure gave crude product that was purified by reverse phase HPLC to afford 123 mg of 2-(R)-7-methyl-2,3-dihydro-1H-indolizino[7,6-b]indol-10(5H,11H,11aH)-yl)-1-(pyridin-3-yl)ethanol as the TFA salt. ¹H NMR (CD₃OD, TFA salt): 8.60 (brs, 1H), 8.54 (brs, 1H), 8.22-8.32 (m, 1H), 7.72-7.80 (m, 1H), 7.24 (d, 1H), 7.00-7.15 (m, 1H), 6.91-7.02 (m, 1H), 5.20-5.29 (m, 1H), 4.82 (brs, 1H), 4.24-4.52 (m, 3H), 3.72-3.98 (m, 2H), 3.61-3.71 (m, 1H), 3.42-3.58 (m, 1H), 3.25-3.42 (m, 1H), 3.02-3.21 (m, 1H), 2.61 (brs, 1H), 2.39 (s, 3H), 2.18-2.42 (m, 1H), 1.92-2.04 (m, 1H).

### Example 28

### Preparation of Compound Nos. 28, 28a and 28b

To a solution of 4-(1-(2,8-dimethyl-3,4-dihydro-1H-pyrido[4,3-b]indol-5(2H)-yl)-2-hydroxypropan-2-yl)benzoic acid (100 mg, 0.263 mmol) in DCM (20 mL) at RT was added EDC.HCl (60 mg, 0.316 mmol). After 5 minute of stirring, was added a solution pyrrolidine (28 mg, 0.394 mmol) in DCM (1 mL) and the reaction mixture was allowed to stir at RT for 18 h. The reaction mixture was diluted with saturated solution of NaHCO₃ (50 mL) and extracted with DCM (2×100 mL). The organic layer was dried over sodium sulfate concentrated under vacuum to obtain crude product that was purified by column chromatography on silica gel (100-200 mesh) using MeOH-DCM (0-50%) system as eluent to afford 30 mg of (4-(1-(2,8-dimethyl-3,4-dihydro-1H-pyrido[4,3-b]indol-5(2H)-yl)-2-hydroxypropan-2-yl)phenyl) (pyrrolidin-1-yl)methanone. ¹H NMR (CDCl₃, freebase): 8.67 (s, 1H), 7.81 (d, 1H), 7.71 (d, 1H), 7.12 (s, 1H), 7.06 (s, 1H), 6.89 (d, 1H), 4.20 (d, 1H), 4.13 (d, 1H), 3.52-3.78 (m, 6H), 2.62-2.82 (m, 4H), 2.54 (s, 3H), 2.41 (s, 3H), 1.80-2.01 (m, 4H), 1.65 (s, 3H).

### Example 29

### Preparation of Compound Nos. 29, 29, 29b, 29c and 29d

To a solution of ethyl 7-(2-hydroxy-2-(pyridin-4-yl)ethyl)-2,3,5,6,7,11c-hexahydro-1H-indolizino[7,8-b]indole-10-carboxylate (200 mg, 0.49 mmol) in dry THF (8 mL) was added lithiumaluminum hydride (56 mg, 1.4 mmol) at RT under nitrogen and the reaction mixture was allowed to stir for 1 h. The reaction was quenched with ice at −78° C. and the product was extracted with EtOAc (3×20 mL). The combined organic layer was dried over sodium sulfate and concentrated under reduced pressure to obtain 140 mg of 2410-(hydroxymethyl)-2,3,5,6-tetrahydro-1H-indolizino[7,8-b]indol-7(11cH)-yl)-1-(pyridin-4-yl)ethanol. ¹H NMR (CD₃OD, TFA salt): 8.63 (brs, 2H), 7.89 (brs, 2H), 7.48 (s, 1H), 7.22-7.32 (m, 1H), 7.17-7.21 (m, 1H), 5.26 (brs, 1H), 5.09 (t, 1H), 4.67 (s, 2H), 4.41-4.56 (m, 1H), 4.36-4.41 (m, 1H), 3.60-3.81 (m, 3H), 3.10-3.46 (m, 3H), 3.71 (brs, 1H), 2.10-2.31 (m, 3H).

### Example 30

### Preparation of Compound Nos. 30, 30a and 30b

To a solution of 4,4-difluoro-2,3,4,5-tetrahydro-2,7-dimethyl-1H-pyrido[4,3-b]indole (10 mg, 0.0423 mmol) in DMF (2 mL) was added NaH (4 mg, 0.0847 mmol) at 0° C. After 5 min. of stirring, 3-(oxiran-2-yl)pyridine (10 mg, 0.0847 mmol) in DMF (1 mL) was added dropwise and the reaction mixture was stirred at RT overnight. The reaction was monitored by LCMS. After complete consumption of starting material, the reaction mixture was quenched with ice and the aqueous layer was extracted with EtOAc. The organic layer was dried over sodium sulfate and concentrated under reduced pressure to obtain crude product that was purified by preparative HPLC to afford 1.6 mg of 2-(4,4-difluoro-2,8-dimethyl-3,4-dihydro-1H-pyrido[4,3-b]indol-5(2H)-yl)-1-(pyridin-3-yl)ethanol. ¹HNMR (CD₃OD, TFA salt): 8.59 (d, 1H), 8.48 (d, 1H), 8.14 (d, 1H), 7.69-7.76 (m, 1H), 7.40 (s, 1H), 7.33 (d, 1H), 7.16 (d, 1H), 5.25-5.36 (m, 1H), 4.60-4.70 (m, 1H), 4.20-4.60 (m, 3H), 3.98-4.16 (m, 2H), 3.18 (s, 3H), 2.42 (s, 3H).

### Example 31

### Preparation of Compound Nos. 31, 31a, 31b, 31c and 31d

To a solution of (R)-10-fluoro-2,3,5,6,7,11c-hexahydro-1H-indolizino[7,8-b]indole (240 mg, 1.043 mmol) in DMF (3 mL) at 0° C. was added sodium hydride (125 mg, 3.125 mmol). After 5 min. of stirring, to this was added a solution of 4-(2-methyl-oxiranyl)pyridine (225 mg, 1.66 mmol) in DMF (1 mL) and the reaction mixture was allowed to stir at RT for 5 h. The reaction mixture was poured into ice-cold water and extracted with EtOAc (4×50 mL). The combined organic layer was washed with water (5×20 mL) and dried over sodium sulfate. Removal of solvent under reduced pressure gave crude product that was recrystallized from ether-hexane to obtain 120 mg of diastereomeric mixture of 1-(9-Fluoro-1,2,3,4,5,10c-hexahydro-3a,6-diaza-cyclopenta[c]fluoren-6-yl)-2-pyridin-4-yl-propan-2-ol. The mixture was subjected to chiral separation to afford 50 mg of (R)-1-((R)-10-fluoro-2,3,5,6-tetrahydro-1H-indolizino[7,8-b]indol-7(11cH)-yl)-2-(pyridin-4-yl)propan-2-ol. ¹H NMR (CDCl₃, freebase): 8.5 (d, 2H), 7.3 (d, 2H), 7.19 (m, 1H), 7.0 (d, 1H), 6.8 (t, 1H), 4.2 (dd, 2H), 3.8 (t, 1H), 3.3-3.1 (m, 1H), 2.9-2.8 (m, 2H), 2.8-2.7 (m, 1H), 2.7-2.5 (m, 3H), 2.4-2.3 (m, 1H), 2-1.8 (m, 2H), 1.65 (s, 3H).

### Example 32

### Preparation of Compound Nos. 32, 32a and 32b

2,8-Dimethyl-2,3,4,5-tetrahydro-1H-pyrido[4,3-b]indole (80 mg, 0.4 mmol) was charged in a reaction vessel with DMF (2 mL). To this was added NaH (48 mg, 1.2 mmol) and stirred for 5 min. Then 3-(2-methyloxiran-2-yl)quinoline (88 mg, 0.48 mmol) was added and the reaction was stirred at RT overnight. The reaction was monitored by LCMS. The reaction was quenched with ice cool water and extracted with EtOAc (4×50 mL). The organic layer was concentrated and the crude thus obtained was purified by preparative HPLC to get 13 mg of required product as the TFA salt. ¹HNMR (CD₃OD, TFA salt): 8.96 (s, 1H), 8.70 (d, 1H), 8.18 (d, 1H), 8.05 (m, 2H), 7.85 (m, 1H), 7.11 (s, 1H), 6.60 (d, 1H), 6.40 (d, 1H), 4.82 (m, 1H), 4.50-4.30 (m, 3H), 3.82 (m, 1H), 3.80-3.60 (m, 2H), 3.50 (m, 1H), 3.10 (s, 3H), 2.20 (s, 3H), 1.90 (s, 3H).

### Example 33

### Preparation of Compound Nos. 33, 33a and 33b

To a solution of 5-(1-(2,8-dimethyl-3,4-dihydro-1H-pyrido[4,3-b]indol-5(2H)-yl)-2-hydroxypropan-2-yl)picolinic acid (100 mg, 2.638 mmol) in DCM (5 mL) was added EDC.HCl (60 mg, 3.166 mmol) and 1-cyclopropyl-N-methylmethanamine (33 mg, 3.9577 mmol) at RT. The reaction mixture was allowed to stir at RT for 3 h. The DCM was removed under reduced pressure to obtain crude product that was purified by reverse phase HPLC to afford 16 mg of N-(cyclopropylmethyl)-5-(1-(2,8-dimethyl-3,4-dihydro-1H-pyrido[4,3-b]indol-5(2H)-yl)-2-hydroxypropan-2-yl)-N-methylpicolinamide as a TFA salt. ¹H NMR (CD₃OD, TFA salt): 8.27-8.45 (dd, 1H), 7.98 (d, 1H), 7.38-7.45 (m, 1H), 7.18-7.22 (m, 1H), 6.80-6.90 (m, 2H), 4.62-4.72 (dd, 1H), 4.38 (s, 3H), 3.80 (brs, 1H), 3.41-3.60 (m, 3H), 3.40 (s, 3H), 3.02 (s, 6H), 2.80 (d, 2H), 2.40 (d, 2H), 1.80 (s, 1H), 1.00 (d, 1H), 0.60 (m, 2H), 0.20-0.40 (m, 2H).

### Example 34

### Preparation of Compound Nos. 34, 34a, 34b, 34c and 34d

To a stirred solution of beta-BOC-carboline (50 mg, 0.20 mmol) in dry DMF (2 mL) at 25° C. was added sodium hydride (5.53 g, 0.138 mol 60%) portionwise under nitrogen atmosphere. After 5 min., to this was added a solution of 4-(oxiran-2-yl)pyridine (56 mg, 0.619 mmol) in DMF (0.5 mL) dropwise at 25° C. After complete addition, the reaction mixture was stirred at 25° C. overnight. The desired product was detected by LCMS. The reaction mixture was slowly poured into ice-cold water and extracted with EtOAc, organic layer washed with water (5 times). The organic layer was dried over anhydrous sodium sulfate, and concentrated under vacuum to obtain crude product that was used in the next step without any purification. The crude BOC compound was dissolved in 2M HCl solution (20 mL) and stirred at RT overnight. The reaction mixture was concentrated under vacuum to obtain crude product that purified by reverse phase HPLC to obtain 20 mg of mixture of desired products. The optical isomers were separated by chiral HPLC to obtain 5 mg of desired product. ¹HNMR (freebase, CDCl₃): 8.60 (d, 2H), 7.30 (d, 2H), 7.28-7.18 (m, 2H), 7.02 (d, 1H), 5.10 (m, 1H), 4.40 (d, 1H), 4.18 (dd, 1H), 4.05-3.95 (m, 2H), 2.62 (m, 1H), 2.42 (s, 3H), 2.40 (m, 2H), 1.90 (m, 2H), 1.70 (m, 2H), 1.50 (m, 2H).

### Example 35

### Preparation of Compound Nos. 35, 35a and 35b

To a solution of 2-(2,8-dimethyl-3,4-dihydro-1H-pyrido[4,3-b]indol-5(2H)-yl)-1-(pyridin-4-yl)ethanol (500 mg, 1.5 mmol) in DMF (5 mL) at 0° C. was added sodium hydride (300 mg, 5.5 mmol) portionwise. After 5 min. of stirring, to this was added bromocyclopentane (680 mg, 4.6 mmol) at the same temperature and the reaction mixture was allowed to stir at RT for 1 h. The reaction mixture was poured into ice-cold water and extracted with EtOAc (2×100 mL). The combined organic layer was washed with water (3×50 mL) and dried over sodium sulfate. Removal of water under reduced pressure gave crude product that was purified by column chromatography on silica gel (100-200 mesh) using 10% MeOH-DCM system as eluent to obtain 100 mg of 5-(2-(cyclopentyloxy)-2-(pyridin-4-yl)ethyl)-2,8-dimethyl-2,3,4,5-tetrahydro-1H-pyrido[4,3-b]indole as racemic mixture. The enantiomers were separated by chiral HPLC to afford 40 mg of (S)-5-(2-(cyclopentyloxy)-2-(pyridin-4-yl)ethyl)-2,8-dimethyl-2,3,4,5-tetrahydro-1H-pyrido[4,3-b]indole. ¹H NMR (freebase, CDCl₃): 8.60 (d, 2H), 7.25-7.15 (m, 4H), 7.01 (d, 1H), 4.60 (t, 1H), 4.20-4.01 (m, 2H), 3.81-3.61 (m, 3H), 2.95-2.85 (m, 4H), 2.61 (S, 3H), 2.50 (s, 3H), 1.72-1.51 (m, 3H), 1.50-1.40 (m, 3H), 1.35-1.21 (m, 2H).

### Example 36

### Preparation of Compound Nos. 36, 36a, 36b, 36c and 36d

To a stirred solution of Beta carboline (50 mg, 0.20 mmol) in dry DMF (2 mL) at 25° C. was added sodium hydride (5.53 g, 0.138 mol 60%) portionwise under nitrogen atmosphere. After 5 min. was added a solution of 4-(2-methyloxiran-2-yl)pyridine (56 mg, 0.41 mmol) in DMF (0.5 mL) drops wise at 25° C. After complete addition, the reaction mixture was stirred at 25° C. overnight. The reaction mixture was slowly poured in ice-cold water and extracted with EtOAc, organic layer washed with water (5 times). The organic layer dried over anhydrous sodium sulfate, concentrated under vacuum to obtain a crude product, which was purified by reverse phase HPLC to obtain the desired product as the TFA salt. The mixture was separated by chiral HPLC to afford 5 mg of desired product. ¹HNMR (CD₃OD, TFA salt): 8.48 (d, 2H), 7.82 (d, 2H), 7.21 (s, 1H), 6.84 (d, 1H), 6.78 (d, 1H), 5.10 (d, 1H), 4.75 (d, 1H), 4.35 (dd, 2H), 3.40 (m, 2H), 3.18 (s, 3H), 2.70 (m, 1H), 2.36 (m, 4H), 2.18 (m, 1H), 2.05 (m, 1H), 1.82 (m, 1H), 1.70 (s, 3H), 1.62 (m, 1H).

### Example 37

### Preparation of Compound Nos. 37, 37a, 37b, 37c and 37d

To a solution of 7-methyl-2,3,5,10,11,11a-hexahydro-1H-indolizino[7,6-b]indole (200 mg, 0.877 mmol) in DMF (5 mL) was added NaH (105 mg, 2.631 mmol) at RT and the mixture was allowed to stir for 5 min. To this was added 4-(2-methyloxiran-2-yl)pyridine (153 mg, 1.140 mmol) and the reaction mixture was allowed to stir overnight. The reaction was quenched and diluted with water and the solid mass thus obtained was filtered to get 176 mg of a mixture of stereoisomers. ¹H NMR (CDCl₃, free base) δ (ppm): 8.6 (d, 2H), 7.41 (d, 2H), 7.24 (s, 1H), 7.19 (d, 1H), 6.9 (d, 1H), 4.2 (dd, 1H), 4.1 (dd, 1H), 3.2-3.3 (m, 2H), 2.7 (d, 1H), 2.7-2.8 (m, 1H), 2.3-2.6 (m, 3H), 2.45 (s, 3H), 1.5 (s, 3H). Separation by chiral HPLC provided diastereomers 37a, 37b, 37c and 37d.

### Example 38

### Preparation of Compound Nos. 38, 38a, 38b, 38c and 38d

To a solution of 7-methyl-2,3,5,10,11,11a-hexahydro-1H-indolizino[7,6-b]indole (200 mg, 0.877 mmol) in DMF (5 mL) was added NaH (105 mg, 2.631 mmol) at RT and the mixture was allowed to stir for 5 min. To this was added 3-(2-methyloxiran-2-yl)pyridine (153 mg, 1.140 mmol) and the reaction mixture was allowed to stir overnight. The reaction was quenched and diluted with water and the solid mass thus obtained was filtered to get 176 mg of a mixture of stereoisomers. ¹H NMR (CDCl₃, free base) δ (ppm): 8.8 (s, 1H), 8.6 (s, 1H), 7.8 (d, 1H), 7.3 (d, 1H), 7.2 (s, 1H), 7.15 (d, 1H), 6.95 (d, 1H), 4.1-4.3 (m, 2H), 3.3-3.4 (m, 2H), 2.75 (d, 1H), 2.3-2.5 (m, 2H), 2.4 (s, 3H), 1.8-2.1 (m, 2H), 1.5 (s, 3H). Separation by chiral HPLC provided diastereomers 38a, 38b, 38c and 38d.

### Example 39

### Preparation of Compound Nos. 39, 39a and 39b

To a solution of 2-(6-bromopyridin-3-yl)-1-(2,8-dimethyl-3,4-dihydro-1H-pyrido[4,3-b]indol-5(2H)-yl)propan-2-ol (100 mg, 0.242 mmol) in ethanol (1.0 mL) was added diethylamine (1.0 mL) the reaction mixture was heated under microwave condition at 120° C. for 1 h. The progress of reaction was monitored by ¹H NMR spectroscopy. The solvent was removed under reduced pressure to obtain crude product which was purified by reverse phase HPLC to afford 80 mg of 1-(2,8-dimethyl-3,4-dihydro-1H-pyrido[4,3-b]indol-5(2H)-yl)-2-(6-(dimethylamino)pyridin-3-yl)propan-2-ol as a TFA salt. ¹H NMR (CD₃OD, TFA salt) δ (ppm): 7.94 (d, 1H), 7.58 (s, 1H), 7.18 (s, 1H), 7.03 (d, 1H), 6.84 (brs, 2H), 4.69 (d, 2H), 4.19-4.33 (m, 3H), 3.86 (brs, 1H), 3.52 (brs, 2H), 3.19 (s, 6H), 3.11 (s, 3H), 1.95-2.36 (s, 3H), 1.66 (s, 3H). Separation by chiral HPLC provided enantiomers 39a and 39b.

### Example 40

### Preparation of Compound Nos. 40, 40a and 40b

To a solution of 2-(4-chloropyridin-3-yl)-1-(2,8-dimethyl-3,4-dihydro-1H-pyrido[4,3-b]indol-5(2H)-yl)propan-2-ol (1 g, 2.71 mmol) in DMF (50 mL) was added cesium fluoride (4.10 g, 27.1 mmol) and the reaction mixture was allowed to stir at 140° C. for 3 h. The progress of reaction mixture was monitored by LCMS. The reaction mixture was allowed to cool to RT and poured into ice-cold water (150 mL). The aqueous layer was extracted with EtOAc (3×200 mL). The combined organic layer was washed with water (5×100 mL) and dried over anhydrous sodium sulfate. Removal of EtOAc under reduced pressure gave crude product which was purified by reverse phase HPLC to afford 3 mg of 1-(2,8-dimethyl-3,4-dihydro-1H-pyrido[4,3-b]indol-5(2H)-yl)-2-(4-fluoropyridin-3-yl)propan-2-ol as a free base. ¹H NMR (CDCl₃, free base) δ (ppm): 8.94 (d, 1H), 8.57 (t, 1H), 7.29 (d, 1H), 7.17 (s, 1H), 7.03 (d, 2H), 4.47 (d, 1H), 4.15 (d, 1H), 3.95-4.05 (m, 2H), 2.90-3.05 (m, 4H), 2.76 (s, 3H), 2.43 (s, 3H), 1.55 (s, 3H). Separation by chiral HPLC provides enantiomers 40a and 40b.

### Example 41

### Preparation of Compound Nos. 41, 41a, 41b, 41c and 41d

To a solution of 7-fluoro-2,3,5,10,11,11a-hexahydro-1H-indolizino[7,6-b]indole (100 mg, 0.4 mmol) in DMF (2 mL) was added NaH (48 mg, 1.2 mmol) at RT and the mixture was allowed to stir for 5 min. To this was added 3-(2-methyl-oxiranyl)-pyridine (98 mg, 0.6 mmol) and the reaction mixture was allowed to stir for 16 h. The reaction mixture was diluted with ice-cold water and extracted with EtOAc (3×50 mL). The combined organic layer was washed with water (4×50 mL) and dried over anhydrous sodium sulfate. Removal of solvent under reduced pressure gave crude product which was recrystallized in an ether-hexane system to obtain 90 mg of a diastereomeric mixture. ¹H NMR (CD₃OD, HCl salt) δ (ppm): 8.70 (d, 3H), 7.98 (s, 1H), 7.13 (d, 1H), 6.96 (s, 1H), 6.76 (t, 1H), 4.82 (d, 2H), 4.30-4.44 (m, 2H), 3.84-3.90 (m, 2H), 3.31-3.40 (m, 1H), 3.09-3.13 (m, 1H), 2.60 (d, 1H), 2.30-2.60 (m, 2H), 1.96-2.05 (m, 1H), 1.79 (s, 3H). Separation by chiral HPLC provided diastereomers 41a, 41b, 41c and 41d.

### Example 42

### Preparation of Compound Nos. 42, 42a and 42b

A solution of 5-(2-(allyloxy)-2-(pyridin-4-yl)ethyl)-2,8-dimethyl-2,3,4,5-tetrahydro-1H-pyrido[4,3-b]indole (125 mg, 0.34 mmol) in MeOH (100 mL) was subjected to hydrogenation in H-cube at 60 psi pressure. Removal of solvent under reduced pressure gave crude product which was purified by reverse phase chromatography to obtain 65 mg of an enantiomeric mixture of (R) and (S)-2,8-dimethyl-5-(2-propoxy-2-(pyridin-4-yl)ethyl)-2,3,4,5-tetrahydro-1H-pyrido[4,3-b]indole. The enantiomers were separated by chiral HPLC to afford 12 mg of (S)-2,8-dimethyl-5-(2-propoxy-2-(pyridin-4-yl)ethyl)-2,3,4,5-tetrahydro-1H-pyrido[4,3-b]indole. ¹H NMR (CD₃OD, HCl salt) δ (ppm): 8.72-8.78 (m, 2H), 8.06 (d, 1H), 8.01 (d, 1H), 7.23 (s, 1H), 7.14 (dd, 1H), 6.90-6.98 (m, 1H), 5.06 (t, 1H), 4.68 (d, 2H), 4.42-4.48 (m, 2H), 4.33 (d, 1H), 3.86-2.90 (m, 2H), 3.52-3.62 (m, 2H), 3.19-3.24 (m, 2H), 3.13 (s, 3H), 2.38 (s, 3H), 1.48-1.58 (m, 2H), 0.84 (t, 2H). Separation by chiral HPLC provided enantiomers 42a and 42b.

### Example 43

### Preparation of Compound Nos. 43, 43a and 43b

A solution of 5-(2-ethoxy-2-(pyridin-4-yl)vinyl)-2,8-dimethyl-2,3,4,5-tetrahydro-1H-pyrido[4,3-b]indole (200 mg, 0.57 mmol) in MeOH (100 mL) was passed through H-cube (10% Pd/C) at 60 psi pressure. MeOH was removed under reduced pressure and the crude was purified by reverse phase chromatography to obtain 60 mg of an enantiomeric mixture. ¹H NMR (CD₃OD, HCl salt) δ (ppm): 8.75 (d, 2H), 8.04 (d, 2H), 7.23 (s, 1H), 7.12 (d, 1H), 6.90-6.99 (m, 1H), 5.06 (t, 1H), 4.65-4.75 (m, 1H), 4.40-4.56 (m, 2H), 4.16-4.20 (m, 1H), 3.85-3.3.95 (m, 1H), 3.50-3.63 (m, 1H), 3.20-3.50 (m, 4H), 3.13 (s, 3H), 2.39 (s, 3H), 1.13 (t, 3H). Separation by chiral HPLC provided enantiomers 43a and 43b.

### Example 44

### Preparation of Compound Nos. 44, 44a, 44b, 44c and 44d

To a solution of 8,10-dimethyl-2,3,5,6,7,11c-hexahydro-1H-indolizino[7,8-b]indole (200 mg, 0.834 mmol) in DMF (3 mL) was added sodium hydride (167 mg, 4.17 mmol) at 0° C. After 5 min of stirring, a solution of 3-(2-methyloxiran-2-yl)pyridine (250 mg, 1.67 mmol) in DMF (2 mL) was added dropwise. The reaction mixture was allowed to stir at RT for 18 h. The progress of reaction was monitored by TLC and LCMS. After completion of the reaction, ice-cold water was added to the reaction mixture and it was extracted with EtOAc (3×50 mL). The combined organic layer was washed with water (4×100 mL) and dried over sodium sulfate. Removal of the solvent under reduced pressure afforded a crude product which was column chromatography on neutral alumina using 2-3% MeOH-DCM system as eluent to obtain 160 mg of a diastereomeric mixture. ¹H NMR (CD₃OD, free base) δ (ppm): 8.28 (s, 2H), 7.64 (d, 1H), 7.18-7.22 (m, 1H), 6.99 (s, 1H), 6.55 (s, 1H), 4.40-4.58 (m, 2H), 4.24 (brs, 1H), 3.18-3.22 (m, 1H), 2.80-3.12 (m, 3H), 2.48 (s, 3H), 2.38-2.61 (m, 2H), 2.31 (s, 3H), 1.98 (brs, 4H), 1.58 (s, 3H). Separation by chiral HPLC provided diastereomers 44a, 44b, 44c and 44d.

### Example 45

### Preparation of Compound Nos. 45, 45a, 45b, 45c and 45d

To a solution of 7-methyl-2,3,5,10,11,11a-hexahydro-1H-indolizino[7,6-b]indole (200 mg, 0.834 mmol) in DMF (3 mL) was added sodium hydride (106 mg, 2.654 mmol) at 0° C. After 5 min of stirring, a solution of 2-(oxiran-2-yl)pyridine (160 mg, 1.327 mmol) in DMF (2 mL) was added dropwise. The reaction mixture was allowed to stir at RT overnight. The progress of reaction was monitored by TLC and LCMS. After completion of the reaction, ice-cold water was added to the reaction mixture to get a precipitate which was filtered to afford a diastereomeric mixture. ¹H NMR (CDCl₃, free base) δ (ppm): 8.58 (d, 1H), 7.45 (t, 1H), 7.10-7.25 (m, 2H), 7.04 (d, 1H), 6.89 (d, 1H), 6.68 (d, 1H), 5.00-5.05 (m, 1H), 4.10-4.40 (m, 4H), 3.33 (d, 2H), 2.42 (s, 3H), 2.25-2.40 (m, 4H), 1.80-2.10 (5H). Separation by chiral HPLC provided diastereomers 45a, 45b, 45c and 45d.

### Example 46

### Preparation of Compound Nos. 46, 46a and 46b

To a suspension of sodium hydride (240 mg, 6.00 mmol) in DMF (3 mL) was added 2,8-dimethyl-2,3,4,5-tetrahydro-1H-pyrido[4,3-b]indole (600 mg, 3 mmol) at 0° C. After 15 min of stirring at the same temperature, to this was added a solution of 3-fluoro-5-(2-methyloxiran-2-yl)pyridine (550 mg, 3.59 mmol) in DMF (2 mL) and the reaction mixture was allowed to stir at RT for 16 h. The DMF was removed, the residue was diluted with water and the product was extracted with EtOAc (3×50 mL). The combined organic layer was washed with water (50 mL) and dried over anhydrous sodium sulfate. Removal of solvent under reduced pressure gave crude product which was purified by crystallization in diethyl ether to afford 350 mg of (S)-1-(2,8-dimethyl-3,4-dihydro-1H-pyrido[4,3-b]indol-5(2H)-yl)-2-(5-fluoropyridin-3-yl)propan-2-ol and (R)-1-(2,8-dimethyl-3,4-dihydro-1H-pyrido[4,3-b]indol-5(2H)-yl)-2-(5-fluoropyridin-3-yl)propan-2-ol as racemate. ¹H NMR (CDCl₃, free base) δ (ppm): 8.51 (s, 1H), 8.33 (d, 1H), 7.49 (d, 1H), 7.07 (s, 1H), 7.05 (d, 1H), 6.92 (d, 1H), 4.09 (q, 2H), 3.72 (q, 2H), 2.70-2.96 (m, 4H), 2.55 (s, 3H), 2.40 (s, 3H), 1.54 (s, 3H). Separation by chiral HPLC provided enantiomers 46a and 46b.

### Example 47

### Preparation of Compound Nos. 47, 47a and 47b

To a solution of 2-(6-bromopyridin-3-yl)-1-(2,8-dimethyl-3,4-dihydro-1H-pyrido[4,3-b]indol-5(2H)-yl)propan-2-ol (200 mg, 0.483 mmol) in MeOH (2 mL) was added ammonium hydroxide (2 mL) and Cu(II)oxide (0.011 mg, 0.145 mmol) and the reaction mixture was heated on oil bath at 160° C. for 2 h. The progress of reaction was monitored by LCMS. The reaction mixture was concentrated and purified by preparative HPLC to afford 15 mg of 2-(6-aminopyridin-3-yl)-1-(2,8-dimethyl-3,4-dihydro-1H-pyrido[4,3-b]indol-5(2H)-yl)propan-2-ol as the free base. ¹H NMR (CDCl₃, free base) δ (ppm): 7.96 (s, 1H), 7.45 (d, 1H), 7.14 (s, 2H), 6.99 (d, 1H), 6.51 (d, 1H), 4.30 (d, 1H), 4.10-4.30 (m, 2H), 4.00 (d, 1H), 3.39 (s, 2H), 3.31 (brs, 1H), 2.86-3.20 (m, 4H), 2.42 (s, 3H), 2.03 (s, 3H), 1.54 (s, 3H). Separation by chiral HPLC provides enantiomers 47a and 47b.

### Example 48

### Preparation of Compound Nos. 48, 48a, 48b, 48c and 48d

To a stirred solution of 2,3,5,6,7,11c-hexahydro-10-methyl-1H-indolizino[7,8-b]indole (590 mg, 2.61 mmol) in dry DMF (6 mL) at 0° C. was added sodium hydride (0.26 g, 6.52 mmol, 60%) portionwise under nitrogen atmosphere. After 15 min was added solution of compound 2-fluoro-4-(2-methyloxiran-2-yl)pyridine (0.600 g, 3.91 m mol) in DMF (1 mL) drops wise at 0° C. After complete addition, the reaction mixture stirred at RT for 1.5 h. The desired product was detected by NMR & LCMS. The reaction mixture was slowly poured into ice-cold water and extracted with EtOAc (5×75 mL). The combined organic layer was washed with water (5×50 mL) and dried over anhydrous sodium sulfate. Removal of solvent under reduced pressure gave a diastereomeric mixture. ¹H NMR (CD₃OD, HCl salt) δ (ppm): 8.04 (d, 1H), 7.38 (d, 1H), 7.20 (s, 1H), 7.00-7.17 (m, 2H), 6.88 (d, 1H), 5.03 (brs, 1H), 4.33 (dd, 1H), 4.25 (dd, 1H), 3.50-3.70 (m, 3H), 3.02-3.40 (m, 3H), 2.65-2.75 (m, 1H), 2.38 (s, 3H), 2.10-2.30 (m, 3H), 1.64 (s, 3H). Separation by chiral HPLC provided diastereomers 48a, 48b, 48c and 48d.

### Example 49

### Preparation of Compound Nos. 49, 49a and 49b

To a solution of 5-(2-isopropoxy-2-(pyridin-4-yl)vinyl)-2,8-dimethyl-2,3,4,5-tetrahydro-1H-pyrido[4,3-b]indole (200 mg, 0.5 mmol) in MeOH (20 mL) was added 10% Pd/C and the reaction mixture was hydrogenated for 18 h. The reaction mixture was passed through a Celite bed and the filtrate was concentrated under vacuum to give crude product which was purified by reverse phase HPLC to afford 12 mg of 5-(2-isopropoxy-2-(pyridin-4-yl)ethyl)-2,8-dimethyl-2,3,4,5-tetrahydro-1H-pyrido[4,3-b]indole. ¹H NMR (CDCl₃, free base) δ (ppm): 8.55 (d, 2H), 7.15 (m, 4H), 7.00 (d, 1H), 4.65 (t, 1H), 4.15 (dd, 1H), 4.05 (dd, 1H), 3.78 (m, 2H), 3.35 (m, 1H), 2.90 (m, 3H), 2.65 (m, 1H), 2.60 (s, 3H), 2.40 (s, 3H), 1.05 (d, 3H), 0.85 (d, 3H). Separation by chiral HPLC provides enantiomers 49a and 49b.

### Example 50

### Preparation of Compound Nos. 50, 50a, 50b, 50c and 50d

To a suspension of sodium hydride (69 mg, 1.7 mmol) in DMF (2 mL) was added 9-chloro-10-methyl-2,3,5,6,7,11c-hexahydro-1H-indolizino[7,8-b]indole (150 mg, 0.5 mmol) at 0° C. After 5 min of stirring at the same temperature, to this was added a solution of 4-(2-methyl-oxiranyl)-pyridine (132 mg, 0.9 mmol) in DMF (2 mL) and the reaction mixture was allowed to stir at RT for 18 h. The DMF was removed, the residue was diluted with water and the product was extracted with EtOAc (3×50 mL). The combined organic layer was washed with water (4×50 mL) and dried over anhydrous sodium sulfate. Removal of solvent under reduced pressure gave crude product which was purified by crystallization in diethyl ether to afford 250 mg of a diastereomeric mixture. ¹H NMR (CDCl₃, free base) δ (ppm): 8.58 (d, 2H), 7.35 (d, 2H), 7.33 (s, 1H), 7.32 (s, 1H), 4.10 (dd, 2H), 3.95 (m, 1H), 3.20 (m, 1H), 2.85-2.70 (m, 4H), 2.60 (m, 1H), 2.45 (m, 1H), 2.40 (s, 3H), 1.90 (m, 3H), 1.60 (s, 3H). Separation by chiral HPLC provided diastereomers 50a, 50b, 50c and 50d.

### Example 51

### Preparation of Compound Nos. 51, 51a, 51b, 51c and 51d

To a suspension of sodium hydride (125 mg, 3.125 mmol) in DMF (3 mL) was added 10-fluoro-2,3,5,6,7,11c-hexahydro-1H-indolizino[7,8-b]indole (240 mg, 1.043 mmol) at 0° C. After 5 min of stirring at the same temperature, to this was added a solution of 4-(2-methyl-oxiranyl)-pyridine (225 mg, 1.66 mmol) in DMF (2 mL) and the reaction mixture was allowed to stir at RT for 5 h. The DMF was removed, the residue was diluted with water and the product was extracted with EtOAc (3×50 mL). The combined organic layer was washed with water (4×50 mL) and dried over anhydrous sodium sulfate. Removal of solvent under reduced pressure gave crude product which was purified by crystallization in diethyl ether to afford 240 mg of a diastereomeric mixture. ¹H NMR (CDCl₃, free base) δ (ppm): 8.70 (s, 1H), 8.50 (d, 1H), 7.55 (d, 1H), 7.05 (m, 3H), 6.80 (m, 1H), 4.75 (bs, 1H), 4.20 (dd, 2H), 3.85 (t, 1H), 3.18 (m, 1H), 2.85 (m, 2H), 2.75 (m, 1H), 2.58 (m, 2H), 2.35 (m, 1H), 1.85 (m, 3H), 1.70 (s, 3H). Separation by chiral HPLC provided diastereomers 51a, 51b, 51c and 51d.

### Example 52

### Preparation of Compound Nos. 52, 52a, 52b, 52c and 52d

To a suspension of sodium hydride (90 mg, 2.2 mmol) in DMF (2.5 mL) was added 10-chloro-8-fluoro-2,3,5,6,7,11c-hexahydro-1H-indolizino[7,8-b]indole (200 mg, 0.7 mmol) at 0° C. After 5 min of stirring at the same temperature, to this was added a solution of 4-(2-methyl-oxiranyl)-pyridine (173 mg, 1.2 mmol) in DMF (2 mL) and the reaction mixture was allowed to stir at RT for 18 h. The DMF was removed, the residue was diluted with water and the product was extracted with EtOAc (3×50 mL). The combined organic layer was washed with water (4×50 mL) and dried over anhydrous sodium sulfate. Removal of solvent under reduced pressure gave crude product which was purified by crystallization in diethyl ether to afford 170 mg of a diastereomeric mixture. ¹H NMR (CDCl₃, free base) δ (ppm): 8.58 (d, 2H), 7.35 (d, 2H), 7.20 (s, 1H), 6.85 (d, 1H), 4.50 (d, 1H), 4.15 (d, 1H), 3.80 (t, 1H), 3.25 (m, 1H), 2.90 (m, 2H), 2.70 (m, 2H), 2.45 (m, 2H), 1.90 (m, 3H), 1.55 (s, 3H). Separation by chiral HPLC provided diastereomers 52a, 52b, 52c and 52d.

### Example 53

### Preparation of Compound Nos. 53, 53a, 53b, 53c and 53d

To a suspension of sodium hydride (90 mg, 2.2 mmol) in DMF (2.5 mL) was added 10-chloro-8-fluoro-2,3,5,6,7,11c-hexahydro-1H-indolizino[7,8-b]indole (200 mg, 0.7 mmol) at 0° C. After 5 min of stirring at the same temperature, to this was added a solution of 4-(2-methyl-oxiranyl)-pyridine (173 mg, 1.2 mmol) in DMF (2 mL) and the reaction mixture was allowed to stir at RT for 18 h. The DMF was removed, the residue was diluted with water and the product was extracted with EtOAc (3×50 mL). The combined organic layer was washed with water (4×50 mL) and dried over anhydrous sodium sulfate. Removal of solvent under reduced pressure gave crude product which was purified by crystallization in diethyl ether to afford 140 mg of a diastereomeric mixture. ¹H NMR (CDCl₃, free base) δ (ppm): 8.70 (s, 1H), 8.55 (d, 1H), 7.65 (d, 1H), 7.20 (m, 2H), 6.82 (d, 1H), 4.55 (d, 1H), 4.20 (d, 1H), 3.85 (t, 1H), 3.25 (m, 1H), 2.90-2.70 (m, 4H), 2.45 (m, 2H), 1.90 (m, 3H), 1.60 (s, 3H). Separation by chiral HPLC provided diastereomers 53a, 53b, 53c and 53d.

### Example 54

### Preparation of Compound Nos. 54, 54a and 54b

To a suspension of 5-(2-(2,8-dimethyl-3,4-dihydro-1H-pyrido[4,3-b]indol-5(2H)-yl)-1-hydroxyethyl)picolinic acid (100 mg, 0.273 mmol) and EDC.HCl (75 mg, 0.391 mmol) DCM (5 mL) was added 2M dimethylamine solution in THF (0.3 mL). The reaction mixture was allowed to stir RT for 3 h. The progress of reaction was monitored by LCMS. The reaction mixture was diluted with water (10 mL) and the product was extracted with EtOAc (3×20 mL). The combined organic layer was washed with water (3×10 mL) and dried over anhydrous sodium sulfate. Removal of solvent under reduced pressure gave crude product which was purified by reverse phase HPLC to afford 1 mg of 5-(2-(2,8-dimethyl-3,4-dihydro-1H-pyrido[4,3-b]indol-5(2H)-yl)-1-hydroxyethyl)-N,N-dimethylpicolinamide as the free base. ¹H NMR (CDCl₃, freebase) δ (ppm): 8.21 (s, 1H), 7.78 (d, 1H), 7.43 (s, 1H), 7.17 (s, 1H), 7.02 (d, 1H), 6.87 (d, 1H), 5.14 (t, 1H), 4.16-4.45 (m, 4H), 3.21-3.42 (m 2H), 2.85-3.20 (2H), 3.09 (s, 3H), 2.90 (s, 3H), 2.84 (s, 3H), 2.36 (s, 3H). Separation by chiral HPLC provides enantiomers 54a and 54b.

### Example 55

### Preparation of Compound Nos. 55, 55a and 55b

To a suspension of 5-(2-(2,8-dimethyl-3,4-dihydro-1H-pyrido[4,3-b]indol-5(2H)-yl)-1-hydroxyethyl)picolinic acid (200 mg, 0.548 mmol) and EDC.HCl (158 mg, 0.822 mmol) in DCM (5 mL) was added cyclopropyl amine (0.057 mL, 0.822 mmol). The reaction mixture was allowed to stir RT for 3 h. The progress of reaction was monitored by LCMS. The reaction mixture was diluted with water (10 mL) and the product was extracted with EtOAc (3×20 mL). The combined organic layer was washed with water (3×10 mL) and dried over anhydrous sodium sulfate. Removal of solvent under reduced pressure gave crude product which was purified by reverse phase HPLC to afford 8 mg of N-cyclopropyl-5-(2-(2,8-dimethyl-3,4-dihydro-1H-pyrido[4,3-b]indol-5(2H)-yl)-1-hydroxyethyl)picolinamide as the free base. ¹H NMR (CD₃OD, TFA salt) δ (ppm): 8.40 (s, 1H), 7.98 (d, 1H), 7.82 (s, 1H), 7.10-7.25 (m, 2H), 6.93-7.10 (m, 1H), 5.16 (t, 1H), 4.60-4.72 (m, 1H), 4.20-4.40 (m 2H), 3.60-3.95 (m, 3H), 3.09 (s, 3H), 2.80-2.95 (m, 1H), 2.40 (s, 3H), 0.84 (d, 2H), 0.66 (brs, 2H). Separation by chiral HPLC provides enantiomers 55a and 55b.

### Example 56

### Preparation of Compound Nos. 56, 56a and 56b

To a solution of cyclopropylmethanol (52 mg, 0.726 mmol) in DMF (10 mL) was added sodium hydride (38 mg, 0.968 mmol) at RT. After 10 min of stirring was added slowly a solution of 2-(6-bromopyridin-3-yl)-1-(2,8-dimethyl-3,4-dihydro-1H-pyrido[4,3-b]indol-5(2H)-yl)propan-2-ol (200 mg, 0.484) in DMF (3 mL) and the reaction mixture was allowed to stir at 100° C. overnight. The progress of the reaction was monitored by NMR and LCMS. The reaction mixture was diluted with ice-cold water, the precipitate was filtered, washed with water and dried under vacuum to get crude product which was purified reverse phase HPLC to afford 2-(6-(cyclopropylmethoxy)pyridin-3-yl)-1-(2,8-dimethyl-3,4-dihydro-1H-pyrido[4,3-b]indol-5(2H)-yl)propan-2-ol as the TFA salt. ¹H NMR (CD₃OD, TFA salt) δ (ppm): 8.00 (brs, 1H), 7.74 (d, 1H), 7.16 (s, 1H), 6.80-6.97 (m, 2H), 6.74 (t, 1H), 4.66 (d, 1H), 4.17-4.38 (m, 4H), 4.04 (t, 2H), 3.79 (brs, 1H), 3.05-3.58 (m, 3H), 3.09 (s, 3H), 2.36 (s, 3H), 1.66 (s, 3H), 0.59 (d, 2H), 0.33 (d, 2H). Separation by chiral HPLC provides enantiomers 56a and 56b.

### Example 57

### Preparation of Compound Nos. 57, 57a, 57b, 57c and 57d

To a solution of 10-fluoro-2,3,5,6,7,11c-hexahydro-1H-indolizino[7,8-b]indole (230 mg, 1.0 mmol) in DMF (3 mL) was added sodium hydride (120 mg, 3.0 mmol) at 0° C. After 5 min of stirring, a solution of 3-(2-methyloxiran-2-yl)pyridine (216 mg, 1.6 mmol) in DMF (2 mL) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (2×100 mL). The combined organic layer was washed with water (4×50 mL) and dried over sodium sulfate. Removal of solvent under reduced pressure afforded a crude product which was purified by crystallization in EtOAc-hexane system to afford 270 mg of a diastereomeric mixture. ¹H NMR (CDCl₃, freebase) δ (ppm): 8.71 (s, 1H), 8.53 (d, 1H), 7.66 (d, 1H), 7.16-7.21 (m, 2H), 7.57 (d, 1H), 6.84 (t, 1H), 4.13 (q, 2H), 3.90 (brs, 1H), 3.20-3.26 (m, 1H), 2.81-2.92 (m, 1H), 2.70-2.81 (m, 3H), 2.38-2.42 (m, 2H), 1.80-1.97 (m, 3H), 1.66 (s, 3H). Separation by chiral HPLC provided diastereomers 57a, 57b, 57c and 57d.

### Example 58

### Preparation of Compound Nos. 58, 58a and 58b

To a suspension of 1-(8-methyl-3,4-dihydro-1H-pyrido[4,3-b]indol-5(2H)-yl)-2-(pyridin-3-yl)propan-2-ol (150 mg, 0.467 mmol) and potassium carbonate (198 mg, 1.42 mmol) in acetonitrile (1.5 mL) was added 1,1-difluoro-2-iodo-ethane (107 mg, 0.557 mmol). The reaction mixture was allowed to stir at 80 de c for 1 h. The progress of the reaction was monitored by LCMS. The reaction mixture was cooled to RT and diluted water (20 mL). The product was extracted with EtOAc (3×20 mL). The combined organic layer was dried over anhydrous sodium sulfate. Removal of solvent under reduced pressure gave crude product which was purified by reverse phase HPLC to afford 20 mg of 1-(2-(2,2-difluoroethyl)-8-methyl-3,4-dihydro-1H-pyrido[4,3-b]indol-5(2H)-yl)-2-(pyridin-3-yl)propan-2-ol as the free base. ¹H NMR (CD₃OD, TFA salt) δ (ppm): 8.48-8.58 (m, 2H), 8.36 (d, 1H), 7.67 (t, 1H), 7.16 (s, 1H), 6.79 (s, 2H), 4.61 (d, 1H), 4.55 (d, 1H), 4.33 (d, 1H), 3.90 (d, 1H), 3.69-3.90 (m, 4H), 3.41-3.57 (m, 1H), 3.20-3.36 (m, 2H), 2.34 (s, 3H), 1.77 (s, 3H). Separation by chiral HPLC provides enantiomers 58a and 58b.

### Example 59

### Preparation of Compound Nos. 59, 59a and 59b

To a solution of 2-(2,8-dimethyl-3,4-dihydro-1H-pyrido[4,3-b]indol-5(2H)-yl)-1-(pyridin-4-yl)ethanol (500 mg, 1.5 mmol) in DMF (4 mL) was added sodium hydride (180 mg, 4.5 mmol) at 0° C. After 20 min of stirring, a solution of bromomethyl-cyclobutane (1.16 g, 7.7 mmol) in DMF (2 mL) was added dropwise. The reaction mixture was allowed to stir at RT for 2 h. The progress of reaction was monitored by TLC and LCMS. After completion of the reaction, ice-cold water was added to the reaction mixture and the product was extracted with EtOAc (3×100 mL). The combined organic layer was washed with water (4×50 mL) and dried over anhydrous sodium sulfate. Removal of solvent under reduced pressure afforded a crude product that was purified by reverse phase HPLC to afford 280 mg of racemic mixture of (S)-5-(2-(cyclobutylmethoxy)-2-(pyridin-4-yl)ethyl)-2,8-dimethyl-2,3,4,5-tetrahydro-1H-pyrido[4,3-b]indole and (R)-5-(2-(cyclobutylmethoxy)-2-(pyridin-4-yl)ethyl)-2,8-dimethyl-2,3,4,5-tetrahydro-1H-pyrido[4,3-b]indole. ¹H NMR (CDCl₃, freebase) δ (ppm): 8.56 (d, 2H), 7.19 (s, 1H), 7.13 (d, 2H), 7.11 (d, 1H), 6.96 (d, 1H), 4.50-4.59 (m, 1H), 4.13 (dd, 1H), 4.05 (dd, 1H), 3.62 (d, 1H), 3.60 (d, 1H), 3.21-3.30 (m 1H), 3.10-3.20 (m, 1H), 2.70-2.82 (m, 3H), 2.51 (s, 3H), 2.44 (s, 3H), 2.40-2.57 (m, 1H), 1.57-1.99 (m, 7H). Separation by chiral HPLC provided enantiomers 59a, and 59b.

### Example 60

### Preparation of Compound Nos. 60, 60a and 60b

To a solution of 2-(2-methyl-8-(trifluoromethyl)-3,4-dihydro-1H-pyrido[4,3-b]indol-5(2H)-yl)-1-(pyridin-4-yl)ethanol (400 mg, 1.06 mmol) in DMF (3 mL) was added sodium hydride (127 mg, 5.3 mmol) at 0° C. After 20 min of stirring, a solution of bromocyclopentane (790 mg, 5.3 mmol) in DMF (2 mL) was added dropwise. The reaction mixture was allowed to stir at RT for 5 h. The progress of reaction was monitored by TLC and LCMS. After completion of the reaction, ice-cold water was added to the reaction mixture and extracted with EtOAc (3×100 mL). The combined organic layer was washed with water (4×50 mL) and dried over anhydrous sodium sulfate. Removal of solvent under reduced pressure afforded a crude products that were purified by reverse phase HPLC to afford 30 mg of racemic mixture of (S)-5-(2-(cyclopentyloxy)-2-(pyridin-4-yl)ethyl)-2-methyl-8-(trifluoromethyl)-2,3,4,5-tetrahydro-1H-pyrido[4,3-b]indole and (R)-5-(2-(cyclopentyloxy)-2-(pyridin-4-yl)ethyl)-2-methyl-8-(trifluoromethyl)-2,3,4,5-tetrahydro-1H-pyrido[4,3-b]indole. ¹H NMR (CDCl₃, freebase) δ (ppm): 8.58 (d, 2H), 7.69 (s, 1H), 7.30-7.40 (m, 2H), 7.20 (d, 2H), 4.52-4.62 (m, 1H), 4.07-4.23 (m, 2H), 3.57-3.81 (m, 3H), 2.58-2.95 (m, 4H), 2.54 (s, 3H), 1.86 (brs, 2H), 1.15-1.60 (m, 6H). Separation by chiral HPLC provided enantiomers 60a and 60b.

### Example 61

### Preparation of Compound Nos. 61, 61a, 61b, 61c and 61d

To a solution of 9-fluoro-7-methyl-2,3,5,10,11,11a-hexahydro-1H-indolizino[7,6-b]indole (150 mg, 0.614 mmol) in DMF (2 mL) was added sodium hydride (73 mg, 1.82 mmol) at 0° C. After 20 min of stirring, 3-(2-methyloxiran-2-yl)pyridine (165 mg, 1.22 mmol) in DMF (1 mL) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (4×50 mL). The combined organic layer was washed with water (6×10 mL) and dried over anhydrous sodium sulfate. Removal of solvent under reduced pressure gave a crude product that was purified by reverse phase HPLC to afford 20 mg of 1-(9-fluoro-7-methyl-2,3-dihydro-1H-indolizino[7,6-b]indol-10(5H,11H,11aH)-yl)-2-(pyridin-3-yl)propan-2-ol. ¹H NMR (CDCl₃, freebase) δ (ppm): 8.66 (brs, 1H), 8.41 (brs, 1H), 7.56 (brs, 1H), 7.02 (brs, 1H), 6.83 (s, 1H), 6.53 (d, 1H), 4.20-4.50 (m, 3H), 3.40-3.60 (m, 2H), 2.80-3.12 (m, 3H), 2.58-2.70 (m, 1H), 2.34 (s, 3H), 1.80-2.22 (m, 4H), 1.64 (s, 3H). Separation by chiral HPLC provided diastereomers 61a, 61b, 61c and 61d.

### Example 62

### Preparation of Compound Nos. 62, 62a, 62b, 62c and 62d

To a solution of 9-fluoro-7-methyl-2,3,5,10,11,11a-hexahydro-1H-indolizino[7,6-b]indole (150 mg, 0.614 mmol) in DMF (2 mL) was added sodium hydride (73 mg, 1.82 mmol) at 0° C. After 20 min of stirring, 4-(2-methyloxiran-2-yl)pyridine (165 mg, 1.22 mmol) in DMF (1 mL) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (4×50 mL). The combined organic layer was washed with water (6×10 mL) and dried over anhydrous sodium sulfate. Removal of solvent under reduced pressure gave a crude product that was purified by reverse phase HPLC to afford 20 mg of 1-(9-fluoro-7-methyl-2,3-dihydro-1H-indolizino[7,6-b]indol-10(5H,11H,11aH)-yl)-2-(pyridin-4-yl)propan-2-ol. ¹H NMR (CDCl₃, freebase) δ (ppm): 8.40 (brs, 2H), 7.30 (brs, 2H), 6.85 (s, 1H), 6.55 (d, 1H), 4.15-4.40 (m, 3H), 3.40-3.60 (m, 2H), 3.05-3.17 (m, 1H), 2.88 (brs, 2H), 2.58-2.63 (m, 1H), 2.34 (s, 3H), 2.02-2.21 (m, 2H), 1.79-2.00 (m, 2H), 1.57 (s, 3H). Separation by chiral HPLC provided diastereomers 62a, 62b, 62c and 62d.

### Example 63

### Preparation of Compound Nos. 63, 63a, 63b, 63c and 63d

To a solution of 7-chloro-9-fluoro-2,3,5,10,11,11a-hexahydro-1H-indolizino[7,6-b]indole (150 mg, 0.56 mmol) in DMF (2 mL) was added sodium hydride (68 mg, 1.7 mmol) at 0° C. After 5 min of stirring, 3-(2-methyloxiran-2-yl)pyridine (130 mg, 0.96 mmol) in DMF (1 mL) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (4×50 mL). The combined organic layer was washed with water (4×30 mL) and dried over anhydrous sodium sulfate. Removal of solvent under reduced pressure gave crude that was purified by reverse phase HPLC to afford 25 mg of 1-(7-chloro-9-fluoro-2,3-dihydro-1H-indolizino[7,6-b]indol-10(5H,11H,11aH)-yl)-2-(pyridin-3-yl)propan-2-ol. ¹H NMR (CDCl₃, freebase) δ (ppm): 8.66 (s, 1H), 8.45 (d, 1H), 7.43 (brs, 1H), 7.00 (s, 1H), 6.91 (brs, 1H), 6.68 (d, 1H), 4.36 (d, 1H), 4.23 (d, 1H), 3.97 (d, 1H), 3.22-3.38 (m 2H), 2.81-3.11 (m, 2H), 2.40-2.62 (m, 2H), 1.70-2.20 (m, 4H), 1.70 (s, 3H). Separation by chiral HPLC provided diastereomers 63a, 63b, 63c and 63d.

### Example 64

### Preparation of Compound Nos. 64, 64a, 64b, 64c and 64d

To a solution of 10-(trifluoromethyl)-2,3,5,6,7,11c-hexahydro-1H-indolizino[7,8-b]indole (150 mg, 0.53 mmol) in DMF (2 mL) was added sodium hydride (68 mg, 1.7 mmol) at 0° C. After 5 min of stirring, 3-(2-methyloxiran-2-yl)pyridine (122 mg, 0.9 mmol) in DMF (1 mL) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (4×50 mL). The combined organic layer was washed with water (4×30 mL) and dried over anhydrous sodium sulfate. Removal of solvent under reduced pressure gave a crude product that was purified by reverse phase HPLC to afford 200 mg of compound 64. ¹H NMR (CDCl₃, freebase) δ (ppm): 8.66 (d, 1H), 8.42 (d, 1H), 7.59 (s, 1H), 7.48 (d, 1H), 7.33 (s, 2H), 6.95 (brs, 1H), 4.28 (d, 1H), 4.14 (d, 1H), 4.12 (brs, 1H), 3.17-3.21 (m, 1H), 2.88-3.11 (m, 2H), 2.70-2.2.81 (m, 3H), 2.40-2.53 (m, 2H), 1.80-2.00 (m, 3H), 1.67 (s, 3H). Separation by chiral HPLC provided diastereomers 64a, 64b, 64c and 64d.

### Example 65

Compound Nos. 65 to 74, 80, 83 to 84, 90, 92 to 95, 97 to 100, 103 to 133, 169 to 176, and individual stereoisomers thereof, can be prepared in an analogous fashion to the Examples described both herein and in the PCT applications presented above.

### Example 66

### Preparation of Compound Nos. 75, 75a, 75b, 75c and 75d

(11cS)-10-Fluoro-8-methyl-2,3,5,6,7,11c-hexahydro-1H-indolizino[7,8-b]indole (150 mg, 0.614 mmol) was dissolved in DMF (2 mL) and sodium hydride (73 mg, 1.82 mmol) was added at 0° C. and stirred for 5 min. 3-(2-Methyl-oxiranyl)-pyridine (165 mg, 1.22 mmol) was dissolved in DMF (1 mL) and added dropwise into the reaction mixture. The reaction mixture was allowed to come to RT and stirred for 18 h. After consumption of starting material, the reaction mixture was poured in to ice cold water and extracted with EtOAc (4×20 mL). The combined organic layer was washed with water (4×10 mL) and dried over sodium sulfate and concentrated under reduced pressure to obtain the crude product, which was purified by reverse phase chromatography to obtain 1-[(11cS)-10-fluoro-8-methyl-1,2,3,5,6,11c-hexahydroindolizino[7,8-b]indol-7-yl]-2-(3-pyridyl)propan-2-ol (Cpd. No. 75) (34 mg). The mixture was separated by chiral chromatography to obtain (2S)-1-[(11cS)-10-fluoro-8-methyl-1,2,3,5,6,11c-hexahydroindolizino[7,8-b]indol-7-yl]-2-(3-pyridyl)propan-2-ol (14 mg). ¹H NMR (CDCl₃, freebase) δ (ppm): 8.65 (s, 1H), 8.55 (d, 1H), 7.60 (m, 1H), 7.25 (m, 1H), 6.95 (d, 1H), 6.62 (d, 1H), 4.42 (m, 2H), 4.00 (m, 1H), 3.22 (m, 1H), 2.90-2.75 (m, 4H), 2.70 (s, 3H), 2.40 (m, 2H), 1.90 (m, 3H), 1.62 (s, 3H). Other diastereomers can be prepared by using appropriate chiral starting materials.

### Example 67

### Preparation of Compound Nos. 76, 76a, 76b, 76c and 76d

(11aR)-7-Fluoro-9-methyl-2,3,5,10,11,11a-hexahydro-1H-indolizino[7,6-b]indole (150 mg, 0.614 mmol) was dissolved in DMF (3 mL) and sodium hydride (73 mg, 1.82 mmol) was added at 0° C. and stirred for 5 min. 3-(2-Methyl-oxiranyl)-pyridine (165 mg, 1.22 mmol) was dissolved in 1 mL DMF and added dropwise into the reaction mixture, which was allowed to come to RT and was stirred for 18 h. After consumption of starting material, the reaction mixture was poured into ice cold water and extracted with EtOAc (4×20 mL). The combined organic layer was washed with water (4×10 mL) and dried over sodium sulfate and concentrated under vacuum to obtain the crude product, which was purified by reverse phase chromatography to obtain (2R)-1-[(11aR)-7-fluoro-9-methyl-1,2,3,5,11,11a-hexahydroindolizino[7,6-b]indol-10-yl]-2-(3-pyridyl)propan-2-ol (30 mg). ¹H NMR (CDCl₃, freebase) δ (ppm): 8.60 (s, 1H), 8.40 (d, 1H), 7.30 (d, 1H), 6.90 (m, 1H), 6.70 (d, 1H), 6.50 (d, 1H), 4.35 (m, 2H), 3.80 (m, 1H), 3.30 (m, 1H), 3.20 (m, 1H), 2.80 (m, 2H), 2.40 (s, 3H), 2.38 (m, 1H), 2.10 (m, 2H), 1.90 (m, 2H), 1.70 (m, 1H), 1.62 (s, 3H). Other diastereomers can be prepared by using appropriate chiral starting materials.

### Example 68

### Preparation of Compound Nos. 77, 77a, 77b, 77c and 77d

(11cS)-10-Fluoro-8-methyl-2,3,5,6,7,11c-hexahydro-1H-indolizino[7,8-b]indole (150 mg, 0.614 mmol) was dissolved in DMF (3 mL) and sodium hydride (73 mg, 1.82 mmol) was added at 0° C. and stirred for 5 min. 4-(2-Methyl-oxiranyl)-pyridine (165 mg, 1.22 mmol) was dissolved in 1 mL DMF and added dropwise into the reaction mixture was allowed to RT and stirred for 18 h. After consumption of starting material, the reaction mixture was poured into ice cold water and extracted with EtOAc (4×20 mL). The combined organic layer was washed with water (4×10 mL) and dried over sodium sulfate and concentrated under vacuum to obtain the crude product, which was purified by reverse phase chromatography to obtain 1-[(11cS)-10-fluoro-8-methyl-1,2,3,5,6,11c-hexahydroindolizino[7,8-b]indol-7-yl]-2-(4-pyridyl)propan-2-ol (54 mg). The racemic mixture was separated by chiral chromatography to obtain(2S)-1-[(11cS)-10-fluoro-8-methyl-1,2,3,5,6,11c-hexahydroindolizino[7,8-b]indol-7-yl]-2-(4-pyridyl)propan-2-ol (10 mg). ¹H NMR (CDCl₃, freebase) δ (ppm): 8.58 (d, 2H), 7.32 (d, 2H), 6.95 (d, 1H), 6.65 (d, 1H), 4.40 (m, 2H), 4.05 (m, 1H), 3.22 (m, 1H), 2.85 (m, 4H), 2.70 (s, 3H), 2.50 (m, 3H), 1.90 (m, 2H), 1.45 (s, 3H). Other diastereomers can be prepared by using appropriate chiral starting materials.

### Example 69

### Preparation of Compound Nos. 78, 78a, 78b, 78c and 78d

(11aR)-7-Fluoro-9-methyl-2,3,5,10,11,11a-hexahydro-1H-indolizino[7,6-b]indole (150 mg, 0.614 mmol) was dissolved in DMF (3 mL) and sodium hydride (73 mg, 1.82 mmol) was added at 0° C. and stirred for 5 min. 4-(2-Methyl-oxiranyl)-pyridine (165 mg, 1.22 mmol) was dissolved in DMF (1 mL) and added dropwise into the reaction mixture and was allowed to come to room and the reaction mixture was stirred for 18 h. After consumption of starting material, the reaction mixture was poured in to ice cold water and extracted with EtOAc (4×20 mL). The combined organic layer was washed with water (4×10 mL) and dried over sodium sulfate and concentrated under vacuum to obtain the crude product, which was purified by reverse phase chromatography to obtain 1-[(11aR)-7-fluoro-9-methyl-1,2,3,5,11,11a-hexahydroindolizino[7,6-b]indol-10-yl]-2-(4-pyridyl)propan-2-ol (58 mg). The racemic mixture was separated by chiral chromatography to obtain (2R)-1-[(11aR)-7-fluoro-9-methyl-1,2,3,5,11,11a-hexahydroindolizino[7,6-b]indol-10-yl]-2-(4-pyridyl)propan-2-ol (18 mg). ¹H NMR (CDCl₃, freebase) δ (ppm): 8.62 (d, 2H), 7.40 (d, 2H), 6.90 (d, 1H), 6.65 (d, 1H), 4.55 (d, 1H), 4.25 (d, 1H), 4.20 (d, 1H), 3.30 (m, 2H), 2.80 (m, 1H), 2.70 (s, 3H), 2.45 (m, 2H), 2.10 (m, 2H), 2.00 (m, 2H), 1.90 (m, 1H), 1.40 (s, 3H). Other diastereomers can be prepared by using appropriate chiral starting materials.

### Example 70

### Preparation of Compound Nos. 79, 79a, 79b, 79c and 79d

(11cR)-8-Fluoro-10-methyl-2,3,5,6,7,11c-hexahydro-1H-indolizino[7,8-b]indole (150 mg, 0.614 mmol) was dissolved in DMF (2 mL) and sodium hydride (73 mg, 1.82 mmol) was added at 0° C. and stirred for 5 min. 3-(2-Methyl-oxiranyl)-pyridine (165 mg, 1.22 mmol) was dissolved in DMF (1 mL) and added dropwise into the reaction mixture and was allowed to come to RT and the reaction mixture was stirred for 18 h. After consumption of starting material, the reaction mixture was poured in to ice cold water and extracted with EtOAc (4×20 mL). The combined organic layer was washed with water (6×10 mL) and dried over sodium sulfate and concentrated under vacuum to obtain the crude product, which was purified by reverse phase chromatography to obtain 1-[(11cR)-8-fluoro-10-methyl-1,2,3,5,6,11c-hexahydroindolizino[7,8-b]indol-7-yl]-2-(3-pyridyl)propan-2-ol (45 mg). The racemic mixture was separated by chiral chromatography to obtain (2S)-1-[(11cR)-8-fluoro-10-methyl-1,2,3,5,6,11c-hexahydroindolizino[7,8-b]indol-7-yl]-2-(3-pyridyl)propan-2-ol (18 mg). ¹H NMR (CDCl₃, freebase) δ (ppm): 8.75 (s, 1H), 8.52 (d, 1H), 7.70 (d, 1H), 7.25 (m, 1H), 7.00 (s, 1H), 6.65 (d, 1H), 4.55 (d, 1H), 4.18 (d, 1H), 3.98 (m, 1H), 3.28 (m, 1H), 2.85 (m, 1H), 2.75 (m, 3H), 2.42 (m, 2H), 2.40 (s, 3H), 1.90 (m, 3H), 1.60 (s, 3H). Other diastereomers can be prepared by using appropriate chiral starting materials.

### Example 71

### Preparation of Compound Nos. 81, 81a, 81b, 81c and 81d

(11cR)-8-Fluoro-10-methyl-2,3,5,6,7,11c-hexahydro-1H-indolizino[7,8-b]indole (150 mg, 0.614 mmol) was dissolved in DMF (3 mL) and sodium hydride (73 mg, 1.82 mmol) was added at 0° C. and stirred for 5 min. 4-(2-Methyl-oxiranyl)-pyridine (165 mg, 1.22 mmol) was dissolved in 1 mL DMF and added dropwise into the reaction mixture and was allowed to come to RT and the resultant solution was stirred for 18 h. After consumption of starting material, the reaction mixture was poured in to ice cold water and extracted with EtOAc (4×20 mL). The combined organic layer was washed with water (4×10 mL) and dried over sodium sulfate and concentrated under reduced pressure to obtain the crude product, which was purified by reverse phase chromatography to obtain 1-[(7R)-11-fluoro-7,9-dimethyl-2,3,4,5,6,7-hexahydro-1H-azonino[4,5-b]indol-12-yl]-2-(4-pyridyl)propan-2-ol (40 mg). The racemic mixture was separated by chiral chromatography to obtain (2S)-1-[(11cR)-8-fluoro-10-methyl-1,2,3,5,6,11c-hexahydroindolizino[7,8-b]indol-7-yl]-2-(4-pyridyl)propan-2-ol (9 mg). ¹H NMR (CDCl₃, freebase) δ (ppm): 8.58 (d, 2H), 7.40 (d, 2H), 7.00 (s, 1H), 6.65 (d, 1H), 4.58 (d, 1H), 4.10 (d, 1H), 3.95 (m, 1H), 3.25 (m, 1H), 2.88 (m, 1H), 2.80 (m, 2H), 2.45 (m, 2H), 2.40 (s, 3H), 1.90 (m, 4H), 1.55 (s, 3H). Other diastereomers can be prepared by using appropriate chiral starting materials.

### Example 72

### Preparation of Compound Nos. 82, 82a, 82b, 82c and 82d

(1S)-2-[(11cR)-10-Methyl-1,2,3,5,6,11c-hexahydroindolizino[7,8-b]indol-7-yl]-1-(4-pyridyl)ethanol (400 mg, 1.1 mmol) was dissolved in DMF (4 mL), and cooled to 0° C. Sodium hydride (138 mg, 3.4 mmol) was added portionwise and bromo-cyclopentane (850 mg, 5.7 mmol) was added at the same temperature, and the reaction mixture was allowed to come to RT and stirred for 4 h. After consumption of starting material, the reaction mixture was poured in to ice cold water and extracted with EtOAc (3×100 mL). The combined organic layer was washed with water (4×50 mL) and the organic layer was dried over sodium sulfate and concentrated under vacuum to obtain the crude product, which was purified by reverse phase chromatography to obtain (11cR)-7-[(25)-2-(cyclopentoxy)-2-(4-pyridyl)ethyl]-10-methyl-1,2,3,5,6,11c-hexahydroindolizino[7,8-b]indole (20 mg). ¹H NMR (CDCl₃, freebase) δ (ppm): 8.55 (d, 2H), 7.18 (m 4H), 7.00 (d, 1H), 4.62 (t, 1H), 4.20-4.00 (m, 3H), 3.65 (m, 1H), 3.25 (m, 1H), 2.90 (m, 1H), 2.78 (m, 2H), 2.65 (m, 1H), 2.45 (s, 3H), 2.35 (m, 1H), 1.90 (m, 3H), 1.40 (m, 4H), 1.25 (m, 2H). Other diastereomers can be prepared by using appropriate chiral starting materials.

### Example 73

### Preparation of Compound Nos. 85, 85a, 85b, 85c and 85d

(11aR)-7-(Trifluoromethyl)-2,3,5,10,11,11a-hexahydro-1H-indolizino[7,6-b]indole (150 mg, 0.53 mmol) was dissolved in DMF (2 mL) and sodium hydride (64 mg, 1.6 mmol) was added at 0° C. and stirred for 5 min. 3-(2-Methyl-oxiranyl)-pyridine (122 mg, 0.9 mmol) was added into the reaction mixture and was allowed to come to RT and the reaction mixture was stirred for 18 h. After consumption of starting material, the reaction mixture was poured into ice cold water and extracted with EtOAc (3×50 mL). The combined organic layer was washed with water (4×50 mL) and dried over sodium sulfate and concentrated under vacuum to obtain the crude product, which was purified by reverse phase chromatography to obtain 1-[(11aR)-7-(trifluoromethyl)-1,2,3,5,11,11a-hexahydroindolizino[7,6-b]indol-10-yl]-2-(3-pyridyl)propan-2-ol (200 mg). The mixture was separated by chiral chromatography to obtain (2R)-1-[(11aR)-7-(trifluoromethyl)-1,2,3,5,11,11a-hexahydroindolizino[7,6-b]indol-10-yl]-2-(3-pyridyl)propan-2-ol (30 mg). ¹H NMR (CDCl₃, freebase) δ (ppm): 8.78 (s, 1H), 8.55 (d, 1H), 7.75 (d, 1H), 7.65 (s, 1H), 7.25 (m, 3H), 4.20 (m, 3H), 3.35 (d, 1H), 3.25 (t, 1H), 2.85 (m, 2H), 2.60 (m, 1H), 2.50 (m, 1H), 2.35 (m, 1H), 2.10 (m, 1H), 2.00 (m, 1H), 1.90 (m, 1H), 1.50 (s, 3H). Other diastereomers can be prepared by using appropriate chiral starting materials.

### Example 74

### Preparation of Compound Nos. 86, 86a, 86b, 86c and 86d

(11aS)-7-Chloro-9-fluoro-2,3,5,10,11,11a-hexahydro-1H-indolizino[7,6-b]indole (150 mg, 0.56 mmol) was dissolved in DMF (2 mL) and sodium hydride (68 mg, 1.7 mmol) was added at 0° C. and stirred for 5 min. 4-(2-Methyl-oxiranyl)-pyridine (130 mg, 0.96 mmol) was added into the reaction mixture and was allowed to come to RT and the reaction mixture was stirred for 18 h. After consumption of starting material, the reaction mixture was poured into ice cold water and extracted with EtOAc (3×50 mL). The combined organic layer was washed with water (4×30 mL) and dried over sodium sulfate and concentrated to give a crude product, which was purified by reverse phase chromatography to obtain 1-[(11aS)-7-chloro-9-fluoro-1,2,3,5,11,11a-hexahydroindolizino[7,6-b]indol-10-yl]-2-(4-pyridyl)propan-2-ol (25 mg). ¹H NMR (CDCl₃, freebase) δ (ppm): 8.22 (d, 2H), 7.10 (d, 2H), 6.95 (s, 1H), 6.62 (d, 1H), 4.30 (m, 2H), 3.70 (m, 1H), 3.20 (t, 1H), 3.15 (m, 1H), 2.98 (m, 1H), 2.80 (m, 1H), 2.42 (m, 1H), 2.30 (m, 1H), 2.10 (m, 1H), 2.00 (m, 1H), 1.90 (m, 2H), 1.62 (s, 3H). Other diastereomers can be prepared by using appropriate chiral starting materials.

### Example 75

### Preparation of Compound Nos. 87, 87a, 87b, 87c and 87d

(11cS)-11-Fluoro-10-methyl-2,3,5,6,7,11c-hexahydro-1H-indolizino[7,8-b]indole (200 mg, 0.8 mmol) was dissolved in DMF (2 mL) and sodium hydride (96 mg, 2.4 mmol) was added at 0° C. and stirred for 5 min. 3-(2-Methyl-oxiranyl)-pyridine (188 mg, 1.3 mmol) was added into the reaction mixture and was allowed to come to RT and the reaction mixture was stirred for 18 h. After consumption of starting material, the reaction mixture was poured into ice cold water and extracted with EtOAc (3×50 mL). The combined organic layer was washed with water (4×30 mL) and dried over sodium sulfate and concentrated to give a crude product, which was purified by reverse phase chromatography to obtain (2R)-1-[(11cS)-11-fluoro-10-methyl-1,2,3,5,6,11c-hexahydroindolizino[7,8-b]indol-7-yl]-2-(3-pyridyl)propan-2-ol (40 mg), and 1-(10-fluoro-9-methyl-1,2,3,4,5,10c-hexahydro-3a,6-diaza-cyclopenta[c]fluoren-6-yl)-2-pyridin-3-yl-propan-2-(30 mg). ¹H NMR (CDCl₃, freebase) δ (ppm): 8.70 (s, 1H), 8.55 (d, 1H), 7.72 (d, 1H), 7.25 (m, 1H), 7.15 (d, 1H), 7.00 (d, 1H), 4.10 (m, 3H), 3.25 (m, 1H), 2.85 (m, 3H), 2.65 (m, 1H), 2.45 (m, 2H), 2.35 (s, 3H), 1.90 (m, 3H), 1.65 (s, 3H). Other diastereomers can be prepared by using appropriate chiral starting materials.

### Example 76

### Preparation of Compound Nos. 88, 88a, 88b, 88c and 88d

(11cR)-8-Chloro-10-fluoro-2,3,5,6,7,11c-hexahydro-1H-indolizino[7,8-b]indole (150 mg, 0.56 mmol) was dissolved in DMF (4 mL), and sodium hydride (68 mg, 1.7 mmol) was added at 0° C. and stirred for 5 min. 4-(2-Methyl-oxiranyl)-pyridine (153 mg, 1.13 mmol) was added and the reaction mixture was allowed to RT and stirred for 18 h. After consumption of starting material, the reaction mixture was poured into ice cold water and extracted with EtOAc (4×20 mL). The combined organic layer was washed with water (6×10 mL) and dried over sodium sulfate and concentrated under vacuum to obtain the crude product, which was purified by reverse phase chromatography to obtain 1-[(11cR)-8-chloro-10-fluoro-1,2,3,5,6,11c-hexahydroindolizino[7,8-b]indol-7-yl]-2-(4-pyridyl)propan-2-ol (30 mg). The racemic mixture was separated by chiral chromatography to obtain (2S)-1-[(11cR)-8-chloro-10-fluoro-1,2,3,5,6,11c-hexahydroindolizino[7,8-b]indol-7-yl]-2-(4-pyridyl)propan-2-ol (11 mg). ¹H NMR (CDCl₃, freebase) δ (ppm): 8.55 (d, 2H), 7.35 (d, 2H), 7.00 (d, 1H), 6.90 (d, 1H), 3.95 (t, 1H), 3.22 (m, 1H), 2.95-2.75 (m, 7H), 2.60 (m, 1H), 2.42 (m, 1H), 1.90 (m, 2H), 1.50 (s, 3H). Other diastereomers can be prepared by using appropriate chiral starting materials.

### Example 77

### Preparation of Compound Nos. 89, 89a, 89b, 89c and 89d

(11cS)-8-Chloro-10-fluoro-2,3,5,6,7,11c-hexahydro-1H-indolizino[7,8-b]indole (150 mg, 0.56 mmol) was dissolved in DMF (2 mL) and sodium hydride (68 mg, 1.7 mmol) was added at 0° C. and stirred for 5 min. 3-(2-Methyl-oxiranyl)-pyridine (153 mg, 1.13 mmol) and added dropwise into the reaction mixture and was allowed to come to RT and stirred for 18 h. After consumption of starting material, the reaction mixture was poured into ice cold water and extracted with EtOAc (4×20 mL). The combined organic layer was washed with water (6×10 mL) and dried over sodium sulfate and concentrated under vacuum to obtain the crude product, which was purified by reverse phase chromatography to obtain 1-[(11cS)-8-chloro-10-fluoro-1,2,3,5,6,11c-hexahydroindolizino[7,8-b]indol-7-yl]-2-(3-pyridyl)propan-2-ol (30 mg). The racemic mixture was separated by chiral chromatography to obtain (2S)-1-[(11cS)-8-chloro-10-fluoro-1,2,3,5,6,11c-hexahydroindolizino[7,8-b]indol-7-yl]-2-(3-pyridyl)propan-2-ol (9 mg). ¹H NMR (CDCl₃, freebase) δ (ppm): 8.65 (s, 1H), 8.45 (m, 1H), 7.60 (m, 1H), 7.10 (m, 1H), 6.95 (m, 1H), 6.85 (m, 1H), 4.10 (m, 1H), 3.25 (m, 1H), 3.00 (m, 5H), 2.48 (m, 2H), 2.32 (m, 1H), 1.98 (m, 3H), 1.60 (s, 3H). Other diastereomers can be prepared by using appropriate chiral starting materials.

### Example 78

### Preparation of Compound Nos. 91, 91a, 91b, 91c and 91d

To a solution of (S)-8,10-dimethyl-2,3,5,6,7,11c-hexahydro-1H-indolizino[7,8-b]indole (200 mg, 0.769 mmol) in DMF (5 mL) was added sodium hydride (120 mg, 3.08 mmol) at 0° C. After 20 min of stirring, a solution of 3-(2-methyloxiran-2-yl)pyridine (207 mg, 1.54 mmol) in DMF (1 mL) was added dropwise. The reaction mixture was allowed to stir at RT for 16 h. The progress of reaction was monitored by TLC and LCMS. After completion of the reaction, ice-cold water was added to the reaction mixture and the product was extracted with EtOAc (3×30 mL). The combined organic layer was washed with water (6×40 mL) and dried over sodium sulfate. Removal of solvent under reduced pressure afforded a crude product which was purified by column chromatography on neutral alumina, eluting a pure compound in 2-3% MeOH:DCM as eluent to afford a diastereomeric mixture which was purified by chiral HPLC to afford 50 mg of (S)-1-((R)-8-chloro-10-methyl-2,3,5,6-tetrahydro-1H-indolizino[7,8-b]indol-7(11cH)-yl)-2-(pyridin-4-yl)propan-2-ol as free base. ¹H NMR (CDCl₃, freebase) δ (ppm): 8.56 (d, 2H), 7.38 (d, 2H), 7.13 (s, 1H), 6.96 (s, 1H), 5.15 (brs, 1H), 4.20 (brs, 1H), 3.98 (brs, 1H), 3.22 (brs, 1H), 3.18-3.22 (m, 1H), 2.75-2.92 (m, 4H), 2.39-2.58 (m, 2H), 2.40 (s, 3H), 1.85-2.18 (m, 2H), 1.51 (s, 3H). Other diastereomers can be prepared by using appropriate chiral starting materials.

### Example 79

### Preparation of Compound Nos. 96, 96a and 96b

5-[2-(2,8-Dimethyl-3,4-dihydro-1H-pyrido[4,3-b]indol-5-yl)-1-hydroxy-ethyl]pyridine-2-carboxylic acid (20 mg, 0.054 mmol) was dissolved in DCM (0.3 mL) then PyBOP (31 mg, 0.060 mmol) was added. To this reaction mixture, triethylamine (11 mg, 0.108 mmol) and pyrrolidine (5.7 mg, 0.081 mmol) were added. The resultant reaction mixture was allowed to stir at RT for 4 h. Progress of the reaction was monitored by LCMS. Water (2 mL) was added and the mixture was extracted with DCM (3×5 mL). The combined organic layer was washed with water (2×3 mL), dried over sodium sulfate, and evaporated to obtain the crude product, which was purified by preparative HPLC to obtain [5-[2-(2,8-dimethyl-3,4-dihydro-1H-pyrido[4,3-b]indol-5-yl)-1-hydroxy-ethyl]-2-pyridyl]-pyrrolidin-1-yl-methanone (4 mg). ¹H NMR (CD₃OD, freebase) δ (ppm): 8.20 (s, 1H), 7.80 (d, 1H), 7.50 (d, 1H), 7.20 (s, 1H), 7.00 (d, 1H), 6.80 (d, 1H), 5.18 (t, 1H), 4.22-(dd, 1H), 4.40 (dd, 1H), 4.00 (q, 2H), 3.60 (t, 2H), 3.40 (t, 3H), 3.20 (m, 4H), 2.70 (s, 3H), 2.35 (s, 3H), 1.90 (m, 4H). The other enantiomer can be prepared by using appropriate chiral starting materials.

### Example 80

### Preparation of Compound Nos. 101, 101a, 101b, 101c and 101d

To the corresponding carboline (200 mg, 0.884 mmol) was added DMF (5 mL) and the mixture was stirred for 2 min. To this, sodium hydride (106 mg, 2.652 mmol) was added and the reaction mixture was stirred for 5 min. 3-(2-Methyloxiran-2-yl)pyridine (143 mg, 1.061 mmol) was added and the reaction was stirred overnight. Progress of the reaction was monitored by LCMS. Ice cold water was added to the reaction mixture, which was then filtered to get the desired product (270 mg). The racemic compound was separated by chiral HPLC to get Cpd. No. 101a, (86 mg) & Cpd. No. 101b (39 mg). ¹H NMR (CDCl₃, freebase) δ (ppm): 8.75 (s, 1H), 8.56 (d, 1H), 7.64 (d, 1H), 7.25-7.20 (m, 3H), 6.96 (d, 1H), 4.16 (m, 3H), 3.50 (m, 1H), 2.90 (d, 1H), 2.42 (s, 3H), 2.30 (s, 3H), 2.20 (m, 2H), 1.95-1.80 (m, 2H), 1.70 (s, 3H). Other diastereomers can be prepared by using appropriate chiral starting materials.

### Example 81

### Preparation of Compound Nos. 102, 102a, 102b, 102c and 102d

To the corresponding carboline (200 mg, 0.884 mmol) was added DMF (5 mL) and the reaction was stirred for 2 min. To this, sodium hydride (106 mg, 2.652 mmol) was added and the reaction mixture was allowed to stir for 5 min. 2-(4-Fluorophenyl)-2-methyloxirane (161 mg, 1.061 mmol) was added and the reaction was stirred overnight. Progress of the reaction was monitored by LCMS. Ice cold water was added to the reaction mixture and the mixture was filtered to get the desired product (260 mg). ¹H NMR (CDCl₃, freebase) δ (ppm): 7.40 (m, 2H), 2.27-7.20 (m, 3H), 7.04 (m, 2H), 6.97 (d, 1H), 4.19 (d, 1H), 4.12 (d, 1H), 4.06 (d, 1H), 3.45 (m, 1H), 2.70 (d, 1H), 2.44 (s, 3H), 2.24 (s, 3H), 2.22 (m, 3H), 2.0 (d, 1H), 1.82 (m, 1H), 1.61 (s, 3H). The racemic compound was separated by chiral HPLC to get Cpd. No. 102a (82 mg) and Cpd. No. 102b (72 mg). Other diastereomers can be prepared by using appropriate chiral starting materials.

### Example 82

### Preparation of Compound Nos. 134, 134a, 134b, 134c and 134d

The carboline (200 mg, 0.942 mmol) was dissolved in DMF (10 mL), sodium hydride (67 mg, 2.82 mmol) was added and stirred for 5 min. 2-(4-Fluoro-phenyl)-2-methyl-oxirane (229 mg, 1.50 mmol) was added and the reaction mixture was allowed to stir at RT for 12 h. After consumption of starting material, the reaction mixture was poured in to ice water and extracted with EtOAc (3×75 mL). The combined organic layer was washed with water (4×50 mL), dried over sodium sulfate and concentrated to obtain the crude product that was crystallized in ether/hexane to obtain 240 mg of desired compound. This was separated by chiral HPLC to obtain 134a (12 mg), 134b (12 mg), 134c (12 mg) and 134d (12 mg). 134a: ¹H NMR (CDCl₃, freebase) δ (ppm): 7.30-7.20 (m, 4H), 7.0 (m, 2H), 6.96 (d, 1H), 4.10 (d, 1H), 3.93 (d, 1H), 3.79 (d, 1H), 3.29 (m, 1H), 3.20 (m, 1H), 2.92 (m, 2H), 2.60 (m, 1H), 2.42 (s, 3H), 2.0 (m, 2H), 1.65 (s, 3H). 134b: ¹H NMR (CDCl₃, freebase) δ (ppm): 7.30-7.20 (m, 4H), 7.0 (m, 2H), 6.96 (d, 1H), 3.98 (m, 2H), 3.30 (m, 1H), 3.20 (m, 2H), 2.90 (d, 1H), 2.80 (d, 1H), 2.50 (m, 1H), 2.42 (s, 3H), 1.98 (m, 2H), 1.62 (s, 3H). 134c: ¹H NMR (CDCl₃, freebase) δ (ppm): 7.30-7.20 (m, 4H), 7.0 (m, 2H), 6.96 (d, 1H), 4.0-3.90 (m, 3H), 3.30 (m, 1H), 3.20 (m, 2H), 2.90 (d, 1H), 2.80 (d, 1H), 2.50 (m, 1H), 2.42 (s, 3H), 1.98 (m, 2H), 1.62 (s, 3H). 134d: ¹H NMR (CDCl₃, freebase) δ (ppm): 7.30-7.20 (m, 4H), 7.0 (m, 2H), 6.96 (d, 1H), 4.10 (d, 1H), 3.93 (d, 1H), 3.78 (d, 1H), 3.30 (m, 2H), 3.20 (m, 1H), 2.94 (d, 1H), 2.88 (d, 1H), 2.60 (m, 1H), 2.42 (s, 3H), 1.98 (m, 2H), 1.62 (s, 3H).

### Example 83

### Preparation of Compound Nos. 135, 135a, 135b, 135c and 135d

(11cR)-10-Chloro-8-fluoro-2,3,5,6,7,11c-hexahydro-1H-indolizino[7,8-b]indole (200 mg, 0.7 mmol) was dissolved in DMF (2.5 mL), and sodium hydride (90 mg, 2.2 mmol) was added at 0° C. and stirred for 5 min. 4-Oxiranyl-pyridine (155 mg, 1.2 mmol) was added and the reaction mixture was allowed to RT and stirred for 18 h. After consumption of starting material, the reaction mixture was poured into ice cold water and extracted with EtOAc (3×75 mL). The combined organic layer was washed with water (4×50 mL) and dried over sodium sulfate and concentrated under vacuum to obtain the crude product, which was purified by reverse phase chromatography to obtain 2-[(11cR)-10-chloro-8-fluoro-1,2,3,5,6,11c-hexahydroindolizino[7,8-b]indol-7-yl]-1-(4-pyridyl)ethanol (120 mg), the racemic mixture was separated by chiral chromatography to obtain (1S)-2-[(11cR)-10-chloro-8-fluoro-1,2,3,5,6,11c-hexahydroindolizino[7,8-b]indol-7-yl]-1-(4-pyridyl)ethanol (26 mg). ¹H NMR (CDCl₃, freebase) δ (ppm): 8.60 (d, 2H), 7.30 (d, 2H), 7.22 (s, 1H), 6.88 (d, 1H), 5.05 (m, 1H), 4.40 (m, 1H), 4.15 (m, 1H), 3.78 (t, 1H), 3.30 (m, 1H), 3.08 (m, 1H), 2.95 (m, 1H), 2.70 (m, 2H), 2.22 (m, 2H), 1.90 (m, 3H). Other diastereomers can be prepared by using appropriate chiral starting materials.

### Example 84

### Preparation of Compound Nos. 136, 136a, 136b, 136c and 136d

(11cR)-10-Fluoro-8-methyl-2,3,5,6,7,11c-hexahydro-1H-indolizino[7,8-b]indole (150 mg, 0.614 mmol) was dissolved in DMF (2 mL) and sodium hydride (73 mg, 1.82 mmol) was added at 0° C. and stirred for 5 min. 4-Oxiranyl-pyridine (148 mg, 1.22 mmol) was dissolved in DMF (1 mL) and added dropwise into the reaction mixture was allowed to RT and stirred for 18 h. After consumption of starting material, the reaction mixture was poured in to ice cold water and extracted with EtOAc (4×20 mL). The combined organic layer was washed with water (4×10 mL) and dried over sodium sulfate and concentrated under vacuum to obtain the crude product, which was purified by reverse phase chromatography to obtain 2-[(11cR)-10-fluoro-8-methyl-1,2,3,5,6,11c-hexahydroindolizino[7,8-b]indol-7-yl]-1-(4-pyridyl)ethanol (62 mg). The racemic mixture was separated by chiral chromatography to obtain (1S)-2-[(11cR)-10-fluoro-8-methyl-1,2,3,5,6,11c-hexahydroindolizino[7,8-b]indol-7-yl]-1-(4-pyridyl)ethanol (19 mg). ¹H NMR (CDCl₃, freebase) δ (ppm): 8.58 (d, 2H), 7.20 (d, 2H), 6.92 (d, 1H), 6.68 (d, 1H), 4.90 (m, 1H), 4.15 (m, 2H), 3.85 (t, 1H), 3.20 (m, 1H), 2.85 (m, 1H), 2.80 (m, 2H), 2.75 (s, 3H), 2.60 (m, 1H), 2.38 (m, 1H), 1.85 (m, 4H). Other diastereomers can be prepared by using appropriate chiral starting materials.

### Example 85

### Preparation of Compound Nos. 137, 137a, 137b, 137c and 137d

(11cR)-8-Fluoro-10-methyl-2,3,5,6,7,11c-hexahydro-1H-indolizino[7,8-b]indole (150 mg, 0.614 mmol) was dissolved in DMF (2 mL) and sodium hydride (73 mg, 1.82 mmol) was added at 0° C. and stirred for 5 min. 4-Oxiranyl-pyridine (148 mg, 1.22 mmol) was dissolved in DMF (1 mL) and added dropwise into the reaction mixture was allowed to RT and stirred for 18 h. After consumption of starting material, the reaction mixture was poured in to ice cold water and extracted with EtOAc (4×20 mL). The combined organic layer was washed with water (6×10 mL) and dried over sodium sulfate and concentrated under vacuum to obtain the crude product, which was purified by reverse phase chromatography to obtain 2-[(11cR)-8-fluoro-10-methyl-1,2,3,5,6,11c-hexahydroindolizino[7,8-b]indol-7-yl]-1-(4-pyridyl) ethanol (93 mg). The racemic mixture was separated by chiral chromatography to obtain (1S)-2-[(11cR)-8-fluoro-10-methyl-1,2,3,5,6,11c-hexahydroindolizino[7,8-b]indol-7-yl]-1-(4-pyridyl)ethanol (25 mg). ¹H NMR (CDCl₃, freebase) δ (ppm): 8.58 (d, 2H), 7.32 (d, 2H), 7.00 (s, 1H), 6.70 (d, 1H), 5.10 (m, 1H), 4.40 (m, 1H), 4.10 (m, 1H), 3.85 (t, 1H), 3.22 (m, 1H), 3.05 (m, 1H), 2.95 (m, 1H), 2.75 (m, 2H), 2.48 (m, 1H), 2.42 (s, 3H), 1.90 (m, 4H). Other diastereomers can be prepared by using appropriate chiral starting materials.

### Example 86

### Preparation of Compound Nos. 138, 138a, 138b, 138c and 138d

2-[(11cR)-10-Methyl-1,2,3,5,6,11c-hexahydroindolizino[7,8-b]indol-7-yl]-1-(4-pyridyl)ethanol (200 mg, 0.576 mmol) was dissolved in DMF (2 mL), and cooled to 0° C. Sodium hydride (69 mg, 1.725 mmol) was added portionwise and bromomethyl-cyclobutane (429 mg, 2.879 mmol) was added at the same temperature, and the reaction mixture was warmed to RT and stirred for 2 h. After consumption of starting material, the reaction mixture was poured in to ice cold water and extracted with EtOAc (4×20 mL). The combined organic layer was washed with water (6×10 mL) and dried over sodium sulfate and concentrated under vacuum to obtain the crude product, which was purified by reverse phase chromatography to obtain (11cR)-7-[2-(cyclobutylmethoxy)-2-(4-pyridyl)ethyl]-10-methyl-1,2,3,5,6,11c-hexahydroindolizino[7,8-b]indole (68 mg). The mixture was separated by chiral chromatography to obtain (11cR)-7-[(2R)-2-(cyclobutylmethoxy)-2-(4-pyridyl)ethyl]-10-methyl-1,2,3,5,6,11c-hexahydroindolizino[7,8-b]indole (11 mg). ¹H NMR (CDCl₃, freebase) δ (ppm): 8.55 (d, 2H), 7.26 (s, 1H), 7.18 (d, 1H), 7.10 (d, 2H), 6.98 (d, 1H), 4.55 (t, 1H), 4.25 (m, 1H), 4.10 (m, 2H), 3.22 (m, 3H), 2.82 (m, 4H), 2.48 (m, 1H), 2.45 (s, 3H), 2.18 (m, 1H), 2.00-1.82 (m, 7H), 1.65 (m, 3H). Other diastereomers can be prepared by using appropriate chiral starting materials.

### Example 87

### Preparation of Compound Nos. 139, 139a and 139b

To a stirred solution of 1-(8-(2-(tert-butyldimethylsilyloxy)ethyl)-2-methyl-3,4-dihydro-1H-pyrido[4,3-b]indol-5(2H)-yl)-2-(pyridin-3-yl)propan-2-ol (350 mg, 0.730 mmol) in THF (5 mL) at 0° C. was added TBAF in THF solution (1M, 2.2 mL, 2.192 mmol) dropwise. The reaction mixture was allowed to stir at RT for 2 h. The reaction mixture was poured on ice-water and extracted with EtOAc (2×25 mL). The organic layer dried over anhydrous sodium sulfate, concentrated under vacuum to obtain a crude product, which was purified by column chromatography using neutral alumina (eluent system 5% MeOH:DCM) to obtain the desired product, which was separated by chiral column to obtain (S)-1-(8-(2-hydroxyethyl)-2-methyl-3,4-dihydro-1H-pyrido[4,3-b]indol-5(2H)-yl)-2-(pyridin-3-yl)propan-2-ol and (R)-1-(8-(2-hydroxyethyl)-2-methyl-3,4-dihydro-1H-pyrido[4,3-b]indol-5(2H)-yl)-2-(pyridin-3-yl)propan-2-ol (18 mg). ¹H NMR (CDCl₃, freebase) δ (ppm): 8.75 (s, 1H), 8.42 (d, 1H), 7.62 (d, 1H), 7.2 (s, 1H), 7.15 (d, 2H), 6.95 (d, 1H), 4.25-4.15 (dd, 2H), 3.8 (t, 2H), 3.6 (s, 2H), 2.88 (t, 2H), 2.8 (t, 2H), 2.75 (t, 2H), 2.6 (s, 3H), 1.7 (s, 3H). Other diastereomers can be prepared by using appropriate chiral starting materials.

### Example 88

### Preparation of Compound Nos. 140, 140a and 140b

To a stirred solution of 2-(8-(2-(tert-butyldimethylsilyloxy)ethyl)-2-methyl-3,4-dihydro-1H-pyrido[4,3-b]indol-5(2H)-yl)-1-(pyridin-4-yl)ethanol (280 mg, 0.602 mmol) in THF (4 mL) at 0° C. was added TBAF in THF Solution (1M, 1.8 mL, 1.806 mmol) dropwise. The reaction mixture was allowed to stir at RT for 2 h. The reaction mixture was poured into ice-water and extracted with EtOAc (2×20 mL). The organic layer dried over anhydrous sodium sulfate, concentrated under vacuum to obtain a crude product, which was purified by column chromatography using neutral alumina (eluent system 5% MeOH:DCM) to obtain the desired product, which was separated by chiral column to obtain (S)-2-(8-(2-hydroxyethyl)-2-methyl-3,4-dihydro-1H-pyrido[4,3-b]indol-5(2H)-yl)-1-(pyridin-4-yl)ethanol and (R)-2-(8-(2-hydroxyethyl)-2-methyl-3,4-dihydro-1H-pyrido[4,3-b]indol-5(2H)-yl)-1-(pyridin-4-yl)ethanol (7 mg). ¹H NMR (CDCl₃, freebase) δ (ppm): 8.3 (d, 2H), 7.1 (d, 2H), 7.05 (s, 1H), 6.98 (d, 1H), 6.9 (d, 1H), 4.95 (t, 1H), 4.1 (d, 2H), 3.8 (t, 2H), 3.6 (d, 1H), 3.4 (d, 1H), 3.0 (t, 1H), 2.98 (t, 1H), 2.92 (t, 2H), 2.85 (t, 2H), 3.05 (s, 3H). The other enantiomer can be prepared by using appropriate chiral starting materials.

### Example 89

### Preparation of Compound Nos. 141, 141a, 141b, 141c and 141d

(R)-8,10-Dimethyl-2,3,5,6,7,11c-hexahydro-1H-indolizino[7,8-b]indole (100 mg, 0.416 mmol) was dissolved in DMF (2 mL). Sodium hydride (50 mg, 1.24 mmol) was added into the reaction mixture and stirred at RT for 10 min. 2-Cyclohexyloxirane (78 mg, 0.63 mmol) was added dropwise over 10 min and reaction mixture was stirred at RT for 12 h. The progress of reaction was monitored by TLC and LCMS. Ice cold water was added into the reaction mixture and extracted with EtOAc (3×100 mL). The combined organic layer was washed with water (4×100 mL), dried over sodium sulfate and concentrated to obtain the crude product that was purified by reverse phase chromatography to get the racemic compound. The racemic compound was separated by chiral HPLC to obtain (R)-1-cyclohexyl-2-((R)-8,10-dimethyl-2,3,5,6-tetrahydro-1H-indolizino[7,8-b]indol-7(11cH)-yl)ethanol (30 mg). ¹H NMR (CDCl₃, freebase) δ (ppm): 7.10 (s, 1H), 6.70 (s, 1H), 4.38 (d, 1H), 4.10 (m, 1H), 4.0 (m, 1H), 3.62 (m, 1H), 3.30 (m, 1H), 3.08 (m, 1H), 2.90 (m, 2H), 2.80 (m, 1H), 2.64 (s, 3H), 2.58 (m, 1H), 2.42 (m, 1H), 2.40 (s, 3H), 1.90 (m, 4H), 1.78 (m, 4H), 1.30-1.10 (m, 6H). Other diastereomers can be prepared by using appropriate chiral starting materials.

### Example 90

### Preparation of Compound Nos. 142, 142a, 142b, 142c and 142d

To a solution of (R)-8-chloro-10-methyl-2,3,5,6,7,11c-hexahydro-1H-indolizino[7,8-b]indole (20 mg, 0.769 mmol) in DMF (4 mL) was added sodium hydride (123 mg, 3.076 mmol) at 0° C. After 20 min of stirring, a solution of 4-(oxiran-2-yl)pyridine (186 mg, 1.538 mmol) in DMF (1 mL) was added dropwise. The reaction mixture was allowed to stir at RT for 16 h. The progress of reaction was monitored by TLC and LCMS. After completion of the reaction, ice-cold water was added to the reaction mixture and the product was extracted with EtOAc (3×30 mL). The combined organic layer was washed with water (6×40 mL) and dried over sodium sulfate. Removal of solvent under reduced pressure afforded a crude product that was purified by column chromatography on neutral alumina eluting pure compound in 2-3% MeOH:DCM as eluent to afford a diastereomeric mixture, which was purified by chiral HPLC to afford 10 mg of (S)-2-((R)-8-chloro-10-methyl-2,3,5,6-tetrahydro-1H-indolizino[7,8-b]indol-7(11cH)-yl)-1-(pyridin-4-yl)ethanol. ¹H NMR (CDCl₃, freebase) δ (ppm): 8.6 (d, 2H), 7.25 (d, 2H), 7.19 (s, 1H), 7.0 (s, 1H), 5.2 (t, 1H), 4.8 (d, 1H), 4.2 (t, 1H), 3.95 (bs, 1H), 3.25 (t, 1H), 3.1 (t, 1H), 2.95 (t, 1H), 2.8 (t, 2H), 2.45 (m, 2H), 2.4 (s, 3H), 1.95 (m, 4H). Other diastereomers can be prepared by using appropriate chiral starting materials.

### Example 91

### Preparation of Compound Nos. 143, 143a, 143b, 143c and 143d

(11cR)-10-Chloro-2,3,5,6,7,11c-hexahydro-1H-indolizino[7,8-b]indole (200 mg, 0.813 mmol) was dissolved in DMF (3 mL) and sodium hydride (49 mg, 2.0 mmol) was added at 0° C. and stirred for 5 min. 4-Oxiranyl-pyridine (148 mg, 1.2 mmol) was added and the reaction mixture was allowed to stir at RT for 18 h. After consumption of starting material, the reaction mixture was poured in to ice cold water and extracted with EtOAc, dried over sodium sulfate and concentrated under vacuum to obtain the crude product, which was recrystalised from ether/hexane to obtain 2-[(11cR)-10-chloro-1,2,3,5,6,11c-hexahydroindolizino[7,8-b]indol-7-yl]-1-(4-pyridyl) ethanol (120 mg). The racemic mixture was separated by chiral chromatography to obtain (1R)-2-[(11cR)-10-chloro-1,2,3,5,6,11c-hexahydroindolizino[7,8-b]indol-7-yl]-1-(4-pyridyl)ethanol (35 mg). ¹H NMR (CDCl₃, freebase) δ (ppm): 8.58 (d, 2H), 7.42 (s, 1H), 7.22 (m, 3H), 7.10 (d, 1H), 5.00 (t, 1H), 4.18 (m, 2H), 3.82 (t, 1H), 3.25 (m, 1H), 2.92 (m, 2H), 2.75 (m, 2H), 2.42 (m, 2H), 1.90 (m, 3H). Other diastereomers can be prepared by using appropriate chiral starting materials.

### Example 92

### Preparation of Compound Nos. 144, 144a, 144b, 144c and 144d

(11cR)-8,10-Difluoro-2,3,5,6,7,11c-hexahydro-1H-indolizino[7,8-b]indole (200 mg, 0.806 mmol) was dissolved in DMF (2 mL) and sodium hydride (48 mg, 2.0 mmol) was added at 0° C. and stirred for 5 min. 4-Oxiranyl-pyridine (146 mg, 1.2 mmol) was added and the reaction mixture was allowed to RT and stirred for 18 h. After consumption of starting material, the reaction mixture was poured in to ice cold water and extracted with EtOAc, dried over sodium sulfate and concentrated under vacuum to obtain the crude product, which was recrystalised in ether hexane to obtain 2-[(11cR)-8,10-difluoro-1,2,3,5,6,11c-hexahydroindolizino[7,8-b]indol-7-yl]-1-(4-pyridyl)ethanol (110 mg). The racemic mixture was separated by chiral chromatography to obtain (1S)-2-[(11cR)-8,10-difluoro-1,2,3,5,6,11c-hexa hydro indolizino[7,8-b]indol-7-yl]-1-(4-pyridyl)ethanol (25 mg). ¹H NMR (CDCl₃, freebase) δ (ppm): 8.58 (d, 2H), 7.28 (d, 2H), 6.90 (d, 1H), 6.65 (t, 1H), 5.05 (m, 1H), 4.20 (m, 1H), 4.10 (m, 1H), 3.78 (t, 1H), 3.30 (m, 1H), 3.05 (m, 1H), 2.92 (m, 1H), 2.70 (m, 2H), 2.40 (m, 2H), 1.90 (m, 3H). Other diastereomers can be prepared by using appropriate chiral starting materials.

### Example 93

### Preparation of Compound Nos. 145, 145a, 145b, 145c and 145d

To a solution of 2-(2,6-dimethyl-3,4-dihydro-1H-pyrido[3,4-b]indol-9(2H)-yl)-1-(pyridin-4-yl)ethanol (250 mg, 0.77 mmol) in THF/Water/Acetic acid (24 mL, 1:1:1), and N-bromosuccinimide (138 mg, 0.77 mmol) was added. The reaction mixture was stirred in the dark at RT for 1 h. The reaction mixture was neutralized by adding to it a saturated solution of aqueous sodium bicarbonate. The aqueous layer was extracted with EtOAc (2×150 mL). The combined organic extract was dried over anhydrous sodium sulfate and concentrated to obtain the crude product. The crude mixture was purified by reverse phase HPLC to obtain 1-(2-hydroxy-2-(pyridin-4-yl)ethyl)-1′,5-dimethylspiro[indoline-3,3′-pyrrolidin]-2-one (75 mg) followed by chiral separation to obtain 145a (5 mg), 145b (5 mg), 145c (5 mg) and 145d (5 mg). 145a: ¹H NMR (CD₃OD, freebase) δ (ppm): 8.62 (d, 2H), 7.76 (d, 2H), 7.30 (m, 1H), 7.18 (d, 1H), 7.10 (d, 1H), 5.20 (m, 1H), 4.05 (m, 1H), 3.95 (m, 1H), 3.60-3.40 (m, 2H), 3.18 (s, 3H), 2.50 (m, 1H), 2.39 (m, 1H), 2.36 (s, 3H). 145b: ¹H NMR (CDCl₃, freebase) δ (ppm): 8.53 (d, 2H), 7.55 (s, 1H), 7.45 (d, 2H), 7.09 (d, 1H), 6.84 (d, 1H), 5.01 (m, 1H), 3.99 (m, 1H), 3.89 (m, 1H), 3.84 (d, 2H), 3.48 (m, 1H), 3.15 (d, 1H), 2.97 (s, 3H), 2.59 (m, 1H), 2.32 (s, 3H), 2.25 (m, 1H). 145c: ¹H NMR (CD₃OD, freebase) δ (ppm): 8.67 (d, 2H), 7.89 (d, 2H), 7.37 (s, 1H), 7.20 (d, 1H), 7.11 (d, 1H), 4.06-3.92 (m, 4H), 3.70-3.50 (m, 2H), 3.09 (s, 3H), 2.48 (m, 1H), 2.36 (s, 3H), 2.32 (m, 1H). 145d: ¹H NMR (CDCl₃, freebase) δ (ppm): 8.51 (d, 2H), 7.56 (s, 1H), 7.38 (d, 2H), 7.10 (d, 1H), 6.85 (d, 1H), 5.0 (m, 1H), 4.0 (d, 1H), 3.97-3.84 (m, 3H), 3.50 (m, 1H), 3.18 (d, 1H), 2.98 (s, 3H), 2.58 (m, 1H), 2.32 (s, 3H), 2.23 (m, 1H).

### Example 94

### Preparation of Compound Nos. 146, 146a, 146b, 146c and 146d

(11cR)-10-(Trifluoromethyl)-2,3,5,6,7,11c-hexahydro-1H-indolizino[7,8-b]indole (200 mg, 0.714 mmol) was dissolved in DMF (2 mL) and sodium hydride (43 mg, 1.79 mmol) was added at RT and stirred for 10 min. 4-(Oxiran-2-yl)pyridine (173 mg, 1.43 mmol) was dissolved in DMF (1 mL) and added dropwise at RT and stirred for 18 h. After consumption of starting material, the reaction mixture was poured in to ice water and extracted with EtOAc (4×20 mL). The combined organic layer was washed with water (4×10 mL) and dried over sodium sulfate and concentrated under vacuum to obtain the crude product, which was purified by reverse phase chromatography to obtain 160 mg of desired compound. The racemic mixture was separated by chiral chromatography to obtain (1S)-2-[(11cR)-10-(trifluoromethyl)-1,2,3,5,6,11c-hexahydroindolizino[7,8-b]indol-7-yl]-1-(4-pyridyl)ethanol (40 mg). ¹H NMR (CDCl₃, freebase) δ (ppm): 8.55 (d, 2H), 7.70 (s, 1H), 7.40 (m, 2H), 7.22 (d, 2H), 5.05 (t, 1H), 4.22 (d, 2H), 3.95 (t, 1H), 3.28 (m, 1H), 3.00-2.75 (m, 4H), 2.50 (m, 2H), 1.90 (m, 3H). Other diastereomers can be prepared by using appropriate chiral starting materials.

### Example 95

### Preparation of Compound Nos. 147, 147a, 147b, 147c and 147d

(11cR)-10-(Trifluoromethyl)-2,3,5,6,7,11c-hexahydro-1H-indolizino[7,8-b]indole (200 mg, 0.714 mmol) was dissolved in DMF (2 mL) and sodium hydride (43 mg, 1.79 mmol) was added at RT and stirred for 10 min. 3-(Oxiran-2-yl)pyridine (173 mg, 1.43 mmol) was dissolved in DMF (1 mL) and added dropwise at RT and stirred for 18 h. After consumption of starting material, the reaction mixture was poured in to ice cold water and extracted with EtOAc (4×20 mL). The combined organic layer was washed with water (4×10 mL) and dried over sodium sulfate and concentrated under vacuum to obtain the crude product, which was purified by reverse phase chromatography to obtain 160 mg of desired compound. The racemic mixture was separated by chiral chromatography to obtain(1S)-2-[(11cR)-10-(trifluoromethyl)-1,2,3,5,6,11c-hexahydroindolizino[7,8-b]indol-7-yl]-1-(3-pyridyl)ethanol (50 mg). ¹H NMR (CDCl₃, freebase) δ (ppm): 8.52 (s, 1H), 8.30 (d, 1H), 7.58 (s, 1H), 7.42 (d, 1H), 7.35 (m, 2H), 7.10 (m, 1H), 5.05 (t, 1H), 4.30 (m, 1H), 4.15 (m, 1H), 3.75 (t, 1H), 3.18 (m, 1H), 2.88 (m, 2H), 2.75 (m, 2H), 2.50 (m, 1H), 2.39 (m, 1H), 1.90 (m, 2H), 1.80 (m, 1H). Other diastereomers can be prepared by using appropriate chiral starting materials.

### Example 96

### Preparation of Compound Nos. 148, 148a, 148b, 148c and 148d

(11cR)-10-Chloro-8-fluoro-2,3,5,6,7,11c-hexahydro-1H-indolizino[7,8-b]indole (150 mg, 0.56 mmol) was dissolved in DMF (2 mL) and sodium hydride (67 mg, 1.16 mmol) was added at 0° C. and stirred for 5 min. 3-(oxiran-2-yl)pyridine (116 mg, 0.96 mmol) was dissolved in DMF (1 mL) and added dropwise into the reaction mixture was allowed to RT and stirred for 18 h. After consumption of starting material, the reaction mixture was poured into ice cold water and extracted with EtOAc (3×50 mL). The combined organic layer was washed with water (3×40 mL) and dried over sodium sulfate and concentrated under vacuum to obtain the crude product, which was purified by reverse phase chromatography to obtain 130 mg of desired compound. The racemic mixture was separated by chiral chromatography to obtain (1S)-2-[(11cR)-10-chloro-8-fluoro-1,2,3,5,6,11c-hexahydroindolizino[7,8-b]indol-7-yl]-1-(3-pyridyl)ethanol (15 mg). ¹H NMR (CDCl₃, freebase) δ (ppm): 8.60 (s, 1H), 8.52 (d, 1H), 7.62 (s, 1H), 7.27 (m, 1H), 7.19 (m, 1H), 6.88 (d, 1H), 5.10 (m, 1H), 4.38 (m, 1H), 4.22 (m, 1H), 3.85 (t, 1H), 3.30 (m, 1H), 3.05 (m, 1H), 2.95 (m, 1H), 2.80 (m, 2H), 2.45 (m, 2H), 1.85 (m, 3H). Other diastereomers can be prepared by using appropriate chiral starting materials.

### Example 97

### Preparation of Compound Nos. 149, 149a, 149b, 149c and 149d

(11cS)-10-chloro-8-methyl-2,3,5,6,7,11c-hexahydro-1H-indolizino[7,8-b]indole (150 mg, 0.56 mmol) was dissolved in DMF (2 mL) and sodium hydride (67 mg, 1.16 mmol) was added at 0° C. and stirred for 5 min. 4-(oxiran-2-yl)pyridine (116 mg, 0.96 mmol) was dissolved in DMF (1 mL) and added dropwise into the reaction mixture was allowed to RT and stirred for 18 h. After consumption of starting material, the reaction mixture was poured into ice cold water and extracted with EtOAc (3×50 mL). The combined organic layer was washed with water (3×40 mL) and dried over sodium sulfate and concentrated under vacuum to obtain the crude product, which was purified by reverse phase chromatography to obtain 100 mg of desired compound. The racemic mixture was separated by chiral chromatography to obtain (1S)-2-[(11cS)-10-chloro-8-methyl-1,2,3,5,6,11c-hexahydroindolizino[7,8-b]indol-7-yl]-1-(4-pyridyl)ethanol (25 mg). ¹H NMR (CDCl₃, freebase) δ (ppm): 8.52 (d, 2H), 7.25 (s, 1H), 7.20 (d, 2H), 6.90 (s, 1H), 4.85 (t, 1H), 4.32 (m, 2H), 3.92 (t, 1H), 3.22 (m, 1H), 3.00 (m, 1H), 2.85 (m, 3H), 2.70 (s, 3H), 2.45 (m, 2H), 1.90 (m, 3H). Other diastereomers can be prepared by using appropriate chiral starting materials.

### Example 98

### Preparation of Compound Nos. 150, 150a, 150b, 150c and 150d

(11cS)-10-Chloro-8-methyl-2,3,5,6,7,11c-hexahydro-1H-indolizino[7,8-b]indole (150 mg, 0.56 mmol) was dissolved in DMF (2 mL) and sodium hydride (67 mg, 1.16 mmol) was added at 0° C. and stirred for 5 min. 3-(Oxiran-2-yl)pyridine (116 mg, 0.96 mmol) was dissolved in DMF (1 mL) and added dropwise into the reaction mixture was allowed to RT and stirred for 18 h. After consumption of starting material, the reaction mixture was poured into ice cold water and extracted with EtOAc (3×50 mL). The combined organic layer was washed with water (3×40 mL) and dried over sodium sulfate and concentrated under vacuum to obtain the crude product, which was purified by reverse phase chromatography to obtain the desired compound. The racemic mixture was separated by chiral chromatography to obtain (1S)-2-[(11cS)-10-chloro-8-methyl-1,2,3,5,6,11c-hexahydroindolizino[7,8-b]indol-7-yl]-1-(3-pyridyl)ethanol (3 mg). ¹H NMR (CDCl₃, freebase) δ (ppm): 8.50 (s, 1H), 8.40 (d, 1H), 7.48 (d, 1H), 7.20 (m, 2H), 6.90 (s, 1H), 4.92 (m, 1H), 4.48 (m, 1H), 4.25 (m, 1H), 3.80 (t, 1H), 3.18 (m, 1H), 2.92 (m, 2H), 2.80 (m, 1H), 2.72 (s, 3H), 2.40 (m, 2H), 1.90 (m, 4H). Other diastereomers can be prepared by using appropriate chiral starting materials.

### Example 99

### Preparation of Compound Nos. 151, 151a, 151b, 151c and 151d

(1S)-2-[(11cR)-9-Chloro-10-methyl-1,2,3,5,6,11c-hexahydroindolizino[7,8-b]indol-7-yl]-1-(4-pyridyl)ethanol (150 mg, 0.56 mmol) was dissolved in DMF (2 mL) and sodium hydride (67 mg, 1.16 mmol) was added at 0° C. and stirred for 5 min. 3-(Oxiran-2-yl)pyridine (116 mg, 0.96 mmol) was dissolved in DMF (1 mL) and added dropwise into the reaction mixture was allowed to RT and stirred for 18 h. After consumption of starting material, the reaction mixture was poured in to ice cold water and extracted with EtOAc (3×50 mL). The combined organic layer was washed with water (3×40 mL) and dried over sodium sulfate and concentrated under vacuum to obtain the crude product, which was purified by reverse phase chromatography to obtain 100 mg of desired compound. The racemic mixture was separated by chiral chromatography to obtain (1S)-2-[(11cR)-9-chloro-10-methyl-1,2,3,5,6,11c-hexahydroindolizino[7,8-b]indol-7-yl]-1-(3-pyridyl)ethanol (30 mg). ¹H NMR (CDCl₃, freebase) δ (ppm): 8.48 (s, 1H), 8.40 (d, 1H), 7.50 (d, 1H), 7.30 (s, 1H), 7.18 (m, 2H), 5.02 (t, 1H), 4.19 (m, 1H), 4.09 (m, 1H), 3.78 (t, 1H), 3.18 (m, 1H), 2.85 (m, 1H), 2.75 (m, 2H), 2.42 (s, 3H), 2.38 (m, 2H), 1.85 (m, 4H). Other diastereomers can be prepared by using appropriate chiral starting materials.

### Example 100

### Preparation of Compound Nos. 152, 152a, 152b, 152c and 152d

(R)-8,10-Dimethyl-2,3,5,6,7,11c-hexahydro-1H-indolizino[7,8-b]indole (228 mg, 0.95 mmol) was dissolved in 4 mL of DMF, sodium hydride (114 mg, 2.85 mmol) added and stirred for 5 min. 2-cyclohexyl-2-methyloxirane (200 mg, 1.42 mmol) was added and the reaction mixture was heated at 70° C. for 18 h. After consumption of starting material, the reaction mixture was poured in to ice water (20 mL) and extracted with EtOAc (3×50 mL). The combined organic layer was washed with water (3×50 mL), dried over sodium sulfate and concentrated to obtain the crude product that was purified by reverse phase chromatography to obtain 100 mg of 2-cyclohexyl-1-((R)-8,10-dimethyl-2,3,5,6-tetrahydro-1H-indolizino[7,8-b]indol-7(11cH)-yl)propan-2-ol. The racemate was separated by chiral HPLC to obtain 10 mg of (S)-2-cyclohexyl-1-((R)-8,10-dimethyl-2,3,5,6-tetrahydro-1H-indolizino[7,8-b]indol-7(11cH)-yl)propan-2-ol. ¹H NMR (CDCl₃, freebase) δ (ppm): 7.06 (s, 1H), 6.71 (s, 1H), 4.36 (m, 2H), 4.20 (m, 1H), 3.30 (m, 1H), 3.10-2.92 (m, 4H), 2.70 (m, 1H), 2.64 (s, 3H), 2.50 (m, 1H), 2.38 (s, 3H), 2.0-1.80 (m, 8H), 1.50 (m, 2H), 1.30 (m, 2H), 1.18 (m, 2H), 0.9 (s, 3H). Other diastereomers can be prepared by using appropriate chiral starting materials.

### Example 101

### Preparation of Compound Nos. 153, 153a, 153b, 153c and 153d

The carboline (100 mg, 0.416 mmol) was dissolved in DMF (2 mL), sodium hydride (50 mg, 1.25 mmol) was added at RT and stirred at RT for 5 min. 4-(Oxiran-2-yl) pyridine (90 mg, 0.742 mmol) was added and the reaction was allowed RT stir at RT for 3 h. The reaction was monitored with LCMS. The reaction mixture was poured into 20 mL ice cold water and extracted with EtOAc (3×20 mL). The organic layer was washed with water (2×50 mL), dried over sodium sulfate and concentrated under reduced pressure to obtain the product that was washed with hexane and purified with reverse phase column chromatography to obtain the product as mixture of distereoisomers. The mixture of distreoisomers was separated with chiral chromatography to obtained 5 mg of desired product. ¹H NMR (CDCl₃, freebase) δ (ppm): 8.4 (d, 2H), 7.30 (m, 3H), 7.2 (d, 1H), 6.9 (d, 1H), 5.1 (t, 1H), 4.3-4.1 (m, 3H), 2.9 (m, 1H), 2.6 (s, 3H), 2.4 (s, 3H), 2.1 (m, 2H), 1.9 (m, 1H), 1.6 (m, 2H). Other diastereomers can be prepared by using appropriate chiral starting materials.

### Example 102

### Preparation of Compound Nos. 154, 154a, 154b, 154c and 154d

(11cR)-9-Chloro-10-methyl-2,3,5,6,7,11c-hexahydro-1H-indolizino[7,8-b]indole (150 mg, 0.57 mmol) was dissolved in DMF (3 mL) and sodium hydride (68 mg, 1.7 mmol) was added at 0° C. and stirred for 5 min. 3-(2-Methyloxiran-2-yl)pyridine (132 mg, 0.9 mmol) was dissolved in DMF (2 mL) and added dropwise into the reaction mixture was allowed to RT and stirred for 18 h. After consumption of starting material, the reaction mixture was poured into ice cold water and extracted with EtOAc (2×100 mL). The combined organic layer was washed with water (4×50 mL) and dried over sodium sulfate and concentrated under vacuum to obtain the crude product, which was purified by reverse phase chromatography to obtain 110 mg of desired compound. The racemic mixture was separated by chiral chromatography to obtain (2S)-1-[(11cR)-9-chloro-10-methyl-1,2,3,5,6,11c-hexahydroindolizino[7,8-b]indol-7-yl]-2-(3-pyridyl) propan-2-ol (15 mg). ¹H NMR (CDCl₃, freebase) δ (ppm): 8.70 (s, 1H), 8.55 (d, 1H), 7.70 (d, 1H), 7.30 (s, 1H), 7.26 (m, 2H), 4.10 (dd, 2H), 3.95 (t, 1H), 3.22 (m, 1H), 2.85 (m, 1H), 2.82-2.65 (m, 3H), 2.45 (s, 3H), 2.39 (m, 2H), 1.90 (m, 3H), 1.65 (s, 3H). Other diastereomers can be prepared by using appropriate chiral starting materials.

### Example 103

### Preparation of Compound Nos. 155, 155a, 155b, 155c and 155d

(11cR)-10,11c-Dimethyl-1,2,3,5,6,7-hexahydroindolizino[7,8-b]indole (150 mg, 0.6 mmol) was dissolved in DMF (2 mL) and sodium hydride (75 mg, 1.8 mmol) was added at 0° C. and stirred for 5 min. 4-(Oxiran-2-yl)pyridine (121 mg, 1.0 mmol) was dissolved in DMF (2 mL) and added dropwise into the reaction mixture was allowed to RT and stirred for 18 h. After consumption of starting material, the reaction mixture was poured into ice cold water and extracted with EtOAc (4×50 mL). The combined organic layer was washed with water (4×25 mL) and dried over sodium sulfate and concentrated under vacuum to obtain the crude product, which was purified by reverse phase chromatography to obtain 60 mg of desired compound. The racemic mixture was separated by chiral chromatography to obtain (1S)-2-[(11cR)-10,11c-dimethyl-2,3,5,6-tetrahydro-1H-indolizino[7,8-b]indol-7-yl]-1-(4-pyridyl) ethanol (15 mg). ¹H NMR (CDCl₃, freebase) δ (ppm): 8.58 (d, 2H), 7.38 (s, 1H), 7.28 (d, 1H), 7.20 (d, 2H), 7.05 (d, 1H), 5.10 (t, 1H), 4.25 (m, 1H), 4.15 (m, 1H), 3.22 (m, 2H), 2.98 (m, 1H), 2.62 (m, 1H), 2.50 (s, 3H), 2.40 (m, 2H), 2.12 (m, 2H), 1.85 (m, 2H), 1.58 (s, 3H). Other diastereomers can be prepared by using appropriate chiral starting materials.

### Example 104

### Preparation of Compound Nos. 156, 156a, 156b, 156c and 156d

(11cR)-10-(Trifluoromethyl)-2,3,5,6,7,11c-hexahydro-1H-indolizino[7,8-b]indole (250 mg, 0.89 mmol) was dissolved in DMF (2 mL) and sodium hydride (52 mg, 2.23 mmol) was added at 0° C. and stirred for 5 min. 4-(2-Methyloxiran-2-yl)pyridine (180 mg, 1.34 mmol) was dissolved in DMF (2 mL) and added dropwise into the reaction mixture was allowed to RT and stirred for 12 h. After consumption of starting material, the reaction mixture was poured into ice cold water and extracted with EtOAc (3×50 mL). The combined organic layer was washed with water (5×25 mL) and dried over sodium sulfate and concentrated under vacuum to obtain the crude product, which was recrystalised in ether in hexane to obtained 190 mg of desired compound. The racemic mixture was separated by chiral chromatography to obtain (2S)-1-[(11cR)-10-(trifluoromethyl)-1,2,3,5,6,11c-hexahydroindolizino[7,8-b]indol-7-yl]-2-(4-pyridyl)propan-2-ol (80 mg). ¹H NMR (CDCl₃, freebase) δ (ppm): 8.55 (d, 2H), 7.70 (s, 1H), 7.32 (m, 4H), 4.25 (d, 1H), 4.18 (d, 1H), 3.95 (t, 1H), 3.20 (m, 1H), 2.82 (m, 2H), 2.75 (m, 1H), 2.65 (m, 1H), 2.48 (m, 2H), 1.90 (m, 3H), 1.60 (s, 3H). Other diastereomers can be prepared by using appropriate chiral starting materials.

### Example 105

### Preparation of Compound Nos. 157, 157a, 157b, 157c and 157d

To a solution of 2-[(11cR)-8,10-dimethyl-1,2,3,5,6,11c-hexahydroindolizino[7,8-b]indol-7-yl]-1-(4-pyridyl)ethanol (300 mg, 0.831 mmol) in DMF (3 mL) was added sodium hydride (100 mg, 2.49 mmol) at 0° C. After 10 min of stirring, bromomethylcyclobutane (372 mg, 2.49 mmol) was added dropwise. The reaction mixture was allowed to stir at RT for 2 h. The progress of reaction was monitored by TLC& LCMS. After completion of the reaction, ice-cold water was added to the reaction mixture and the product was extracted with EtOAc (3×50 mL). The combined organic layer was washed with water (5×50 mL) and dried over anhydrous sodium sulfate. Removal of solvent under reduced pressure gave a crude product, which was purified by reverse phase HPLC to afford 70 mg desired compound which was purified by chiral HPLC to obtain (11cR)-7-[(2S)-2-(cyclobutylmethoxy)-2-(4-pyridyl)ethyl]-8,10-dimethyl-1,2,3,5,6,11 c-hexahydroindolizino[7,8-b]indole (10 mg). ¹H NMR (CDCl₃, freebase) δ (ppm): 8.55 (d, 2H), 7.10 (m, 3H), 6.95 (s, 1H), 4.45 (m, 2H), 4.22 (m, 1H), 4.15 (t, 1H), 3.22 (m, 2H), 3.08 (m, 1H), 2.90 (m, 1H), 2.80 (m, 1H), 2.70 (s, 3H), 2.65 (m, 1H), 2.45 (m, 1H), 2.42 (s, 3H), 1.90 (m, 4H), 1.82-1.62 (m, 6H), 1.58 (m, 2H). Other diastereomers can be prepared by using appropriate chiral starting materials.

### Example 106

### Preparation of Compound Nos. 158, 158a, 158b, 158c and 158d

8,10-Dimethyl-2,3,5,6,7,11c-hexahydro-1H-indolizino[7,8-b]indole (100 mg, 0.272 mmol) in 2 mL DCM was stirred at 0° C. Dess-Martin periodinane (188 mg, 0.435 mmol) was added and reaction mixture was stirred at RT for 12 h. The reaction was monitored by LCMS, and upon completion. sodium thiosulfate sulfate solution (2 mL) was added, followed by sodium bicarbonate solution (2 mL). The mixture was extracted with DCM (3×250 mL). The combined organic layer was washed with water (3×20 mL), dried over sodium sulfate and concentrated to obtain the crude product that was purified by preparative HPLC, to obtain 65 mg of 1-cyclohexyl-2-(8,10-dimethyl-1,2,3,5,6,11c-hexahydroindolizino[7,8-b]indol-7-yl)ethanone as a racemate. The racemate was separated by chiral HPLC to obtain 8 mg of 2-[(11cR)-8,10-dimethyl-1,2,3,5,6,11c-hexahydroindolizino[7,8-b]indol-7-yl]-1-cyclohexyl-ethanone (Cpd. No. 158a) and 11 mg of 2-[(11cS)-8,10-dimethyl-1,2,3,5,6,11c-hexahydroindolizino[7,8-b]indol-7-yl]-1-cyclohexyl-ethanone (Cpd. No. 158b). ¹H NMR (CDCl₃, freebase) δ (ppm): 7.10 (s, 1H), 6.64 (s, 1H), 5.0 (m, 2H), 4.05 (m, 1H), 3.30 (m, 1H), 2.98-2.82 (m, 2H), 2.74 (m, 2H), 2.50 (s, 3H), 2.42 (m, 1H), 2.38 (s, 3H), 1.90 (m, 3H), 1.80 (m, 4H), 1.68 (m, 1H), 1.40 (m, 2H), 1.24 (m, 3H). Other diastereomers can be prepared by using appropriate chiral starting materials.

### Example 107

### Preparation of Compound Nos. 159, 159a, 159b, 159c and 159d

The carboline (100 mg, 0.44 mmol) was dissolved in DMF (3 mL), sodium hydride (53 mg, 1.32 mmol) was added and stirred for 5 min. 2-Cyclohexyloxirane (73 mg, 0.57 mmol) was added and the reaction mixture was heated at 60° C. for 12 h. The reaction was monitored by LCMS. The reaction mixture was poured in to ice water (30 mL), extracted with EtOAc (3×25 mL), dried over sodium sulfate and concentrated to obtain the crude product that was purified by preparative HPLC to obtain 180 mg of the product as a racemate. The racemate was separated by chiral HPLC to obtain 24 mg of Cpd. No. 159a, 9 mg of Cpd. No. 159b, 23 mg of Cpd. No. 159c and 11 mg of Cpd. No. 159d. 159a: ¹H NMR (CDCl₃, freebase) δ (ppm): 7.98 (s, 1H), 7.58 (s, 1H), 4.18 (m, 2H), 3.90 (m, 1H), 3.70 (m, 1H), 3.40 (m, 1H), 3.0 (m, 2H), 2.78 (m, 1H), 2.62 (m, 1H), 2.40 (s, 3H), 1.90 (m, 3H), 1.80 (m, 2H), 1.64 (m, 1H), 1.62-1.50 (m, 5H), 1.30-1.10 (m, 5H). 159b: ¹H NMR (CDCl₃, freebase) δ (ppm): 7.98 (s, 1H), 7.56 (s, 1H), 4.18 (m, 1H), 3.86 (m, 1H), 3.64 (m, 1H), 3.38 (d, 1H), 2.98 (m, 2H), 2.78 (m, 2H), 2.39 (s, 3H), 1.90 (m, 3H), 1.80 (m, 2H), 1.64 (m, 1H), 1.50 (m, 4H), 1.30-1.10 (m, 6H). 159c: ¹H NMR (CDCl₃, freebase) δ (ppm): 7.98 (s, 1H), 7.54 (s, 1H), 4.16 (m, 2H), 3.89 (m, 1H), 3.68 (m, 1H), 3.41 (m, 1H), 2.96 (m, 3H), 2.74 (m, 1H), 2.60 (m, 1H), 2.42 (m, 1H), 2.40 (s, 3H), 1.90 (m, 3H), 1.78 (m, 2H), 1.70 (m, 1H), 1.45 (m, 2H), 1.30-1.10 (m, 6H). 159d: ¹H NMR (CDCl₃, freebase) δ (ppm): 7.98 (s, 1H), 7.54 (s, 1H), 4.16 (d, 2H), 3.89 (m, 1H), 3.66 (m, 1H), 3.38 (m, 1H), 2.98 (m, 3H), 2.76 (m, 2H), 2.42 (m, 1H), 2.40 (s, 3H), 1.90 (m, 3H), 1.78 (m, 3H), 1.50 (m, 2H), 1.30-1.10 (m, 6H).

### Example 108

### Preparation of Compound Nos. 160, 160a, 160b, 160c and 160d

To a solution of (11cR)-11-bromo-10-methyl-2,3,5,6,7,11c-hexahydro-1H-indolizino[7,8-b]indole (150 mg, 0.49 mmol) in DMF (2 mL) was added sodium hydride (58 mg, 1.47 mmol) at 0° C. After 5 min of stirring, 4-(oxiran-2-yl)pyridine (95 mg, 0.78 mmol) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (4×50 mL). The combined organic layer was washed with water (4×50 mL) and dried over anhydrous sodium sulfate. Removal of solvent under reduced pressure gave a crude product, which was purified by reverse phase HPLC to afford 80 mg of desired compound which was purified by chiral HPLC to obtain 8 mg of (1S)-2-[(11cR)-11-bromo-10-methyl-1,2,3,5,6,11c-hexahydroindolizino[7,8-b]indol-7-yl]-1-(4-pyridyl)ethanol. ¹H NMR (CDCl₃, freebase) δ (ppm): 8.50 (d, 2H), 7.20 (d, 2H), 6.95 (m, 2H), 4.82 (t, 1H), 4.42 (t, 1H), 4.05 (m, 2H), 2.95 (m, 3H), 2.75 (m, 2H), 2.58 (m, 1H), 2.42 (s, 3H), 1.95-1.75 (m, 4H). Other diastereomers can be prepared by using appropriate chiral starting materials.

### Example 109

### Preparation of Compound Nos. 161, 161a, 161b, 161c and 161d

To a solution of (11cR)-9-bromo-10-methyl-2,3,5,6,7,11c-hexahydro-1H-indolizino[7,8-b]indole (150 mg, 0.49 mmol) in DMF (2 mL) was added sodium hydride (59 mg, 1.47 mmol) at 0° C. After 5 min of stirring, 4-(oxiran-2-yl)pyridine (101 mg, 0.83 mmol) was added dropwise. The reaction mixture was allowed to stir at RT for 18 h. The progress of reaction was monitored by TLC and LCMS. After completion of the reaction, ice-cold water was added to the reaction mixture and the product was extracted with EtOAc (4×50 mL). The combined organic layer was washed with water (4×50 mL) and dried over anhydrous sodium sulfate. Removal of solvent under reduced pressure gave a crude product, which was purified by reverse phase HPLC to afford 100 mg of desired compound, which was purified by chiral HPLC to obtain 20 mg of (1S)-2-[(11cR)-9-bromo-10-methyl-1,2,3,5,6,11c-hexahydroindolizino[7,8-b]indol-7-yl]-1-(4-pyridyl)ethanol. ¹H NMR (CDCl₃, freebase) δ (ppm): 8.58 (d, 2H), 7.52 (s, 1H), 7.25 (m, 3H), 5.05 (t, 1H), 4.15 (m, 2H), 3.50 (m, 2H), 3.38 (m, 1H), 2.95 (m, 3H), 2.62 (m, 2H), 2.45 (s, 3H), 2.10-1.90 (m, 3H). Other diastereomers can be prepared by using appropriate chiral starting materials.

### Example 110

### Preparation of Compound Nos. 162, 162a, 162b, 162c and 162d

(R)-10-Methyl-8-vinyl-2,3,5,6,7,11c-hexahydro-1H-indolizino[7,8-b]indole (200 mg, 0.793 mmol) was charged in DMF (3 mL). Sodium hydride (95 mg, 2.3808 mmol) was added at 0° C. and stirred for 10 min. 4-(Oxiran-2-yl)pyridine (154 mg, 1.269 mmol) was added and the reaction mixture was stirred at RT overnight. The reaction was monitored by LCMS. The reaction mixture was quenched with ice cold water. The compound precipitated and was filtered and purified by column chromatography to get the racemic compound (80 mg). The racemate was purified by Chiral HPLC to give 10 mg of Cpd. No. 162a and 15 mg of Cpd. No. 162b. ¹H NMR (CDCl₃, freebase) δ (ppm): 8.60 (d, 2H), 7.54 (m, 1H), 7.25 (m, 3H), 6.99 (s, 1H), 5.69 (d, 1H), 5.49 (d, 1H), 5.03 (m, 1H), 4.45 (dd, 1H), 4.20 (m, 1H), 4.0 (m, 1H), 3.30 (m, 1H), 3.05 (m, 1H), 2.96 (m, 2H), 2.83 (m, 1H), 2.70 (m, 2H), 2.46 (m, 1H), 2.42 (s, 3H), 1.90 (m, 3H). Other diastereomers can be prepared by using appropriate chiral starting materials.

### Example 111

### Preparation of Compound Nos. 163, 163a and 163b

1,1,2,8-Tetramethyl-2,3,4,5-tetrahydro-1H-pyrido[4,3-b]indole (80 mg, 0.350 mmol) was dissolved in DMF (1.5 ml), sodium hydride (42 mg, 1.05 mmol) was added and stirred for 5 min. 4-Oxiranyl-pyridine (63.6 mg, 0.52 mmol) was added and the reaction mixture was allowed to RT and stirred for 4 h. The progress of reaction was monitored by LCMS. The reaction mixture was poured in to ice water (50 mL), the product extracted, dried over sodium sulfate, and evaporated to obtain the crude product that was purified by preparative HPLC to obtain 18 mg of 1-(pyridin-4-yl)-2-(1,1,2,8-tetramethyl-3,4-dihydro-1H-pyrido[4,3-b]indol-5(2H)-yl)ethanol as a racemate. The racemate was separated by chiral HPLC to obtain 7 mg of (1R)-2-[3-[1-(dimethylamino)-1-methyl-ethyl]-5-methyl-indol-1-yl]-1-(4-pyridyl)ethanol (Cpd. No. 163a) and 6 mg of (1S)-1-(4-pyridyl)-2-(1,1,2,8-tetramethyl-3,4-dihydropyrido[4,3-b]indol-5-yl)ethanol (Cpd. No. 163b). ¹H NMR (CDCl₃, freebase) δ (ppm): 8.58 (d, 2H), 7.42 (s, 1H), 7.25-7.22 (m, 3H), 7.02 (d, 1H), 5.04 (t, 1H), 4.16 (d, 2H), 2.90 (m, 4H), 2.48 (s, 3H), 2.42 (s, 3H), 1.50 (s, 3H), 1.48 (s, 3H).

### Example 112

### Preparation of Compound No. 164

To a solution of carboline (200 mg, 0.9 mmol) in NMP (2 mL) was added powdered potassium hydroxide (100 mg, 1.8 mmol), 2-(trifluoromethyl)-5-vinyl-pyridine (234 mg, 1.33 mmol) was added dropwise. The reaction mixture was heated at 40° C. for 1 h. The progress of reaction was monitored by TLC and LCMS. After completion of the reaction, water was added to the reaction mixture and the product was extracted with EtOAc (4×50 mL). The combined organic layer was washed with brine solution (6×50 mL) and dried over anhydrous sodium sulfate. Removal of solvent under reduced pressure gave a crude product, which was purified by reverse phase HPLC to afford 20 mg desired compound. ¹H NMR (CDCl₃, freebase) δ (ppm): 8.4 (s, 1H), 8.15 (s, 1H), 7.65 (s, 1H), 7.45 (d, 1H), 7.41 (d, 1H), 4.4 (t, 2H), 3.58 (s, 2H), 3.2 (t, 2H), 2.78 (t, 2H), 2.58 (t, 2H), 2.41 (s, 3H).

### Example 113

### Preparation of Compound No. 165

To a solution of carboline (500 mg, 2.26 mmol) in DMF (3 mL) was added sodium hydride (271 mg, 6.78 mmol) at 0° C. After 5 min of stirring, 2-(6-methyl-3-pyridyl) ethyl 4-methylbenzenesulfonate (1.05 g, 3.61 mmol) was added portionwise. The reaction mixture was allowed to stir at RT for 5 h. The progress of reaction was monitored by TLC and LCMS. After completion of the reaction, ice-cold water was added to the reaction mixture and the product was extracted with EtOAc (5×50 mL). The combined organic layer was washed with water (4×50 mL) and dried over anhydrous sodium sulfate. Removal of solvent under reduced pressure gave a crude product, which was purified by reverse phase HPLC to afford 250 mg desired compound. ¹H NMR (CDCl₃, freebase) δ (ppm): 8.2 (d, 2H), 7.62 (s, 1H), 7.19 (d, 1H), 7.0 (d, 1H), 4.3 (t, 2H), 3.60 (s, 2H), 3.12 (t, 4H), 2.78 (t, 2H), 2.42 (s, 3H), 2.41 (s, 3H).

### Example 114

### Preparation of Compound Nos. 166, 166a, 166b, 166c and 166d

5-[1-Hydroxy-2-(10-methyl-1,2,3,5,6,11c-hexahydroindolizino[7,8-b]indol-7-yl)ethyl]pyridine-2-carbonitrile (4.0 g, 10.72 mmol) and KOH (2.40 g, 42.895 mmol) in (t-butanol 15 mL) was heated for 100° C. for 45 min. The reaction was monitored by LCMS. The reaction mixture was concentrated, water was added, extracted with EtOAc and concentrated to obtain the crude product that was purified by preparative HPLC to get 300 mg of the required compound. This was separated by chiral HPLC to get 32 mg of Cpd. No. 166a, 30 mg of Cpd. No. 166b, 20 mg of Cpd. No. 166c and, 30 mg of Cpd. No. 166d. 166a: ¹H NMR (CDCl₃, freebase) δ (ppm): 8.44 (s, 1H), 8.20 (d, 1H), 7.86 (d, 1H), 7.80 (bs, 1H), 7.25 (m, 2H), 7.02 (d, 1H), 5.50 (bs, 1H), 5.20 (m, 1H), 4.24 (dd, 1H), 4.19 (dd, 1H), 4.0 (m, 1H), 3.28 (m, 1H), 2.90 (m, 1H), 2.80 (m, 2H), 2.60 (m, 2H), 2.44 (s, 3H), 2.40 (m, 1H), 1.90 (m, 3H). 166b: ¹H NMR (CDCl₃, freebase) δ (ppm): 8.46 (d, 1H), 8.18 (d, 1H), 7.83 (d, 1H), 7.80 (bs, 1H), 7.28 (s, 1H), 7.18 (d, 1H), 7.0 (d, 1H), 5.54 (bs, 1H), 5.15 (t, 1H), 4.20 (d, 2H), 3.90 (m, 1H), 3.26 (m, 1H), 2.90 (m, 2H), 2.82-2.70 (m, 3H), 2.42 (s, 3H), 2.40 (m, 1H), 1.90 (m, 3H). 166c: ¹H NMR (CDCl₃, freebase) δ (ppm): 8.50 (s, 1H), 8.19 (d, 1H), 7.86 (d, 1H), 7.80 (bs, 1H), 7.25-7.20 (m, 2H), 7.0 (d, 1H), 5.54 (bs, 1H), 5.19 (t, 1H), 4.22 (d, 2H), 4.0 (m, 1H), 3.31 (m, 1H), 2.94 (m, 2H), 2.90-2.70 (m, 3H), 2.45 (m, 1H), 2.40 (s, 3H), 1.94 (m, 3H). 166d: ¹H NMR (CDCl₃, freebase) δ (ppm): 8.43 (s, 1H), 8.18 (d, 1H), 7.84 (d, 1H), 7.81 (bs, 1H), 7.24-7.20 (m, 2H), 7.0 (d, 1H), 5.52 (bs, 1H), 5.16 (m, 1H), 4.22 (dd, 1H), 4.17 (dd, 1H), 4.0 (m, 1H), 3.25 (m, 1H), 2.92 (m, 1H), 2.79 (m, 2H), 2.68 (m, 1H), 2.61 (m, 2H), 2.45 (s, 3H), 2.43 (m, 1H), 1.90 (m, 3H)

### Example 115

### Preparation of Compound Nos. 167, 167a, 167b, 167c and 167d

(11cR)-9-Methoxy-10-methyl-2,3,5,6,7,11c-hexahydro-1H-indolizino[7,8-b]indole (80 mg, 0.313 mmol) was charged in DMF (2 mL). NaH (38 mg, 1.560 mmol) was added under at 0° C. and stirred for 10 min. 4-(Oxiran-2-yl)pyridine (76 mg, 0.625 mmol) was added and the reaction mixture was allowed to stir at RT for 18 h. The progress of reaction was monitored by LCMS. After completion of the reaction, ice-cold water was added to the reaction mixture and the product was extracted with EtOAc. The combined organic layer was washed with water and dried over anhydrous sodium sulfate. Removal of solvent under reduced pressure gave a crude product, which was purified by reverse phase HPLC to afford 50 mg desired compound which was purified by chiral HPLC to obtain 10 mg of (1S)-2-[(11cR)-9-methoxy-10-methyl-1,2,3,5,6,11c-hexahydroindolizino[7,8-b]indol-7-yl]-1-(4-pyridyl)ethanol. ¹H NMR (CDCl₃, freebase) δ (ppm): 8.58 (d, 2H), 7.22 (d, 2H), 7.19 (s, 1H), 6.70 (s, 1H), 5.02 (t, 1H), 4.15 (d, 2H), 4.05 (t, 1H), 3.85 (s, 3H), 3.28 (m, 1H), 2.90 (m, 4H), 2.45 (m, 2H), 2.30 (s, 3H), 1.95-1.80 (m, 3H). Other diastereomers can be prepared by using appropriate chiral starting materials.

### Example 116

### Preparation of Compound Nos. 168, 168a, 168b, 168c and 168d

(2R)-1-[(11cR)-8-Fluoro-10-methyl-1,2,3,5,6,11c-hexahydroindolizino[7,8-b]indol-7-yl]-2-(3-pyridyl)propan-2-ol (250 mg, 0.659 mmol) was dissolved in DMF (2 mL). Cesium carbonate (644 mg, 1.98 mmol) and sodium iodide (50 mg, 0.333 mmole) were added portionwise. Bromomethyl-cyclobutane (198 mg, 1.32 mmol) was added at RT, and the reaction mixture was heated at 80° C. for 90 min. After consumption of starting material, the reaction mixture was poured in to water and extracted with EtOAc (2×50 mL). The combined organic layer was washed with water (4×25 mL) and dried over sodium sulfate and concentrated under vacuum to obtain the crude product, which was purified by reverse phase chromatography to obtain 80 mg of (11cR)-7-[(2R)-2-(cyclobutylmethoxy)-2-(3-pyridyl)propyl]-8-fluoro-10-methyl-1,2,3,5,6,11c-hexahydroindolizino[7,8-b]indole. ¹H NMR (CDCl₃, freebase) δ (ppm): 8.70 (s, 1H), 8.50 (d, 1H), 7.60 (m, 1H), 7.18 (m, 1H), 6.70 (d, 1H), 6.55 (m, 1H), 5.48 (d, 1H), 5.32 (d, 1H), 4.62 (d, 1H), 4.38 (d 1H), 3.62 (m, 1H), 3.30 (m, 2H), 2.50 (m, 2H), 2.40 (s, 3H), 2.18 (m, 1H), 2.10-1.90 (m, 8H), 1.80 (m, 2H), 1.58 (s, 3H), 1.45 (m, 2H). Other diastereomers can be prepared by using appropriate chiral starting materials.

### Example 117

### Preparation of Compound Nos. 177, 177a, 177b, 177c and 177d

Compound No. 177, and individual stereoisomers thereof, can be prepared in an analogous fashion to the Examples described both herein and in the PCT applications presented above, for example, following a process as described in Example 59 using the appropriate starting materials.

### Example 118

### Preparation of Compound Nos. 178, 178a, 178b, 178c and 178d

To a solution of 9-methyl-2,3,4,5,6,10c-hexahydro-1H-3a,6-diaza-cyclopenta[c]fluorene (100 mg, 0.442 mmol) in DMF (2 mL) was added sodium hydride (60%, 53 mg, 1.32 mmol,) at 0° C. After stirring for 5 min, 4-oxiranyl-pyridine (81 mg, 0.669 mmol) was added at 0° C. and the mixture stirred at RT for 12 h. The progress of reaction was monitored by TLC and LCMS. The reaction mixture was poured into ice-cold water and extracted with EtOAc (2×25 mL). The combined organic layer was washed with water (5×25 mL), dried over anhydrous sodium sulfate and concentrated under reduced pressure. The residue was purified by reverse phase HPLC to Compound No. 178 (90 mg), which was separated by chiral prep HPLC to give compounds 178a, 178b, 178c and 178d. Compound No. 178a: ¹HNMR (CDCl₃, freebase) δ (ppm): 8.58 (d, 2H), 7.25 (m, 4H), 7.04 (d, 1H), 5.08 (t, 1H), 4.3 (bs, 1H), 4.18 (d, 2H), 3.3 (d, 1H), 3.07 (m, 2H), 2.85 (m, 2H), 2.6 (m, 1H), 2.42 (m, 1H), 2.4 (s, 3H), 2.01 (m, 3H), 1.82 (m, 1H). Compound No. 178b: ¹HNMR (CDCl₃, freebase) δ (ppm): 8.55 (d, 2H), 7.25 (m, 4H), 7.0 (d, 1H), 5.0 (t, 1H), 4.3 (bs, 1H), 4.19 (m, 2H), 3.32 (d, 1H), 3.0 (m, 4H), 2.5 (m, 2H), 2.45 (s, 3H), 2.0 (m, 2H), 1.9 (m, 1H). Compound No. 178c: ¹HNMR (CDCl₃, freebase) δ (ppm): 8.6 (d, 2H), 7.25 (m, 4H), 7.0 (d, 1H), 5.05 (t, 1H), 4.2 (m, 2H), 3.9 (t, 1H), 3.3 (m, 1H), 2.91 (m, 2H), 2.8 (t, 1H), 2.7 (q, 1H), 2.43 (s, 3H), 2.4 (m, 2H), 1.9 (m, 3H). Compound No. 178d: ¹HNMR (CDCl₃, freebase) δ (ppm): 8.58 (d, 2H), 7.25 (m, 4H), 7.04 (d, 1H), 5.08 (t, 1H), 4.3 (bs, 1H), 4.18 (d, 2H), 3.3 (d, 1H), 3.07 (m, 2H), 2.85 (m, 2H), 2.6 (m, 1H), 2.42 (m, 1H), 2.4 (s, 3H), 2.01 (m, 3H), 1.82 (m, 1H).

### Example B1

### Determination of the Ability of Compounds of the Invention to Bind an Adrenergic Receptor

### Protocol Group A

### Adrenergic α_{2B}

To evaluate in radioligand binding assays the activity of compounds of the invention, human recombinant adrenergic α_{2B} receptor expressed in Chinese hamster ovary (CHO) K1 cells (Uhlen, S. et al, Eur. J. Pharmacol. 343(1):93, 1998) in a modified Tris-HCl buffer (50 mM Tris-HCl, pH 7.4, 12.5 mM MgCl2, 1 mM EDTA, 0.2% BSA) was used. Compounds of the invention were incubated with 2.5 nM [3H]Rauwolscine for 60 min at 25° C. Non-specific binding was estimated in the presence of 10 μM Prazosin. Receptor proteins were filtered and washed, the filters were then counted to determine [3H]Rauwolscine specifically bound. Compounds were screened at 1 μM or lower, using 1% DMSO as vehicle. Compounds of the invention were tested in this biochemical assay and percent inhibition of specific binding was determined. Biochemical assay results are presented as the percent inhibition of specific binding in Table B1a.

### Adrenergic α_{2A}

To evaluate in radioligand binding assays the activity of compounds of the invention, human recombinant adrenergic α_{2A} receptor expressed in insect Sf9 cells (Uhlen, S. et al, J. Pharmacol. Exp. Ther. 271:1558, 1994) in a modified Tris-HCl buffer (50 mM Tris-HCl, pH 7.4, 12.5 mM MgCl₂, 2 mM EDTA) was used. Compounds of invention were incubated with 1 nM [³H]MK-912 for 60 min at 25° C. MK912 is (2S-trans)-1,3,4,5′,6,6′,7,12b-octahydro-1′,3′-dimethyl-spiro[2H-benzofuro[2,3-a]quinolizine-2,4′(1′H)-pyrimidin]-2′(3′H)-one hydrochloride. Non-specific binding was estimated in the presence of 10 μM WB-4101 (2-(2,6-Dimethoxyphenoxyethyl)aminomethyl-1,4-benzodioxane hydrochloride). Receptor proteins were filtered and washed, the filters were then counted to determine [³H]MK-912 specifically bound. Compounds were screened at 1 μM or lower, using 1% DMSO as vehicle. Compounds of the invention were tested in this biochemical assay and percent inhibition of specific binding was determined. Biochemical assay results are presented as the percent inhibition of specific binding in Table B1a.

### Adrenergic α_{1B}

To evaluate in radioligand binding assays the activity of compounds of the invention, rat adrenergic α_{1B} receptor obtained from Wistar Rat liver (Garcia-S'ainz, J. et al, Biochem. Biophys. Res. Commun. 186:760, 1992; Michel, A. et al, Br. J. Pharmacol. 98:883, 1989) in a modified Tris-HCl buffer (50 mM Tris-HCl buffer, pH 7.4, 0.5 mM EDTA) was used. Compounds of the invention were incubated with 0.25 nM [³H]Prazosin for 60 min at 25° C. Non-specific binding was estimated in the presence of 10 μM phentolamine. Receptor proteins were filtered and washed, the filters were then counted to determine [³H]Prazosin specifically bound. Compounds were screened at 1 μM or lower, using 1% DMSO as vehicle. Compounds of the invention were tested in this biochemical assay and percent inhibition of specific binding was determined. Biochemical assay results are presented as the percent inhibition of specific binding in Table B1a.

Table B1a: Percentage inhibition of ligand binding to aminergic G protein-coupled receptors by compounds of the invention (Protocol Group A):

Protocol Group B

| | | | Adrenergic (0.03 |
|---|---|---|---|
| | Adrenergic (0.1 μM) | | μM) |
| Compound No. | α_{1B} | α_{2A} | α_{2B} |
| 1 | 18 | 3 | 64 |
| 2a | 28 | 31 | 102 |
| 2b | 4 | 13 | 1 |
| 3 | −5 | 5 | 7 |
| 4 | 27 | 33 | 78 |
| 4a | 11 | 21 | 39 |
| 4b | 35 | 28 | 86 |
| 5 | 5 | 10 | 13 |
| 6a | 91 | 59 | 107 |
| 6b | 61 | 29 | 93 |
| 7 | 99 | 93 | 108 |
| 8 | 1 | 11 | 42 |
| 9 | 77 | 85 | 107 |
| 10a | −3 | 0 | 4 |
| 10b | −5 | −1 | 16 |

### Adrenergic α_{2B}

To evaluate the activity of compounds in radioligand binding assays, Human adrenergic α_{2B} receptor obtained from recombinant cell membrane from Milipore. Binding experiments were carried out in Tris-HCl buffer (50 mM Tris-HCl buffer, pH 7.4, 5 mM Mgcl2, 1 mM Cacl2, 0.2% BSA) as recommended by Milipore. Compounds were incubated with 3.5 nM [³H]Rauwolscine for 60 min at 25° C. Non-specific binding was estimated in the presence of 10 μM Rauwolscine. Receptor proteins were harvested on 0.33% PEI(poly-ethylenemine) soaked GFC filter mat, the specifically bound [³H]Rauwolscine were counted to determine total binding. Compounds were screened at various concentrations, using 1% DMSO as vehicle. Biochemical assay results are presented as the percent Inhibition of specific binding in Table B1b, or estimated Ki values in Table B1c.

### Adrenergic α_{2A}

To evaluate the activity of compounds in radioligand binding assays, Human adrenergic α_{2A} receptor obtained from recombinant cell membrane from Milipore. Binding experiments were carried out in Tris-HCl buffer (50 mM Tris-HCl buffer, pH 7.4, 5 mM Mgcl2, 1 mM Cacl2, 0.2% BSA) as recommended by Milipore. Compounds were incubated with 2 nM [³H]MK-912 for 60 min at 25° C. Non-specific binding was estimated in the presence of 10 μM Rauwolscine. Receptor proteins were harvested on 0.33% PEI(poly-ethylenemine) soaked GFC filter mat, the specifically bound [³H]MK-912 were counted to determine total binding. Compounds were screened at various concentrations, using 1% DMSO as vehicle. percent inhibition of specific binding was determined. Biochemical assay results are presented as the percent Inhibition of specific binding in Table B1b, or estimated Ki values in Table B1c.

### Adrenergic α_{1B}

To evaluate the activity of compounds in radioligand binding assays, Human adrenergic α_{1B} receptor obtained from recombinant cell membrane from Milipore. Binding experiments were carried out in Tris-HCl buffer (50 mM Tris-HCl buffer, pH 7.4, 10 mM Mgcl2, 1 mM EDTA) as recommended by Milipore. Compounds were incubated with 0.3 nM [³H]Prazosin for 60 min at 25° C. Non-specific binding was estimated in the presence of 10 μM prazosin. Receptor proteins were harvested on 0.33% PEI(poly-ethylenemine) soaked GFC filter mat, the specifically bound [³H]Prazosin were counted to determine total binding. Compounds were screened at various concentrations, using 1% DMSO as vehicle. percent inhibition of specific binding was determined. Biochemical assay results are presented as the percent Inhibition of specific binding in Table B1b, or estimated Ki values in Table B1c.

TABLE B1b
Percentage inhibition of ligand binding to aminergic G protein-coupled receptors
by compounds of the invention (Protocol Group B):

| | | | | Adrenergic (0.3 | | Adrenergic | Adrenergic | | | |
|---|---|---|---|---|---|---|---|---|---|---|
| Compound | Adrenergic (0.1 μM) | | | μM) | | (10 nM) | (30 nM) | | | |
| No. | α_{1B} | α_{2A} | α_{2B} | α_{1B} | α_{2A} | α_{2B} | α_{2B} | | | |
| 8a | 18 | 20 | 48 | 22 | 14 | 12 | 21 | | | |
| 8b | 9 | 7 | 26 | 14 | 10 | 13 | 27 | | | |
| 13c | 9 | 11 | 74 | 28 | 21 | −1 | 30 | | | |
| 13d | −7 | 3 | 76 | 9 | 20 | 7 | 27 | | | |
| 14 | 74 | 71 | 101 | 90 | 87 | 81 | 99 | | | |
| 14a | 70 | 1, 21 | 89 | 87 | 39 | 60 | 81 | | | |
| 14b | 85 | 80 | 96 | 95 | 89 | 98 | 100 | | | |
| 15a | 17 | 14 | 68 | 25 | 14 | 19 | 48 | | | |
| 15b | 12 | 1 | 16 | 16 | 0 | 6 | −11 | | | |
| 16a | 16 | 8 | 1 | 9 | 9 | −1 | −14 | | | |
| 16b | −3 | −2 | 7 | 5 | 10 | 4 | −11 | | | |
| 17 | 10 | 10 | −13 | 11 | 15 | 4 | −12 | | | |
| 18 | 20 | 12 | 95 | 51 | 38 | 54 | 81 | | | |
| 18a | 20 | −7 | 80 | 55 | 38 | 41 | 58 | | | |
| 18b | 45 | 45 | 100 | 63 | 71 | 94 | 97 | | | |
| 18c | 5 | 7 | 22 | 6 | 0 | 17 | 13 | | | |
| 18d | 15 | 8 | 79 | 24 | 20 | 11 | 32, 36 | | | |
| 19 | 77 | 59 | 100 | 91 | 79 | 81 | 95 | | | |
| 19a | 82 | 72 | 100 | 93 | 87 | 94 | 99 | | | |
| 19b | 60 | 51 | 99 | 85 | 77 | 91 | 97 | | | |
| 19c | −5 | −7 | 6 | −1 | 1 | −13 | −12 | | | |
| 19d | −8 | −8 | −1 | −5 | −5 | −9 | −4 | | | |
| 20 | 10 | 12 | 54 | 15 | 23 | 6 | 35 | | | |
| 21 | 84 | 78 | 96 | 95 | 89 | 85 | 96 | | | |
| 22 | 11 | 7 | 72 | 30 | 15 | 30 | 61 | | | |
| 23 | 53 | 55 | 92 | 65 | 64 | 65 | 88 | | | |
| 24 | −3 | −15 | 29 | 15 | 4 | −25 | 0 | | | |
| 27a | 0 | 3 | 33 | 15 | 20 | 6 | 10 | | | |
| 27b | 13 | 10 | 67 | 41 | 20 | 26 | 48 | | | |
| 28 | −7 | −8 | −6 | 3 | −2 | 3 | 7 | | | |
| 29 | 15 | 20 | 80 | 46 | 49 | 31 | 66 | | | |
| 30 | 0 | −18 | 12 | 17 | −5 | 13 | 8 | | | |
| 31a | 24 | 33 | 96 | 47 | 48 | 72 | 90 | | | |
| 31b | 30 | 26 | 97 | 50 | 38 | 68 | 90 | | | |
| 31c | 1 | 0 | 45 | −4 | −21 | 3 | −2 | | | |
| 31d | 0 | −11 | 17 | 11 | 6 | 4 | −11 | | | |
| 32 | −1 | 5 | 69 | 18 | 31 | 14 | 34 | | | |
| 32a | −1 | 10 | | 26 | 37 | 28 | 54 | | | |
| 32b | −1 | −5 | | 13 | 9 | 0 | 14 | | | |
| 33 | −12 | −10 | 38 | −1 | 4 | 6 | 8 | | | |
| 34a | −2 | −9 | −11 | −10 | −5 | 21 | −12 | | | |
| 34b | −16 | −7 | 32 | 12 | −2 | 15 | 6 | | | |
| 34c | 3 | 4 | 90 | 28 | 11 | 52 | 80 | | | |
| 34d | 6 | −2 | 25 | 22 | −1 | 14 | 2 | | | |
| 35a | 40 | 67 | 96 | 71 | 86 | 63 | 86 | | | |
| 35b | 5 | 25 | 96 | 17 | 45 | 65 | 86 | | | |
| 36a | −2 | −16 | −3 | 12 | −5 | 8 | −5 | | | |
| 36b | 0 | −7 | −10 | 5 | −5 | −19 | −14 | | | |
| 37a | 26 | 14 | 69 | 34 | 21 | 27 | 50 | | | |
| 37b | 40 | 15 | 75 | 68 | 17 | 25 | 48 | | | |
| 37c | 15 | −2 | 73 | 37 | 14 | 19 | 46 | | | |
| 37d | 5 | 4 | 68 | 14 | 17 | 15 | 36 | | | |
| 38a | 14 | −2 | 30 | 21 | 11 | −2 | 11 | | | |
| 38b | 15 | 16 | 90 | 33 | 42 | 38 | 72 | | | |
| 38c | 2 | 7 | 92 | 23 | 31 | 43 | 71 | | | |
| 38d | 1 | −4 | 22 | 12 | 3 | 1 | 14 | | | |
| 39 | 17 | 17 | 64 | 34 | 19 | 11 | 35 | | | |
| 39a | 15 | 12 | 62 | 36 | 26 | 15 | 38 | | | |
| 39b | 10 | 6 | 32 | 10 | 5 | 5 | 20 | | | |
| 40 | −6 | 5 | 74 | 3 | 19 | 19 | 44 | | | |
| 41a | −2 | −7 | 80 | −3 | 6 | 24 | 51 | | | |
| 41b | −11 | −16 | −2 | −4 | −5 | −14 | −14 | | | |
| 41c | 1 | −12 | 0 | −5 | 1 | −8 | −10 | | | |
| 41d | −2 | −7 | 48 | −1 | 13 | 9 | 21 | | | |
| 42a | 44 | 37 | 97 | 70 | 63 | 74 | 89 | | | |
| 42b | 27 | 32 | 100 | 45 | 48 | 86 | 97 | | | |
| 43a | 29 | 46 | 96 | 55 | 63 | 64 | 85 | | | |
| 43b | 14 | 14 | 98 | 30 | 28 | 71 | 89 | | | |
| 44a | 40 | 47 | 95 | 71 | 70 | 71 | 87 | | | |
| 44b | 69 | 89 | 100 | 85 | 98 | 98 | 98 | | | |
| 44c | 7 | 1 | | 4 | 4 | −9 | 0 | | | |
| 44d | −5 | −17 | | 0 | −16 | −17 | −11 | | | |
| 45a | 12 | −1 | 43 | 35 | 12 | −4 | 16 | | | |
| 45b | 18 | −3 | 23 | 52 | 9 | 4 | 7 | | | |
| 45c | −5 | 0 | 19 | 3 | −11 | 11 | 4 | | | |
| 45d | 4 | −7 | 34 | 18 | −2 | −10 | 0 | | | |
| 46a | 6 | 20 | 82 | 27 | 35 | 33 | 60 | | | |
| 46b | 0 | −1 | 37 | 10 | 3 | 0 | 15 | | | |
| 47 | 3 | 33 | 83 | 32 | 50 | 50 | 65 | | | |
| 48a | 85 | 73 | 94 | 94 | 82 | 93 | 94 | | | |
| 48b | 71 | 83 | 91 | 84 | 87 | 92 | 93 | | | |
| 49 | 9 | 28 | 91 | 40 | 53 | 50 | 74 | | | |
| 50a | 89 | 72 | 100 | 96 | 83 | 95 | 99 | | | |
| 50b | 80 | 73 | 100 | 93 | 86 | 98 | 100 | | | |
| 50c | 19 | 22 | 91 | 43 | 49 | 15 | 42 | | | |
| 50d | −8 | −9 | 13 | 4 | −4 | −8 | −2 | | | |
| 51a | −6 | 16 | 97 | 19 | 39 | 79 | 93 | | | |
| 51b | −8 | 13 | 86 | 4 | 34 | 45 | 69 | | | |
| 52a | 75 | 80 | 101 | 90 | 89 | 99 | 100 | | | |
| 52b | 35 | 72 | 99 | 73 | 78 | 94 | 96 | | | |
| 52c | −6 | 7 | 10 | 0 | 12 | 9 | 11 | | | |
| 52d | −1 | −6 | 1 | 27 | 1 | 9 | 6 | | | |
| 53a | 12 | 60 | 98 | 50 | 74 | 87 | 97 | | | |
| 53b | 4 | 34 | 97 | 33 | 54 | 74 | 91 | | | |
| 53c | 1 | −10 | 1 | −6 | −15 | −4 | 4 | | | |
| 53d | −16 | 0 | −5 | −8 | 5 | −3 | −4 | | | |
| 54 | −5 | −8 | 66 | 8 | −3 | 15 | 43 | | | |
| 55 | 19 | −4 | 50 | 44 | 3 | 6 | 24 | | | |
| 56 | 12 | 1 | 74 | 38 | 9 | 17 | 48 | | | |
| 57a | −4 | −12 | 14 | −4 | −11 | −7 | 0 | | | |
| 57b | −10 | −6 | 8 | −7 | −7 | −7 | −4 | | | |
| 58 | −6 | −6 | −2 | −4 | −1 | −10 | −4 | | | |
| 59a | 69 | 84 | 100 | 91 | 91 | 93 | 98 | | | |
| 59b | 28 | 39 | 99 | 62 | 65 | 84 | 97 | | | |
| 60a | 12 | 26 | 72 | 40 | 50 | 23 | 47 | | | |
| 60b | −9 | 3 | 59 | 8 | 12 | 6 | 28 | | | |
| 61a | 1 | 9 | 88 | 18 | 34 | 38 | 68 | | | |
| 61b | −7 | 6 | 28 | 11 | 22 | −8 | 3 | | | |
| 61c | 4 | 2 | 92 | 15 | 41 | 51 | 77 | | | |
| 61d | 0 | −10 | 23 | 1 | −4 | 0 | 11 | | | |
| 62a | 22 | 14 | 72 | 43 | 30 | 21 | 45 | | | |
| 62b | 2 | 16 | 62 | 21 | 26 | 12 | 32 | | | |
| 62c | 10 | 5 | 65 | 15 | 21 | 47 | 52 | | | |
| 62d | 12 | 11 | 82 | 27 | 25 | 21 | 57 | | | |
| 63a | 2 | 3 | 84 | 24 | 32 | 25 | 55 | | | |
| 63b | −1 | 0 | 22 | 1 | 13 | −6 | −3 | | | |
| 63c | 8 | 8 | | 14 | 36 | 42 | 71 | | | |
| 63d | −5 | −13 | | −4 | −12 | −10 | 5 | | | |
| 64a | 18 | 37 | 96 | 47 | 61 | 73 | 91 | | | |
| 64b | 4 | 10 | 76 | 27 | 37 | 15 | 47 | | | |
| 75a | −1 | 3 | 87 | 12 | 22 | 8 | 40 | | | |
| 75b | −1 | −3 | −1 | 1 | 5 | −10 | −4 | | | |
| 75c | 41 | 67 | | 69 | 84 | 90 | 96 | | | |
| 75d | 15 | 25 | | 33 | 41 | 55 | 79 | | | |
| 76a | −8 | −3 | 53 | 11 | 12 | 12 | 21 | | | |
| 76b | 4 | 5 | 20 | 3 | 0 | 11 | 5 | | | |
| 76c | 19 | 19 | | 33 | 18 | −3 | −3 | | | |
| 76d | 6 | 24 | | 11 | 30 | 51 | 74 | | | |
| 77a | 7 | 3 | | 32 | 19 | 12 | 30 | | | |
| 77b | −3 | 5 | | 14 | 9 | 6 | 12 | | | |
| 78a | 2 | −11 | 5 | 12 | −7 | −1 | −1 | | | |
| 78b | 2 | −7 | 22 | 12 | 8 | 0 | 7 | | | |
| 78c | −11 | 3 | | −2 | −1 | 20 | 33 | | | |
| 78d | 1 | 2 | | 7 | −4 | 1 | −3 | | | |
| 79a | 47 | 81 | 97 | 79 | 93 | 97 | 96 | | | |
| 79b | 28 | 55 | 97 | 57 | 74 | 86 | 95 | | | |
| 79c | 1 | −3 | | 6 | −1 | −6 | 7 | | | |
| 79d | −4 | −7 | | 1 | 5 | −7 | 3 | | | |
| 81a | 72 | 71 | 99 | 89 | 90 | 96 | 99 | | | |
| 81b | 49 | 68 | 100 | 76 | 85 | 98 | 100 | | | |
| 81c | 2 | 3 | 17 | 11 | 8 | −2 | 0 | | | |
| 81d | 5 | 11 | 18 | 9 | 6 | −7 | 8 | | | |
| 82 | 56 | 92 | | 81 | 97 | 98 | 99 | | | |
| 85a | −9 | −12 | | 4 | −4 | 9 | 42 | | | |
| 85b | −12 | −14 | | −3 | −17 | −7 | −5 | | | |
| 86a | 2 | −4 | | 30 | 11 | −2 | 23 | | | |
| 86b | 3 | 11 | | 15 | 14 | 14 | 17 | | | |
| 86c | −5 | −10 | | 15 | 9 | 24 | 53 | | | |
| 86d | 3 | 7 | | 13 | 11 | 13 | 28 | | | |
| 87a | 13 | 19 | | 27 | 33 | 30 | 63 | | | |
| 87b | 16 | 31 | | 35 | 53 | 37 | 66 | | | |
| 87c | 62 | 85 | | 86 | 94 | 96 | 99 | | | |
| 87d | 34 | 57 | | 67 | 79 | 87 | 97 | | | |
| 88a | 15 | 52 | | 43 | 72 | 86 | 93 | | | |
| 88b | −1 | 19 | | 33 | 44 | 70 | 90 | | | |
| 89a | 12 | 23 | | 13 | 9 | −9 | −9 | | | |
| 89b | 3 | 8 | | 9 | 6 | 0 | 1 | | | |
| 89c | 0 | 24 | | 22 | 40 | 54 | 78 | | | |
| 89d | 15 | 74 | | 54 | 99 | 104 | 115 | | | |
| 91a | 59 | 81 | | 81 | 91 | 93 | 95 | | | |
| 91b | 43 | 67 | | 68 | 86 | 95 | 97 | | | |
| 91c | −20 | −14 | | −7 | −3 | −16 | −4 | | | |
| 91d | −19 | −2 | | 6 | 11 | −1 | 24 | | | |
| 96 | −1 | −7 | | 9 | −9 | 1 | 21 | | | |
| 101a | 6 | 7 | 71 | 17 | 21 | 23 | 38 | | | |
| 101b | −3 | 1 | 32 | 5 | 8 | 2 | 3 | | | |
| 101c | 12 | 6 | 16 | 7 | 7 | 7 | 5 | | | |
| 101d | 4 | −10 | 9 | 8 | 3 | −2 | −3 | | | |
| 102a | 5 | −8 | 38 | 22 | 5 | 2 | 13 | | | |
| 102b | 5 | −1 | 70 | 26 | 23 | 28 | 56 | | | |
| 102c | −4 | −12 | −8 | 5 | −5 | −14 | −5 | | | |
| 102d | −1 | −8 | −8 | −5 | 4 | −8 | 6 | | | |
| 134a | −6 | −5 | | | | | −8 | | | |
| 134b | 29 | 7 | | | | | 40 | | | |
| 134c | 8 | 4 | | | | | 8 | | | |
| 134d | 28 | 15 | | | | | 22 | | | |
| 135a | 71 | 57 | | 92 | 77 | 79 | 93 | | | |
| 135b | 64 | 79 | | 81 | 84 | 84 | 83 | | | |
| 135c | 1 | 12 | | 12 | 18 | 4 | 10 | | | |
| 135d | −7 | 0 | | 9 | 2 | −1 | 8 | | | |
| 136a | 31 | 69 | | 70 | 87 | 91 | 95 | | | |
| 136b | 24 | 45 | | 63 | 72 | 76 | 96 | | | |
| 136c | 0 | 3 | | | | | 9 | | | |
| 136d | −2 | −12 | | | | | −8 | | | |
| 137a | 58 | 56 | | 85 | 79 | 83 | 96 | | | |
| 137b | 74 | 94 | | 94 | 102 | 106 | 107 | | | |
| 137c | 12 | 10 | | 14 | 2 | 14 | 10 | | | |
| 137d | 19 | 15 | | 18 | 16 | 21 | 24 | | | |
| 138a | 84 | 91 | | 88 | 88 | 91 | 90 | | | |
| 138b | 57 | 82 | | 80 | 84 | 82 | 81 | | | |
| 138c | 4 | 0 | | | | | −9 | | | |
| 138d | 0 | 4 | | | | | 11 | | | |
| 139a | 9 | 7 | | | | | 31 | | | |
| 139b | 1 | 0 | | | | | 3 | | | |
| 140a | −9 | −5 | | | | | −12 | | | |
| 140b | −4 | −11 | | | | | 56 | | | |
| 141a | 55 | 85 | | | | | 93 | | | |
| 141b | 57 | 81 | | | | | 92 | | | |
| 142a | 46 | 52 | | | | | 92 | | | |
| 142b | 76 | 95 | | | | | 100 | | | |
| 143a | 91 | 44 | | | | | 89 | | | |
| 143b | 94 | 93 | | | | | 100 | | | |
| 143c | 9 | 1 | | | | | 5 | | | |
| 143d | 15 | 7 | | | | | 4 | | | |
| 144a | 28 | 16 | | | | | 73 | | | |
| 144b | 18 | 61 | | | | | 100 | | | |
| 144c | −2 | 1 | | | | | −12 | | | |
| 144d | 1 | 1 | | | | | 1 | | | |
| 145a | 46 | 39 | | | | | 84 | | | |
| 145b | 68 | 89 | | | | | 100 | | | |
| 178a | 73 | 46 | | | | | 86 | | | |
| 178b | 69 | 55 | | | | | 89 | | | |
| 178c | 94,96 | 29,35 | | | | | 100 | | | |
| 178d | 94,99 | 90,96 | | | | | 97,104 | | | |
| | Adrenergic α_{1B} | | | | | | | | | |
| Compound | 0.32 | 1 | 3.2 | 10 | 0.03 | 0.032 | 0.32 | 1 | 3.2 | 10 |
| No. | nM | nM | nM | nM | μM | μM | μM | μM | μM | μM |
| 18b | | | 14 | 17 | 14 | | 74 | 87 | 96 | 99 |
| 19a | 13 | 2 | 7 | 24 | | 61 | 96 | 95 | | |
| 19b | | 3 | 6 | 1 | | 15 | 76 | 87 | 92 | |
| 23 | | | 21 | 22 | 32 | | 64 | 72 | 85 | 93 |

| | Adrenergic α_{2A} | | | | | | | | |
|---|---|---|---|---|---|---|---|---|---|
| Compound | 1 | 3.2 | 10 | 0.03 | 0.032 | 0.32 | 1 | 3.2 | 10 |
| No. | nM | nM | nM | μM | μM | μM | μM | μM | μM |
| 18b | | 8 | 12 | 27 | | 76 | 90 | 96 | 99 |
| 19a | 3 | 0 | 22 | | 49 | 92 | 98 | 100 | |
| 19b | | 12 | 4 | | 27 | 67 | 84 | 95 | 100 |
| 23 | | 14 | 21 | 37 | | 67 | 76 | 88 | 92 |

| | Adrenergic α_{2B} | | | | | | | |
|---|---|---|---|---|---|---|---|---|
| Compound | 0.03 | 0.032 | 0.1 | 0.32 | 1 | 3.2 | 0.032 | 0.32 |
| No. | nM | nM | nM | nM | nM | nM | μM | μM |
| 14a | | | −28 | | | | | |
| 14b | | | 18 | | | | | |
| 18b | 19 | | 6 | 14 | 29 | 78 | | |
| 19a | | 3 | 8 | 9 | 18 | 64 | 99 | |
| 19b | | 6 | 0 | 2 | 4 | 31 | 95 | |
| 23 | | | 13 | 5 | 2 | 40 | | 105 |

* Where shown, some compounds were tested in repeat assays, each datapoint is shown.

TABLE B1c
Ki values of compounds of the invention:

| Compound No. | α_{1B} (nM) | α_{2A} (nM) | α_{2B} (nM) |
|---|---|---|---|
| 4b | 269 | 126 | 3.6 |
| 6a | 25 | 55 | 1.4 |
| 13a | 10 | 50 | 1.85 |
| 13b | 35 | 13 | 1.22 |
| 14a | 18.2 | 305 | 6.5 |
| 14b | 4.5 | 6.8 | 1.1 |
| 18b | 65.8 | 55.9 | 1.03 |
| 19a | 7.5 | 16.2 | 1 |
| 19b | 37.5 | 36 | 2.4 |
| 23 | 56.4 | 20.8 | 2.24 |
| 31a | 145 | 279 | 2.2 |
| 31b | 134 | 362 | 2.6 |
| 32a | | | 7.9 |
| 34c | 379 | 5160 | 4.6 |
| 35a | 60 | 28.5 | 2.9 |
| 35b | 456 | 241 | 3.49 |
| 38b | 179 | 291 | 7.16 |
| 42a | 54 | 55 | 2.3 |
| 42b | 199 | 154 | 1.3 |
| 43a | 111 | 50 | 2.8 |
| 43b | 278 | 374 | 2.1 |
| 44a | 63.7 | 114 | 2.2 |
| 44b | 14 | 6.2 | 0.81 |
| 48a | 8.15 | 31.1 | 1.25 |
| 48b | 25.9 | 16.4 | 0.89 |
| 50a | 3.7 | 6.7 | 0.75 |
| 50b | 6.9 | 5.69 | 0.75 |
| 51 | | | 1.58 |
| 52a | 11.4 | 13 | 0.84 |
| 52b | 39.8 | 21.7 | 0.71 |
| 53a | | | 1.7 |
| 53b | | | 1.8 |
| 59a | 16 | 10 | 1 |
| 61c | | | 5.4 |
| 63c | | | 12.4 |
| 75c | | 24.5 | 0.98 |
| 75d | | | 8.65 |
| 76d | | | 4.55 |
| 79a | 42.7 | 10.6 | 0.77 |
| 79b | | 47.6 | 1.55 |
| 81a | 14.6 | 19.6 | 0.91 |
| 81b | 35.6 | 27.3 | 0.93 |
| 82 | 32.7 | 4.5 | 1.59 |
| 85a | | | 32.6 |
| 86c | | | 13 |
| 87a | | | 20.6 |
| 87b | | | 23.8 |
| 87c | 26.2 | 8.9 | 0.91 |
| 87d | | 54.9 | 1.54 |
| 88a | | 43.9 | 1.45 |
| 88b | | | 2.85 |
| 89c | | | 7.6 |
| 89d | | 26.2 | 0.85 |
| 91a | 20.9 | 8.8 | 0.7 |
| 91b | 38.2 | 28.4 | 0.93 |
| 101a | | | 16.2 |
| 102b | | | 19.3 |
| 135a | 23 | 66.1 | 2.95 |
| 135b | | 3.1 | 0.69 |
| 136a | 14.7 | 14.4 | 1.5 |
| 136b | | | 2.45 |
| 137a | 15 | 43.6 | 1.5 |
| 137b | 7.3 | 2.4 | 0.79 |
| 138a | 2.4 | 2.2 | 0.95 |
| 138b | 14 | 9.9 | 1.45 |
| 178c | | 88.55 | 0.77 |
| 178d | | 1.87 | 0.35 |

### Example B2

### Functional Activity on Recombinant Adrenergic α_{1B}, Adrenergic α_{2A}, Adrenergic α_{2B} and Adrenergic α_{1D} Receptors Using Aequorin and GTPγS Functional Assays

### Protocol A

To study the functional activity of compounds of the invention on the human recombinant adrenergic α_{2B}, adrenergic α_{2A}, adrenergic α_{1D} or adrenergic αₘ with Aequorin functional assays and on the human recombinant adrenergic α_{2B} receptor with GTPγS assay, CHO-K1 cell lines expressing adrenergic α_{2B}, adrenergic α_{2A}, adrenergic αₘ or adrenergic α_{1D} recombinant receptor, mitochondrial apoaequorin and Gα16 are used for the Aequorin assay. CHO-K1 cell line expressing the recombinant α_{2B} receptor is amplified to prepare membranes used for the GTPγS assay.

The following reference agonists are used as both the reference ligand in agonist mode and as the agonist that needs to be inhibited in antagonist mode.

| | α_{1B} | α_{1D} | α_{2A} | α_{2B} | α_{2B} |
|---|---|---|---|---|---|
| Assay | (aeq) | (aeq) | (aeq) | (aeq) | (GTPgS) |
| Agonist | Cira- | Cira- | UK | Oxymeta- | Guan- |
| ligand | zoline | zoline | 14304 | zoline | facine |

Aequorin Assay Procedure:

Aequorin adrenergic α_{1B}, adrenergic α_{2A} or adrenergic α_{2B} cells are grown 18 h prior to the test in media without antibiotics. They are then detached by gentle flushing with PBS-EDTA (5 mM EDTA), recovered by centrifugation and re-suspended in “assay buffer” (DMEM/HAM's F12 with HEPES+0.1% BSA protease free). Cells are incubated at RT for at least 4 h with Coelenterazine h (Molecular Probes). Dose response curves with reference compounds are performed before testing the compounds of the invention. The α_{1B} reference agonist and antagonist are cirazoline and qinazoline, respectively. The α_{2A} reference agonist and antagonist are UK14,304 and rauwolscine, respectively. The α_{2B} reference agonist and antagonist are oxymetazoline and rauwolscine, respectively.

For agonist testing, 50 μL of cell suspension are injected on 50 μL of test compound or reference agonist plated in a 96-well plate. The resulting emission of light is recorded using the Hamamatsu Functional Drug Screening System 6000 (FDSS 6000). For antagonist testing, following an incubation of 15 min. after the first injection, 100 μL of reference agonist at a concentration corresponding to its EC₈₀ is injected on the 100 μL of the mixture of cell suspension and test compound. The resulting emission of light is recorded using the same luminometer as for agonist testing. To standardize the emission of recorded light (determination of the “100% signal”) across plates and across different experiments, some of the wells contained 100 μM digitonin or a saturating concentration of ATP (20 μM). Plates also contained the reference agonist at a concentration equivalent to the EC₈₀ obtained during the test validation.

Agonist activity of test compound is expressed as a percentage of the activity of the reference agonist at its EC₁₀₀ concentration. Antagonist activity of test compound is expressed as a percentage of the inhibition of reference agonist activity at its EC₈₀ concentration.

Compounds are tested for agonist & antagonist activity at the human adrenergic α_{1B}, adrenergic α_{2A} or adrenergic α_{2B} at the following nanomolar concentrations, in duplicate: Agonist (nM): 0.3, 1, 3, 10, 30, 100, 300, 1000, 3000, 10000; Antagonist (nM): 0.15, 0.5, 1.5, 5, 15, 50, 150, 500, 1500, 5000.

GTPγS Assay Procedure:

The procedure is carried out with the following: assay buffer [20 mM HEPES pH 7.4; 100 mM NaCl, 10 μg/mL saponin, 1 mM MgCl₂]; membranes [Recombinant CHO-K1-adrenergic α_{2B} membrane extracts thawed on ice and diluted in assay buffer to give 10 μg/well and kept on ice]; GDP [diluted in assay buffer to give 3 μM final concentration]; beads [PVT-WGA (Amersham, RPNQ0001), diluted in assay buffer at 0.5 mg/well]; GTPγ³⁵S [(PerkinElmer NEG030X), diluted in assay buffer to give 0.1 nM final concentration]; ligand [Guanfacine (Tocris, 1030) as reference agonist and Rauwolscine (Tocris, 891) as reference antagonist, diluted in assay buffer]. Membranes are mixed with GDP (volume:volume) and incubated for at least 15 min. on ice. In parallel, GTPγ[³⁵S] is mixed with the beads (volume:volume) just before starting the reaction.

For agonist testing, the following reagents are successively added in the wells of an Optiplate (Perkin Elmer): 50 μL of test or reference ligand, 20 μL of the membranes:GDP mix, 10 μL of assay buffer and 20 μL of the GTPγ[³⁵S]:beads mix. For antagonist testing, the following reagents are successively added in the wells of an Optiplate (Perkin Elmer): 50 μL of test or reference ligand, 20 μL of the membranes:GDP mix, and then after an incubation of 15 min. at RT, 10 μL of reference ligand at historical EC₈₀ concentration and 20 μL of the GTPγ[³⁵S]:beads mix.

The plates are covered with a top seal, mixed on an orbital shaker for 2 min, and then incubated for 1 h at RT. Then the plates are centrifuged for 10 min. at 2000 rpm, incubated at RT 4 h and counted for 1 min/well with a Perkin Elmer TopCount reader.

Compounds are tested for antagonist activity at the human adrenergic α_{2B} receptor at the following nanomolar concentrations, in duplicate: Agonist and antagonist (nM): 0.3, 1, 3, 10, 30, 100, 300, 1000, 3000, 10000.

### Inverse Agonist Activity

SPA 35S-GTPgS and Radioligand Binding experiments are conducted with Euroscreen membrane preparations. Compound is tested for inverse agonist activity at the human Adrenergic a2A receptor using GTPg35S binding functional assay (FAST-006G) in dose-response and in duplicates.

### Protocol B

### Adrenergic α_{1B}

To evaluate the activity of compounds in β-Arrestin GPCR functional assays, recombinant CHO cell line (DiscoveRX), over expressed with human adrenergic α_{1B} receptor were used. The experiment was done with a kit of DiscoveRX as per recommended protocol of the supplier. Cells were harvested from culture flask using cell dissociation solution (CDS) and plated at a density of 12,000 cells/well in CP reagent (supplied with the kit) in a half area 96-well white polystyrene plate. After 24 h incubation at 37° C., compounds at various concentrations in 1% (final) DMSO were charged to the cells for 30 min at 37° C. Active nor-epinephrine (-NE) at its EC80 concentrations (50 nM) were then added to the cells and incubated for another 90 min at 37° C. Detection mix was prepared by mixing Path Hunter cell assay buffer, Emerald II solution and Galactone star solution in a ratio of 19:5:1 and incubated with the cells for 60 min at RT in the dark. The reading was taken in Envision at luminescence mode. Biochemical assay results are presented as the percent Inhibition in Table B2a, or IC₅₀ values in Table B2b.

### Adrenergic α_{2A}

To evaluate the activity of compounds in β-Arrestin GPCR functional assays, recombinant CHO cell line (DiscoveRX), over expressed with human adrenergic α_{2A} receptor were used. The experiment was done with kit of DiscoveRX as per recommended protocol of the supplier. Cells were harvested from culture flask using cell dissociation solution (CDS) and plated at a density of 12,000 cells/well in CP reagent (supplied with the kit) in a half area 96-well white polystyrene plate. After 24 h incubation at 37° C., compounds at various concentrations in 1% (final) DMSO were charged to the cells for 30 min at 37° C. Active nor-epinephrine (-NE) at its EC80 concentrations (270 nM) were then added to the cells and incubated for another 90 min at 37° C. Detection mix was prepared by mixing of Path Hunter cell assay buffer, Emerald II solution and Galactone star solution in a ratio of 19:5:1 and incubated with the cells for 60 min at RT in the dark. The reading was taken in Envision at luminescence mode. Biochemical assay results are presented as the percent Inhibition in Table B2a, or IC₅₀ values in Table B2b.

### Adrenergic α_{2B}

To evaluate the activity of compounds in β-Arrestin GPCR functional assays, recombinant CHO cell line (DiscoveRX), over expressed with human adrenergic α_{2B} receptor were used. The experiment was done with kit of DiscoveRX as per recommended protocol of the supplier. Cells were harvested from culture flask using cell dissociation solution (CDS) and plated at a density of 12,000 cells/well in CP reagent (supplied with the kit) in a half area 96-well white polystyrene plate. After 24 h incubation at 37° C., compounds at various concentrations in 1% (final) DMSO were charged to the cells for 30 min at 37° C. Active nor-epinephrine (-NE) at its EC80 concentrations (50 nM) were then added to the cells and incubated for another 90 min at 37° C. Detection mix was prepared by mixing of Path Hunter cell assay buffer, Emerald II solution and Galactone star solution in a ratio of 19:5:1 and incubated with the cells for 60 min at RT in the dark. The reading was taken in Envision at luminescence mode. Biochemical assay results are presented as the percent Inhibition in Table B2a, or IC₅₀ values in Table B2b.

TABLE B2a
Percentage inhibition of ligand binding to aminergic G protein-coupled receptors
by compounds of the invention (Protocol Group B):

| | | | | Adrenergic (0.3 | | Adrenergic | Adrenergic |
|---|---|---|---|---|---|---|---|
| Compound | Adrenergic (0.1 μM) | | | μM) | | (10 nM) | (30 nM) |
| No. | α_{1B} | α_{2A} | α_{2B} | α_{1B} | α_{2A} | α_{2B} | α_{2B} |
| 13a | 49 | 14 | 88 | 74 | 21 | 9 | 29 |
| 13b | 32 | 23 | 102 | 61 | 30 | 4 | 30 |
| 14a | 49 | −1 | 41 | 76 | −2 | 13 | 9 |
| 14b | 76 | 13 | 97 | 86 | 44 | 11 | 93 |
| 18b | 19 | 14 | 103 | 46 | 18 | 37 | 94 |
| 19a | 45 | 13 | 101 | 74 | 23 | 33 | 71 |
| 19b | 25 | 24 | 86 | 52 | 20 | 15 | 34 |
| 23 | 15 | 1 | 27 | 15 | 1 | 10 | 12 |

TABLE B2b
IC₅₀ values of compounds of the invention (μM):

| | Adrenergic | | |
|---|---|---|---|
| Compound No. | α_{1B} | α_{2A} | α_{2B} |
| 4b | 0.662 | 6.08 | 0.163 |
| 6a | 0.121 | 1.79 | 0.024 |
| 13a | 0.1 | 2.24 | 0.03 |
| 13b | 0.15 | 0.806 | 0.012 |
| 14a | 0.099 | | 0.124 |
| 14b | 0.025 | 0.424 | 0.014 |
| 18b | 0.357 | 2.78 | 0.013 |
| 19a | 0.107 | 1.26 | 0.018 |
| 19b | 0.351 | 5.01 | 0.052 |
| 23 | 1.21 | | 0.382 |
| 31a | 0.982 | 11.4 | 0.087 |
| 31b | 0.782 | 11.3 | 0.066 |
| 32a | | | 0.396 |
| 34c | | | 0.251 |
| 35a | 0.672 | 2.39 | 0.255 |
| 35b | | | 0.212 |
| 38b | | | 0.237 |
| 38c | | 7.82 | 0.364 |
| 42a | 0.636 | 3.31 | 0.181 |
| 42b | 1.14 | 5.22 | 0.041 |
| 43a | 0.67 | 3.57 | 0.119 |
| 43b | 1.09 | 7.34 | 0.091 |
| 44a | 0.684 | 5.18 | 0.178 |
| 44b | 0.252 | 0.909 | 0.01 |
| 48a | 0.045 | 1.11 | 0.02 |
| 48b | 0.123 | 0.491 | 0.018 |
| 50a | 0.047 | 0.713 | 0.014 |
| 50b | 0.054 | 0.565 | 0.012 |
| 52a | 0.149 | 1.02 | 0.016 |
| 52b | 0.224 | 0.992 | 0.049 |
| 59a | 0.35 | 1.57 | 0.067 |
| 61c | | | 0.313 |
| 63c | | | 0.692 |
| 75a | 0.719 | 2.08 | 0.017 |
| 75d | | | 0.381 |
| 76d | | | 0.261 |
| 79a | | 0.678 | 0.012 |
| 79b | | 2.67 | 0.034 |
| 81a | 0.091 | 1.25 | 0.016 |
| 81b | | 1.65 | 0.015 |
| 82 | 0.382 | 0.675 | 0.0254 |
| 85a | | | 0.832 |
| 86c | | | 0.699 |
| 87a | | | 0.303 |
| 87b | | | 0.284 |
| 87c | | 0.787 | 0.013 |
| 87d | | 3.68 | 0.044 |
| 88a | | | 0.388 |
| 88b | | | 0.166 |
| 89c | | | 0.0455 |
| 89d | | 1.97 | |
| 91a | 0.213 | 0.865 | 0.0164 |
| 91b | 0.283 | 1.78 | 0.0187 |
| 102b | | | 0.412 |
| 135a | 0.189 | 4.35 | 0.0531 |
| 135b | 0.102 | 0.295 | 0.00927 |
| 136a | | 0.896 | 0.0106 |
| 136b | | | 0.0683 |
| 137a | 0.114 | 5.1 | 0.0413 |
| 137b | 0.0356 | 0.185 | 0.00657 |
| 138a | 0.0722 | 0.413 | 0.0172 |
| 138b | 0.926 | 1.33 | 0.0206 |

### Example B3

### Cell Culture and Cell Viability Assay

SH-SY5Y cells cultured in DMEM/F12 media supplemented with 10% FBS are seeded in 96-well microplates at 150,000 cells/cm². After 24 h, cells are depleted from FBS and kept in culture for 24 h before the experiment. Cells are then treated with 4-Br-A23187 (2 μM), hydrogen peroxide (300 μM) or the mitochondrial toxin rotenone (25 μM) in the presence of vehicle or Compound of the Invention for 24 h. Cell death is determined by measurements of LDH release according to the Cytotoxicity Detection KitPlus (Roche, Mannheim, Germany). Cell viability is determined by measuring the capacity of cells to metabolize MTS tetrazolium (MTS) according to the Cytotoxicity Detection KitPlus (Roche, Mannheim, Germany) and MTS reduction is assessed by the CellTiter 96® AQueous One Solution Cell Proliferation assay (Promega Corporation, Madison, Wis., USA). Compounds are screened at 10 nM, using DMSO as vehicle. Assay results for the experiments with hydrogen peroxide are presented as the LDH release (cell death) of untreated cells (control), hydrogen peroxide-treated cells (vehicle), and co-incubation of hydrogen peroxide with Compounds of the Invention treated cells normalized to the vehicle. This assay assesses the ability of the test compounds to protect against cell death that is mediated by mitochondrial dysfunction. In the assay, the calcium ionophore 4-Br-A23187 is used to challenge the cells, causing calcium levels to rise in mitochondria, which leads to depolarization and cell death. Test compounds are assessed for their ability to prevent cell death in response to challenge with 4-Br-A23187.

Assay results for the experiments with Br-A23187 are presented as the MTS reduction capacity (cell viability) of untreated cells (control), 4-Br-A23187-treated cells (vehicle), and co-incubation of Br-A23187 with Compounds of the Invention treated cells and using p-trifluoromethoxyphenylhydrazone (FCCP) at 10 μM for 30 min as a control.

### Example B4

### Cell Culture and Cell Viability Assay

Cell Culture.

SH-SY5Y cells stably transfected with a doxycyline-inducible wild-type α-synuclein (α-syn) gene along with control SH-SY5Y cells over-expressing the β-galactosidase (β-gal) gene (a gift from L. Stefanis, Division of Basic Neurosciences, Biomedical Research Foundation of the Academy of Athens, Athens, Greece) are cultured as described by Vekrellis et al. (Vekrellis K, Xilouri M, Emmanouilidou E, Stefanis L. (2009). Inducible over-expression of α-syn in human neuronal cells leads to caspase-dependent non-apoptotic death. J Neurochem 109, 1348-1362). In accordance with this method, cells are cultured and maintained in RPMI 1640, 10% fetal bovine serum supplemented with 250 μg/mL G418 and 50 μg/mL Hygromycin B. Expression of α-syn is switched off in stock cultures with doxycycline (2 μg/mL). For experimental procedures, cells are plated at (4-8×10⁴ cells/cm²) and differentiated in absence of doxycycline and in the presence of 20 μM all-trans retinoic acid (RA) (Sigma, St Louis, Mo., USA).

Viability Assay:

Cells are cultured in 96-well plates. After 24 h, cells are treated with RA and Compounds of Invention at 0.1 and 10 nM in the absence of doxycyline. Culture medium with RA and drugs is fully replaced after 7 days. Cell viability is measured by the release of lactate dehydrogenase (LDH) from necrotic cells into the culture medium and by measuring the capacity of cells to metabolize MTS tetrazolium (MTS) after 14 days in culture. LDH leakage is assessed according to the Cytotoxicity Detection KitPlus (Roche, Mannheim, Germany) and MTS reduction is assessed by the CellTiter 96® AQueous One Solution Cell Proliferation assay (Promega Corporation, Madison, Wis., USA).

Assay results for the experiments with α-syn over-expression are presented as the MTS reduction capacity (cell viability) of control cells (+dox), cells over-expressing α-syn (−dox), and cells over-expressing α-syn incubated with Compounds of the Invention at 0.1 nM or 10 nM.

Immunoblotting of α-Synuclein and α-Synuclein Aggregates:

Cells stably expressing α-synuclein are cultured in 6-well plates at a density of 4×10⁴ cells/cm² cells per well. Cells are differentiated and treated with Compound of the Invention at 10 nM in absence of dox after 24 h of plating. Drug treatments are repeated after 7 days in freshly prepared medium containing RA. After 14 days, cells are washed twice with cold PBS and lysed in lysis buffer containing 1% Triton X-100, 20 mM HEPES, 150 mM NaCl, 10% glycerol, 1 mM EGTA, 1.5 mM MgCl₂, 1 mM PMSF pH 7.4, and 1× protease inhibitor mixture (Roche, Mannheim, Germany). Lysates are homogenized and subjected to four successive freeze-thaw cycles to disrupt membranes. Triton soluble fractions and triton insoluble pellets are obtained by ultracentrifugation at 100,000×g for 30 min at 4° C. The concentration of protein in each fraction is determined by BCA assay (Thermo Scientific). Samples from total, soluble and triton insoluble fractions, are boiled in 1× sample buffer (20 mM Tris, 1% glycerol, 180 mM β-mercaptoethanol, 0.003% bromophenol blue, and 2% SDS, pH 6.8), loaded on 12% SDS-PAGE gels, and transferred to polyvinylidene difluoride (PVDF) membranes (0.2 μM-pore immobilon Biorad). Membranes are blocked in 1×TBS-Tween (20 mM Tris, pH 7.4, 150 mM NaCl, and 0.2% Tween 20) containing 5% milk for 1 h and incubated overnight at 4° C. with the following primary antibodies in blocking solution at the indicated dilutions: monoclonal anti-α-synuclein α-syn-1 (1:1000; BD Transduction Laboratories). (Perrin, R. J., Payton, J. E., Barnett, D. H., Wraight, C. L., Woods, W. S., Ye, L., and George, J. M. (2003). Epitope mapping and specificity of the anti-α-_synuclein monoclonal antibody Syn-1 in mouse brain and cultured cell lines. Neurosci Lett 349, 133-135), and monoclonal vimentin (1:1000; BD PharMingen). Primary antibodies are detected with secondary anti-mouse antibodies conjugated to HRP (1:5000).

Isolation of RNA and RT-Quantitative PCR (RT-qPCR):

SH-SY5Y cells stably over-expressing α-syn are treated with Compound of the Invention (10 nM). Total RNA from these cells as well as control cells not treated with Compound is extracted using the E.Z.N.A RNA extraction Kit (OMEGAbiotek, Norcross, Ga.). 1 μg of RNA is reverse transcribed to cDNA using the M-Mulv reverse transcriptase enzyme (Promega Corporation, Madison, Wis., USA). RT-qPCR of cDNA templates is carried out using TAQMAN probes for human α-synuclein (Hs00240906_M1) and TAQMAN masterMix (Applied Biosystems) and a Mx3005P real-time PCR system (Agilent Technologies Inc., Santa Clara, Calif.). Levels of alpha-tubulin mRNA are used to normalize the amounts of total RNA between samples. Fold changes are calculated as described by (Pfaffl, M. W. (2001). A new mathematical model for relative quantification in real-time RT-PCR. Nucleic Acids Res 29, e45).

### Example B5

### α_{2B} Pharmacology: Studies in Spontaneously Hypertensive Rat (SHR) Model of Hypertension

Male spontaneously hypertensive rats (SHR), approximately 3 months of age and weighting approximately 250 grams are utilized. Free access to standard lab chow for rats and reverse osmosis (RO) water is granted. All aspects of this work, including housing, experimentation and disposal of animals are performed in general accordance with the Guide for the Care and Use of Laboratory Animals (National Academy Press, Washington, D. C., 1996).

The animals are anaesthetized with sodium pentobarbital (50 mg/kg IP). The left carotid artery when compound dosed orally (PO) or subcutaneously (SC); and both left carotid and femoral artery when compound dosed intravenous (i.v.) are cannulated with a polyethylene catheter (38 cm in length; PE60, Portex, Ltd.) connected with a polyurethane tubing (12 cm in length; PU-40, Cat. # BB520-40, Scientific Commodities, Inc.), which is tunneled under the skin and exited through the nape of the neck. The arterial cannula is connected to a pressure transducer through a swivel system, allowing free roaming during continuous recording of mean arterial pressure and heart rate. The animals are housed individually with food and water freely available during recovery. On the following day, the arterial cannula is connected via a Statham (P 23 x L) pressure transducer to a NEC/San-Ei amplifier and data acquisition and analysis system (Power Lab 8/SP) for direct mean arterial pressure and heart rate measurements.

The test compounds, dissolved in sterile saline, are administered subcutaneously (SC) or orally (PO), or by intravenous (i.v.) bolus administration in two minutes or the escalating doses of compound administration in every 30 minutes, with each dose and its strength delivered over 2 minutes as shown in the respective figures; the internal standard phentolamine is given by oral gavage. The control group received vehicle alone. Immediately before (−10 min and −5 min) and at 15 min, 30 min, 1 hr, 1.5 hr, 2 hr, 2.5 hr, 3 hr, 3.5 hr, and 4 hr post-dosing, systolic pressure blood pressure values are recorded.

### Example B6

### α_{2B} Pharmacology: Studies in Healthy Dogs and Dexmedetomidine (DEX) Induced Beagle Dog Model of Hypertension

These studies are conducted in both acute and chronic modes.

Four adult beagle dogs of both sex and weighted around 10 kg are chosen for the acute studies after a preliminary qualitative electrocardiogram/ECG, clinical pathology and physical examination. Upon arrival at the laboratory, the dogs are weighed and acclimated for a period of one week. Lab Diet certified canine diet #5007, PMI Nutrition International Inc is made available ad libitum to all dogs except during fasting periods. The dogs are surgically implanted with a pressure transducer equipped telemetry transmitter under sodium pentobarbitone anesthesia. The transmitter assembly is secured internally, the fluid-filled catheter is placed into an appropriate artery.

In the acute studies, the test compounds at different doses (dosing sequences provided in Table B6a) is administered by oral gavage, 30 minutes prior to intravenous dexmedetomidine (5 μg/kg) challenge. Dexmedetomidine administration is enabled by prior placement of a peripheral intravenous line. The same four dogs receive all four treatments in the order noted in the table below, with at least a 3-day washout period between treatments.

TABLE B6a
Acute dosing sequence

| Compound of the invention- | Dexmedetomidine | |
|---|---|---|
| 30 minute Pretreatment | Challenge | Number |
| (mg/kg, p.o.) | (μg/kg, i.v.) | of Dogs |
| 0 | 5 | 4 |
| 2 | 5 | 4 |
| 6 | 5 | 4 |
| 18 | 5 | 4 |
| 2 | 0 | 4 |
| 6 | 0 | 4 |
| 18 | 0 | 4 |

For the chronic study mode (see Table B6b), the test compound at 3 doses is administered by oral gavage once on day 1 and then twice/day on days 2 to 14, and finally once on day 15. The dexmedetomidine is administered on day −4 to check its effectiveness in inducing blood pressure, and once following the morning dose of compound 3b or vehicle on days 2, 7 and 14. Blood pressure and heart rate data are collected 1 h prior & 4 h post-morning dose on days 1, 2, 7, 14 and 15 to allow the appropriate data comparisons. Blood aliquots are saved at 4 h post-morning dose for exposure determination.

TABLE B6b
Chronic dosing sequence and study design
for Compound of the invention

| Compound of the invention- | | | | | | |
|---|---|---|---|---|---|---|
| 30 minute Pretreatment | | | Dexmedetomidine | | | |
| (mg/kg, p.o.) with b.i.d. | | | Challenge | | Number | |
| regimen for 14 days | | | (μg/kg, i.v.) | | of Dogs | |
| 0 | | | 5 | | 6 | |
| 6 | | | 5 | | 6 | |
| 18 | | | 5 | | 6 | |
| | Day | | | | | |
| | −4 | 1 | 2 | 7 | 14 | 15 |
| Compound | — | am | am/pm | from day 2 | to day 14 | am |
| dosed on | | | | am/pm | am/pm | |
| DEX* | am | — | am | am | am | — |

*DEX administered 30 min following am dose of compound.

In both acute and chronic studies, dogs are weighed before dosing. Cardiovascular evaluations at each dose of test compound are collected with animals gently restrained in a sling. Dogs are placed in the sling at least 1 hour prior to dose administration, and after at least 30 minutes of stable baseline data collection. The dogs are monitored continuously for 3-4 hours subsequent to test compound administration and summarized in 5-minute bins. The systolic blood pressure is collected. Data is reported as mean±SEM or mean.

Adrenergic receptors α_{2B} and α_{2A} mixed inhibitor's pharmacology—Studies in Spontaneously Hypertensive Rat (SHR) Model of Hypertension: Similar to dosing regimen for selective antagonists of adrenergic receptor α_{2B}, the mixed inhibitors is dosed orally (PO) or intravenous (i.v bolus or escalating doses) to SHR rats.

### Example B7

### Peripheral and Central Effects of Compound of the Invention on Blood Pressure in Conscious Rabbits

Four adult New Zealand White rabbits of both sexes are chosen for these studies. The experiments are conducted in accordance with the Australian code of Practice for the Care and use of Animals for Scientific Purposes and approval is sought from the Animal Experimental Committee of Alfred Hospital, Baker IDI, Melbourne, Australia. The conscious rabbits are implanted with an intravenous catheter in marginal ear vein or by centrally by intracisternal catheter interfaced to a pressure transducer connected to a suitable recorder. To unveil peripheral effects of test compound, two sets of acute studies are conducted in rabbits. In the first set of studies, the test compound is dosed to rabbit intravenously for a dose-response study with cumulative doses starting 0 (Ringer's Lock solution as a vehicle), 0.1, 0.3, 1, 3.2 and 10 mg/kg where each dose is tested on a separate day. A single intravenous bolus dose at 3 mg/kg is given and a time-course study is conducted in the second set of studies. Systolic, diastolic, mean and diastolic blood pressures are recorded in both the studies. Data collections are made for 3 hours in the second set of studies. Heart rate (HR) is derived electronically using an algorithm to determine HR from pulse interval. In a separate set of studies, Clonidine (positive control) is tested where all experimental procedures including dose-regimen are identical to that of the studies with test compound.

In addition to studying the effect of test compound on blood pressure and heart rate when the compound is administered intravenously, the blood pressure and heart rate effect of test compound is also measured following infusion of the compound directly into the brain with the cannula delivering the compound placed directly into the 4th ventricle of the brain. Several doses are tested for cardiovascular effects following direct brain infusion. Comparison of the blood pressure effects following intravenous and ventricular infusion determines whether the compound exerts its cardiovascular actions by the brain.

### Example B8

### Human Clinical Studies

The compound is studied in a clinical trial of hypertensive patients who have not reached their blood pressure goals on current therapy. The target patient population are patients with refractory hypertension that have not reached their blood pressure goals despite use of at least 3 different blood pressure agents. The study compares the active compound against a matched placebo compound with the primary objective of comparing mean blood pressure change from baseline to the end of the study between the active compound and placebo.

### Example B9

### Stability of Compounds of the Invention in the Presence of Dog, Rat and Human Hepatocytes

Compounds of the invention were tested for stability in the presence of dog, rat and human hepatocytes over a 4 hour period by LC/MS/MS. The study was performed by Integrated Analytical Solutions, Berkeley, Calif.

Reference Standards and Solutions: Compounds of the invention were stored as powders at ambient temperature in a desiccator and protected from light. Reference stock solutions of Compound Nos. 50a, 50b, 79a, 82, 91a and 178d, as 20 mM in DMSO were prepared and subsequently diluted to 2 mM in MeOH to provide working stock solutions (WSS). Unused standard solutions were stored at −20° C.

Hepatocyte Preparation: Human (mixed gender), Beagle dog (male) and Sprague Dawley rat (male) cryopreserved hepatocytes were purchased from Life Technologies Corporation. Hepatocytes were removed from liquid nitrogen, quickly thawed in a 37° C. water bath and transferred to Hepatocyte Thawing/Plating Medium (Cryopreserved Hepatocyte Recovery Medium, CHRM, Life Technologies Inc.). The cells were pelleted by slow speed centrifugation (˜100×g, 6 min) and resuspended at a high cell density. Hepatocyte viability was determined by trypan blue exclusion. Hepatocyte incubation medium (Life Technologies, Inc.) was added to generate a cell density of 2.0×10⁶ cells/mL.

Hepatocyte Incubations for Stability: The 2 mM WSS was diluted 1 in 200 in hepatocyte incubation medium (pH 7.4) to 10 μM. A solution containing control compounds was prepared similarly to contain 10 μM each of dextromethorphan and testosterone. The solutions of test compounds and controls at 10 μM in hepatocyte incubation medium were pre-incubated at 37° C. for 10 min. Aliquots (250 μL) of hepatocyte suspensions (at 2.0×10⁶ cells/mL) were transferred to appropriate 48-well plates and pre-incubated at 37° C. for 10 minutes. Metabolism was initiated by adding 250 μL pre-incubated medium (containing test drug) to wells containing cells. A negative control reaction excluding hepatocytes was used to monitor aqueous stability and/or non-specific adsorption. The final reaction mixtures contained 5 μM of test or control compound and 1.0×10⁶ cells/mL. All reactions were performed in duplicate and carried at 37° C. in an incubator. Dextromethorphan and testosterone were used as control drugs to verify hepatocyte activity.

Aliquots were removed from each metabolism reaction at 0, 0.5, 2 and 4 hours. The reactions were terminated by adding each aliquot to a vessel containing 2× volumes (80 μL) of Internal Standard Solution (acetonitrile containing 50 ng/mL 2-(2,8-dimethyl-3,4-dihydro-1H-pyrido[4,3-b]indol-5(2H)-yl)-1-(pyridin-4-yl)ethanol (ISS1), 50 ng/mL of 2-(2,8-dimethyl-3,4-dihydro-1H-pyrido[4,3-b]indol-5(2H)-yl)-1-(piperidin-1-yl)ethanone (ISS2) and 5 ng/mL diphenhydramine. Terminated reactions were centrifuged at 6000 g for 30 mins at 4° C. to remove the precipitated proteins and cell debris. Following centrifugation, 20 μL of each supernatant was transferred to a deep-well microplate and diluted with 5× volumes (100 μL) of 0.2% formic acid in water. Compound 178d samples were diluted with 5× volumes (100 μL) of 0.1% HFBA in water. Samples were analyzed by LC/MS/MS.

Calculation of Metabolism Parameters: Percent remaining was measured by dividing the analyte/IS peak area ratio at the designated sample time by the peak area ratio at time 0. ISS1 served as the IS for Compound No. 178d. ISS2 served as the IS for Compound nos. 50a, 50b, 79a, 82 and 91a. The metabolic half-life (T_{1/2}) was calculated using all time points unless otherwise noted. The T_{1/2} values were calculated as 0.693/k, where k is the slope of the log analyte/internal standard peak area ratio versus time. Metabolism rate was calculated by dividing the starting concentration of substrate by the concentration of hepatocytes, then dividing by the T_{1/2} value.

Summaries of the stability results for compounds tested are presented in Table B9.

TABLE B9
Summary of stability results of test compounds in human
hepatocytes (average of duplicates)

| | T_{1/2} (min) | | |
|---|---|---|---|
| Compound No. | Human | Dog | Rat |
| 50a | 115 | 122^{a} | 146^{a} |
| 50b | 82^{a} | 100^{a} | >240 |
| 79a | >240 | 181^{a} | 210^{a} |
| 82 | 49 | 48^{a} | 46^{a} |
| 91a | 138 | 82^{a} | 169 |
| 178d | 134 | 240^{a} | >240 |

^{a}Half-life determined using 0-120 minute time points

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound selected from the group consisting of Compound Nos. 1-177: 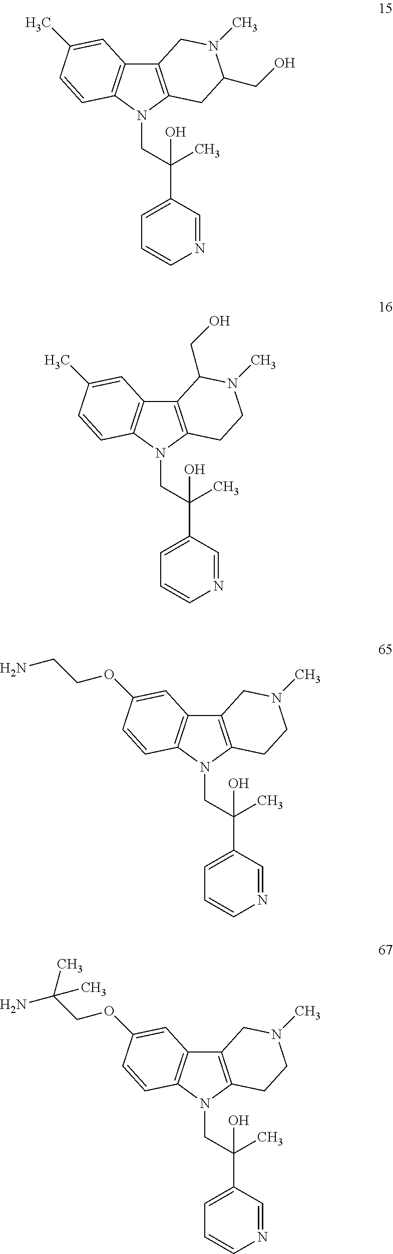 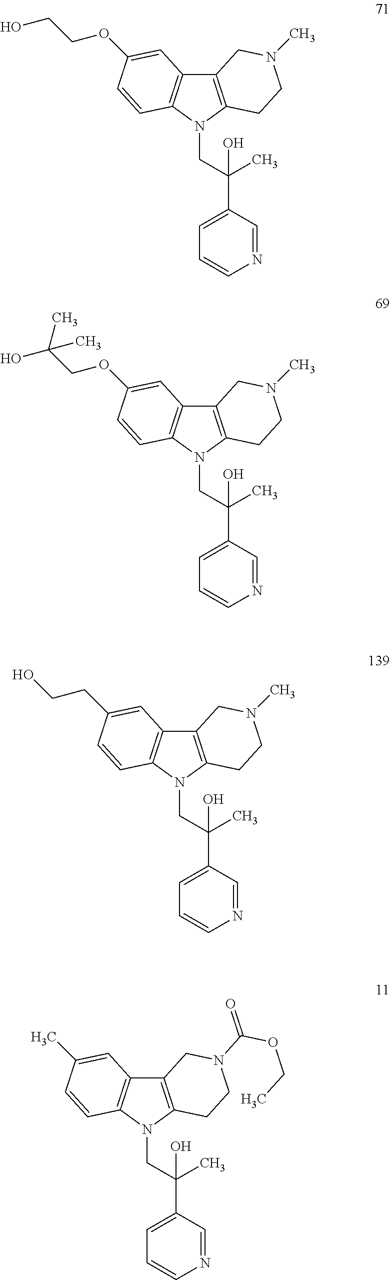 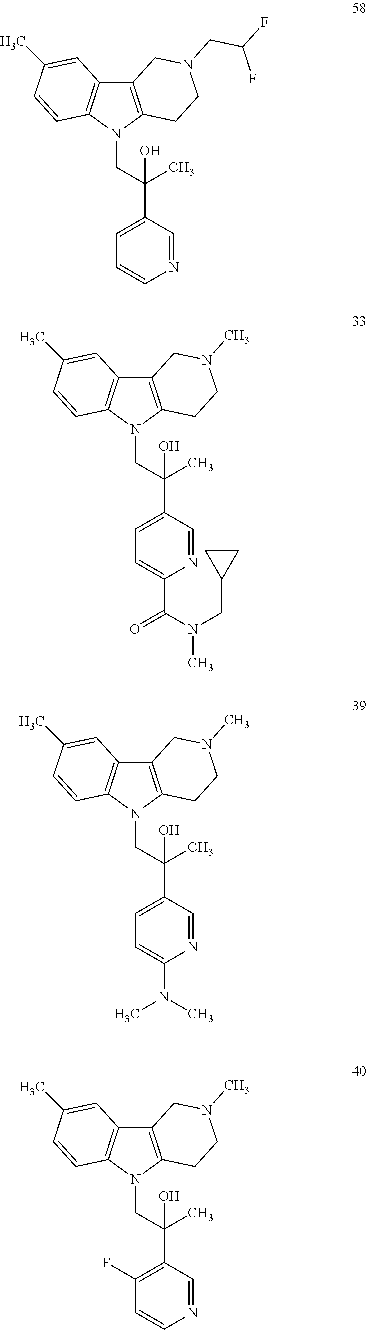 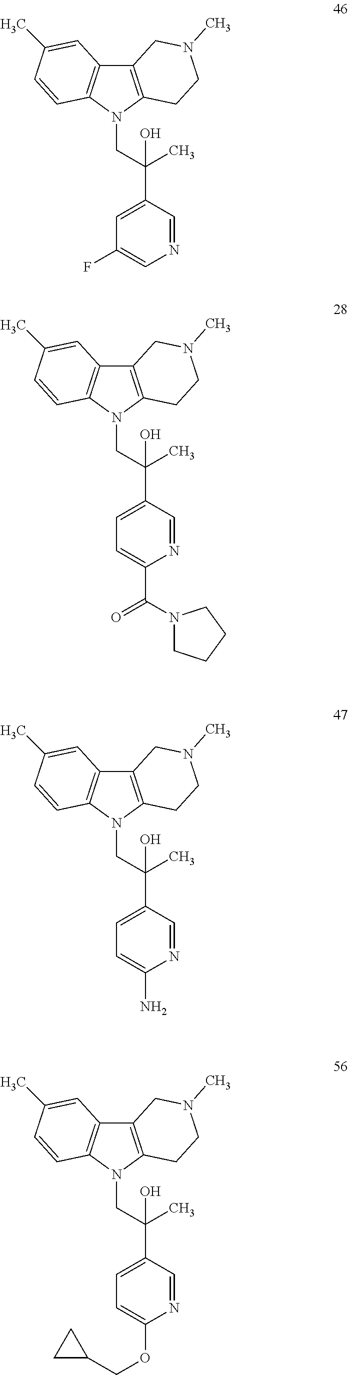 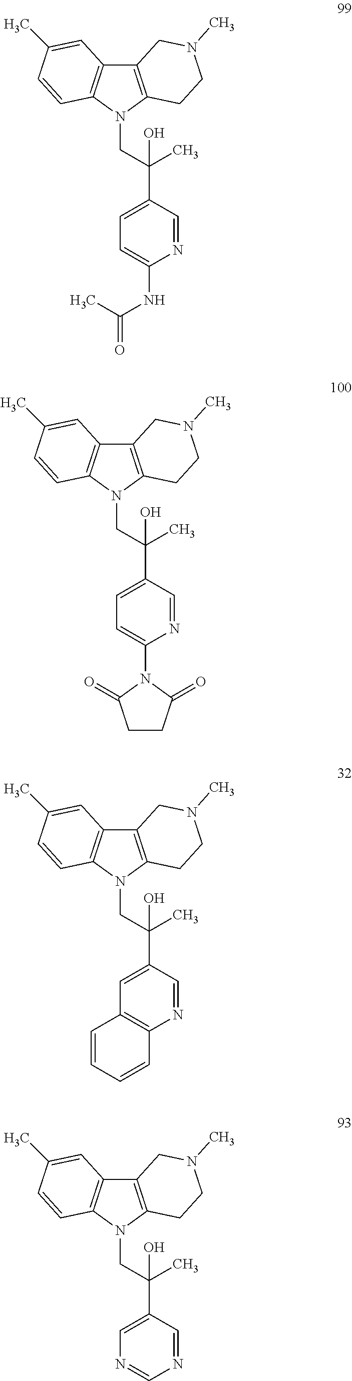 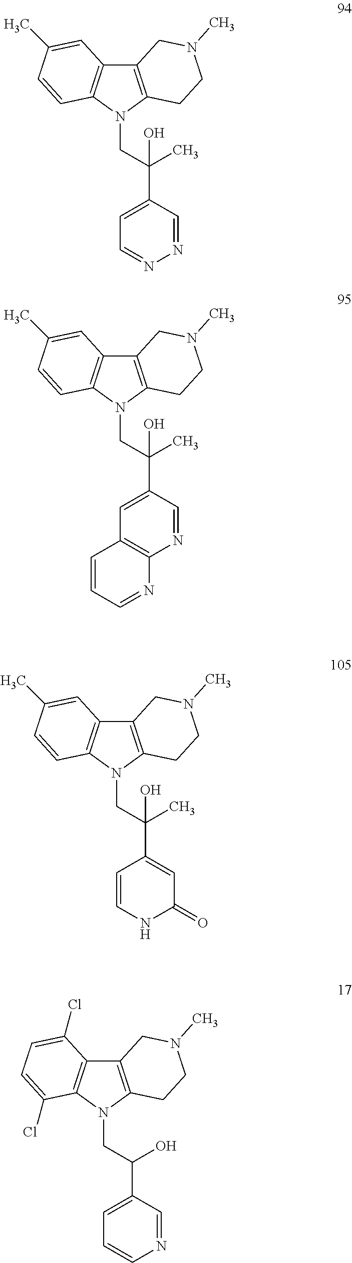 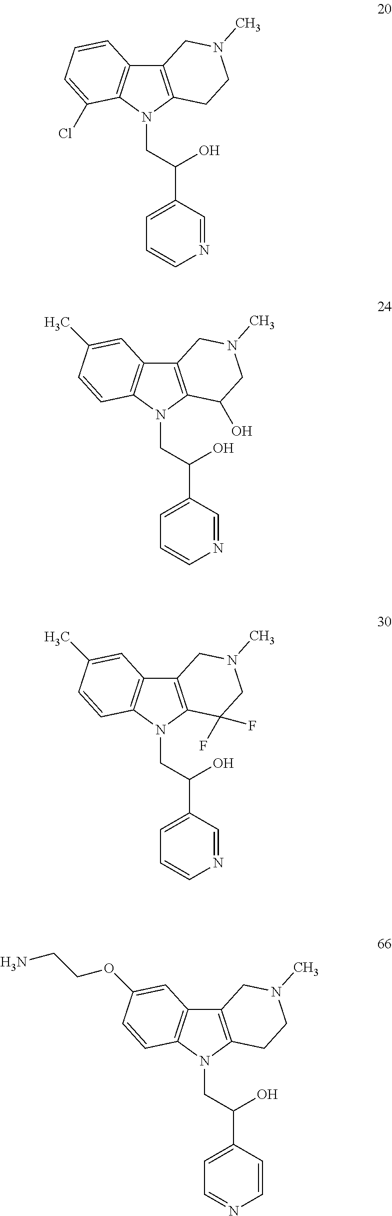 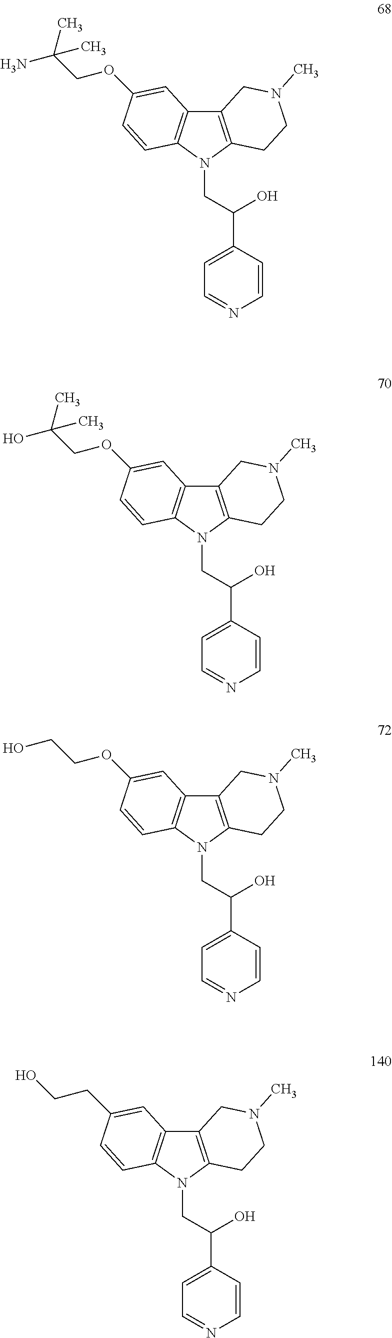 [SMILES: CN1CCc2c(c3cc(OCC(C)(C)N)ccc3n2CC(O)c2ccncc2)C1 ; CN1CCc2c(c3cc(OCC(C)(C)O)ccc3n2CC(O)c2ccncc2)C1 ; CN1CCc2c(c3cc(OCCO)ccc3n2CC(O)c2ccncc2)C1 ; CN1CCc2c(c3cc(CCO)ccc3n2CC(O)c2ccncc2)C1] 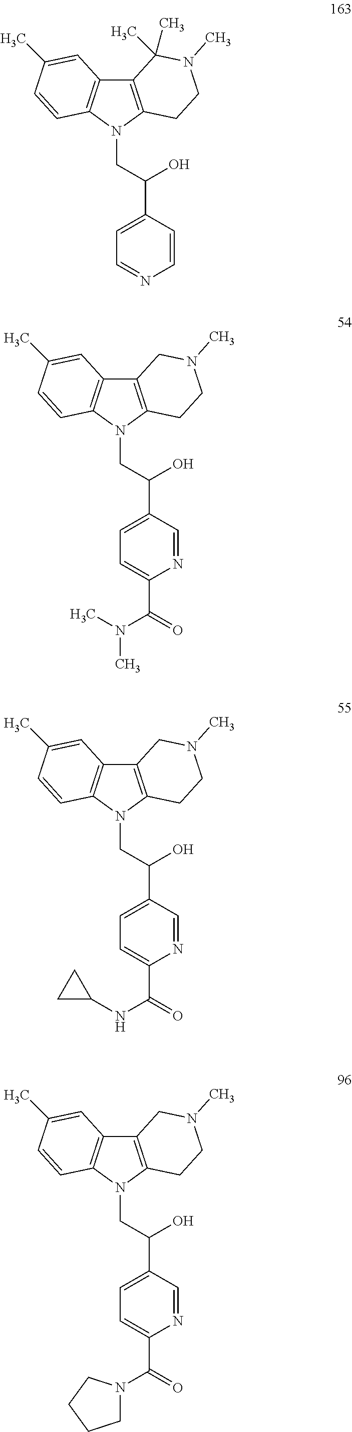 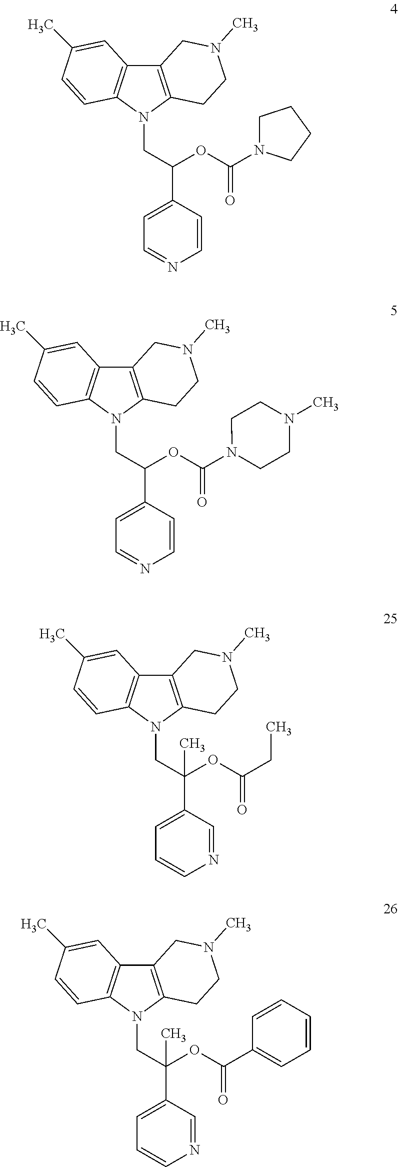 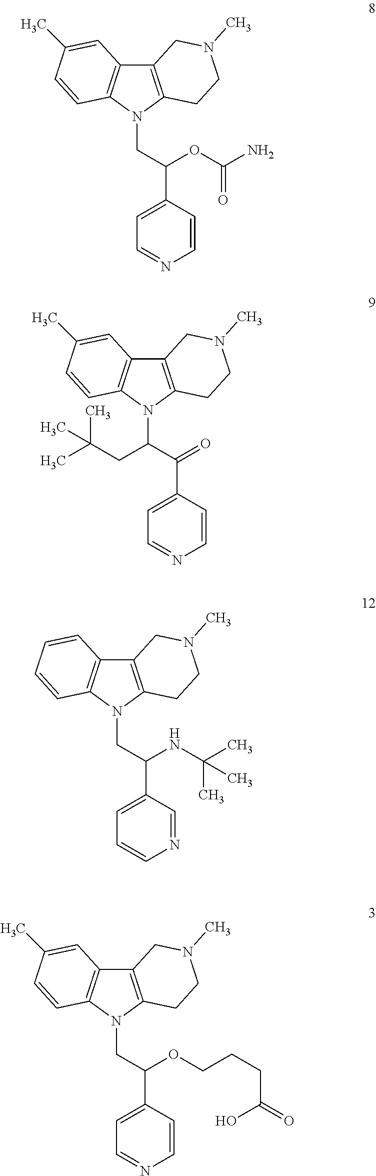 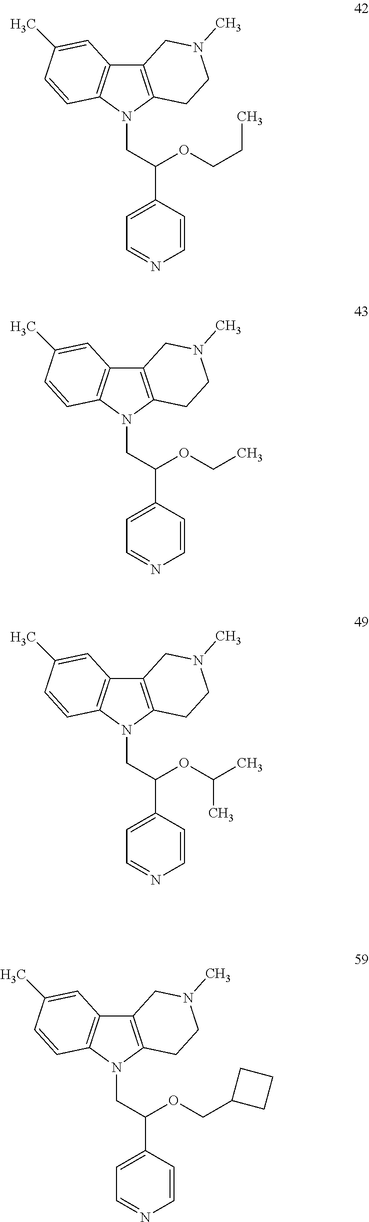 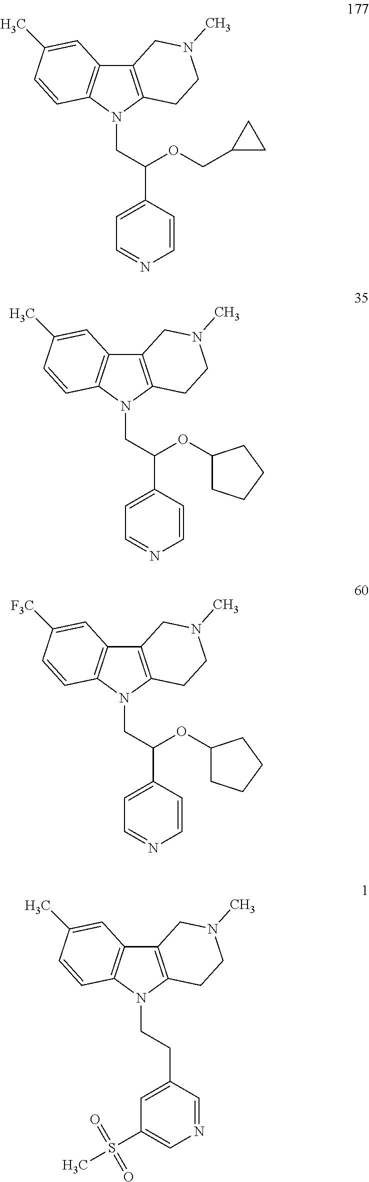 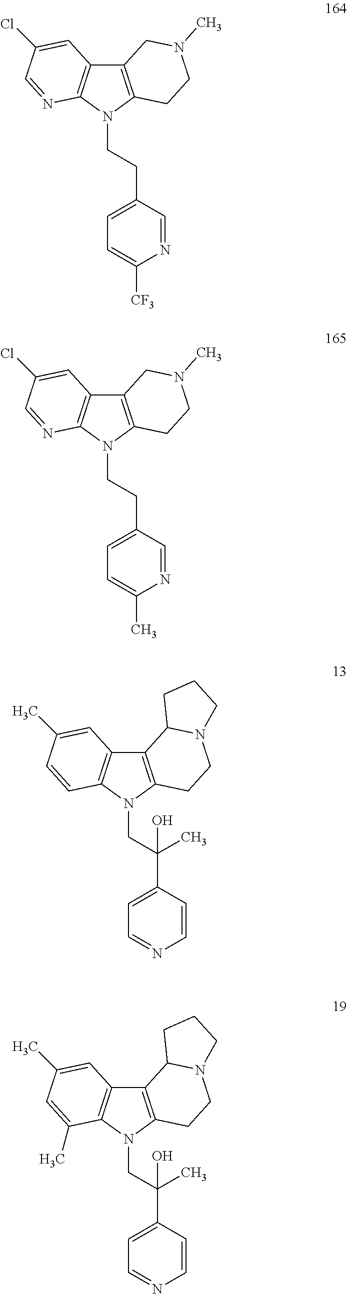 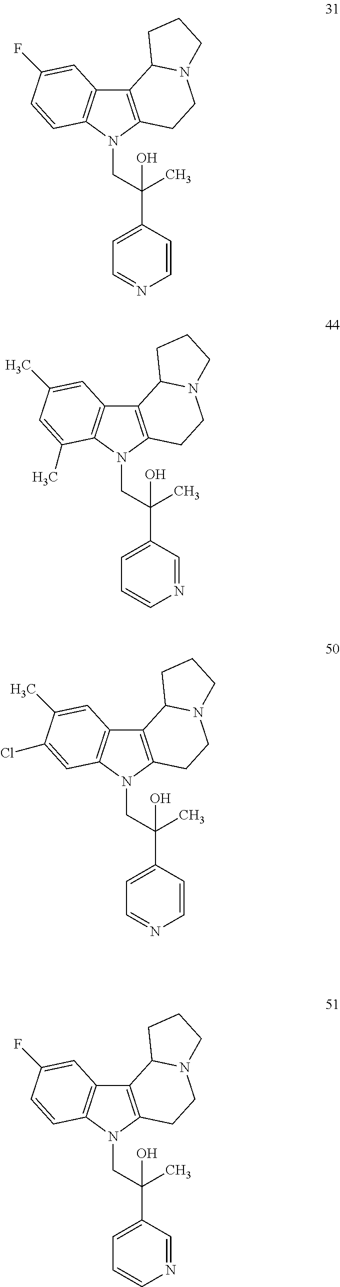 [SMILES: CC(O)(Cn1c2c(c3cc(F)ccc31)C1CCCN1CC2)c1ccncc1 ; Cc1cc(C)c2c(c1)c1c(n2CC(C)(O)c2cccnc2)CCN2CCCC12 ; Cc1cc2c3c(n(CC(C)(O)c4ccncc4)c2cc1Cl)CCN1CCCC31 ; CC(O)(Cn1c2c(c3cc(F)ccc31)C1CCCN1CC2)c1cccnc1] 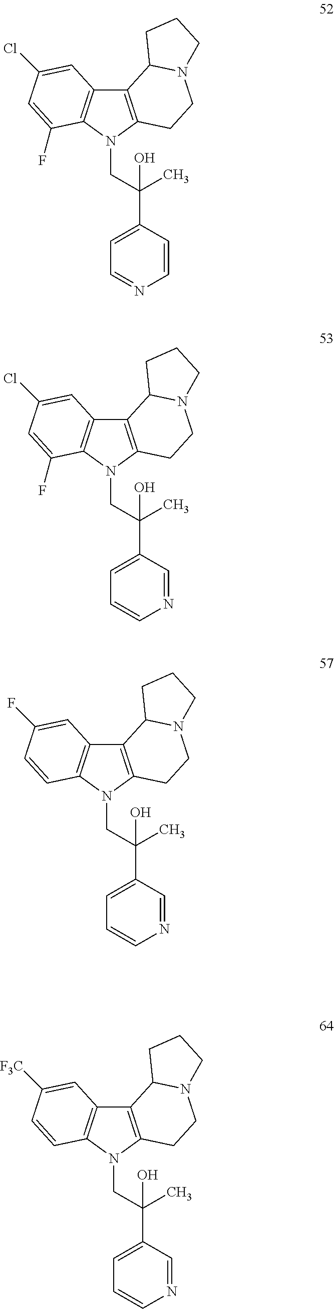 [SMILES: CC(O)(Cn1c2c(c3cc(Cl)cc(F)c31)C1CCCN1CC2)c1ccncc1 ; CC(O)(Cn1c2c(c3cc(Cl)cc(F)c31)C1CCCN1CC2)c1cccnc1 ; CC(O)(Cn1c2c(c3cc(F)ccc31)C1CCCN1CC2)c1cccnc1 ; CC(O)(Cn1c2c(c3cc(C(F)(F)F)ccc31)C1CCCN1CC2)c1cccnc1] 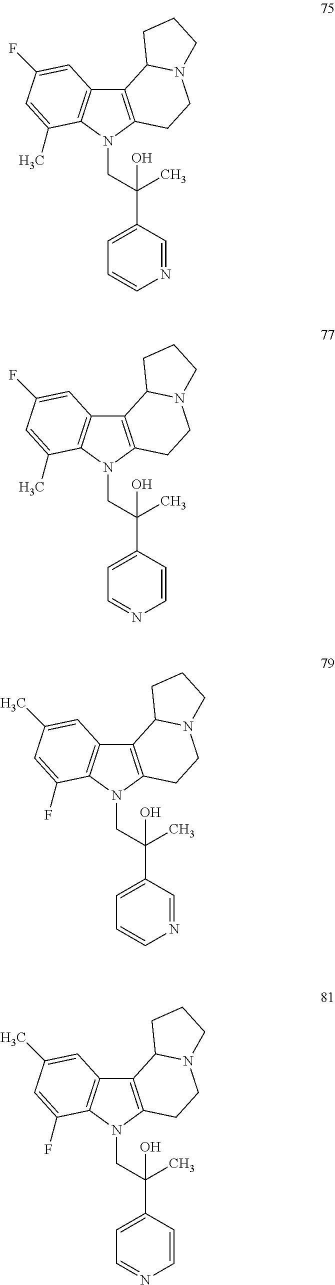 [SMILES: Cc1cc(F)cc2c3c(n(CC(C)(O)c4cccnc4)c12)CCN1CCCC31 ; Cc1cc(F)cc2c3c(n(CC(C)(O)c4ccncc4)c12)CCN1CCCC31 ; Cc1cc(F)c2c(c1)c1c(n2CC(C)(O)c2cccnc2)CCN2CCCC12 ; Cc1cc(F)c2c(c1)c1c(n2CC(C)(O)c2ccncc2)CCN2CCCC12] 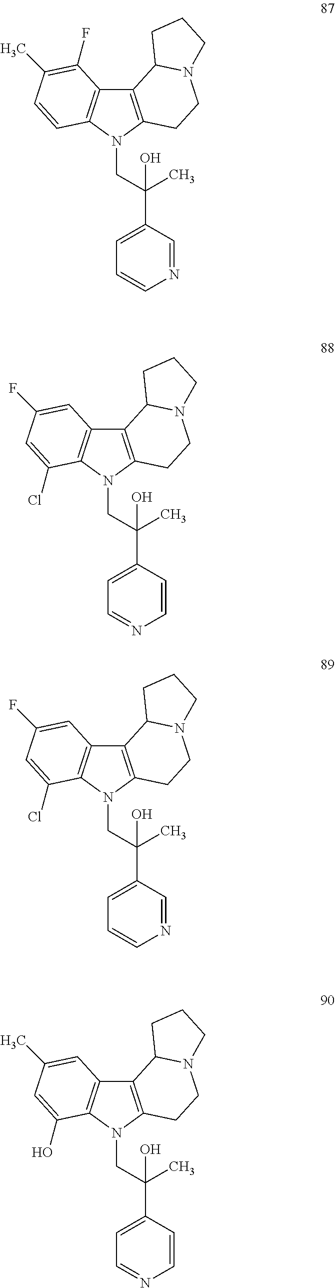 [SMILES: Cc1ccc2c(c1F)c1c(n2CC(C)(O)c2cccnc2)CCN2CCCC12 ; CC(O)(Cn1c2c(c3cc(F)cc(Cl)c31)C1CCCN1CC2)c1ccncc1 ; CC(O)(Cn1c2c(c3cc(F)cc(Cl)c31)C1CCCN1CC2)c1cccnc1 ; Cc1cc(O)c2c(c1)c1c(n2CC(C)(O)c2ccncc2)CCN2CCCC12] 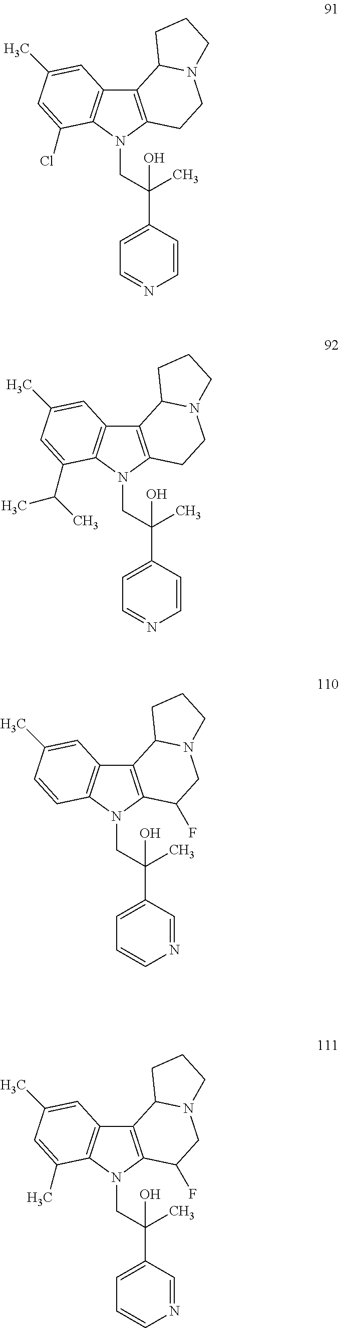 [SMILES: Cc1cc(Cl)c2c(c1)c1c(n2CC(C)(O)c2ccncc2)CCN2CCCC12 ; Cc1cc(C(C)C)c2c(c1)c1c(n2CC(C)(O)c2ccncc2)CCN2CCCC12 ; Cc1ccc2c(c1)c1c(n2CC(C)(O)c2cccnc2)C(F)CN2CCCC12 ; Cc1cc(C)c2c(c1)c1c(n2CC(C)(O)c2cccnc2)C(F)CN2CCCC12] 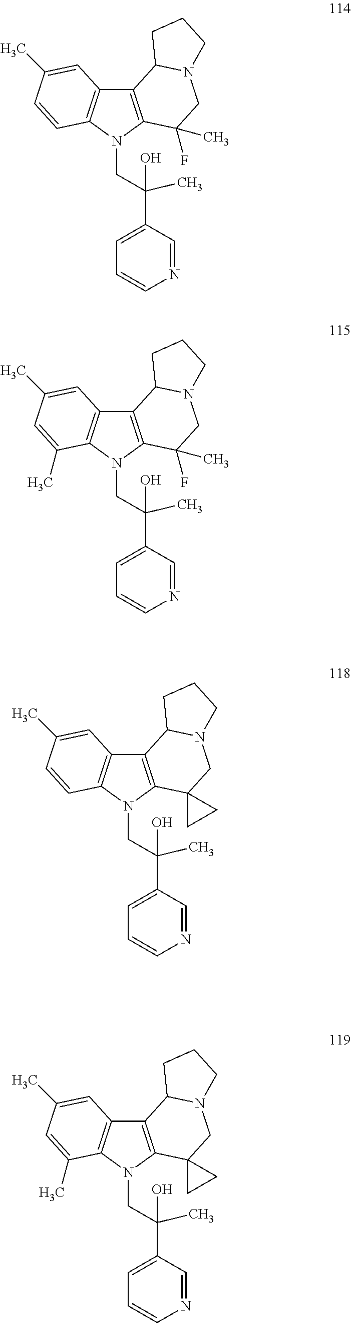 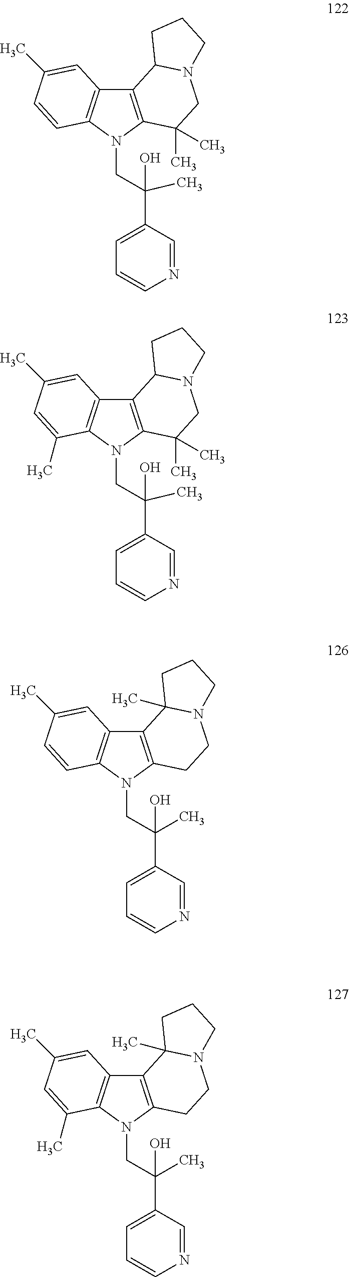 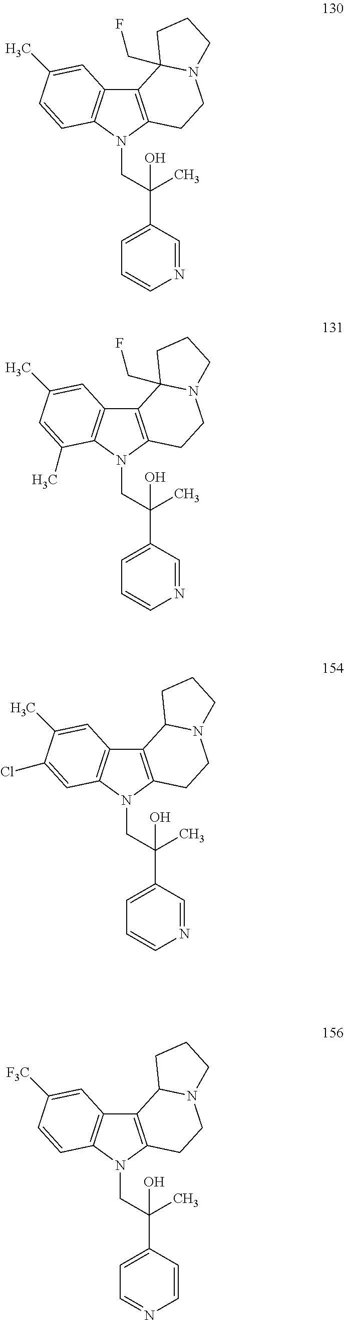 [SMILES: Cc1ccc2c(c1)c1c(n2CC(C)(O)c2cccnc2)CCN2CCCC12CF ; Cc1cc(C)c2c(c1)c1c(n2CC(C)(O)c2cccnc2)CCN2CCCC12CF ; Cc1cc2c3c(n(CC(C)(O)c4cccnc4)c2cc1Cl)CCN1CCCC31 ; CC(O)(Cn1c2c(c3cc(C(F)(F)F)ccc31)C1CCCN1CC2)c1ccncc1] 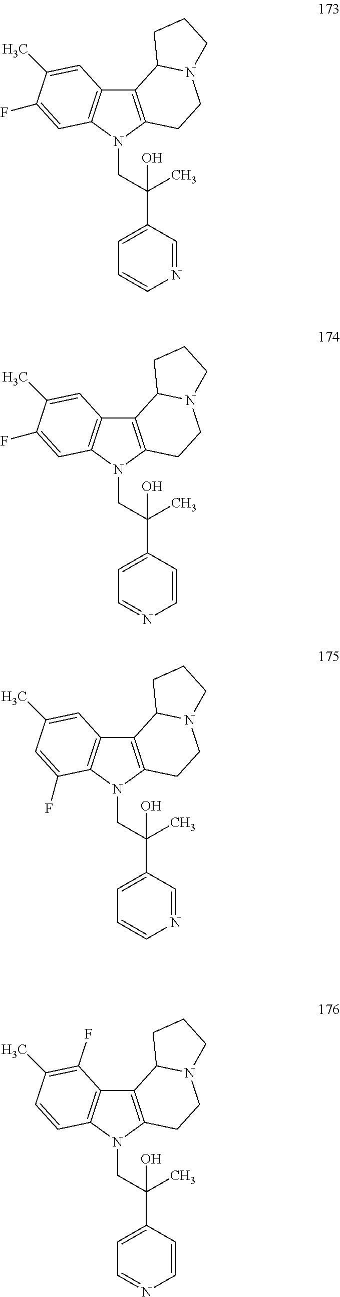 [SMILES: Cc1cc2c3c(n(CC(C)(O)c4cccnc4)c2cc1F)CCN1CCCC31 ; Cc1cc2c3c(n(CC(C)(O)c4ccncc4)c2cc1F)CCN1CCCC31 ; Cc1cc(F)c2c(c1)c1c(n2CC(C)(O)c2cccnc2)CCN2CCCC12 ; Cc1ccc2c(c1F)c1c(n2CC(C)(O)c2ccncc2)CCN2CCCC12] 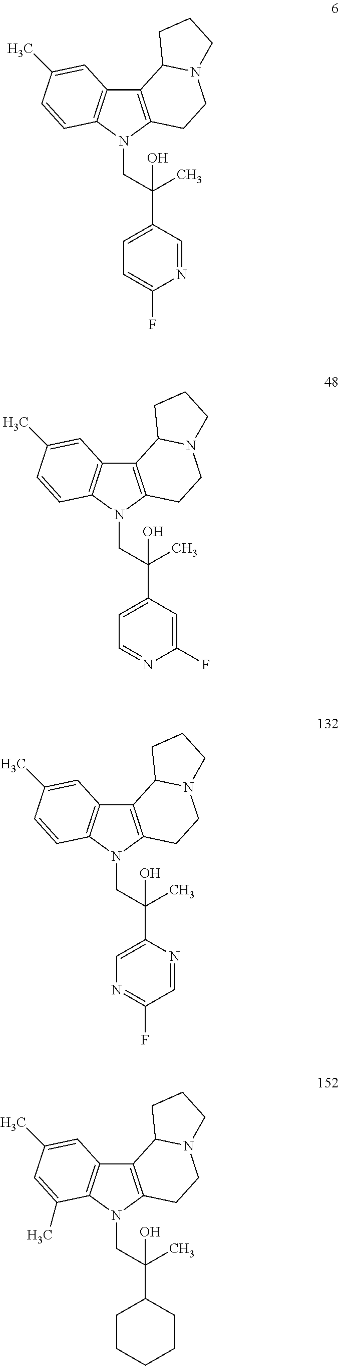 [SMILES: Cc1ccc2c(c1)c1c(n2CC(C)(O)c2ccc(F)nc2)CCN2CCCC12 ; Cc1ccc2c(c1)c1c(n2CC(C)(O)c2ccnc(F)c2)CCN2CCCC12 ; Cc1ccc2c(c1)c1c(n2CC(C)(O)c2cnc(F)cn2)CCN2CCCC12 ; Cc1cc(C)c2c(c1)c1c(n2CC(C)(O)C2CCCCC2)CCN2CCCC12] 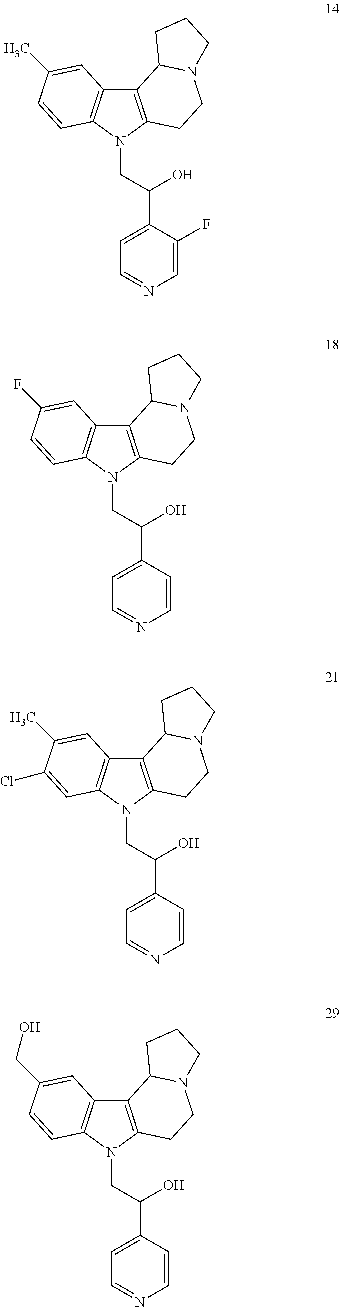 [SMILES: Cc1ccc2c(c1)c1c(n2CC(O)c2ccncc2F)CCN2CCCC12 ; OC(Cn1c2c(c3cc(F)ccc31)C1CCCN1CC2)c1ccncc1 ; Cc1cc2c3c(n(CC(O)c4ccncc4)c2cc1Cl)CCN1CCCC31 ; OCc1ccc2c(c1)c1c(n2CC(O)c2ccncc2)CCN2CCCC12] 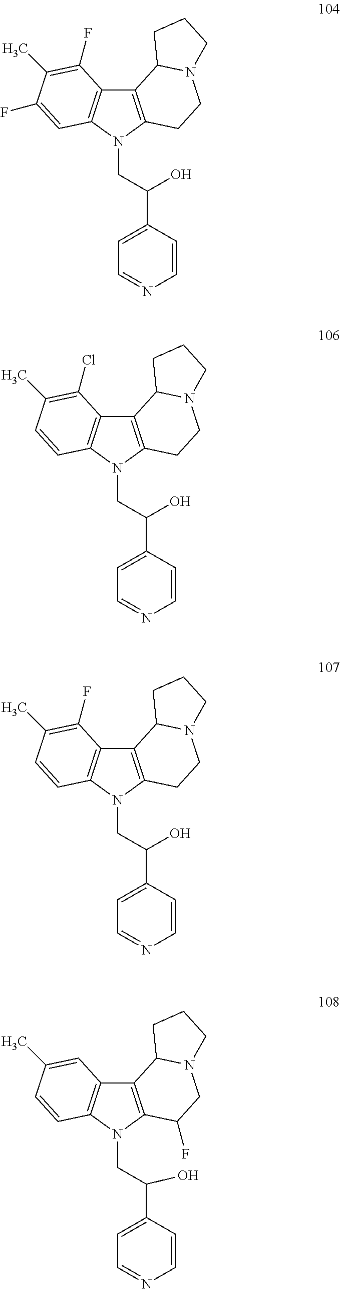 [SMILES: Cc1c(F)cc2c(c1F)c1c(n2CC(O)c2ccncc2)CCN2CCCC12 ; Cc1ccc2c(c1Cl)c1c(n2CC(O)c2ccncc2)CCN2CCCC12 ; Cc1ccc2c(c1F)c1c(n2CC(O)c2ccncc2)CCN2CCCC12 ; Cc1ccc2c(c1)c1c(n2CC(O)c2ccncc2)C(F)CN2CCCC12] 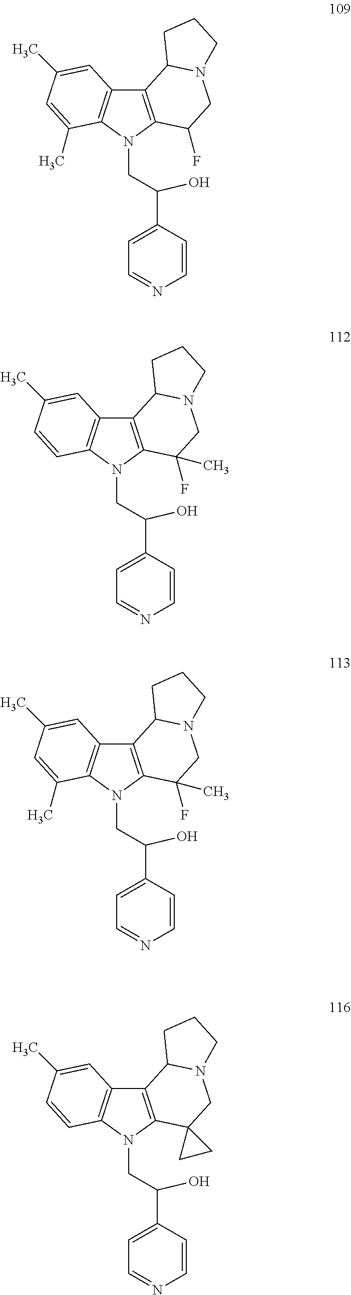 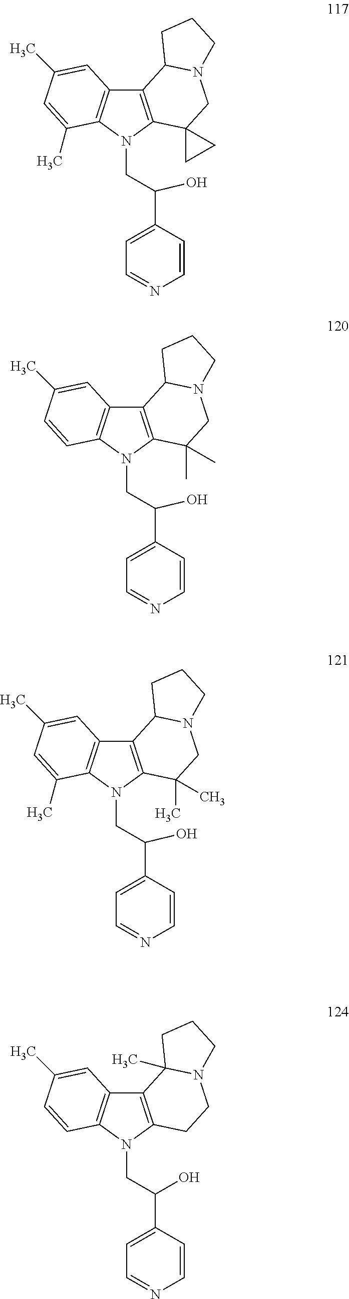 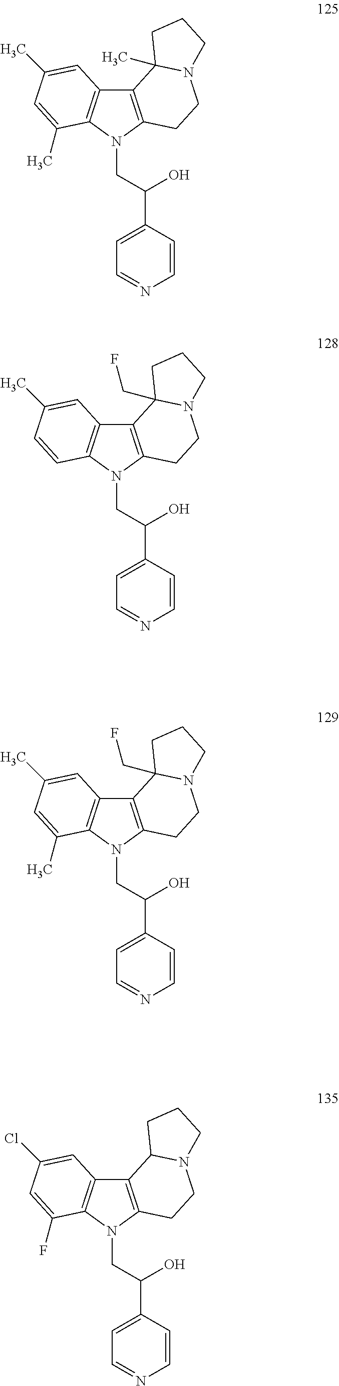 [SMILES: Cc1cc(C)c2c(c1)c1c(n2CC(O)c2ccncc2)CCN2CCCC12C ; Cc1ccc2c(c1)c1c(n2CC(O)c2ccncc2)CCN2CCCC12CF ; Cc1cc(C)c2c(c1)c1c(n2CC(O)c2ccncc2)CCN2CCCC12CF ; OC(Cn1c2c(c3cc(Cl)cc(F)c31)C1CCCN1CC2)c1ccncc1] 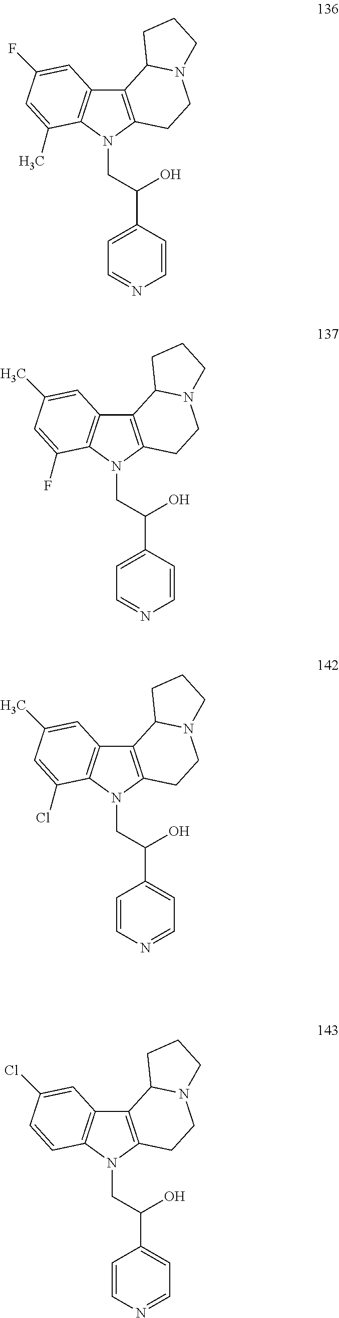 [SMILES: Cc1cc(F)cc2c3c(n(CC(O)c4ccncc4)c12)CCN1CCCC31 ; Cc1cc(F)c2c(c1)c1c(n2CC(O)c2ccncc2)CCN2CCCC12 ; Cc1cc(Cl)c2c(c1)c1c(n2CC(O)c2ccncc2)CCN2CCCC12 ; OC(Cn1c2c(c3cc(Cl)ccc31)C1CCCN1CC2)c1ccncc1] 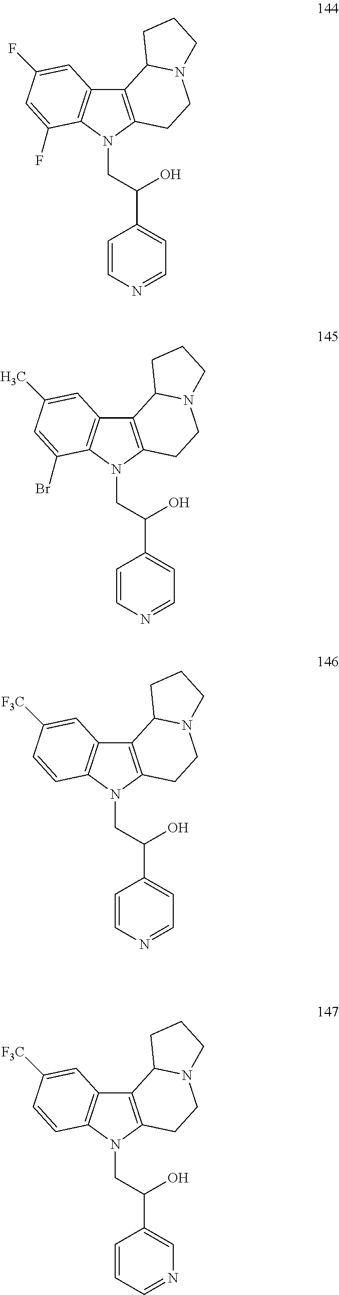 [SMILES: OC(Cn1c2c(c3cc(F)cc(F)c31)C1CCCN1CC2)c1ccncc1 ; Cc1cc(Br)c2c(c1)c1c(n2CC(O)c2ccncc2)CCN2CCCC12 ; OC(Cn1c2c(c3cc(C(F)(F)F)ccc31)C1CCCN1CC2)c1ccncc1 ; OC(Cn1c2c(c3cc(C(F)(F)F)ccc31)C1CCCN1CC2)c1cccnc1] 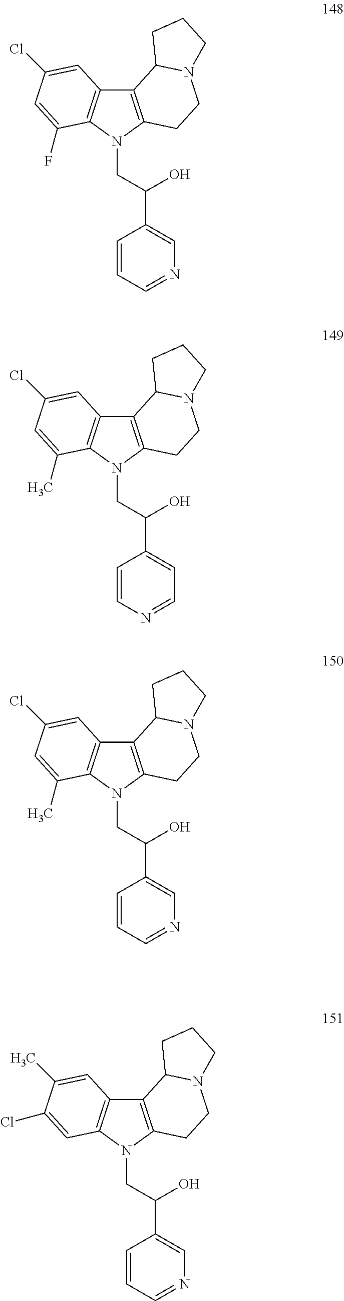 [SMILES: OC(Cn1c2c(c3cc(Cl)cc(F)c31)C1CCCN1CC2)c1cccnc1 ; Cc1cc(Cl)cc2c3c(n(CC(O)c4ccncc4)c12)CCN1CCCC31 ; Cc1cc(Cl)cc2c3c(n(CC(O)c4cccnc4)c12)CCN1CCCC31 ; Cc1cc2c3c(n(CC(O)c4cccnc4)c2cc1Cl)CCN1CCCC31] 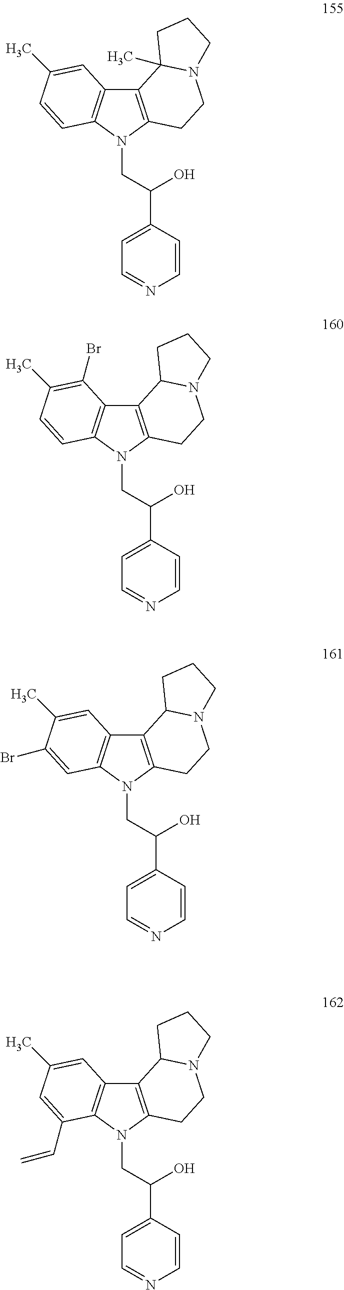 [SMILES: Cc1ccc2c(c1)c1c(n2CC(O)c2ccncc2)CCN2CCCC12C ; Cc1ccc2c(c1Br)c1c(n2CC(O)c2ccncc2)CCN2CCCC12 ; Cc1cc2c3c(n(CC(O)c4ccncc4)c2cc1Br)CCN1CCCC31 ; C=Cc1cc(C)cc2c3c(n(CC(O)c4ccncc4)c12)CCN1CCCC31] 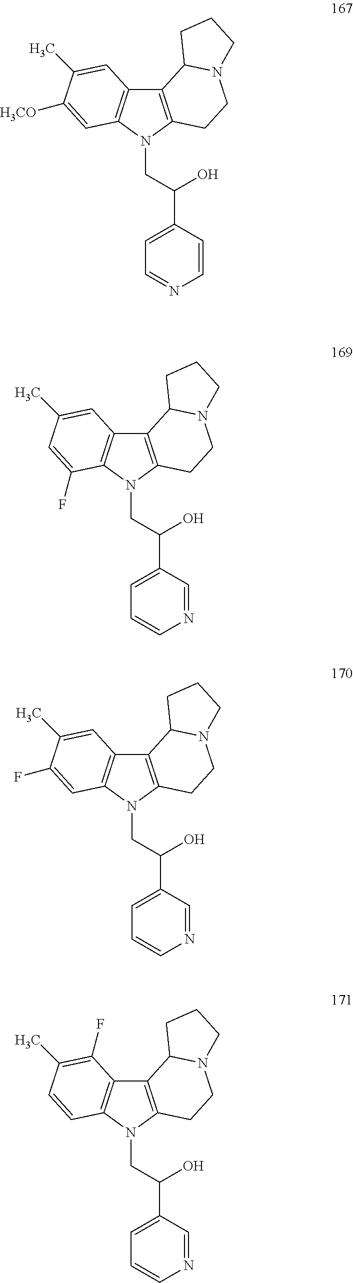 [SMILES: COc1cc2c(cc1C)c1c(n2CC(O)c2ccncc2)CCN2CCCC12 ; Cc1cc(F)c2c(c1)c1c(n2CC(O)c2cccnc2)CCN2CCCC12 ; Cc1cc2c3c(n(CC(O)c4cccnc4)c2cc1F)CCN1CCCC31 ; Cc1ccc2c(c1F)c1c(n2CC(O)c2cccnc2)CCN2CCCC12] 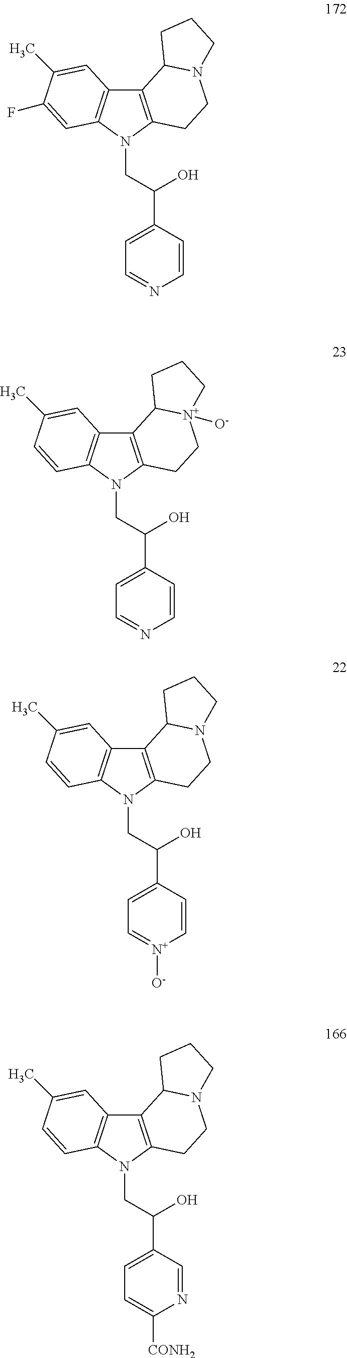 [SMILES: Cc1cc2c3c(n(CC(O)c4ccncc4)c2cc1F)CCN1CCCC31 ; Cc1ccc2c(c1)c1c(n2CC(O)c2ccncc2)CC[N+]2([O-])CCCC12 ; Cc1ccc2c(c1)c1c(n2CC(O)c2cc[n+]([O-])cc2)CCN2CCCC12 ; Cc1ccc2c(c1)c1c(n2CC(O)c2ccc(C(N)=O)nc2)CCN2CCCC12] 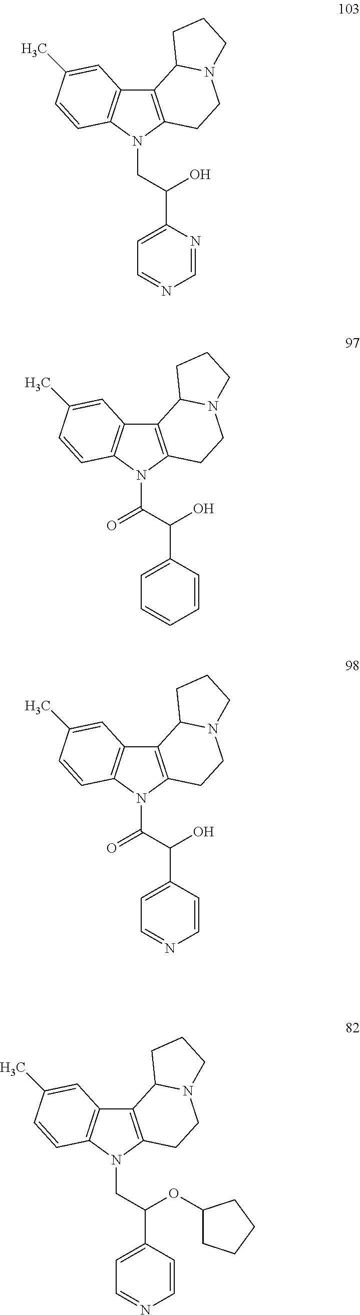 [SMILES: Cc1ccc2c(c1)c1c(n2CC(O)c2ccncn2)CCN2CCCC12 ; Cc1ccc2c(c1)c1c(n2C(=O)C(O)c2ccccc2)CCN2CCCC12 ; Cc1ccc2c(c1)c1c(n2C(=O)C(O)c2ccncc2)CCN2CCCC12 ; Cc1ccc2c(c1)c1c(n2CC(OC2CCCC2)c2ccncc2)CCN2CCCC12] 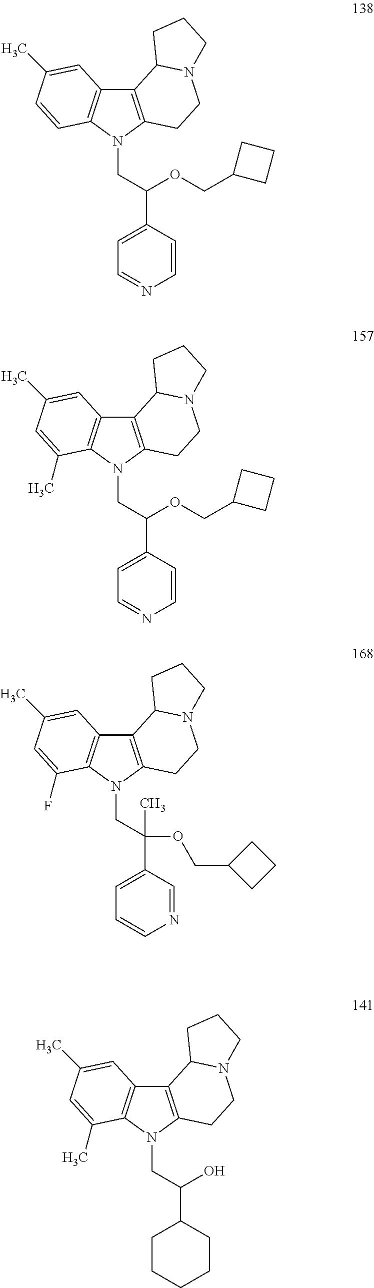 [SMILES: Cc1ccc2c(c1)c1c(n2CC(OCC2CCC2)c2ccncc2)CCN2CCCC12 ; Cc1cc(C)c2c(c1)c1c(n2CC(OCC2CCC2)c2ccncc2)CCN2CCCC12 ; Cc1cc(C)c2c(c1)c1c(n2CC(C)(OCC2CCC2)c2cccnc2)CCN2CCCC12 ; Cc1cc(C)c2c(c1)c1c(n2CC(O)C2CCCCC2)CCN2CCCC12] 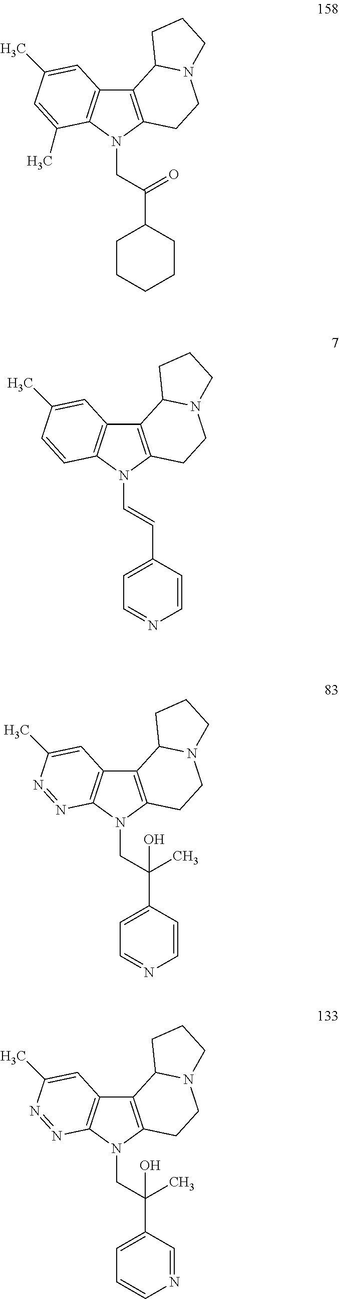 [SMILES: Cc1cc(C)c2c(c1)c1c(n2CC(=O)C2CCCCC2)CCN2CCCC12 ; Cc1ccc2c(c1)c1c(n2/C=C/c2ccncc2)CCN2CCCC12 ; Cc1cc2c3c(n(CC(C)(O)c4ccncc4)c2nn1)CCN1CCCC31 ; Cc1cc2c3c(n(CC(C)(O)c4cccnc4)c2nn1)CCN1CCCC31] 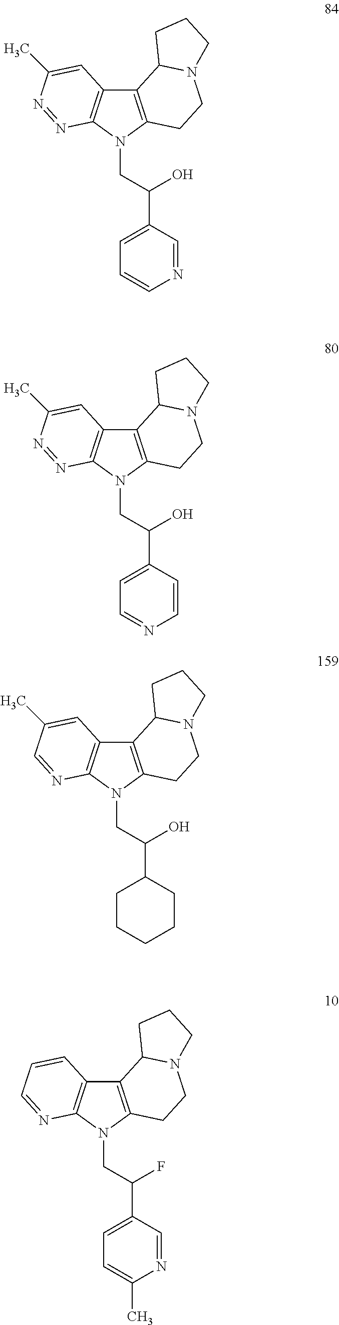 [SMILES: Cc1cc2c3c(n(CC(O)c4cccnc4)c2nn1)CCN1CCCC31 ; Cc1cc2c3c(n(CC(O)c4ccncc4)c2nn1)CCN1CCCC31 ; Cc1cnc2c(c1)c1c(n2CC(O)C2CCCCC2)CCN2CCCC12 ; Cc1ccc(C(F)Cn2c3c(c4cccnc42)C2CCCN2CC3)cn1] 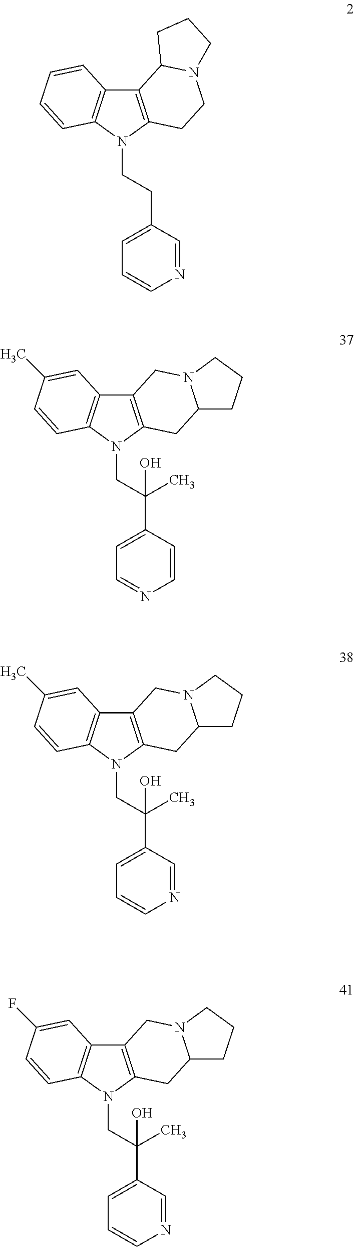 [SMILES: c1cncc(CCn2c3c(c4ccccc42)C2CCCN2CC3)c1 ; Cc1ccc2c(c1)c1c(n2CC(C)(O)c2ccncc2)CC2CCCN2C1 ; Cc1ccc2c(c1)c1c(n2CC(C)(O)c2cccnc2)CC2CCCN2C1 ; CC(O)(Cn1c2c(c3cc(F)ccc31)CN1CCCC1C2)c1cccnc1] 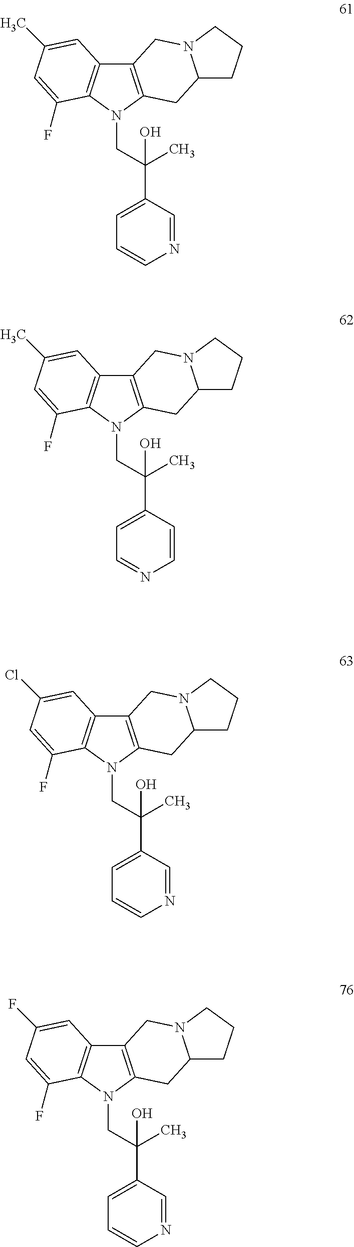 [SMILES: Cc1cc(F)c2c(c1)c1c(n2CC(C)(O)c2cccnc2)CC2CCCN2C1 ; Cc1cc(F)c2c(c1)c1c(n2CC(C)(O)c2ccncc2)CC2CCCN2C1 ; CC(O)(Cn1c2c(c3cc(Cl)cc(F)c31)CN1CCCC1C2)c1cccnc1 ; CC(O)(Cn1c2c(c3cc(F)cc(F)c31)CN1CCCC1C2)c1cccnc1] 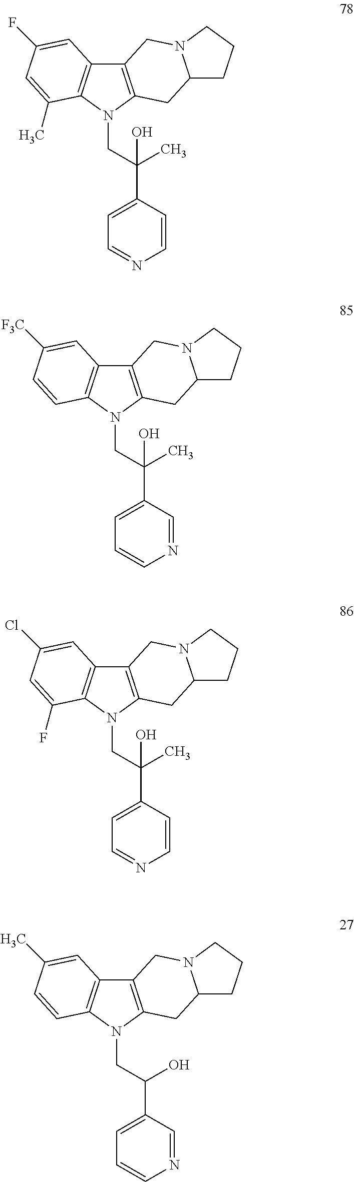 [SMILES: Cc1cc(F)cc2c3c(n(CC(C)(O)c4ccncc4)c12)CC1CCCN1C3 ; CC(O)(Cn1c2c(c3cc(C(F)(F)F)ccc31)CN1CCCC1C2)c1cccnc1 ; CC(O)(Cn1c2c(c3cc(Cl)cc(F)c31)CN1CCCC1C2)c1ccncc1 ; Cc1ccc2c(c1)c1c(n2CC(O)c2cccnc2)CC2CCCN2C1] 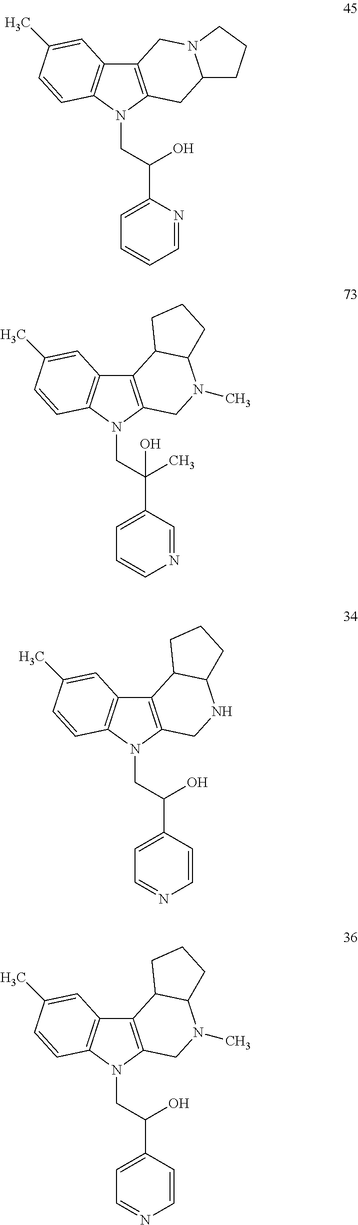 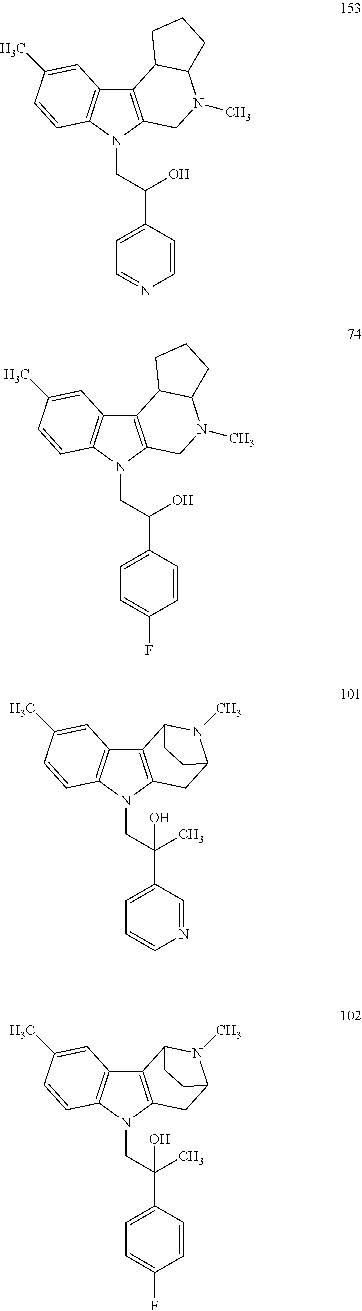 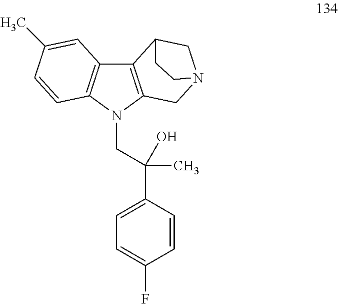 [SMILES: Cc1ccc2c(c1)c1c(n2CC(C)(O)c2ccc(F)cc2)CN2CCC1C2]
or a pharmaceutically acceptable salt thereof.

2. The compound of claim 1, wherein the compound is selected from the group consisting of Compound Nos. 1-133, or a pharmaceutically acceptable salt thereof.

3. A pharmaceutical composition comprising (a) a compound of claim 1, or a pharmaceutically acceptable salt thereof, and (b) a pharmaceutically acceptable carrier.

4. A method of lowering blood pressure in an individual in need thereof comprising administering to the individual an effective amount of a compound of claim 1, or a pharmaceutically acceptable salt thereof.

5-9. (canceled)

10. A method of (i) increasing renal blood flow, and/or (ii) decreasing sodium reabsorption, in an individual in need thereof comprising administering to the individual an effective amount of a compound of a compound of claim 1.

11-13. (canceled)

14. A method of treating a disease or condition that is responsive to any one or more of: (i) a decrease in blood pressure; (ii) an increase in renal blood flow; and (iii) a decrease of sodium reabsorption, comprising administering to an individual in need thereof an effective amount of a compound of claim 1, or a pharmaceutically acceptable salt thereof.

15. The method of claim 14, wherein the disease or condition is hypertension.

16. (canceled)

17. The method of claim 14, wherein the disease or condition is hypertensive emergency.

18. The method of claim 14, wherein the disease or condition is a cardiac or renal disease or condition.

19-21. (canceled)

22. A kit comprising a compound of claim 1, or a pharmaceutically acceptable salt thereof.

23. (canceled)

24. A method of regulating blood glucose levels in an individual in need thereof, comprising administering to the individual an effective amount of a compound of claim 1, or a pharmaceutically acceptable salt thereof.

25. The method of claim 24, wherein the method reduces blood glucose level in the individual.

26. A method of increasing insulin secretion, and/or promoting insulin release into the blood stream, in an individual in need thereof, comprising administering to the individual an effective amount of a compound of claim 1, or a pharmaceutically acceptable salt thereof.

27. A method of treating or delaying the onset of a disease or condition that is responsive to an increase in insulin secretion in an individual in need thereof, comprising administering to the individual an effective amount of a compound of claim 1, or a pharmaceutically acceptable salt thereof.

28. The method of claim 27, wherein the disease or condition is type 2 diabetes.

29. The method of claim 27, wherein the disease or condition is glucose intolerance or metabolic syndrome.

30. The compound of claim 1, or a pharmaceutically acceptable salt thereof, wherein the compound is selected from the group consisting of Compound Nos. 1, 3, 4, 5, 8, 9, 11, 12, 15, 16, 17, 20, 24, 25, 26, 28, 30, 32, 33, 35, 39, 40, 42, 43, 46, 47, 49, 54, 55, 56, 58, 59, 60, 65, 66, 67, 68, 69, 70, 71, 72, 93, 94, 95, 96, 99, 100, 105, 139, 140, 163, 164, 165 and 177:

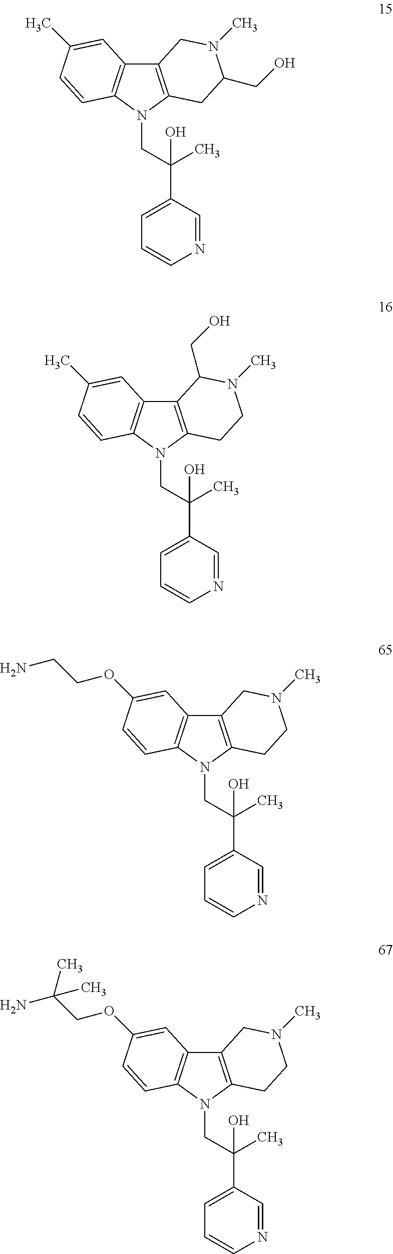

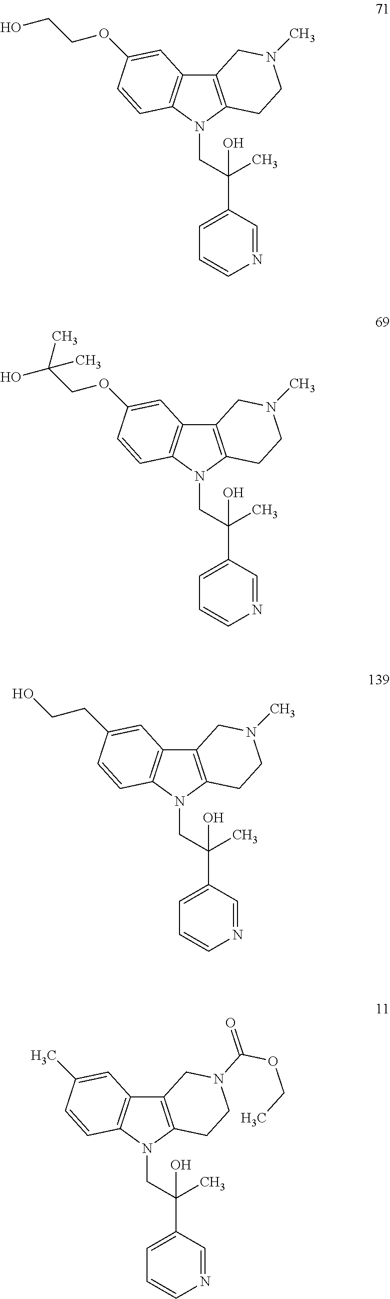

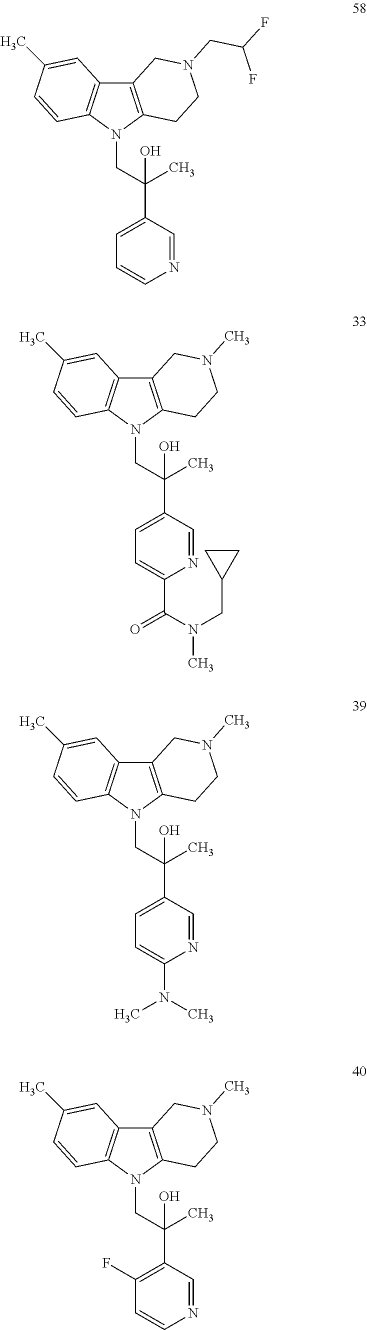

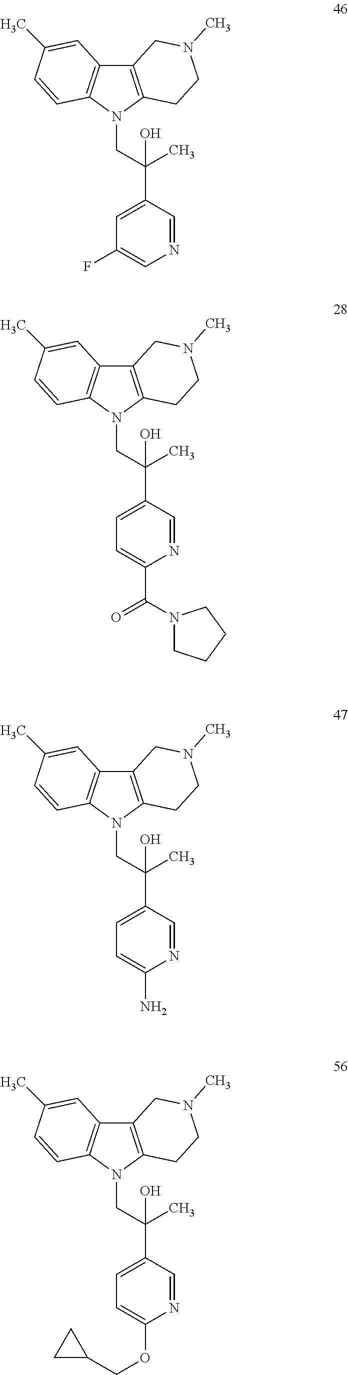

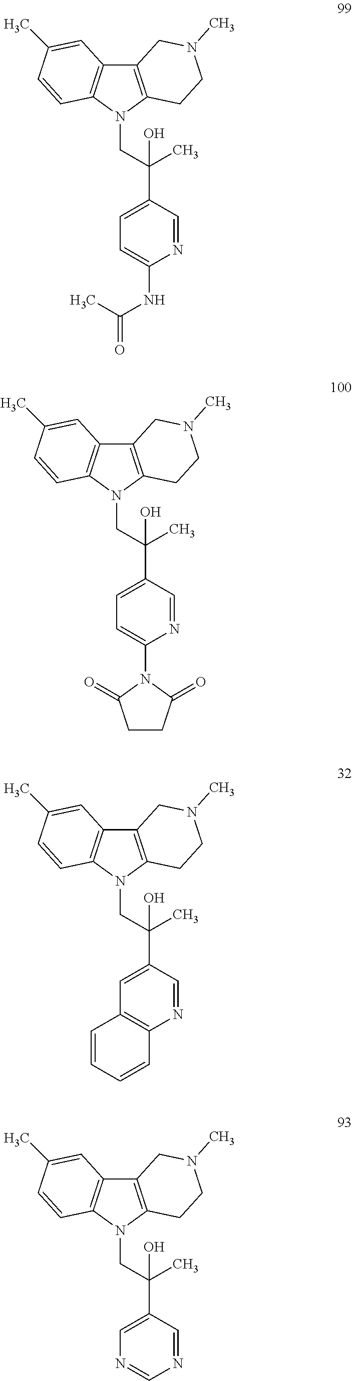

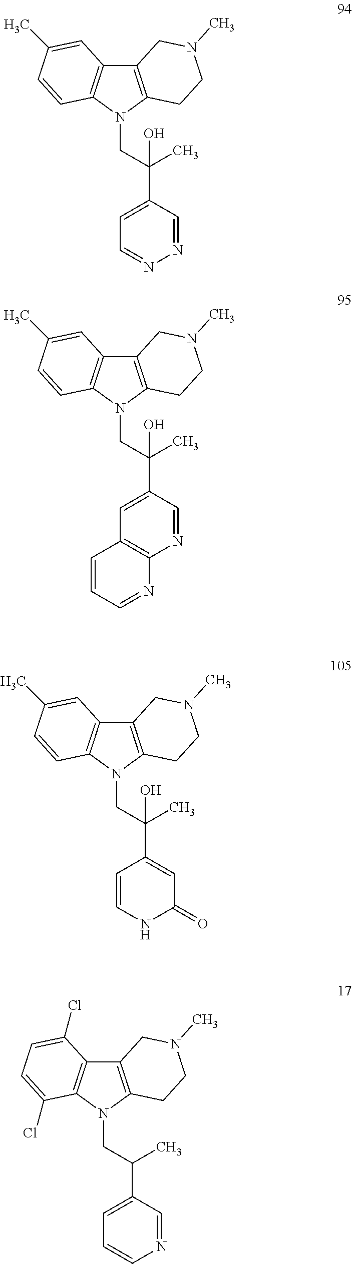

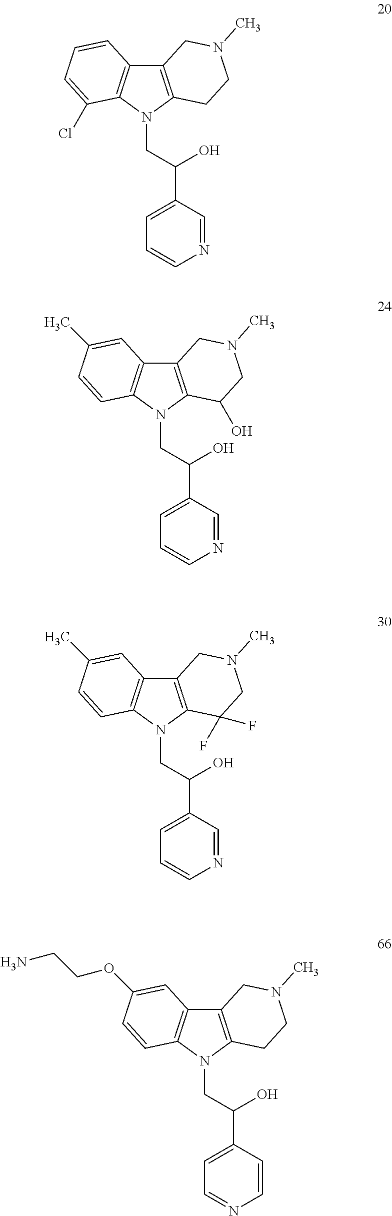

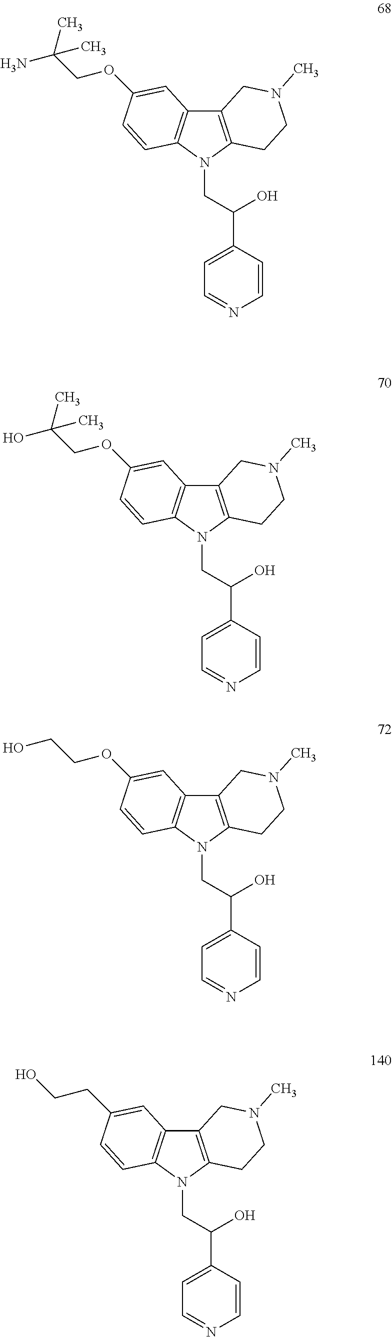 [SMILES: CN1CCc2c(c3cc(OCC(C)(C)N)ccc3n2CC(O)c2ccncc2)C1 ; CN1CCc2c(c3cc(OCC(C)(C)O)ccc3n2CC(O)c2ccncc2)C1 ; CN1CCc2c(c3cc(OCCO)ccc3n2CC(O)c2ccncc2)C1 ; CN1CCc2c(c3cc(CCO)ccc3n2CC(O)c2ccncc2)C1]

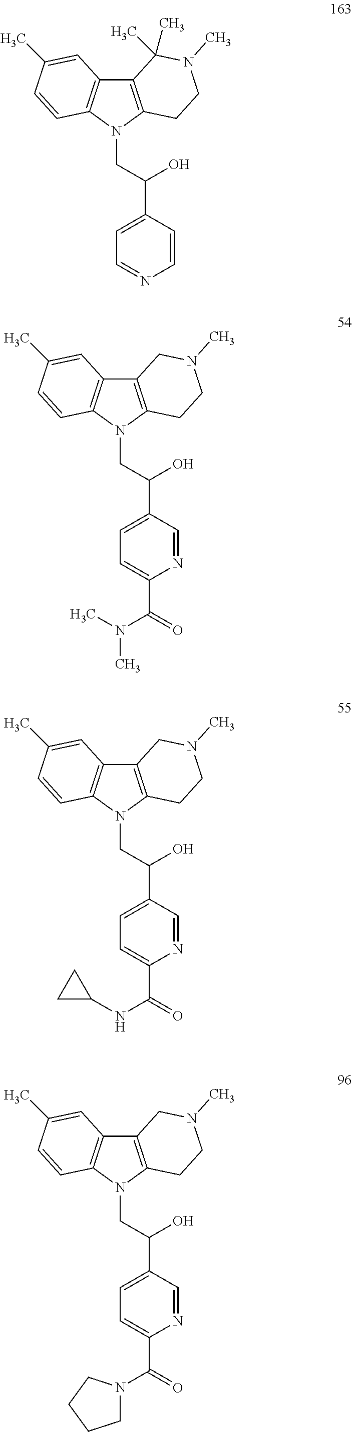

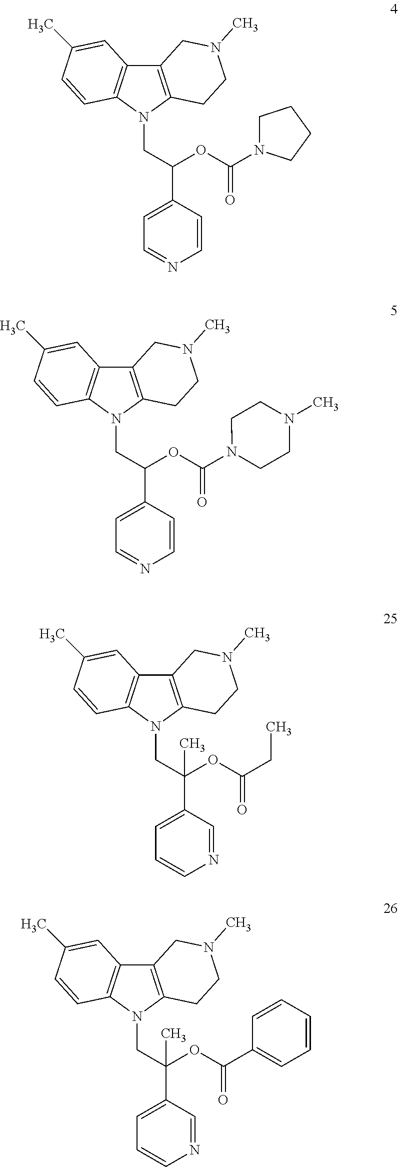

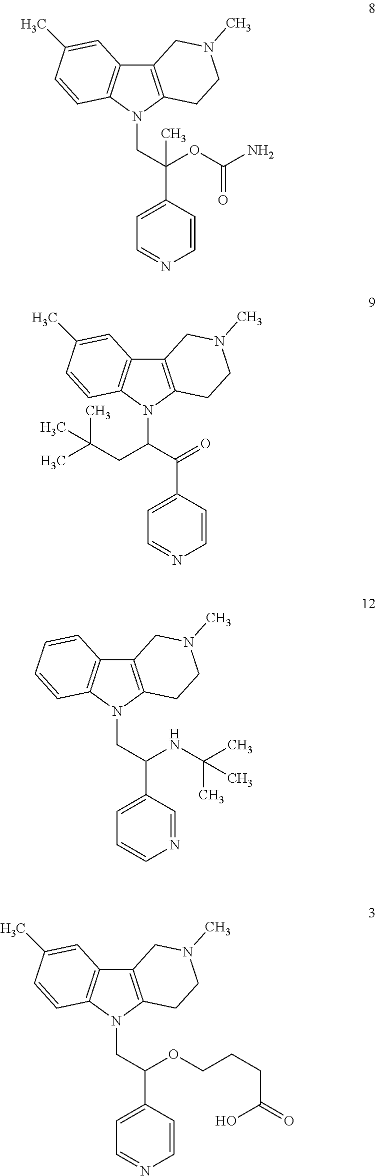

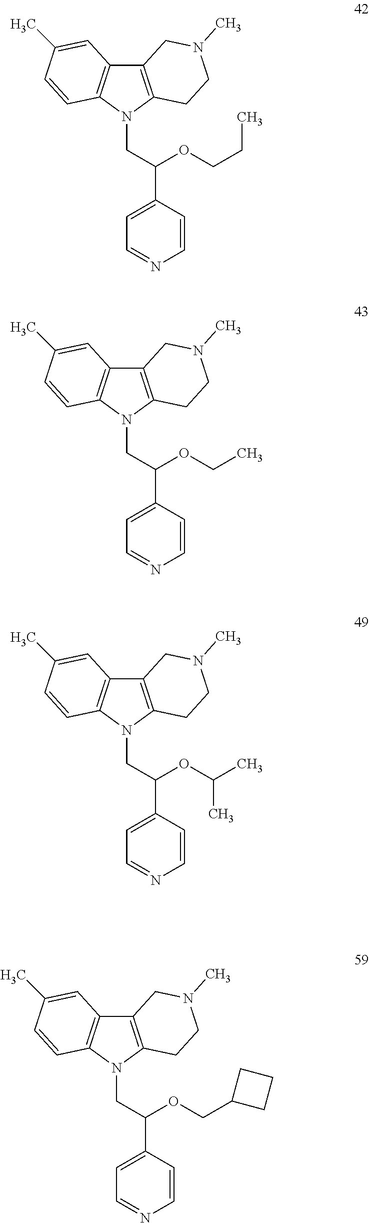

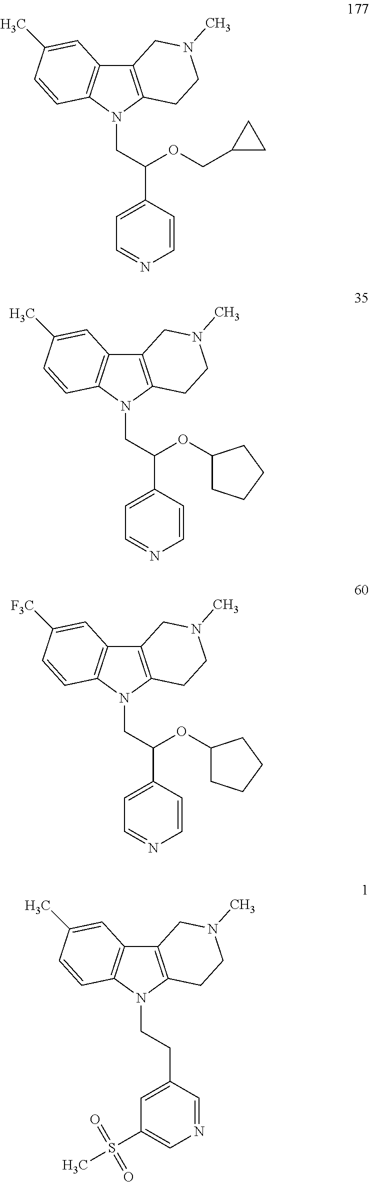

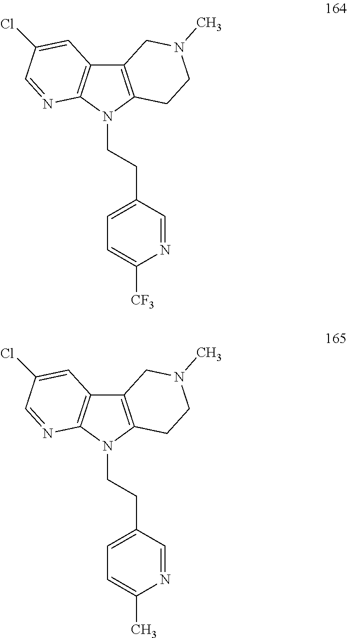 [SMILES: CN1CCc2c(c3cc(Cl)cnc3n2CCc2ccc(C(F)(F)F)nc2)C1 ; Cc1ccc(CCn2c3c(c4cc(Cl)cnc42)CN(C)CC3)cn1]

31. The compound of claim 1, or a pharmaceutically acceptable salt thereof, wherein the compound is selected from the group consisting of Compound Nos. 2, 6, 7, 10, 13, 14, 18, 19, 21, 22, 23, 29, 31, 44, 48, 50, 51, 52, 53, 57, 64, 75, 77, 79, 80, 81, 82, 83, 84, 87, 88, 89, 90, 91, 92, 97, 98, 103, 104, 106, 107, 108, 109, 110, 111, 112, 113, 114, 115, 116, 117, 118, 119, 120, 121, 122, 123, 124, 125, 126, 127, 128, 129, 130, 131, 132, 133, 135, 136, 137, 138, 141, 142, 143, 144, 145, 146, 147, 148, 149, 150, 151, 152, 154, 155, 156, 157, 158, 159, 160, 161, 162, 166, 167, 168, 169, 170, 171, 172, 173, 174, 175 and 176:

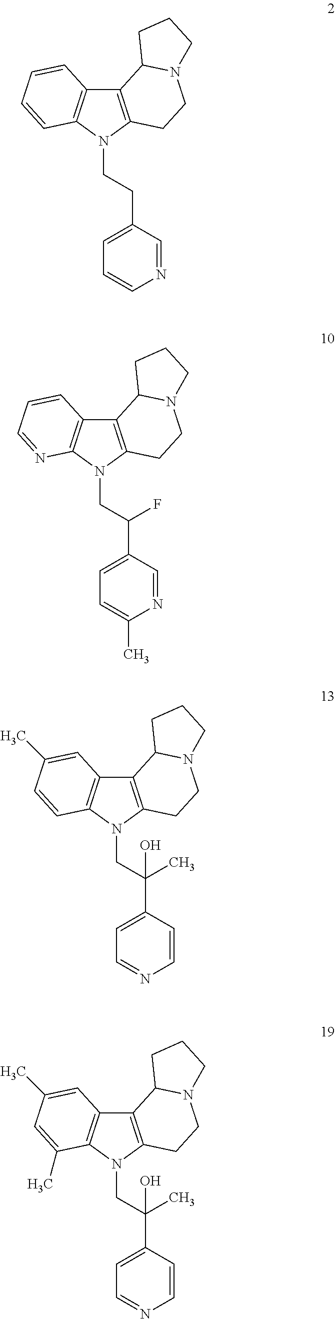 [SMILES: c1cncc(CCn2c3c(c4ccccc42)C2CCCN2CC3)c1 ; Cc1ccc(C(F)Cn2c3c(c4cccnc42)C2CCCN2CC3)cn1 ; Cc1ccc2c(c1)c1c(n2CC(C)(O)c2ccncc2)CCN2CCCC12 ; Cc1cc(C)c2c(c1)c1c(n2CC(C)(O)c2ccncc2)CCN2CCCC12]

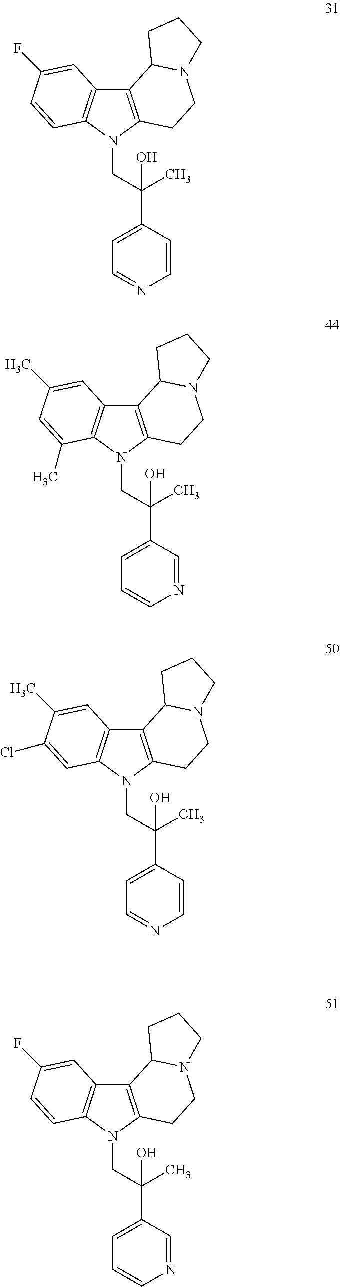 [SMILES: CC(O)(Cn1c2c(c3cc(F)ccc31)C1CCCN1CC2)c1ccncc1 ; Cc1cc(C)c2c(c1)c1c(n2CC(C)(O)c2cccnc2)CCN2CCCC12 ; Cc1cc2c3c(n(CC(C)(O)c4ccncc4)c2cc1Cl)CCN1CCCC31 ; CC(O)(Cn1c2c(c3cc(F)ccc31)C1CCCN1CC2)c1cccnc1]

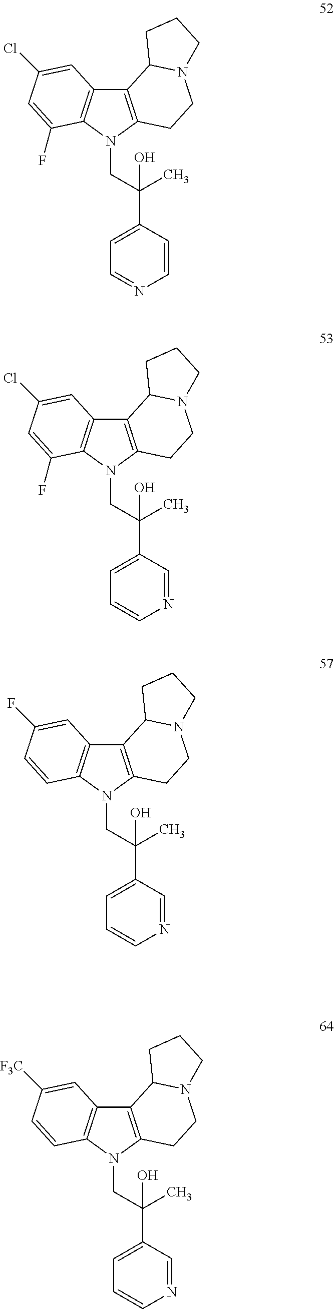 [SMILES: CC(O)(Cn1c2c(c3cc(Cl)cc(F)c31)C1CCCN1CC2)c1ccncc1 ; CC(O)(Cn1c2c(c3cc(Cl)cc(F)c31)C1CCCN1CC2)c1cccnc1 ; CC(O)(Cn1c2c(c3cc(F)ccc31)C1CCCN1CC2)c1cccnc1 ; CC(O)(Cn1c2c(c3cc(C(F)(F)F)ccc31)C1CCCN1CC2)c1cccnc1]

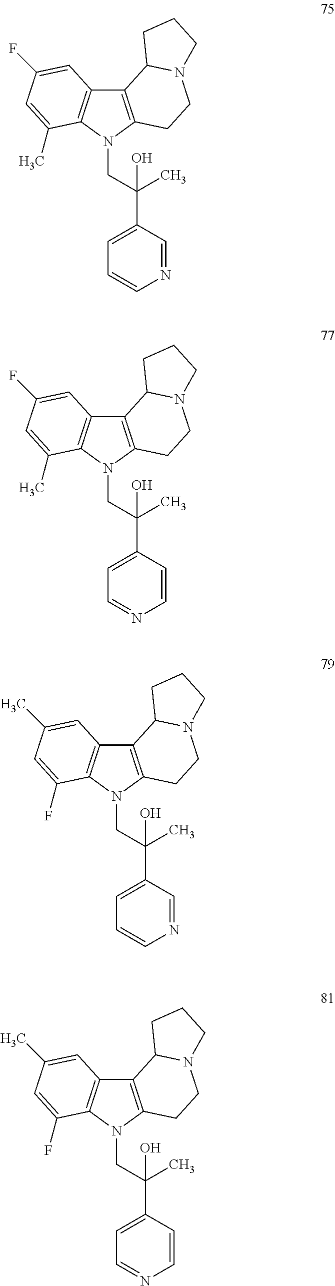 [SMILES: Cc1cc(F)cc2c3c(n(CC(C)(O)c4cccnc4)c12)CCN1CCCC31 ; Cc1cc(F)cc2c3c(n(CC(C)(O)c4ccncc4)c12)CCN1CCCC31 ; Cc1cc(F)c2c(c1)c1c(n2CC(C)(O)c2cccnc2)CCN2CCCC12 ; Cc1cc(F)c2c(c1)c1c(n2CC(C)(O)c2ccncc2)CCN2CCCC12]

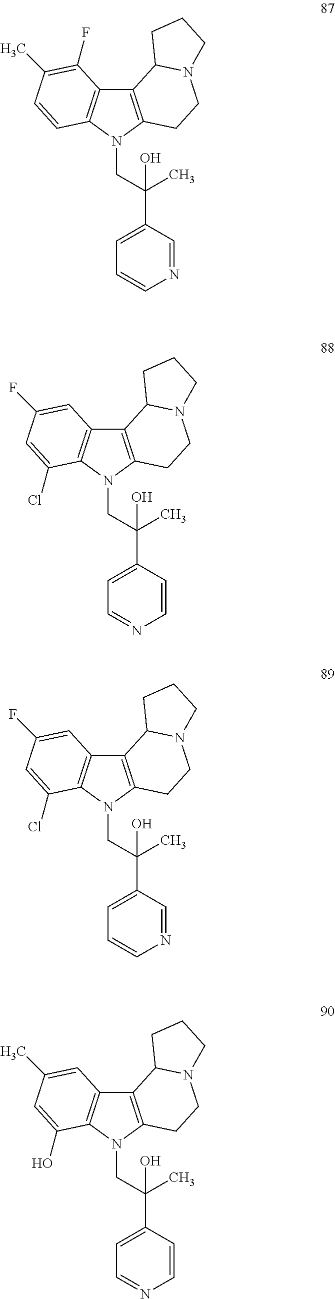 [SMILES: Cc1ccc2c(c1F)c1c(n2CC(C)(O)c2cccnc2)CCN2CCCC12 ; CC(O)(Cn1c2c(c3cc(F)cc(Cl)c31)C1CCCN1CC2)c1ccncc1 ; CC(O)(Cn1c2c(c3cc(F)cc(Cl)c31)C1CCCN1CC2)c1cccnc1 ; Cc1cc(O)c2c(c1)c1c(n2CC(C)(O)c2ccncc2)CCN2CCCC12]

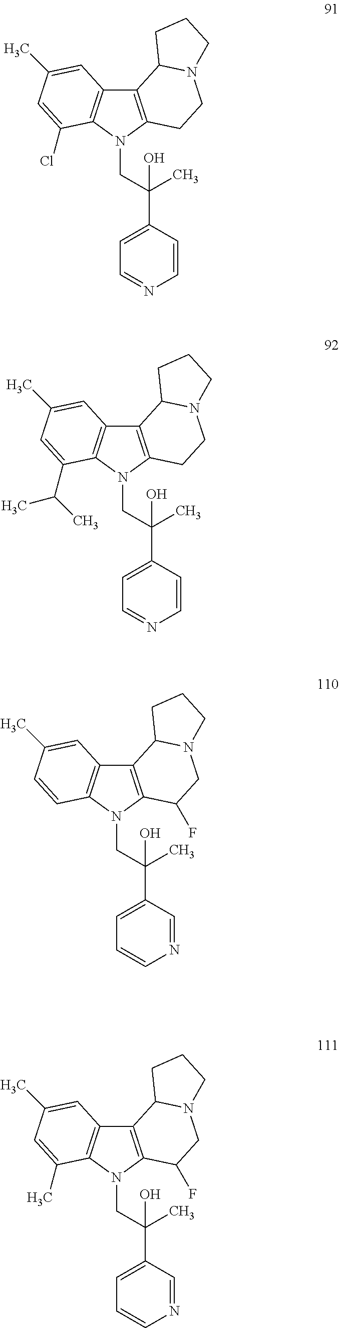 [SMILES: Cc1cc(Cl)c2c(c1)c1c(n2CC(C)(O)c2ccncc2)CCN2CCCC12 ; Cc1cc(C(C)C)c2c(c1)c1c(n2CC(C)(O)c2ccncc2)CCN2CCCC12 ; Cc1ccc2c(c1)c1c(n2CC(C)(O)c2cccnc2)C(F)CN2CCCC12 ; Cc1cc(C)c2c(c1)c1c(n2CC(C)(O)c2cccnc2)C(F)CN2CCCC12]

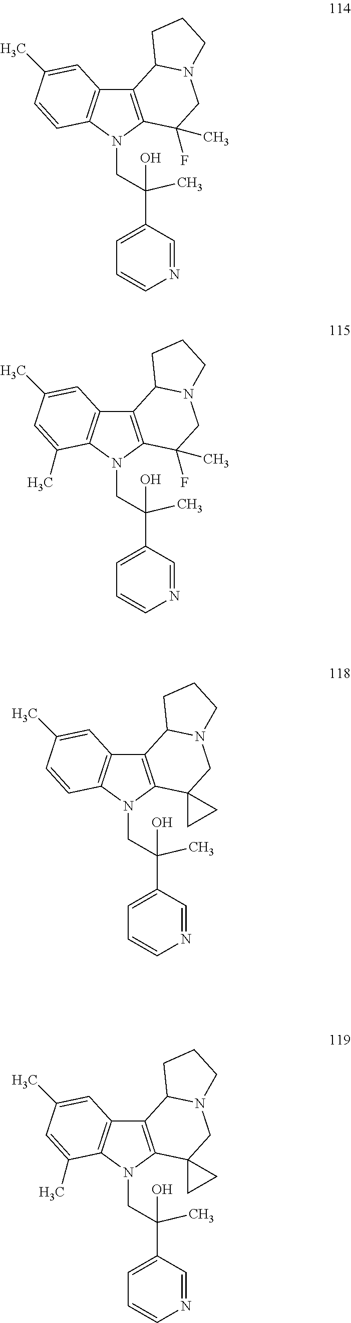

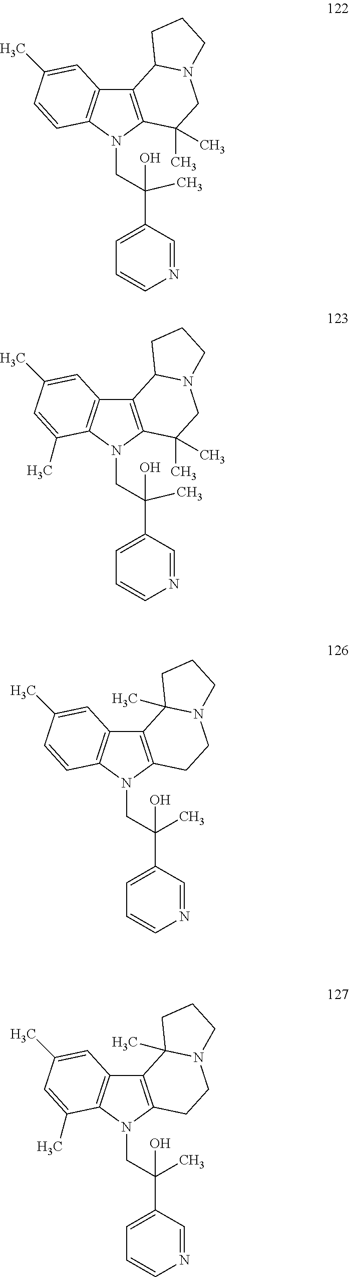

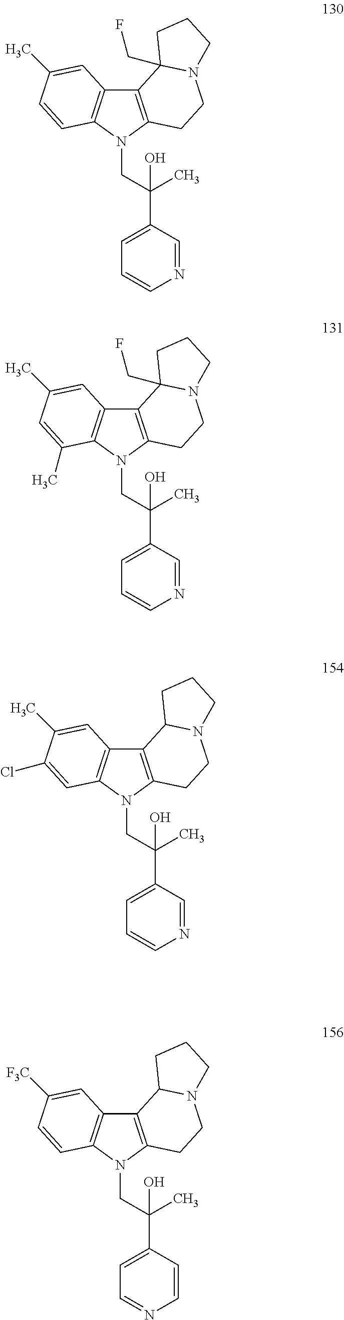 [SMILES: Cc1ccc2c(c1)c1c(n2CC(C)(O)c2cccnc2)CCN2CCCC12CF ; Cc1cc(C)c2c(c1)c1c(n2CC(C)(O)c2cccnc2)CCN2CCCC12CF ; Cc1cc2c3c(n(CC(C)(O)c4cccnc4)c2cc1Cl)CCN1CCCC31 ; CC(O)(Cn1c2c(c3cc(C(F)(F)F)ccc31)C1CCCN1CC2)c1ccncc1]

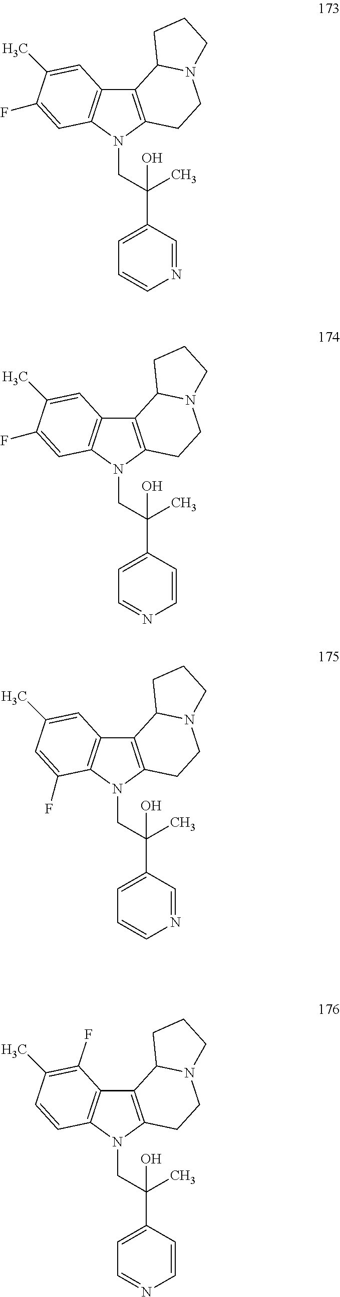 [SMILES: Cc1cc2c3c(n(CC(C)(O)c4cccnc4)c2cc1F)CCN1CCCC31 ; Cc1cc2c3c(n(CC(C)(O)c4ccncc4)c2cc1F)CCN1CCCC31 ; Cc1cc(F)c2c(c1)c1c(n2CC(C)(O)c2cccnc2)CCN2CCCC12 ; Cc1ccc2c(c1F)c1c(n2CC(C)(O)c2ccncc2)CCN2CCCC12]

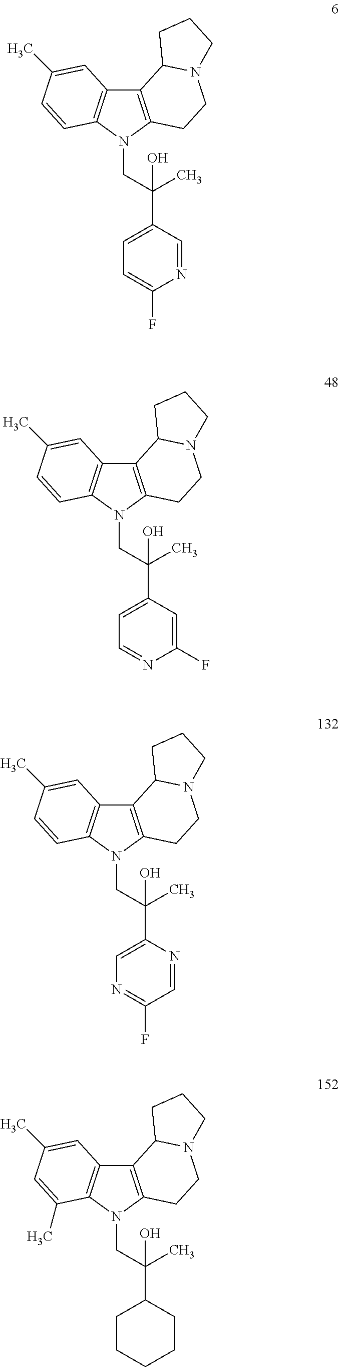 [SMILES: Cc1ccc2c(c1)c1c(n2CC(C)(O)c2ccc(F)nc2)CCN2CCCC12 ; Cc1ccc2c(c1)c1c(n2CC(C)(O)c2ccnc(F)c2)CCN2CCCC12 ; Cc1ccc2c(c1)c1c(n2CC(C)(O)c2cnc(F)cn2)CCN2CCCC12 ; Cc1cc(C)c2c(c1)c1c(n2CC(C)(O)C2CCCCC2)CCN2CCCC12]

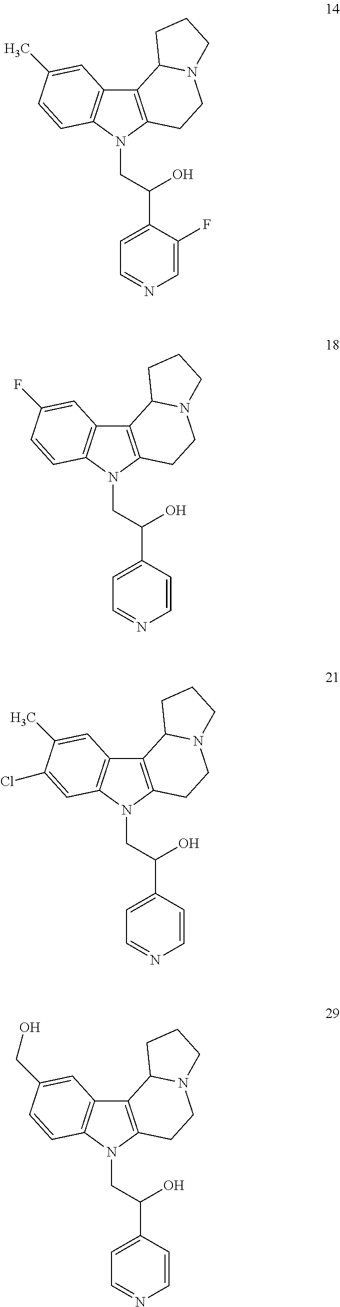 [SMILES: Cc1ccc2c(c1)c1c(n2CC(O)c2ccncc2F)CCN2CCCC12 ; OC(Cn1c2c(c3cc(F)ccc31)C1CCCN1CC2)c1ccncc1 ; Cc1cc2c3c(n(CC(O)c4ccncc4)c2cc1Cl)CCN1CCCC31 ; OCc1ccc2c(c1)c1c(n2CC(O)c2ccncc2)CCN2CCCC12]

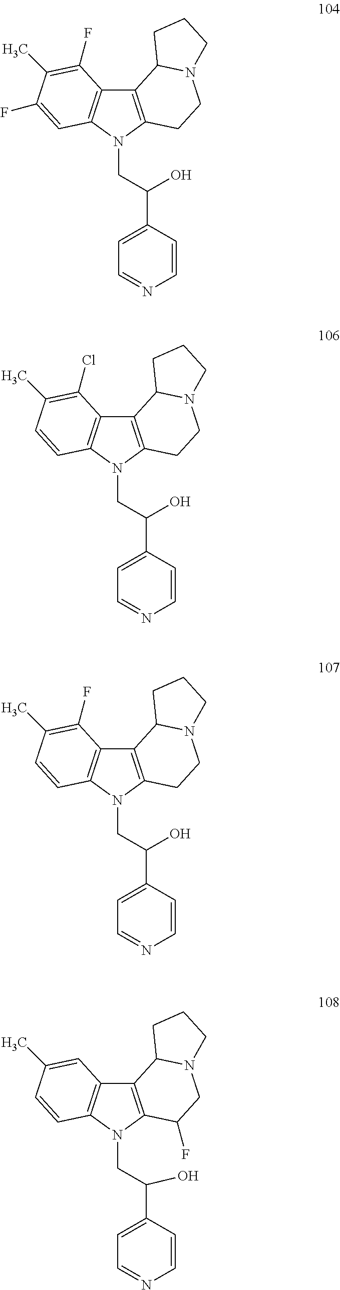 [SMILES: Cc1c(F)cc2c(c1F)c1c(n2CC(O)c2ccncc2)CCN2CCCC12 ; Cc1ccc2c(c1Cl)c1c(n2CC(O)c2ccncc2)CCN2CCCC12 ; Cc1ccc2c(c1F)c1c(n2CC(O)c2ccncc2)CCN2CCCC12 ; Cc1ccc2c(c1)c1c(n2CC(O)c2ccncc2)C(F)CN2CCCC12]

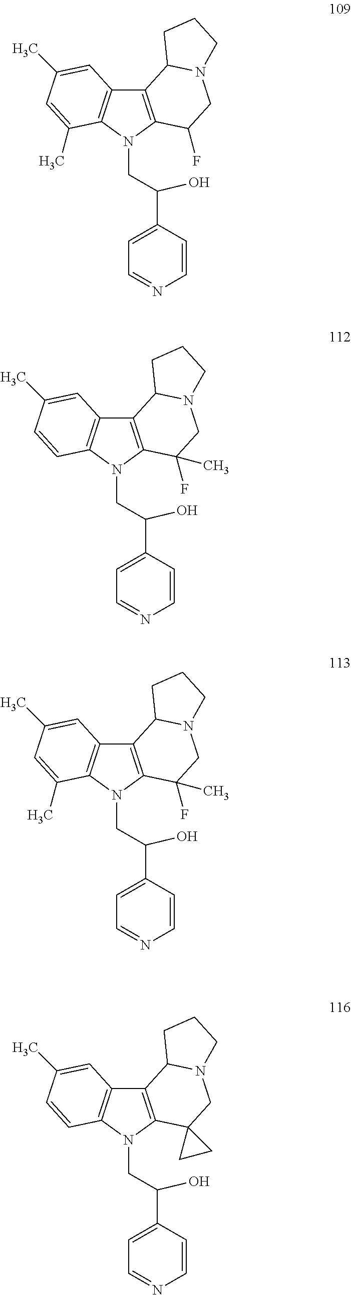

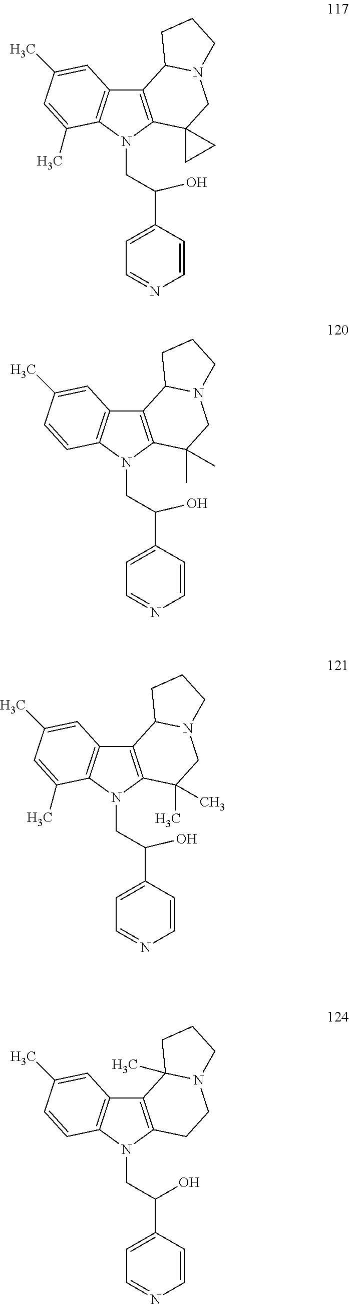

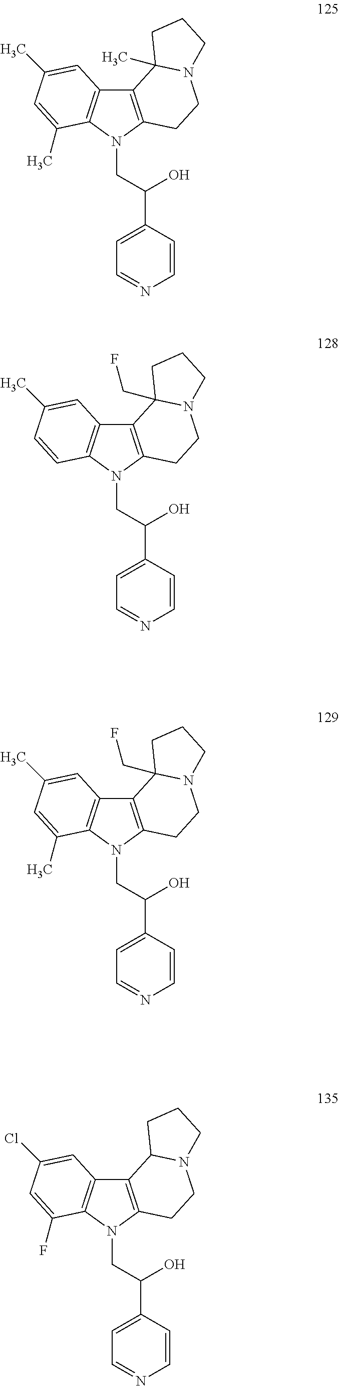 [SMILES: Cc1cc(C)c2c(c1)c1c(n2CC(O)c2ccncc2)CCN2CCCC12C ; Cc1ccc2c(c1)c1c(n2CC(O)c2ccncc2)CCN2CCCC12CF ; Cc1cc(C)c2c(c1)c1c(n2CC(O)c2ccncc2)CCN2CCCC12CF ; OC(Cn1c2c(c3cc(Cl)cc(F)c31)C1CCCN1CC2)c1ccncc1]

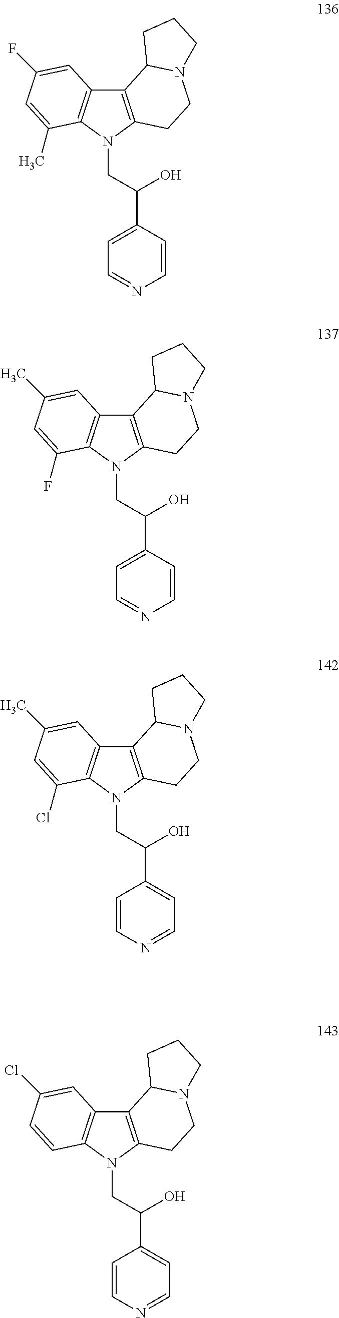 [SMILES: Cc1cc(F)cc2c3c(n(CC(O)c4ccncc4)c12)CCN1CCCC31 ; Cc1cc(F)c2c(c1)c1c(n2CC(O)c2ccncc2)CCN2CCCC12 ; Cc1cc(Cl)c2c(c1)c1c(n2CC(O)c2ccncc2)CCN2CCCC12 ; OC(Cn1c2c(c3cc(Cl)ccc31)C1CCCN1CC2)c1ccncc1]

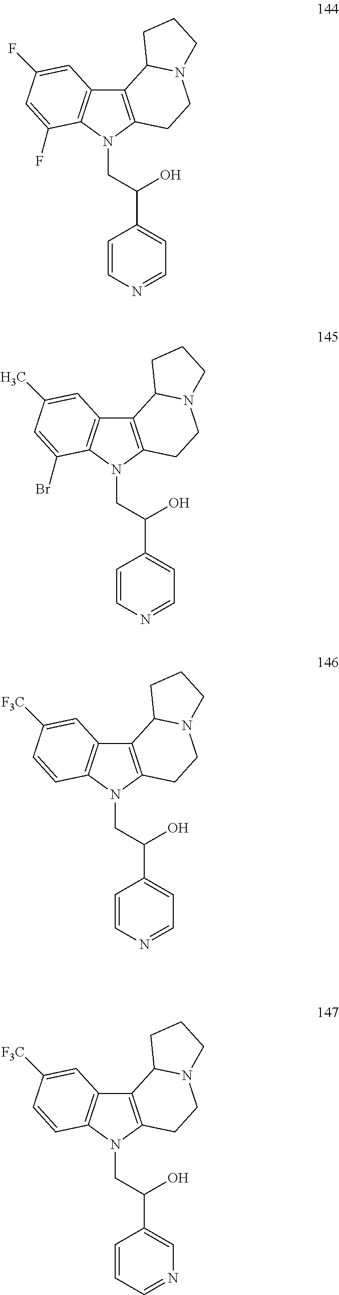 [SMILES: OC(Cn1c2c(c3cc(F)cc(F)c31)C1CCCN1CC2)c1ccncc1 ; Cc1cc(Br)c2c(c1)c1c(n2CC(O)c2ccncc2)CCN2CCCC12 ; OC(Cn1c2c(c3cc(C(F)(F)F)ccc31)C1CCCN1CC2)c1ccncc1 ; OC(Cn1c2c(c3cc(C(F)(F)F)ccc31)C1CCCN1CC2)c1cccnc1]

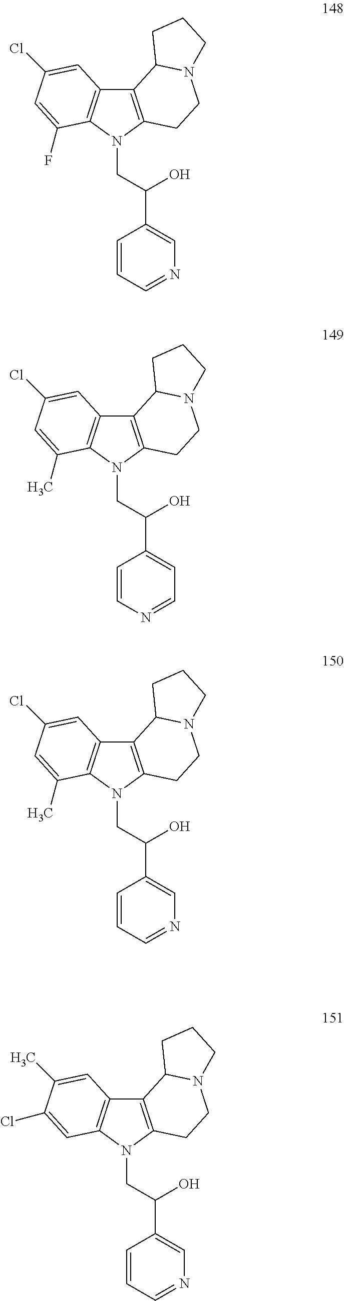 [SMILES: OC(Cn1c2c(c3cc(Cl)cc(F)c31)C1CCCN1CC2)c1cccnc1 ; Cc1cc(Cl)cc2c3c(n(CC(O)c4ccncc4)c12)CCN1CCCC31 ; Cc1cc(Cl)cc2c3c(n(CC(O)c4cccnc4)c12)CCN1CCCC31 ; Cc1cc2c3c(n(CC(O)c4cccnc4)c2cc1Cl)CCN1CCCC31]

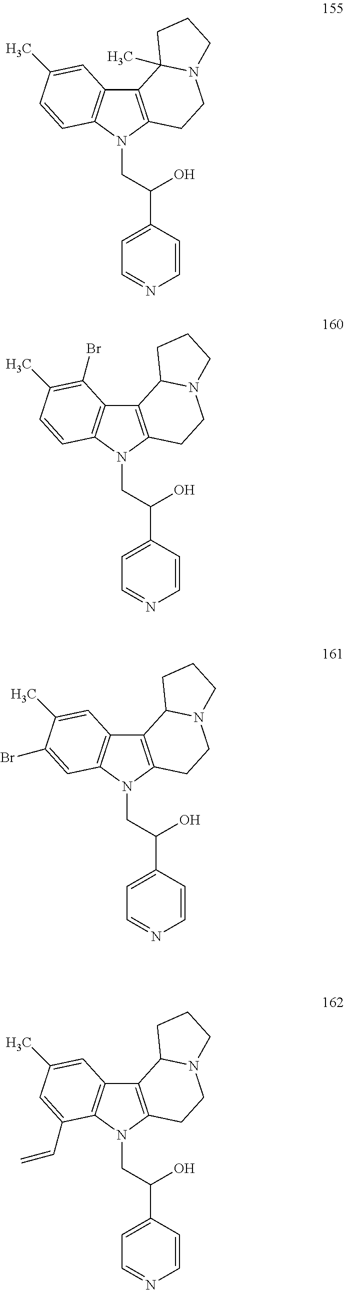 [SMILES: Cc1ccc2c(c1)c1c(n2CC(O)c2ccncc2)CCN2CCCC12C ; Cc1ccc2c(c1Br)c1c(n2CC(O)c2ccncc2)CCN2CCCC12 ; Cc1cc2c3c(n(CC(O)c4ccncc4)c2cc1Br)CCN1CCCC31 ; C=Cc1cc(C)cc2c3c(n(CC(O)c4ccncc4)c12)CCN1CCCC31]

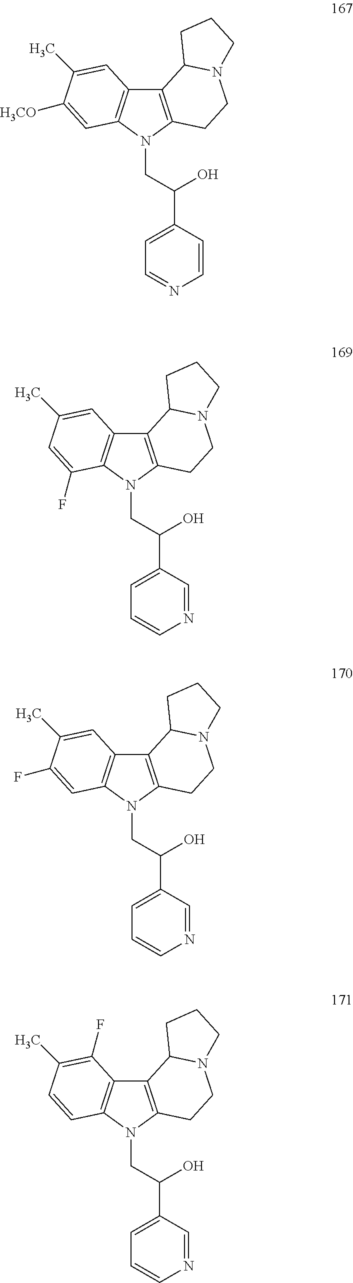 [SMILES: COc1cc2c(cc1C)c1c(n2CC(O)c2ccncc2)CCN2CCCC12 ; Cc1cc(F)c2c(c1)c1c(n2CC(O)c2cccnc2)CCN2CCCC12 ; Cc1cc2c3c(n(CC(O)c4cccnc4)c2cc1F)CCN1CCCC31 ; Cc1ccc2c(c1F)c1c(n2CC(O)c2cccnc2)CCN2CCCC12]

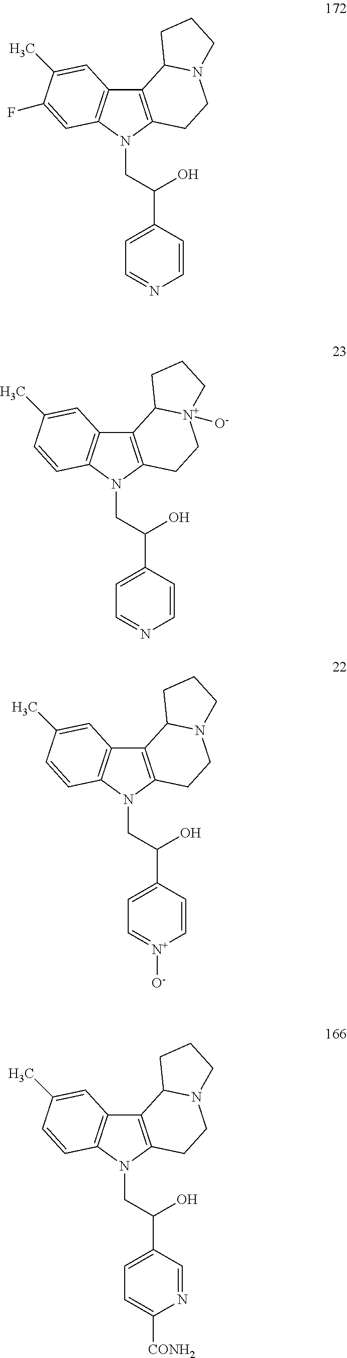 [SMILES: Cc1cc2c3c(n(CC(O)c4ccncc4)c2cc1F)CCN1CCCC31 ; Cc1ccc2c(c1)c1c(n2CC(O)c2ccncc2)CC[N+]2([O-])CCCC12 ; Cc1ccc2c(c1)c1c(n2CC(O)c2cc[n+]([O-])cc2)CCN2CCCC12 ; Cc1ccc2c(c1)c1c(n2CC(O)c2ccc(C(N)=O)nc2)CCN2CCCC12]

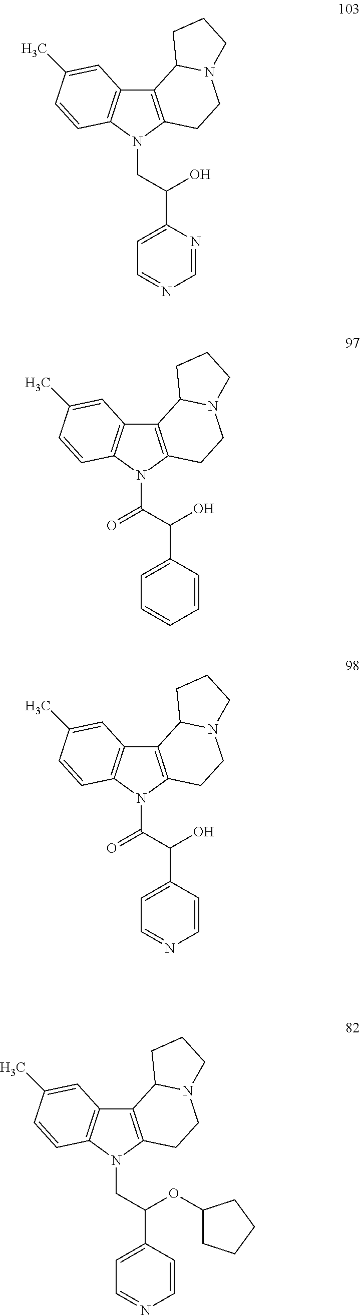 [SMILES: Cc1ccc2c(c1)c1c(n2CC(O)c2ccncn2)CCN2CCCC12 ; Cc1ccc2c(c1)c1c(n2C(=O)C(O)c2ccccc2)CCN2CCCC12 ; Cc1ccc2c(c1)c1c(n2C(=O)C(O)c2ccncc2)CCN2CCCC12 ; Cc1ccc2c(c1)c1c(n2CC(OC2CCCC2)c2ccncc2)CCN2CCCC12]

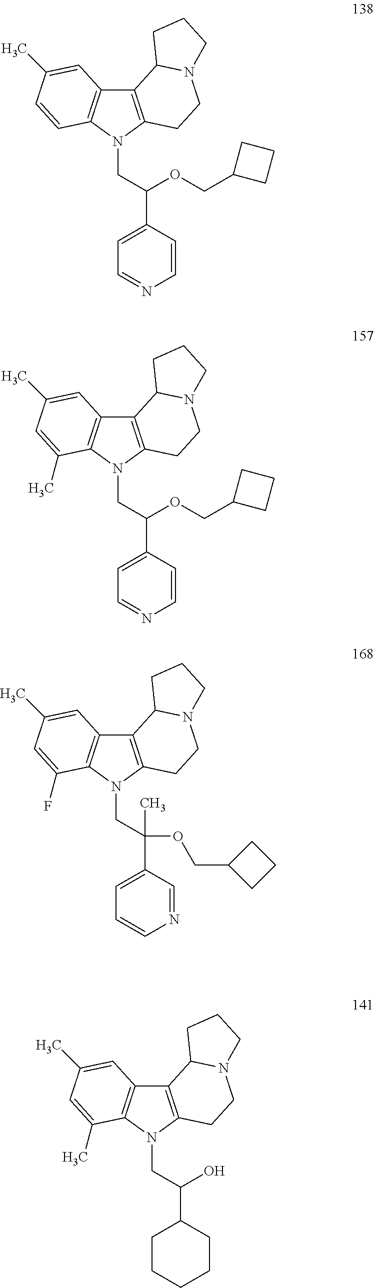 [SMILES: Cc1ccc2c(c1)c1c(n2CC(OCC2CCC2)c2ccncc2)CCN2CCCC12 ; Cc1cc(C)c2c(c1)c1c(n2CC(OCC2CCC2)c2ccncc2)CCN2CCCC12 ; Cc1cc(F)c2c(c1)c1c(n2CC(C)(OCC2CCC2)c2cccnc2)CCN2CCCC12 ; Cc1cc(C)c2c(c1)c1c(n2CC(O)C2CCCCC2)CCN2CCCC12]

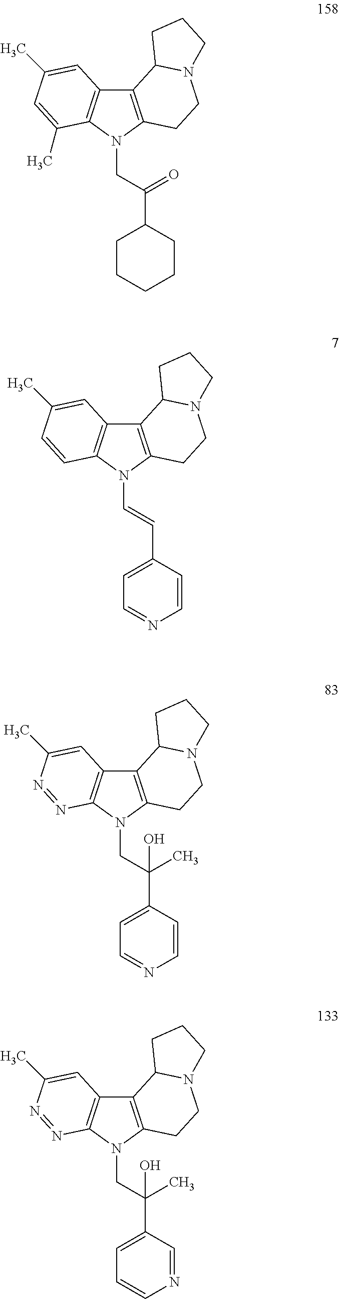 [SMILES: Cc1cc(C)c2c(c1)c1c(n2CC(=O)C2CCCCC2)CCN2CCCC12 ; Cc1ccc2c(c1)c1c(n2/C=C/c2ccncc2)CCN2CCCC12 ; Cc1cc2c3c(n(CC(C)(O)c4ccncc4)c2nn1)CCN1CCCC31 ; Cc1cc2c3c(n(CC(C)(O)c4cccnc4)c2nn1)CCN1CCCC31]

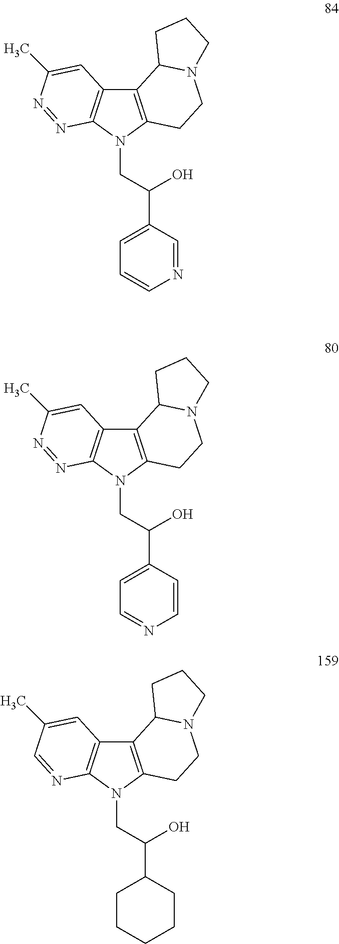 [SMILES: Cc1cc2c3c(n(CC(O)c4cccnc4)c2nn1)CCN1CCCC31 ; Cc1cc2c3c(n(CC(O)c4ccncc4)c2nn1)CCN1CCCC31 ; Cc1cnc2c(c1)c1c(n2CC(O)C2CCCCC2)CCN2CCCC12]

32. The compound of claim 1, or a pharmaceutically acceptable salt thereof, wherein the compound is selected from the group consisting of Compound Nos. 27, 37, 38, 41, 45, 61, 62, 63, 76, 78, 85 and 86:

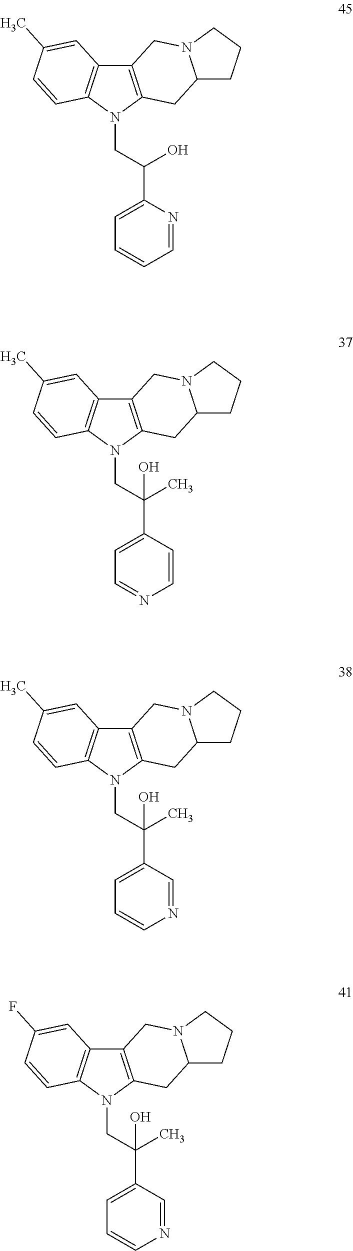 [SMILES: Cc1ccc2c(c1)c1c(n2CC(O)c2ccccn2)CC2CCCN2C1 ; Cc1ccc2c(c1)c1c(n2CC(C)(O)c2ccncc2)CC2CCCN2C1 ; Cc1ccc2c(c1)c1c(n2CC(C)(O)c2cccnc2)CC2CCCN2C1 ; CC(O)(Cn1c2c(c3cc(F)ccc31)CN1CCCC1C2)c1cccnc1]

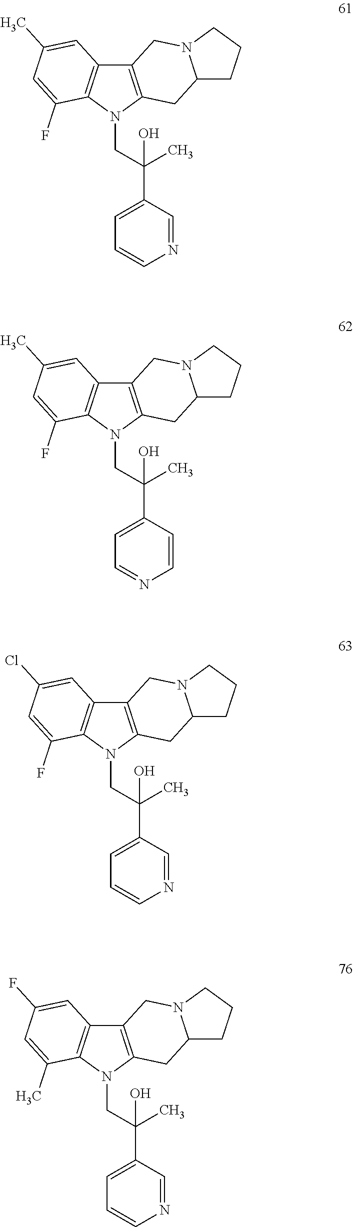 [SMILES: Cc1cc(F)c2c(c1)c1c(n2CC(C)(O)c2cccnc2)CC2CCCN2C1 ; Cc1cc(F)c2c(c1)c1c(n2CC(C)(O)c2ccncc2)CC2CCCN2C1 ; CC(O)(Cn1c2c(c3cc(Cl)cc(F)c31)CN1CCCC1C2)c1cccnc1 ; Cc1cc(F)cc2c3c(n(CC(C)(O)c4cccnc4)c12)CC1CCCN1C3]

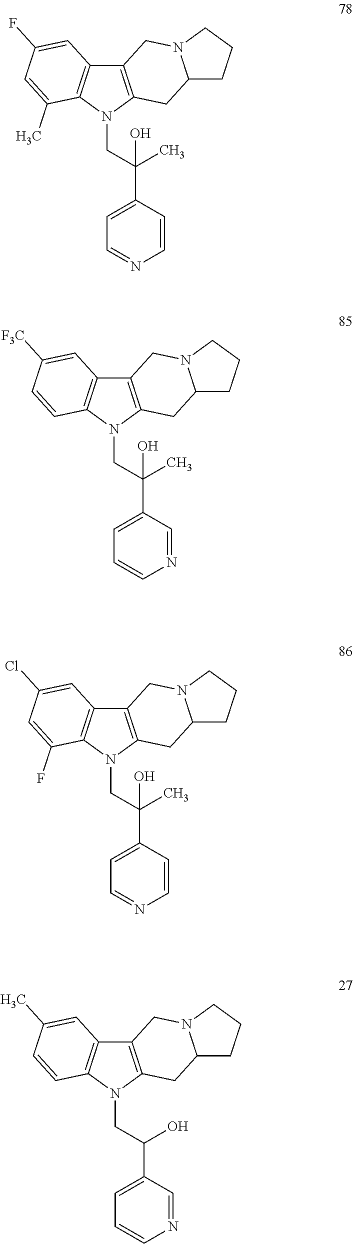 [SMILES: Cc1cc(F)cc2c3c(n(CC(C)(O)c4ccncc4)c12)CC1CCCN1C3 ; CC(O)(Cn1c2c(c3cc(C(F)(F)F)ccc31)CN1CCCC1C2)c1cccnc1 ; CC(O)(Cn1c2c(c3cc(Cl)cc(F)c31)CN1CCCC1C2)c1ccncc1 ; Cc1ccc2c(c1)c1c(n2CC(O)c2cccnc2)CC2CCCN2C1]

33. The compound of claim 1, or a pharmaceutically acceptable salt thereof, wherein the compound is selected from the group consisting of Compound Nos. 34, 36, 73, 74, 101, 102, 134 and 153: 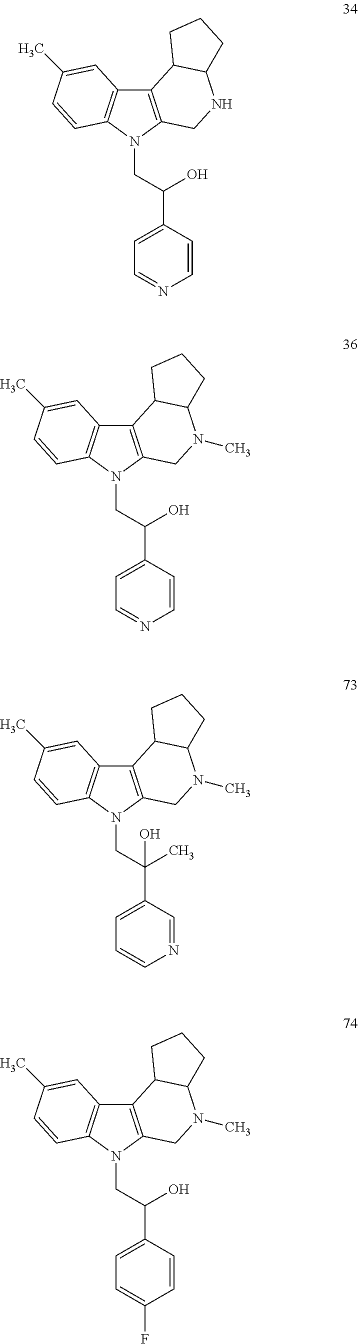 [chemical structure]
